SEQUENCE LISTING (8 sequences)

SEQ ID NO: 1 - RNA, 47 nt, synthetic construct
  misc_feature 1..47: note = 5'-UTR
  source 1..47: mol_type = other RNA
aactagtattcttctggtccccacagactcagagagaacccgccacc

SEQ ID NO: 2 - RNA, 278 nt, synthetic construct
  misc_feature 1..278: note = 3'-UTR
  source 1..278: mol_type = other RNA
ctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 3 - RNA, 110 nt, synthetic construct
  misc_feature 1..110: note = A30L70
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 4 - protein, 26 aa, synthetic construct
  REGION 1..26: note = Secretory sequence
MRVMAPRTLILLLSGALALTETWAGS

SEQ ID NO: 5 - protein, 55 aa, synthetic construct
  REGION 1..55: note = MITD
IVGIVAGLAVLAVVVIGAVVATVMCRRKSSGGKGGSYSQAASSDSAQGSDVSLTA

SEQ ID NO: 6 - protein, 65 aa, synthetic construct
  REGION 1..65: note = P2P16
KKQYIKANSKFIGITELKKLGGGKRGGGKKMTNSVDDALINSTKIYSYFPSVISKVNQGAQGKKL

SEQ ID NO: 7 - protein, 10 aa, synthetic construct
  REGION 1..10: note = GS Linker 1
GGSGGGGSGG

SEQ ID NO: 8 - protein, 14 aa, synthetic construct
  REGION 1..14: note = GS Linker 2
GSSGGGGSPGGGSS

(19) United States
(12) Patent Application Publication
(10) Pub. No.: US 2026/0283970 A1
(43) Pub. Date: Sep. 24, 2026

(54) STABILIZED NUCLEIC ACID COMPOSITIONS AND METHODS FOR PREPARING, STORING AND USING THE SAME

(71) Applicant: BioNTech SE, Mainz (DE)

(72) Inventors: Heinrich HAAS, Mainz (DE); Kaushik THANKI, Mainz (DE); Sukrut SOMANI, Mainz (DE)

(21) Appl. No.: 19/168,038

(22) PCT Filed: Mar. 22, 2024
(86) PCT No.: PCT/EP2024/057732

§ 371 Date: Sep. 23, 2025

(30) Foreign Application Priority Data
Mar. 23, 2023 (WO) PCT/EP2023/057493

(51) Int. Cl. A61K 9/51; A61K 31/7105; A61K 47/02; A61K 47/18; A61K 47/22; A61K 47/26

(52) U.S. Cl. CPC A61K 9/5123; A61K 31/7105; A61K 47/02; A61K 47/183; A61K 47/22; A61K 47/26

(57) ABSTRACT

The present disclosure relates generally to the field of stabilized compositions comprising particles dispersed in an aqueous phase, wherein the aqueous phase comprises a buffer system and has a pH of about 4.0-5.5 and wherein the particles contain (i) nucleic acid (such as DNA or RNA, in particular mRNA or inhibitory RNA, e.g., siRNA); and (ii) a cationic or cationically ionizable lipid, methods for preparing and storing such compositions, and the use of such compositions in therapy.

### TECHNICAL FIELD

The present disclosure relates generally to the field of stabilized compositions comprising particles dispersed in an aqueous phase, wherein the aqueous phase comprises a buffer system and has a pH of about 4.0-5.5 and wherein the particles contain (i) nucleic acid (such as DNA or RNA, in particular mRNA or inhibitory RNA, e.g., siRNA); and (ii) a cationic or cationically ionizable lipid, methods for preparing and storing such compositions, and the use of such compositions in therapy.

### BACKGROUND

The use of a recombinant nucleic acid (such as DNA or RNA) for delivery of foreign genetic information into target cells is well known. A recombinant nucleic acid may be administered in naked form to a subject in need thereof; however, usually a recombinant nucleic acid is administered using a composition. For example, nucleic acid, such as RNA, may be delivered to a subject using different delivery vehicles, based mostly on cationic polymers or lipids which together with the nucleic acid form nanoparticles. The nanoparticles are intended to protect the nucleic acid, such as RNA, from degradation, enable delivery of the nucleic acid, such as RNA, to the target site and facilitate cellular uptake and processing by the target cells. However, it is known that nucleic acid, in particular RNA, in solution or in nanoparticles undergoes slow fragmentation and that lipid-RNA formulations have limited liquid stability causing significant burden on supply chain and storage.

Thus, there remains a need in the art for compositions and methods for introducing nucleic acid, such as RNA, into cells which avoid such disadvantages. Ideally, these compositions and methods should be such that (i) the compositions are stable and can be stored in a temperature range compliant to regular technologies in pharmaceutical practice, in particular in liquid form at temperatures between +2 and +8° C.; (ii) the compositions are ready to use; and/or (iii) the nucleic acid contained in the compositions is in a stable form and is not significantly degraded upon storage. The present disclosure addresses these and other needs.

The inventors surprisingly found that the compositions and methods described herein fulfill the above-mentioned requirements. In particular, it is demonstrated that by adjusting the pH of the compositions to a range of about 4.0-5.5, it is possible to prepare compositions which are stable (in particular with respect to the colloidal size of the particles contained in said compositions and/or with respect to the chemical stability of the nucleic acid), which can be stored in liquid form, which contain nucleic acid that is in a stable form, and which maintain high biological efficacy.

### SUMMARY

In a first aspect, the present disclosure provides a composition comprising particles dispersed in an aqueous phase, wherein the aqueous phase comprises a buffer system and has a pH of about 4.0-5.5 and wherein the particles contain (i) nucleic acid; and (ii) a cationic or cationically ionizable lipid.

As demonstrated in the present application, the degradation of nucleic acid (in particular RNA, such as mRNA) contained in particles (such as lipid nanoparticles (LNPs), liposomes, lipoplexes (LPXs), or mixtures thereof) formed from at least the nucleic acid (in particular RNA, such as mRNA) and a cationic or cationically ionizable lipid (preferably formed from the nucleic acid, a cationic or cationically ionizable lipid, a steroid, and a neutral lipid) can be prevented by adjusting the pH of the composition to a range of about 4.0-5.5. Thus, the claimed composition is stable, can be stored in a temperature range compliant to regular technologies in pharmaceutical practice, provides a ready-to-use composition, and/or maintains high biological efficacy.

In some embodiments of the first aspect, the pH of the aqueous phase is below 5.5 and/or above 4.0. In some embodiments of the first aspect, the pH of the aqueous phase is at least 4.0 and below 5.5, such as at least 4.1 and below 5.5, at least 4.2 and below 5.5, at least 4.3 and below 5.5, at least 4.4 and below 5.5, or at least 4.5 and below 5.5, e.g., at least 5.0 and below 5.5. In some embodiments of the first aspect, the pH of the aqueous phase is at least 4.0 and at most 5.4, such as at least 4.0 and at most 5.3, at least 4.0 and at most 5.2, at least 4.0 and at most 5.1, at least 4.0 and at most 5.0. In some embodiments of the first aspect, the pH of the aqueous phase is at least 4.1 and at most 5.4, at least 4.2 and at most 5.3, at least 4.3 and at most 5.2, at least 4.4 and at most 5.1, or at least 4.5 and at most 5.0. In some embodiments of the first aspect, the pH of the aqueous phase is at least 4.6 and at most 5.4, such as at least 4.7 and at most 5.3, at least 4.8 and at most 5.2, at least 4.9 and at most 5.1, e.g., about 5.0. In some embodiments of the first aspect, the pH of the aqueous phase is from about 4.0 to about 5.2. In some embodiments of the first aspect, the pH of the aqueous phase is from about 4.5 to about 5.2. In some embodiments of the first aspect, the pH of the aqueous phase is from about 4.8 to about 5.2. In some embodiments of the first aspect, the pH of the aqueous phase is from about 5.0 to about 5.2. In some embodiments of the first aspect, the pH of the aqueous phase is between about 4.0 and about 5.0, optionally between about 4.5 and 5.0. In some embodiments of the first aspect, the pH of the aqueous phase is at least 4.5 and below 5.5, optionally at least 4.5 and below 5.2. In some embodiments of the first aspect, the pH of the aqueous phase is at least 5.0 and below 5.5, e.g., at least 5.1 and below 5.5, at least 5.1 and at most 5.4, at least 5.2 and below 5.5, or at least 5.2 and at most 5.4. In some embodiments of the first aspect, the pH of the aqueous phase is about 5.3.

In some embodiments of the first aspect, the concentration of the buffer system in the aqueous phase is between about 1 mM and about 50 mM. In some embodiments of the first aspect, the concentration of the buffer system in the aqueous phase is between about 2 mM and about 40 mM, such as between about 3 mM and about 30 mM, between about 4 mM and about 25 mM, or between about 5 mM and about 20 mM.

In some embodiments of the first aspect, the buffer system comprises or consists essentially of histidine.

In some embodiments of the first aspect, the buffer system comprises or consists essentially of HEPES.

In some embodiments of the first aspect, the buffer system comprises or consists essentially of a combination of HEPES and histidine.

In some embodiments of the first aspect, the aqueous phase further comprises a chelating agent.

Examples of suitable chelating agents include, without limitation, ethylenediaminetetraacetic acid (EDTA), a salt of EDTA, desferrioxamine B, deferoxamine, dithiocarb sodium, penicillamine, pentetate calcium, a sodium salt of pentetic acid, succimer, trientine, nitrilotriacetic acid, trans-diaminocyclohexanetetraacetic acid (DCTA), diethylenetriaminepentaacetic acid (DTPA), and bis(aminoethyl)glycolether-N,N,N′,N′-tetraacetic acid. In certain embodiments, the chelating agent is EDTA or a salt of EDTA. In some embodiments, the concentration of the chelating agent in the aqueous phase is between about 0.1 mM and about 20 mM, such as between about 0.2 mM and about 15 mM, between about 0.3 mM and about 12 mM, between about 0.4 mM and about 11 mM, or between about 0.5 mM and about 10 mM. In some embodiments, the concentration of the chelating agent in the aqueous phase is between about 0.5 mM and about 5 mM, such as between about 0.6 mM and about 4 mM, between about 0.7 mM and about 3.5 mM, between about 0.8 mM and about 3 mM, between about 0.9 mM and about 2.5 mM, or between about 1 mM and about 2 mM. In some embodiments, the concentration of the chelating agent in the aqueous phase is between about 0.5 mM and about 1.5 mM, such as about 1 mM.

In some embodiments of the first aspect, the buffer system comprises histidine and the aqueous phase further comprises a chelating agent, such as EDTA. In some embodiments, the concentration of the chelating agent in the aqueous phase is between about 0.1 mM and about 20 mM, such as between about 0.2 mM and about 15 mM, between about 0.3 mM and about 12 mM, between about 0.4 mM and about 11 mM, or between about 0.5 mM and about 10 mM.

In some embodiments of the first aspect, the buffer system comprises histidine and the aqueous phase is substantially free of EDTA, preferably substantially free of any chelating agent.

In some embodiments of the first aspect, the buffer system comprises HEPES and the aqueous phase further comprises a chelating agent, such as EDTA. In some embodiments, the concentration of the chelating agent in the aqueous phase is between about 0.1 mM and about 20 mM, such as between about 0.2 mM and about 15 mM, between about 0.3 mM and about 12 mM, between about 0.4 mM and about 11 mM, or between about 0.5 mM and about 10 mM.

In some embodiments of the first aspect, the particles have a size of from about 30 nm to about 500 nm.

In some embodiments, the particles have a size from about 50 nm to about 150 nm.

In some embodiments of the first aspect, the particles comprise or are selected from lipid nanoparticles (LNPs), liposomes, lipoplexes (LPXs), and combinations of two or more thereof. In some embodiments, the particles comprise or are LNPs (in particular, when the composition comprises a cationically ionizable lipid). In some embodiments, the particles comprise or are liposomes. In some embodiments, the particles comprise or are LPXs (in particular, when the composition comprises a cationic lipid). In some embodiments, the particles comprise or are mixtures of LNPs and liposomes. In some embodiments, the particles comprise or are mixtures of LNPs and LPXs. In some embodiments, the particles comprise or are mixtures of liposomes and LPXs. In some embodiments, the particles comprise or are mixtures of LNPs, liposomes, and LPXs.

In some embodiments of the first aspect, the particles comprise essentially all of the lipids (in particular all of the cationic/cationically ionizable lipid and, where present, all of the additional lipids (such as steroid and neutral lipid)) present in the composition.

In some embodiments of the first aspect, the aqueous phase is substantially free of the cationic/cationically ionizable lipid and, where present, the additional lipids (such as steroid and neutral lipid), e.g., the aqueous phase is substantially free of lipids.

In some embodiments of the first aspect, the particles comprise at least 50% (such as at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%) of the nucleic acid (in particular RNA) present in the composition. In some embodiments, the particles comprise at least 75%, preferably at least 85% of the nucleic acid (in particular RNA) present in the composition.

In some embodiments of the first aspect, the aqueous phase is substantially free of the nucleic acid.

In some embodiments of the first aspect, the nucleic acid (such as RNA) is encapsulated within and/or associated with the particles.

In some embodiments of the first aspect, water is the main component in the composition and/or the total amount of solvent(s) other than water contained in the composition is less than about 1.0% (v/v), such as less than about 0.5% (v/v). For example, the amount of water contained in the composition may be at least 50% (w/w), such as at least at least 55% (w/w), at least 60% (w/w), at least 65% (w/w), at least 70% (w/w), at least 75% (w/w), at least 80% (w/w), at least 85% (w/w), at least 90% (w/w), or at least 95% (w/w). Additionally, or alternatively, the total amount of solvent(s) other than water contained in the composition may be less than about 0.5% (v/v), such as less than about 0.4% (v/v), less than about 0.3% (v/v), less than about 0.2% (v/v), less than about 0.1% (v/v), less than about 0.05% (v/v), less than about 0.01% (v/v), or less than about 0.005% (v/v). If the composition is substantially free of a cryoprotectant, the amount of water contained in the composition may be at least 95% (w/w). In this respect, a cryoprotectant which is liquid under normal conditions will not be considered as a solvent other than water but as cryoprotectant. In other words, the above optional limitation that the total amount of solvent(s) other than water contained in the composition may be less than about 0.5% (v/v), such as less than about 0.4% (v/v), does not apply to cryoprotectants which are liquids under normal conditions.

In some embodiments of the first aspect, the aqueous phase further comprises one or more tonicity agents. In some embodiments, the one or more tonicity agents are selected from the group consisting of salts and sugars. In some embodiments, the one or more tonicity agents are a salt, such as sodium chloride. In some embodiments, the one or more tonicity agents are a sugar, such as sucrose or glucose.

In some embodiments, the concentration of the one or more tonicity agents in the aqueous phase is such that the composition is at most isotonic, in particular compared to human blood. For example, the compositions may comprise sodium chloride in a concentration of at least up to about 154 mM.

In some embodiments of the first aspect, the concentration of the nucleic acid (in particular RNA) in the composition is about 0.1 mg/l to about 500 mg/l. In some embodiments, the concentration of the nucleic acid (in particular RNA) in the composition is about 0.5 mg/l to about 400 mg/l. In some embodiments, the concentration of the nucleic acid (in particular RNA) in the composition is about 1 mg/l to about 300 mg/l, such as about 2 mg/l to about 200 mg/l, about 3 mg/l to about 150 mg/l, or about 5 mg/l to about 100 mg/l.

In some embodiments of the first aspect, the N/P ratio is at least about 2. In some embodiments, the N/P ratio is between about 2 and about 12, such as between about 4 and about 10, between about 4 and about 8, or between about 5 and about 7. In some embodiments, the N/P ratio is about 6.

In some embodiments of the first aspect, the composition is substantially free of a cryoprotectant.

In some embodiments of the first aspect, the pH of the aqueous phase is at least about 4.0 and below about 5.5 (such as between about 4.0 and about 5.0, optionally between about 4.5 and 5.0, or the pH is at least 5.0 and below 5.5, e.g., at least 5.1 and below 5.5, at least 5.1 and at most 5.4, at least 5.2 and below 5.5, or at least 5.2 and at most 5.4, or the pH of the aqueous phase is about 5.3); and the aqueous phase comprises histidine and optionally a chelating agent, preferably each in the concentration given above or below.

In some embodiments of the first aspect, the pH of the aqueous phase is at least about 4.0 and below about 5.5 (such as between about 4.0 and about 5.0, optionally between about 4.5 and 5.0, or the pH is at least 5.0 and below 5.5, e.g., at least 5.1 and below 5.5, at least 5.1 and at most 5.4, at least 5.2 and below 5.5, or at least 5.2 and at most 5.4, or the pH of the aqueous phase is about 5.3); and the aqueous phase comprises HEPES and optionally a chelating agent, preferably each in the concentration given above or below.

In some embodiments of the first aspect, the cationically ionizable lipid comprises a head group which includes at least one tertiary amine moiety which preferably is capable of being protonated under physiological conditions.

In some embodiments of the first aspect, the cationically ionizable lipid has the structure of Formula (X)

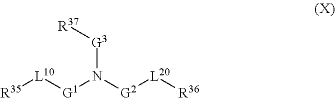

or a pharmaceutically acceptable salt, tautomer, or stereoisomer thereof, wherein L¹⁰, L²⁰ G¹, G² G³R³⁵, R³⁶, and R³⁷ are as defined herein. In some embodiments, the cationically ionizable lipid is selected from the following: the structures X-1 to X-36 (shown herein); the structures A to G (shown herein); or N,N-dimethyl-2,3-dioleyloxypropylamine (DODMA), 1,2-dioleoyl-3-dimethylammonium-propane (DODAP), heptatriaconta-6,9,28,31-tetraen-19-yl-4-(dimethylamino)butanoate (DLin-MC3-DMA), and 4-((di((9Z,12Z)-octadeca-9,12-dien-1-yl)amino)oxy)-N,N-dimethyl-4-oxobutan-1-amine (DPL-14). In some embodiments, the cationically ionizable lipid is the lipid having the structure X-3. In some embodiments, the cationically ionizable lipid is DPL-14 (i.e., the lipid having the structure G).

In some embodiments of the first aspect, the cationically ionizable lipid has the structure of Formula (XI):

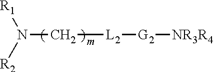

wherein R₁, R₂ R₃, R₄, L₂, G₂, and m are as defined herein. In some embodiments, the cationically ionizable lipid is selected from the structures (XIV-1), (XIV-2), and (XIV-3) (shown herein). In some embodiments, the cationically ionizable lipid is the lipid having the structure XIV-1 In some embodiments, the cationically ionizable lipid is the lipid having the structure XIV-2. In some embodiments, the cationically ionizable lipid is the lipid having the structure XIV-3.

In some embodiments of the first aspect, the cationically ionizable lipid has the structure of Formula (TL-I):

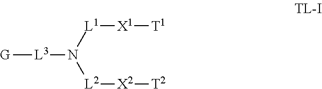

or a pharmaceutically acceptable salt thereof, wherein G¹, L¹, L², L³, X¹, X², T¹, and T² are as defined herein. In some embodiments, the cationically ionizable lipid is selected from the structures BNT-51, BNT-52, BNT-76, and pharmaceutically acceptable salts thereof.

In some embodiments of the first aspect, the cationically ionizable lipid has the structure of Formula (AXL-I):

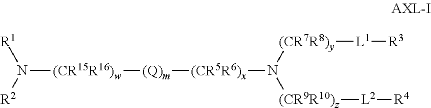

or a pharmaceutically acceptable salt thereof, wherein R¹, R², R₃, R⁴, R⁵, R⁶, R⁷, R⁸, R⁹, R¹⁰, R¹¹, R¹⁶, Q w, m, x, y, z, L¹, and L² are as defined herein. In some embodiments, the cationically ionizable lipid is selected from the structures BHD-C2C2-PipZ, BODD-C2C2-1Me-Pyr, BODD-C2C2-Pyr, BODD-C2C1-1Me-3PipD, BODD-C2C1-1Me-PipD, BODD-C2C2-DMA, BODD-C2C4-PipZ, BODD-C2C4-Pyr, BHD-C2C4-PipZ, and pharmaceutically acceptable salts or mixtures thereof.

In some embodiments of the first aspect, the cationically ionizable lipid is completely or partially replaced by a cationic lipid. In some embodiments, the cationic lipid is selected from the structures XV-1 to XV-6 (shown herein).

In some embodiments of the first aspect, the cationic or cationically ionizable lipid comprises 2,3-dioleyloxy-1-(N,N-dimethylamino)propane (DODMA), N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)-[1,3]-dioxolane (DLin-KC2-DMA), 2,2-dilinoleyl-4-dimethylaminomethyl-[1,3]-dioxolane (DLin-K-DMA), DPL14, or a mixture thereof.

In some embodiments of the first aspect, the cationic or cationically ionizable lipid is selected from the group consisting of [(4-hydroxybutyl)azanediyl]di(hexane-6,1-diyl)bis(2-hexyldecanoate) (ALC-315); 1,2-dioleoyloxy-3-dimethylaminopropane (DODMA); 2,2-dilinoleyl-4-dimethylaminoethyl-[1,3]-dioxolane (DLin-KC2-DMA); heptatriaconta-6,9,28,31-tetraen-19-yl-4-(dimethylamino)butanoate (D-Lin-MC3-DMA); 1,2-dilinoleyloxy-N,N-dimethylaminopropane (DLin-DMA); di((Z)-non-2-en-1-yl)-9-((4-(dimethylaminobutanoyl)oxy)heptadecanedioate (L319); bis-(2-butyloctyl) 10-(N-(3-(dimethylamino)propyl)nonanamido)-nonadecanedioate (A9); (heptadecan-9-yl 8-{(2-hydroxyethyl)[6-oxo-6-(undecyloxy)octyl]amino}-octanoate) (L5); heptadecan-9-yl 8-{(2-hydroxyethyl)[6-oxo-6-(undecyloxy)hexyl]amino}-octanoate) (SM-102); O-[N-{(9Z,12Z)-octadeca-9,12-dien-1-yl)}-N-{7-pentadecylcarbonyloxyoctyl}-amino]4-(dimethylamino)butanoate (HY501); 2-(di-((9Z,12Z)-octadeca-9,12-dien-1-yl)amino)ethyl 4-(dimethylamino)butanoate (EA-2); 4-((di-((9Z,12Z)-octadeca-9,12-dien-1-yl)amino)oxy)-N,N-dimethyl-4-oxobutan-4-amine (HYAM-2); ((2-(4-(dimethylamino)butanoyl)oxy)ethyl)azanediylbis(octane 8,1-diyl)bis(2-hexyldecanoate) (EA-405); (2-(4-(dimethylamino)butanoyl)oxy)azanediylbis(octane 8,1-diyl)bis(2-hexyldecanoate) (HY-405); palmitoyl-oleoyl-nor-arginine (PONA); guanidino-di[(heptadecyl)methyl]carboxylic acid (GUADACA); 4-methylpyridinium-di(heptadecyl)methylcarboxylic acid (MPDACA); 1,2-dioleoyl-3 trimethylammonium propane (DOTAP); 1,2-dioleoyl-3-dimethylammomium propane (DODAP); 1,2-di-O-octadecenyl-3-trimethylammonium propane (DOTMA); and a mixture of any thereof.

In some embodiments of the first aspect, the cationically ionizable lipid is selected from the group consisting of 7,7′-((4-hydroxybutyl)azanediyl)bis(N-hexyl-N-octylheptane-1-sulfonamide) (BNT-51); 7,7′-((4-(3,3-dimethylthioureido)butyl)azanediyl)bis(N-hexyl-N-octylheptane-1-sulfonamide) (BNT-52); BNT-76; [(4-hydroxybutyl)azanediyl]di(hexane-6,1-diyl)bis(2-hexyldecanoate) (ALC-315); 1,2-dioleoyloxy-3-dimethylaminopropane (DODMA); 2,2-dilinoleyl-4-dimethylaminoethyl-[1,3]-dioxolane (DLin-KC2-DMA); heptatriaconta-6,9,28,31-tetraen-19-yl-4-(dimethylamino)butanoate (D-Lin-MC3-DMA); 1,2-dilinoleyloxy-N,N-dimethylaminopropane (DLin-DMA); di((Z)-non-2-en-1-yl)-9-((4-(dimethylaminobutanoyl)oxy)heptadecanedioate (L319); bis-(2-butyloctyl) 10-(N-(3-(dimethylamino)propyl)nonanamido)-nonadecanedioate (A9); (heptadecan-9-yl 8-{(2-hydroxyethyl)[6-oxo-6-(undecyloxy)octyl]amino}-octanoate) (L5); heptadecan-9-yl 8-{(2-hydroxyethyl)[6-oxo-6-(undecyloxy)hexyl]amino}-octanoate) (SM-102); O-[N-{(9Z,12Z)-octadeca-9,12-dien-1-yl)}-N-{7-pentadecylcarbonyloxyoctyl}-amino]4-(dimethylamino)butanoate (HY501); 2-(di-((9Z,12Z)-octadeca-9,12-dien-1-yl)amino)ethyl 4-(dimethylamino)butanoate (EA-2); 4-((di-((9Z,12Z)-octadeca-9,12-dien-1-yl)amino)oxy)-N,N-dimethyl-4-oxobutan-4-amine (HYAM-2); ((2-(4-(dimethylamino)butanoyl)oxy)ethyl)azanediylbis(octane 8,1-diyl)bis(2-hexyldecanoate) (EA-405); (2-(4-(dimethylamino)butanoyl)oxy)azanediylbis(octane 8,1-diyl)bis(2-hexyldecanoate) (HY-405); di(heptadecan-9-yl) 3,3′-((2-(4-methylpiperazin-1-yl)ethyl)azanediyl)dipropionate (BHD-C2C2-PipZ); bis(2-octyldodecyl) 3,3′-((2-(1-methylpyrrolidin-2-yl)ethyl)azanediyl)dipropionate (BODD-C2C2-1Me-Pyr); bis(2-octyldodecyl) 3,3′-((2-(pyrrolidin-1-yl)ethyl)azanediyl)dipropionate (BODD-C2C2-Pyr); bis(2-octyldodecyl) 3,3′-(((1-methylpiperidin-3-yl)methyl)azanediyl)dipropionate (BODD-C2C1-1Me-3PipD); bis(2-octyldodecyl) 3,3′-(((1-methylpiperidin-4-yl)methyl)azanediyl)dipropionate (BODD-C2C1-1Me-PipD); bis(2-octyldodecyl) 3,3′-((2-(dimethylamino)ethyl)azanediyl)dipropionate (BODD-C2C2-DMA); bis(2-octyldodecyl) 3,3′-((4-(4-methylpiperazin-1-yl)butyl)azanediyl)dipropionate (BODD-C2C4-PipZ); bis(2-octyldodecyl) 3,3′-((4-(pyrrolidin-1-yl)butyl)azanediyl)dipropionate (BODD-C2C4-Pyr); bis(2-hexyldecyl) 3,3′-((4-(4-methylpiperazin-1-yl)butyl)azanediyl)dipropionate (BHD-C2C4-PipZ); and a mixture of any thereof.

In some embodiments of the first aspect, the cationically ionizable lipid is selected from the group consisting of 7,7′-((4-hydroxybutyl)azanediyl)-bis(N-hexyl-N-octylheptane-1-sulfonamide) (BNT-51); 7,7′-((4-(3,3-dimethylthioureido)butyl)azanediyl)bis(N-hexyl-N-octylheptane-1-sulfonamide) (BNT-52); BNT-76; [(4-hydroxybutyl)azanediyl]di(hexane-6,1-diyl)bis(2-hexyldecanoate) (ALC-315); 1,2-dioleoyloxy-3-dimethylaminopropane (DODMA); 2,2-dilinoleyl-4-dimethylaminoethyl-[1,3]-dioxolane (DLin-KC2-DMA); heptatriaconta-6,9,28,31-tetraen-19-yl-4-(dimethylamino)butanoate (D-Lin-MC3-DMA); 1,2-dilinoleyloxy-N,N-dimethylaminopropane (DLin-DMA); di((Z)-non-2-en-1-yl)-9-((4-(dimethylaminobutanoyl)oxy)heptadecanedioate (L319); bis-(2-butyloctyl) 10-(N-(3-(dimethylamino)propyl)nonanamido)-nonadecanedioate (A9); (heptadecan-9-yl 8-{(2-hydroxyethyl)[6-oxo-6-(undecyloxy)octyl]amino}-octanoate) (L5); heptadecan-9-yl 8-{(2-hydroxyethyl)[6-oxo-6-(undecyloxy)hexyl]amino}-octanoate) (SM-102); O-[N-{(9Z,12Z)-octadeca-9,12-dien-1-yl)}-N-{7-pentadecylcarbonyloxyoctyl}-amino]4-(dimethylamino)butanoate (HY501); ((2-(4-(dimethylamino)butanoyl)oxy)ethyl)azanediylbis(octane 8,1-diyl)bis(2-hexyldecanoate) (EA-405); (2-(4-(dimethylamino)butanoyl)oxy)azanediylbis(octane 8,1-diyl)bis(2-hexyldecanoate) (HY-405); di(heptadecan-9-yl) 3,3′-((2-(4-methylpiperazin-1-yl)ethyl)azanediyl)dipropionate (BHD-C2C2-PipZ); bis(2-octyldodecyl) 3,3′-((2-(1-methylpyrrolidin-2-yl)ethyl)azanediyl)dipropionate (BODD-C2C2-1Me-Pyr); bis(2-octyldodecyl) 3,3′-((2-(pyrrolidin-1-yl)ethyl)azanediyl)dipropionate (BODD-C2C2-Pyr); bis(2-octyldodecyl) 3,3′-(((1-methylpiperidin-3-yl)methyl)azanediyl)dipropionate (BODD-C2C1-1Me-3PipD); bis(2-octyldodecyl) 3,3′-(((1-methylpiperidin-4-yl)methyl)azanediyl)dipropionate (BODD-C2C1-1Me-PipD); bis(2-octyldodecyl) 3,3′-((2-(dimethylamino)ethyl)azanediyl)dipropionate (BODD-C2C2-DMA); bis(2-octyldodecyl) 3,3′-((4-(4-methylpiperazin-1-yl)butyl)azanediyl)dipropionate (BODD-C2C4-PipZ); bis(2-octyldodecyl) 3,3′-((4-(pyrrolidin-1-yl)butyl)azanediyl)dipropionate (BODD-C2C4-Pyr); bis(2-hexyldecyl) 3,3′-((4-(4-methylpiperazin-1-yl)butyl)azanediyl)dipropionate (BHD-C2C4-PipZ); and a mixture of any thereof.

In some embodiments of the first aspect, the cationically ionizable lipid comprises from about 20 mol % to about 80 mol %, such as from about 25 mol % to about 65 mol %, from about 30 mol % to about 50 mol %, or from about 40 mol % to about 50 mol %, of the total lipid present in the composition. In those embodiments, where the cationically ionizable lipid is partially or completely replaced by a cationic lipid, it is preferred that the same ranges as specified above for the cationically ionizable lipid (e.g., from about 20 mol % to about 80 mol %, etc.) apply to the sum of cationically ionizable lipid and cationic lipid.

In some embodiments of the first aspect, the particles contain a cationically ionizable lipid (in particular, when the composition comprises lipid nanoparticles (LNPs)).

In some embodiments of the first aspect, the particles contain a cationic lipid (in particular, when the composition comprises lipoplexes (LPXs)).

In some embodiments of the first aspect, the particles further comprise one or more additional lipids. In some embodiments, the one or more additional lipids are selected from the group consisting of polymer-conjugated lipids, neutral lipids, steroids, and combinations thereof.

In some embodiments of the first aspect, the polymer-conjugated lipid comprises a pegylated lipid. In some embodiments, the pegylated lipid is selected from the group consisting of DSPE-PEG, DOPE-PEG, DPPE-PEG, and DMPE-PEG. In some embodiments, the pegylated lipid has the following structure:

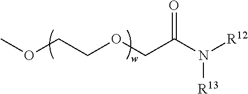

or a pharmaceutically acceptable salt, tautomer or stereoisomer thereof, wherein R¹², R¹³, and w are as defined herein.

In some embodiments of the first aspect, the polymer-conjugated lipid comprises a polysarcosine-lipid conjugate or a conjugate of polysarcosine and a lipid-like material. In some embodiments, the polysarcosine-lipid conjugate or conjugate of polysarcosine and a lipid-like material is a member selected from the group consisting of a polysarcosine-diacylglycerol conjugate, a polysarcosine-dialkyloxypropyl conjugate, a polysarcosine-phospholipid conjugate, a polysarcosine-ceramide conjugate, and a mixture thereof.

In some embodiments of the first aspect, the polymer-conjugated lipid comprises from about 0.5 mol % to about 5 mol % of the total lipid present in the composition. In some embodiments, the polymer-conjugated lipid comprises from about 1 mol % to about 5 mol %, such as from about 1 mol % to about 4.5 mol %, of the total lipid present in the composition.

In some embodiments of the first aspect, the neutral lipid is a phospholipid. In some embodiments, the phospholipid is selected from the group consisting of phosphatidylcholines, phosphatidylethanolamines, phosphatidylglycerols, phosphatidic acids, phosphatidylserines and sphingomyelins. In some embodiments, the phospholipid is selected from the group consisting of distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), and diphytanoyl-phosphatidylethanolamine (DPyPE). In some embodiments, the neutral lipid is selected from the group consisting of DSPC, DOPC, and DOPE. In some embodiments, the neutral lipid is DSPC or DOPC. In some embodiments, the neutral lipid is DSPC. In some embodiments, the neutral lipid is DOPC. In some embodiments, the neutral lipid is DOPE.

In some embodiments of the first aspect, the neutral lipid comprises from about 5 mol % to about 40 mol %, such as from about 5 mol % to about 20 mol % or from about 5 mol % to about 15 mol %, of the total lipid present in the composition.

In some embodiments of the first aspect, the steroid comprises a sterol. In some preferred embodiments of the first aspect, the steroid comprises or is cholesterol.

In some embodiments of the first aspect, the steroid comprises from about 10 mol % to about 65 mol %, such as from about 20 mol % to about 60 mol %, or from about 30 mol % to about 50 mol %, of the total lipid present in the composition.

In some embodiments of the first aspect, the particles comprise the cationic or cationically ionizable lipid, a neutral lipid (e.g., a phospholipid), and a steroid, and optionally a polymer-conjugated lipid. In some embodiments, the cationic or cationically ionizable lipid comprises from about 30 mol % to about 50 mol %, such as from about 40 mol % to about 50 mol %, of the total lipid present in the composition; the neutral lipid (e.g., phospholipid) comprises from about 5 mol % to about 15 mol % of the total lipid present in the composition; and the steroid comprises from about 30 mol % to about 50 mol % of the total lipid present in the composition; and, if present, the polymer-conjugated lipid comprises from about 1 mol % to about 4.5 mol % of the total lipid present in the composition.

In some embodiments of the first aspect, the composition comprises a cationic lipid and a cationically ionizable lipid. In these embodiments, the sum of (1) the amount the cationically ionizable lipid and (2) the amount of cationic lipid is used for calculations. E.g., if the amount of cationically ionizable lipid in a composition should be from about 20 mol % to about 80 mol % and the composition should also contain a cationic lipid, then the sum of (1) the amount the cationically ionizable lipid and (2) the amount of cationic lipid is to be from about 20 mol % to about 80 mol %.

In some embodiments of the first aspect, the only lipids contained in the composition are the cationically ionizable lipid, the steroid and the neutral lipid and optionally a polymer-conjugated lipid, in particular the cationically ionizable lipid, the steroid and the phospholipid and optionally a polymer-conjugated lipid.

In some embodiments of the first aspect, the composition comprises a cationically ionizable lipid, a neutral lipid (e.g., a phospholipid), a steroid, histidine, and optionally (a) a polymer-conjugated lipid, and/or (b) a chelating agent (such as EDTA), wherein the pH of the aqueous phase is at least 4.0 and below 5.5 (such as between 4.0 and 5.0, or the pH is at least 5.0 and below 5.5, e.g., at least 5.1 and below 5.5, at least 5.1 and at most 5.4, at least 5.2 and below 5.5, or at least 5.2 and at most 5.4, or the pH of the aqueous phase is about 5.3). In some embodiments, these compositions (1) neither further comprise a polymer-conjugated lipid nor a chelating agent; (2) comprise a chelating agent; (3) a polymer-conjugated lipid; or (4) a chelating agent and a polymer-conjugated lipid. In some embodiments of these compositions, the neutral lipid is selected from the group consisting of DSPC, DOPC, and DOPE (preferably the neutral lipid is DOPE) and/or the steroid is cholesterol.

In some embodiments of the first aspect, the composition comprises a cationically ionizable lipid, a neutral lipid (e.g., a phospholipid), a steroid, HEPES, and optionally (a) a polymer-conjugated lipid, and/or (b) a chelating agent (such as EDTA), wherein the pH of the aqueous phase is at least 4.0 and below 5.5 (such as between 4.0 and 5.0, or the pH is at least 5.0 and below 5.5, e.g., at least 5.1 and below 5.5, at least 5.1 and at most 5.4, at least 5.2 and below 5.5, or at least 5.2 and at most 5.4, or the pH of the aqueous phase is about 5.3). In some embodiments, these compositions (1) neither further comprise a polymer-conjugated lipid nor a chelating agent; (2) comprise a chelating agent; (3) a polymer-conjugated lipid; or (4) a chelating agent and a polymer-conjugated lipid. In some embodiments of these compositions, the neutral lipid is selected from the group consisting of DSPC, DOPC, and DOPE (preferably the neutral lipid is DOPE) and/or the steroid is cholesterol.

In some embodiments of the first aspect, the nucleic acid is DNA.

In some embodiments of the first aspect, the nucleic acid is RNA, preferably mRNA, inhibitory RNA (e.g. siRNA), or self-replicating RNA.

In some embodiments of the first aspect, the nucleic acid is RNA (such as mRNA) and (i) comprises a modified nucleoside in place of uridine; (ii) has a coding sequence which is codon-optimized; and/or (iii) has a coding sequence whose G/C content is increased compared to the wild-type coding sequence.

In some embodiments, the modified nucleoside is selected from pseudouridine (ψ), N1-methyl-pseudouridine (mlψ), and 5-methyl-uridine (m5U).

In some embodiments of the first aspect, the nucleic acid is RNA (such as mRNA) and comprises at least one or more of the following: a 5′ cap; a 5′ UTR; a 3′ UTR; and a poly-A sequence. In some embodiments, the RNA (such as mRNA) comprises all of the following: a 5′ cap; a 5′ UTR; a 3′ UTR; and a poly-A sequence. In some embodiments, the poly-A sequence comprises at least 100 A nucleotides, wherein the poly-A sequence preferably is an interrupted sequence of A nucleotides. In some embodiments, the 5′ cap is a cap1 or cap2 structure.

In some embodiments of the first aspect, the nucleic acid is RNA (such as mRNA) and encodes one or more polypeptides. In some embodiments, the one or more polypeptides are pharmaceutically active peptides or polypeptides and/or comprise an epitope for inducing an immune response against an antigen in a subject.

In some embodiments of the first aspect, the pharmaceutically active polypeptide and/or the antigen or epitope is derived from or is a protein of a pathogen, an immunogenic variant of the protein, or an immunogenic fragment of the protein or the immunogenic variant thereof. In some embodiments, the pathogen is a pathogen causing an infectious disease.

In some embodiments of the first aspect, the nucleic acid is inhibitory RNA (such as siRNA) and selectively hybridizes to and/or is specific for a target mRNA. In some embodiments, the target mRNA comprises an ORF encoding a pharmaceutically active peptide or polypeptide, in particular a pharmaceutically active peptide or polypeptide whose expression (in particular increased expression, e.g., compared to the expression in a healthy subject) is associated with a disease. In some embodiments, the target mRNA comprises an ORF encoding a pharmaceutically active peptide or polypeptide whose expression (in particular increased expression, e.g., compared to the expression in a healthy subject) is associated with cancer.

In some embodiments of the first aspect, the composition is in liquid form, preferably at a temperature of about 0° C. to about 10° C., such as about 2° C. to about 8° C.

In some embodiments of the first aspect, the nucleic acid integrity (such as the RNA integrity) of the composition after storage for at least one week, preferably at a temperature of 0° C. or higher, such as about 2° C. to about 8° C., is such that the desired effect, e.g., to induce an immune response, can be achieved. In some embodiments, the nucleic acid integrity (such as the RNA integrity) of the composition after storage for at least one week (such as for at least 2 weeks, at least three weeks, at least four weeks, at least one month, at least two months, at least three months, at least 4 months, at least 6 months, at least 9 months, or at least 12 months), preferably at a temperature of 0° C. or higher, such as about 2° C. to about 8° C., is at least 50%, optionally at least 80%, and preferably at least 90%, compared to the nucleic acid integrity before storage. In some embodiments, the nucleic acid integrity (such as the RNA integrity) of the composition after storage for at least four weeks, preferably at a temperature of 0° C. or higher, such as about 2° C. to about 8° C., is at least 50%, optionally at least 80%, and preferably at least 90%, compared to the nucleic acid integrity before storage. In some embodiments, the nucleic acid integrity (such as the RNA integrity) of the composition after storage for at least three months, preferably at a temperature of 0° C. or higher, such as about 2° C. to about 8° C., is at least 50%, optionally at least 80%, and preferably at least 90%, compared to the nucleic acid integrity before storage.

In a second aspect, the present disclosure provides a method for delivering nucleic acid to cells of a subject, the method comprising administering to a subject a composition of the first aspect. It is understood that any embodiment described herein in the context of the first aspect may also apply to any embodiment of the second aspect.

In a third aspect, the present disclosure provides a method for delivering a therapeutic peptide or polypeptide to a subject, the method comprising administering to a subject a composition of the first aspect, wherein the nucleic acid encodes the therapeutic peptide or polypeptide. It is understood that any embodiment described herein in the context of the first or second aspect may also apply to any embodiment of the third aspect.

In a fourth aspect, the present disclosure provides a method for treating or preventing a disease or disorder in a subject, the method comprising administering to a subject a composition of the first aspect, wherein delivering the nucleic acid to cells of the subject is beneficial in treating or preventing the disease or disorder. In a related aspect, the present disclosure relates to a composition of the first aspect for use in a method for treating or preventing a disease or disorder in a subject, wherein delivering the nucleic acid to cells of the subject is beneficial in treating or preventing the disease or disorder. It is understood that any embodiment described herein in the context of the first, second, or third aspect may also apply to any embodiment of the fourth aspect.

In a fifth aspect, the present disclosure provides a method for treating or preventing a disease or disorder in a subject, the method comprising administering to a subject a composition of the first aspect, wherein the nucleic acid encodes a therapeutic peptide or polypeptide and wherein delivering the therapeutic peptide or polypeptide to the subject is beneficial in treating or preventing the disease or disorder. In a related aspect, the present disclosure relates to a composition of the first aspect for use in a method for treating or preventing a disease or disorder in a subject, wherein the nucleic acid encodes a therapeutic peptide or polypeptide and wherein delivering the therapeutic peptide or polypeptide to the subject is beneficial in treating or preventing the disease or disorder. It is understood that any embodiment described herein in the context of the first, second, third, or fourth aspect may also apply to any embodiment of the fifth aspect.

In some embodiments of the second to fifth aspect, the subject is a mammal, such as a human.

In a sixth aspect, the present disclosure provides a composition of the first aspect for use in therapy. It is understood that any embodiment described herein in the context of the first, second, third, fourth, or fifth aspect may also apply to any embodiment of the sixth aspect.

In a seventh aspect, the present disclosure provides a method of transfecting cells, comprising adding a composition of the first aspect to cells; and incubating the mixture of the composition and cells for a sufficient amount of time. In some embodiments, in particular those, where the nucleic acid is DNA or RNA (such as mRNA) and encodes a pharmaceutically active peptide or polypeptide, the mixture of the composition and cells is incubated for a time sufficient to allow the expression of the pharmaceutically active peptide or polypeptide. In some embodiments, in particular those, where the nucleic acid is inhibitory RNA (such as siRNA) directed against a target mRNA, the mixture of the composition and cells is incubated for a time sufficient to allow the inhibition of the transcription and/or translation of the target mRNA. In some embodiments, the sufficient amount of time is at least one hour (such at least about 2 hours, at least about 3 hours, at least about 4 hours, at least about 5 hours, at least about 6 hours, at least about 9 hours, at least about 12 hours) and/or up to about 48 hours (such as up to about 36 or up to about 24 hours).

In some embodiments of the seventh aspect, the method is conducted in vivo (i.e., the cells form part of an organ, a tissue and/or an organism of a subject). In some embodiments of the seventh aspect, the method is conducted in vitro (i.e., the cells do not form part of an organ, a tissue and/or an organism of a subject, e.g., the cells are an ex vivo cell culture).

It is understood that any embodiment described herein in the context of the first, second, third, fourth, fifth, or sixth aspect may also apply to any embodiment of the seventh aspect.

In an eighth aspect, the present disclosure provides a pharmaceutical composition comprising a composition of the first aspect. In some embodiments, the pharmaceutical composition further comprises one or more of pharmaceutically acceptable carriers, diluents and excipients. It is understood that any embodiment described herein in the context of the first, second, third, fourth, fifth, sixth, or seventh aspect may also apply to any embodiment of the eighth aspect.

In a ninth aspect, the present disclosure provides a kit comprising a composition of the first aspect or a pharmaceutical composition as described herein (such as a pharmaceutical composition of the eighth aspect). In some embodiments, the kit is for use in therapy, such as for inducing an immune response.

In some embodiments, the kit is for use in inducing an immune response against a pathogen, such as for treating or preventing an infectious disease. It is understood that any embodiment described herein in the context of the first, second, third, fourth, fifth, sixth, seventh, or eighth aspect may also apply to any embodiment of the ninth aspect.

In a tenth aspect, the present disclosure provides a method of preparing a composition comprising particles dispersed in a final aqueous phase, wherein the final aqueous phase comprises a final buffer system and has a pH of about 4.0-5.5 and wherein the particles contain (i) at least a portion of the nucleic acid; and (ii) at least a portion of the cationic or cationically ionizable lipid;

wherein the method comprises:
(a) providing (e.g., preparing) a nucleic acid solution containing water and a first buffer system;
(b) providing (e.g., preparing) an organic (e.g., ethanolic) solution comprising the cationic or cationically ionizable lipid and, if present, one or more additional lipids;
(c) mixing the nucleic acid solution provided under (a) with the organic solution provided under (b), thereby preparing a (first) intermediate formulation comprising the particles dispersed in a (first) aqueous phase comprising the first buffer system; and
(d) filtrating (e.g., dialyzing, tangential flow filtrating or diafiltrating) and/or diluting the (first) intermediate formulation prepared under (c) using a final aqueous buffer solution comprising the final buffer system, thereby preparing the composition comprising the particles dispersed in the final aqueous phase.

In some embodiments of the tenth aspect, the method comprises:
(a′) providing (e.g., preparing) an aqueous nucleic acid solution;
(b′) providing (e.g., preparing) a first aqueous buffer solution comprising a first buffer system;
(c′) mixing the aqueous nucleic acid solution provided under (a′) with the first aqueous buffer solution provided under (b′) thereby preparing nucleic acid solution containing water and the first buffer system;
(d′) preparing an organic (e.g., ethanolic) solution comprising the cationic or cationically ionizable lipid and, if present, one or more additional lipids;
(e′) mixing the nucleic acid solution prepared under (c′) with the organic solution prepared under (d′), thereby preparing a first intermediate formulation comprising particles dispersed in a first aqueous phase comprising the first buffer system;
(f′) optionally filtrating the first intermediate formulation prepared under (e′) using a further aqueous buffer solution comprising a further buffer system, thereby preparing a further intermediate formulation comprising the particles dispersed in a further aqueous phase comprising the further buffer system, wherein the further aqueous buffer solution may be identical to or different from the first aqueous buffer solution;
(g′) optionally repeating step (f′) once or two or more times, wherein the further intermediate formulation comprising the particles dispersed in the further aqueous phase comprising the further buffer system obtained after step (f′) of one cycle is used as the first intermediate formulation of the next cycle, wherein in each cycle the further aqueous buffer solution may be identical to or different from the first aqueous buffer solution;
(h′) filtrating the first intermediate formulation obtained in step (e′), if step (f′) is absent, or the further intermediate formulation obtained in step (f′), if step (f′) is present and step (g′) is not present, or the further intermediate formulation obtained after step (g′), if steps (f) and (g′) are present, using a final aqueous buffer solution comprising the final buffer system; and
(i′) optionally diluting the formulation obtained in step (h′) with a dilution solution; thereby preparing the composition comprising the particles dispersed in the final aqueous phase.

In some embodiments of the tenth aspect, the pH of the final aqueous phase is below 5.5 and/or above 4.0. In some embodiments of the tenth aspect, the pH of the final aqueous phase is at least 4.0 and below 5.5, such as at least 4.1 and below 5.5, at least 4.2 and below 5.5, at least 4.3 and below 5.5, at least 4.4 and below 5.5, or at least 4.5 and below 5.5, e.g., at least 5.0 and below 5.5. In some embodiments of the tenth aspect, the pH of the final aqueous phase is at least 4.0 and at most 5.4, such as at least 4.0 and at most 5.3, at least 4.0 and at most 5.2, at least 4.0 and at most 5.1, at least 4.0 and at most 5.0. In some embodiments of the tenth aspect, the pH of the final aqueous phase is at least 4.1 and at most 5.4, at least 4.2 and at most 5.3, at least 4.3 and at most 5.2, at least 4.4 and at most 5.1, or at least 4.5 and at most 5.0. In some embodiments of the tenth aspect, the pH of the final aqueous phase is at least 4.6 and at most 5.4, such as at least 4.7 and at most 5.3, at least 4.8 and at most 5.2, at least 4.9 and at most 5.1, e.g., about 5.0. In some embodiments of the tenth aspect, the pH of the aqueous phase is from about 4.0 to about 5.2. In some embodiments of the tenth aspect, the pH of the aqueous phase is from about 4.5 to about 5.2. In some embodiments of the tenth aspect, the pH of the aqueous phase is from about 4.8 to about 5.2. In some embodiments of the tenth aspect, the pH of the aqueous phase is from about 5.0 to about 5.2. In some embodiments of the tenth aspect, the pH of the aqueous phase is at least 5.0 and below 5.5. In some embodiments of the tenth aspect, the pH of the aqueous phase is between about 4.5 and 5.0. In some embodiments, the pH of the aqueous phase is at least 4.5 and below 5.5, optionally at least 4.5 and below 5.2. In some embodiments of the tenth aspect, the pH of the aqueous phase is at least 5.0 and below 5.5, e.g., at least 5.1 and below 5.5, at least 5.1 and at most 5.4, at least 5.2 and below 5.5, or at least 5.2 and at most 5.4. In some embodiments of the tenth aspect, the pH of the aqueous phase is about 5.3.

To this end, the nucleic acid (such as RNA) solution provided/obtained in step (a) or (c′) may further comprises one or more acids (e.g., selected from inorganic acids (such as hydrochloric acid, hydrobromic acid, or nitric acid) and organic acids (such as mono-, di- or polybasic organic acids, e.g., monocarboxylic acids (like acetic acid, propionic acid, or lactic acid), dicarboxylic acids (like oxalic acid, malonic acid, succinic acid, glutaric acid, adipic acid, maleic acid, fumaric acid, tartaric acid, or malic acid), or polycarboxylic acids (like citric acid, isocitric acid, or trimesic acid)). In some preferred embodiments, the acid is a monobasic acid, such as such as a monobasic inorganic acid (like hydrochloric acid) or a monobasic organic acid (like acetic acid). In some embodiments, it is preferred that step (d) is conducted under conditions which remove one or more unwanted substances (e.g., organic solvent (such as ethanol) and/or the one or more one or more acids) resulting in the composition comprising the particles dispersed in a final aqueous phase with the final aqueous phase being substantially free of such one or more unwanted substances. For example, such conditions can include subjecting the intermediate formulation comprising the particles dispersed in the second intermediate aqueous phase obtained in step (c) to at least one step of filtrating, such as dialyzing, tangential flow filtrating or diafiltrating, using a final buffer solution comprising the final buffer system (i.e., the final buffer substance), wherein the final buffer solution does not contain the one or more unwanted substances. Alternatively, such conditions can include (1) subjecting the intermediate formulation comprising the particles dispersed in the first intermediate aqueous phase obtained in step (c) (i.e., a first intermediate formulation) to at least one step of diluting using water or a further aqueous buffer solution comprising a further buffer system, thereby preparing a further intermediate formulation comprising the particles dispersed in a further aqueous phase comprising the first or further buffer system, wherein the further buffer system of the further aqueous buffer solution may be identical to or different from the buffer system used in step (a); and (2) subjecting the further intermediate formulation obtained in step (1) to at least one step of filtrating, such as dialyzing, tangential flow filtrating or diafiltrating, using the final aqueous buffer solution, wherein at least the final aqueous buffer solution (preferably the intermediate and final aqueous buffer solutions) does not contain the one or more unwanted substances.

Similarly, in some embodiments of the tenth aspect, where the method comprises steps (a′) to (e′), and (h′) (and optionally one or more of steps (f′), (g′), and (i′)), the first aqueous buffer solution provided under (b′) (and the first aqueous phase) may further comprises one or more acids (e.g., one or more di- or polybasic acids). In these embodiments, it is preferred that least one of steps (f′) to (h′) is conducted under conditions which remove one or more unwanted substances (e.g., organic solvent (such as ethanol) and/or the one or more di- or polybasic acids) from the first intermediate formulation and/or from the second intermediate formulation and/or from the further intermediate formulation resulting in a further inter formulation comprising the particles dispersed in a further aqueous phase or in the final aqueous phase with the further and/or final aqueous phase being substantially free of the one or more unwanted substances. For example, such conditions can include using a further aqueous buffer solution and/or a final buffer solution, wherein at least one of the further aqueous buffer solution(s) and the final buffer solution (preferably all of the further aqueous buffer solution(s) and the final buffer solution) does not contain the one or more unwanted substances. In some embodiments, the filtrating steps can be independently selected from dialyzing, tangential flow filtrating and diafiltrating, preferably from dialyzing and tangential flow filtrating.

In some embodiments of the tenth aspect, the concentration of the final buffer system in the final aqueous phase is between about 1 mM and about 50 mM. In some embodiments of the tenth aspect, the concentration of the final buffer system in the final aqueous phase is between about 2 mM and about 40 mM, such as between about 3 mM and about 30 mM, between about 4 mM and about 25 mM, or between about 5 mM and about 20 mM.

In some embodiments of the tenth aspect, the final buffer system comprises or consists essentially of histidine.

In some embodiments of the tenth aspect, the final buffer system comprises or consists essentially of HEPES.

In some embodiments of the tenth aspect, the final buffer system comprises or consists essentially of a combination of HEPES and histidine.

In some embodiments of the tenth aspect, the final aqueous phase further comprises a chelating agent, such as EDTA. In some embodiments, the concentration of the chelating agent in the final aqueous phase is between about 0.1 mM and about 20 mM, such as between about 0.2 mM and about 15 mM, between about 0.3 mM and about 12 mM, between about 0.4 mM and about 11 mM, or between about 0.5 mM and about 10 mM.

In some embodiments of the tenth aspect, the final buffer system comprises histidine and the final aqueous phase further comprises a chelating agent, such as EDTA. In some embodiments, the concentration of the chelating agent in the final aqueous phase is between about 0.1 mM and about 20 mM, such as between about 0.2 mM and about 15 mM, between about 0.3 mM and about 12 mM, between about 0.4 mM and about 11 mM, or between about 0.5 mM and about 10 mM.

In some embodiments of the tenth aspect, the final buffer system comprises histidine and the final aqueous phase is substantially free of EDTA, preferably substantially free of any chelating agent.

In some embodiments of the tenth aspect, the final buffer system comprises HEPES and the final aqueous phase further comprises a chelating agent, such as EDTA. In some embodiments, the concentration of the chelating agent in the final aqueous phase is between about 0.1 mM and about 20 mM, such as between about 0.2 mM and about 15 mM, between about 0.3 mM and about 12 mM, between about 0.4 mM and about 11 mM, or between about 0.5 mM and about 10 mM.

In some embodiments of the tenth aspect, the organic solution provided in step (b) and/or (e′) comprises an organic solvent selected from a lower alcohol, such as alcohols (in particular aliphatic alcohols) having up to 6 carbon atoms, and mixtures thereof (such as mixtures of two or more of these alcohols).

In preferred embodiments, the organic solvent is completely miscible with water. In some embodiments, the organic solvent is selected from the group consisting of ethanol, propanol, isopropanol, 1,2-propanediol, and mixtures of two or more of these alcohols. In some embodiments, the organic solvent is ethanol, propanol, or a mixture thereof.

In some embodiments of the tenth aspect, the composition is substantially free of a cryoprotectant.

In some embodiments of the tenth aspect, the first buffer system used in step (a) comprises the final buffer substance used in step (d), preferably the buffer system and pH of the first buffer system used in step (a) are identical to the buffer system and pH of the final aqueous buffer solution used in step (d).

For example, only one aqueous buffer solution is used in this embodiment of the tenth aspect. Similarly, in some embodiments of the tenth aspect, where the method comprises steps (a′) to (e′) and (h′) (and optionally one or more of steps (f′), (g′) and (i′)), each of the first buffer system and every further buffer system used in steps (b′), (f) and (g′) comprises the final buffer substance used in step (h′), preferably the buffer system and pH of each of the first aqueous buffer solution and of every further aqueous buffer solution used in steps (b′), (f) and (g′) are identical to the buffer system and pH of the final aqueous buffer solution. For example, the aqueous buffer solutions used in steps (b′), (f), if present, (g′), if present, and (h′) of this embodiment of the tenth aspect are identical.

It is understood that any embodiment described herein in the context of the first, second, third, fourth, fifth, sixth, seventh, eighth, or ninth aspect may also apply to any embodiment of the tenth aspect.

In an eleventh aspect, the present disclosure provides a method of storing a composition, comprising preparing a liquid composition according to the method of the tenth aspect and storing the liquid composition at a temperature ranging from about 0° C. to about 20° C., such as from about 1° C. to about 15° C., from about 2° C. to about 10° C., or from about 2° C. to about 8° C. In some embodiments of the eleventh aspect, storing the liquid composition is for at least 1 week, such as at least 2 weeks, at least 3 weeks, at least 4 weeks, at least 1 month, at least 2 months, at least 3 months, at least 6 months, at least 9 months, or at least 12 months, preferably at least 4 weeks. In some embodiments of the eleventh aspect, storing the liquid composition is for at least 4 weeks, preferably at least 1 month, more preferably at least 2 months, more preferably at least 3 months, more preferably at least 6 months, more preferably at least 9 months, more preferably at 9 to 12 months at about 2° C. to about 8° C.

In some embodiments of the eleventh aspect, the composition is substantially free of a cryoprotectant.

It is understood that any embodiment described herein in the context of the first, second, third, fourth, fifth, sixth, seventh, eighth, ninth aspect, or tenth aspect may also apply to any embodiment of the eleventh aspect.

In a twelfth aspect, the present disclosure provides a method for preparing a ready-to-use pharmaceutical composition, the method comprising the steps of providing a liquid composition prepared by the method of the tenth or eleventh aspect thereby obtaining the ready-to-use pharmaceutical composition.

It is understood that any embodiment described herein in the context of the first, second, third, fourth, fifth, sixth, seventh, eighth, ninth, tenth, or eleventh aspect may also apply to any embodiment of the twelfth aspect.

In a thirteenth aspect, the present disclosure provides a ready-to-use pharmaceutical composition preparable by the method of the twelfth aspect.

It is understood that any embodiment described herein in the context of the first, second, third, fourth, fifth, sixth, seventh, eighth, ninth, tenth, eleventh, or twelfth aspect may also apply to any embodiment of the thirteenth aspect. Furthermore, it is to be understood that the ready-to-use pharmaceutical composition of the thirteenth aspect can be used in any one of the second, third, fourth, fifth, sixth, seventh, eighth, and ninth aspects.

### BRIEF DESCRIPTION OF THE FIGURES

FIG. 1: Degradation rates of nucleic acid compositions stored in different environments. Compositions comprising sa-RNA (A: naked sa-RNA; B: sa-RNA/LP-2) and histidine (20 mM) were stored for up to 1 month at 4° C. and at different pH values (A, B: pH 4.0-7.0) in the presence of EDTA (1 mM). Shown are degradation rates (% per month)+standard deviation vs. pH value FIG. 2: Integrity of nucleic acid compositions stored in different environments. RNA/particles compositions were prepared (A: LP-1; B: LP-2) and were stored for up to 12 months at 4° C. and at different pH values (pH 5.0, 5.5, 6.0, and 6.5, respectively). Shown are the RNA integrity % values vs. the storage time.

FIG. 3: Integrity of nucleic acid compositions stored in different environments. RNA/LP-2 compositions were prepared with HEPES (A to D) or histidine (E) as buffering substance and stored for up to 9 months at 4° C. and at a pH of 5.5 (A), 6.0 (B), 6.5 (C), and 7.0 (D), respectively, in the absence (filled square) or presence (open triangle) of EDTA (1 mM). Shown are the RNA integrity % values vs. the storage time.

FIG. 4: Integrity of nucleic acid compositions stored in different environments. RNA/LP-1 compositions were prepared using histidine as buffering substance and different phospholipids (DOPE, DOPC, or DSPC) and were stored for up to 9 months at 4° C. and at a pH value of 5.0 (A), 5.5 (B), and 6.0 (C), respectively. Shown are the RNA integrity % values vs. the storage time.

### DESCRIPTION OF THE SEQUENCES

The following table provides a listing of certain sequences referenced herein.

TABLE 1
Description of the sequences

| | | |
|---|---|---|
| SEQ | Des- | |
| ID NO: | cription | Sequence |

5′-UTR (hAg-Kozak)

| | | |
|---|---|---|
| 1 | 5′-UTR | AACUAGUAUUCUUCUGGUCCCCACAGACUCAGAGAGAACCCGCCACC |

3′-UTR (FI element)

| | | |
|---|---|---|
| 2 | 3′-UTR | CUGGUACUGCAUGCACGCAAUGCUAGCUGCCCCUUUCCCGUCCUGGGUACCCCGAGUCUCCCCCGACCUCGGG |
| | | UCCCAGGUAUGCUCCCACCUCCACCUGCCCCACUCACCACCUCUGCUAGUUCCAGACACCUCCCAAGCACGCA |
| | | GCAAUGCAGCUCAAAACGCUUAGCCUAGCCACACCCCCACGGGAAACAGCAGUGAUUAACCUUUAGCAAUAAA |
| | | CGAAAGUUUAACUAAGCUAUACUAACCCCAGGGUUGGUCAAUUUCGUGCCAGCCACACC |

A30L70

| | | |
|---|---|---|
| 3 | A30L70 | AAAAAAAAAAAAAAAAAAAAAAAAAAAAAAGCAUAUGACUAAAAAAAAAAAAAAAAAAAAAAAAAAAAAAAAA |
| | | AAAAAAAAAAAAAAAAAAAAAAAAAAAAAAAAAAAAA |

Sec/MITD

| | | |
|---|---|---|
| 4 | Sec | MRVMAPRTLILLLSGALALTETWAGS |
| 5 | MITD | IVGIVAGLAVLAVVVIGAVVATVMCRRKSSGGKGGSYSQAASSDSAQGSDVSLTA |

P2P16

| | | |
|---|---|---|
| 6 | P2P16 | KKQYIKANSKFIGITELKKLGGGKRGGGKKMTNSVDDALINSTKIYSYFPSVISKVNQGAQGKKL |

GS Linker

| | | |
|---|---|---|
| 7 | GS | GGSGGGGSGG |
| | Linker 1 | |
| 8 | GS | GSSGGGGSPGGGSS |
| | Linker 2 | |

### DETAILED DESCRIPTION OF THE INVENTION

Although the present disclosure is further described in more detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

In the following, the elements of the present disclosure will be described in more detail. These elements are listed with specific embodiments, however, it should be understood that they may be combined in any manner and in any number to create additional embodiments. The variously described examples and preferred embodiments should not be construed to limit the present disclosure to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Preferably, the terms used herein are defined as described in “A multilingual glossary of biotechnological terms: (IUPAC Recommendations)”, H. G. W. Leuenberger, B. Nagel, and H. Kolbl, Eds., Helvetica Chimica Acta, CH-4010 Basel, Switzerland, (1995).

The practice of the present disclosure will employ, unless otherwise indicated, conventional chemistry, biochemistry, cell biology, immunology, and recombinant DNA techniques which are explained in the literature in the field (cf., e.g., Organikum, Deutscher Verlag der Wissenschaften, Berlin 1990; Streitwieser/Heathcook, “Organische Chemie”, VCH, 1990; Beyer/Walter, “Lehrbuch der Organischen Chemie”, S. Hirzel Verlag Stuttgart, 1988; Carey/Sundberg, “Organische Chemie”, VCH, 1995; March, “Advanced Organic Chemistry”, John Wiley & Sons, 1985; Rdmpp Chemie Lexikon, Falbe/Regitz (Hrsg.), Georg Thieme Verlag Stuttgart, New York, 1989; Molecular Cloning: A Laboratory Manual, 2nd Edition, J. Sambrook et al. eds., Cold Spring Harbor Laboratory Press, Cold Spring Harbor 1989.

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps. The term “consisting essentially of” means excluding other members, integers or steps of any essential significance. The term “comprising” encompasses the term “consisting essentially of” which, in turn, encompasses the term “consisting of”. Thus, at each occurrence in the present application, the term “comprising” may be replaced with the term “consisting essentially of” or “consisting of”. Likewise, at each occurrence in the present application, the term “consisting essentially of” may be replaced with the term “consisting of”.

The terms “a”, “an” and “the” and similar references used in the context of describing the present disclosure (especially in the context of the claims) are to be construed to cover both the singular and the plural, unless otherwise indicated herein or clearly contradicted by the context.

All methods described herein can be performed in any suitable order unless otherwise indicated herein or otherwise clearly contradicted by the context.

The use of any and all examples, or exemplary language (e.g., “such as”), provided herein is intended merely to better illustrate the present disclosure and does not pose a limitation on the scope of the present disclosure otherwise claimed. No language in the specification should be construed as indicating any non-claimed element essential to the practice of the present disclosure.

Where used herein, “and/or” is to be taken as specific disclosure of each of the two specified features or components with or without the other. For example, “X and/or Y” is to be taken as specific disclosure of each of (i) X, (ii) Y, and (iii) X and Y, just as if each is set out individually herein.

In the context of the present disclosure, the term “about” denotes an interval of accuracy that the person of ordinary skill will understand to still ensure the technical effect of the feature in question. The term typically indicates deviation from the indicated numerical value by +10%, such as ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, and for example ±0.01%. In some embodiments, “about” indicates deviation from the indicated numerical value by ±10%. In some embodiments, “about” indicates deviation from the indicated numerical value by ±5%. In some embodiments, “about” indicates deviation from the indicated numerical value by ±4%. In some embodiments, “about” indicates deviation from the indicated numerical value by ±3%. In some embodiments, “about” indicates deviation from the indicated numerical value by ±2%. In some embodiments, “about” indicates deviation from the indicated numerical value by ±1%. In some embodiments, “about” indicates deviation from the indicated numerical value by ±0.9%. In some embodiments, “about” indicates deviation from the indicated numerical value by ±0.8%. In some embodiments, “about” indicates deviation from the indicated numerical value by ±0.7%. In some embodiments, “about” indicates deviation from the indicated numerical value by ±0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by ±0.3%. In some embodiments, “about” indicates deviation from the indicated numerical value by ±0.2%. In some embodiments, “about” indicates deviation from the indicated numerical value by ±0.1%. In some embodiments, “about” indicates deviation from the indicated numerical value by ±0.05%. In some embodiments, “about” indicates deviation from the indicated numerical value by ±0.01%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect.

Recitation of ranges of values herein is merely intended to serve as a shorthand method of referring individually to each separate value falling within the range. Unless otherwise indicated herein, each individual value is incorporated into the specification as if it were individually recited herein.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

### Definitions

In the following, definitions will be provided which apply to all aspects of the present disclosure. The following terms have the following meanings unless otherwise indicated. Any undefined terms have their art recognized meanings.

Terms such as “reduce” or “inhibit” as used herein means the ability to cause an overall decrease, for example, of about 5% or greater, about 10% or greater, about 15% or greater, about 20% or greater, about 25% or greater, about 30% or greater, about 40% or greater, about 50% or greater, or about 75% or greater, in the level. The term “inhibit” or similar phrases includes a complete or essentially complete inhibition, i.e. a reduction to zero or essentially to zero.

Terms such as “enhance” and “increase” as used herein means the ability to cause an overall increase, or enhancement, for example, by at least about 5% or greater, about 10% or greater, about 15% or greater, about 20% or greater, about 25% or greater, about 30% or greater, about 40% or greater, about 50% or greater, about 75% or greater, or about 100% or greater in the level. In some embodiments, these terms relate to an increase or enhancement by at least about 10%, at least about 20%, at least about 30%, at least about 40%, at least about 50%, at least about 80%, or at least about 100%.

“Physiological pH” as used herein refers to a pH of about 7.5 or about 7.4. In some embodiments, physiological pH is from 7.3 to 7.5. In some embodiments, physiological pH is from 7.35 to 7.45. In some embodiments, physiological pH is 7.3, 7.35, 7.4, 7.45, or 7.5. “Physiological conditions” as used herein refer to the conditions (in particular pH and temperature) in a living subject, in particular a human. Preferably, physiological conditions mean a physiological pH and/or a temperature of about 37° C.

As used in the present disclosure, “% (w/v)” (or “% w/v”) refers to weight by volume percent, which is a unit of concentration measuring the amount of solute in grams (g) expressed as a percent of the total volume of solution in milliliters (ml).

As used in the present disclosure, “% by volume” or “% (v/v)” (or “% v/v”) refers to volume percent, which is a unit of concentration measuring the amount of a liquid substance in milliliters (ml) expressed as a percent of the total volume of solution in milliliters (ml).

As used in the present disclosure, “% by weight” or “% (w/w)” (or “% w/w”) refers to weight percent, which is a unit of concentration measuring the amount of a substance in grams (g) expressed as a percent of the total weight of the total composition in grams (g).

As used in the present disclosure, “mol %” is defined as the ratio of the number of moles of one component to the total number of moles of all components, multiplied by 100.

As used in the present disclosure, “mol % of the total lipid” is defined as the ratio of the number of moles of one lipid component to the total number of moles of all lipids, multiplied by 100. In this context, in some embodiments, the term “total lipid” includes lipids and lipid-like material.

As used in the present disclosure, the term “buffer system” means a system including a buffer substance.

A “buffer substance” as used herein refers to a mixture of a base and its protonated form (e.g., a mixture of histidine and its protonated form [histidine-H]+). Consequently, the amount of a buffer substance contained in a composition is the sum of the amounts of both the base and the conjugate acid in the composition. In some embodiments a “buffer system” may consist essentially of a “buffer substance”. “Molar ratio”, as used herein, refers to the ratio between the amounts in moles of any two substances.

For example, if a first substance is present in a composition in an amount of 1 millimole (mmol) and a second substance is present in the composition in an amount of 2 millimole (mmol), the molar ratio of the first substance to the second substance is 1:2 or 0.5.

“Osmolality” refers to the concentration of a particular solute expressed as the number of osmoles of solute per kilogram of solvent.

The term “reconstitute” relates to adding a solvent such as water to a dried product to return it to a liquid state such as its original liquid state.

The term “freezing” relates to the solidification of a liquid, usually with the removal of heat. In some embodiments, freezing is reverse action to thawing.

The term “thawing” relates to the liquification of a solid, usually with the addition of heat. In some embodiments, thawing is reverse action to freezing.

The term “aqueous phase” as used herein in relation to a composition/formulation comprising particles, in particular LNPs, liposomes, and/or lipoplexes, means the mobile or liquid phase, i.e., the continuous water phase including all components dissolved therein but (formally) excluding the particles. Thus, if particles, such as LNPs, are dispersed in an aqueous phase and the aqueous phase is to be substantially free of compound X, the aqueous phase is free of X is such manner as it is practically and realistically feasible, e.g., the concentration of compound X in the aqueous composition is less than 1% by weight.

However, it is possible that, at the same time, the particles dispersed in the aqueous phase may comprise compound X in an amount of more than 1% by weight.

The term “recombinant” in the context of the present disclosure means “made through genetic engineering”. In some embodiments, a “recombinant object” in the context of the present disclosure is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring. The term “found in nature” means “present in nature” and includes known objects as well as objects that have not yet been discovered and/or isolated from nature, but that may be discovered and/or isolated in the future from a natural source.

As used herein, the terms “room temperature” and “ambient temperature” are used interchangeably herein and refer to temperatures from at least about 15° C., preferably from about 15° C. to about 35° C., from about 15° C. to about 30° C., from about 15° C. to about 25° C., or from about 17° C. to about 22° C.

Such temperatures will include 15° C., 16° C., 17° C., 18° C., 19° C., 20° C., 21° C. and 22° C. The term “alkyl” refers to a monoradical of a saturated straight or branched hydrocarbon. Preferably, the alkyl group comprises from 1 to 12 (such as 1 to 10) carbon atoms, i.e., 1, 2, 3, 4, 5, 6, 7, 8, 9, 10, 11, or 12 carbon atoms, abbreviated as C₁₋₁₂ alkyl, (such as 1, 2, 3, 4, 5, 6, 7, 8, 9, or 10 carbon atoms, abbreviated as C₁₋₁₀ alkyl), more preferably 1 to 8 carbon atoms, such as 1 to 6 or 1 to 4 carbon atoms.

Exemplary alkyl groups include methyl, ethyl, propyl, iso-propyl (also called 2-propyl or 1-methylethyl), butyl, iso-butyl, tert-butyl, n-pentyl, iso-pentyl, sec-pentyl, neo-pentyl, 1,2-dimethyl-propyl, iso-amyl, n-hexyl, iso-hexyl, sec-hexyl, n-heptyl, iso-heptyl, n-octyl, 2-ethyl-hexyl, n-nonyl, n-decyl, n-undecyl, n-dodecyl, and the like. A “substituted alkyl” means that one or more (such as 1 to the maximum number of hydrogen atoms bound to an alkyl group, e.g., 1, 2, 3, 4, 5, 6, 7, 8, 9, or up to 10, such as between 1 to 5, 1 to 4, or 1 to 3, or 1 or 2) hydrogen atoms of the alkylene group are replaced with a substituent other than hydrogen (when more than one hydrogen atom is replaced the substituents may be the same or different). Preferably, the substituent other than hydrogen is a 1V level substituent, as specified herein. Examples of a substituted alkyl include chloromethyl, dichloromethyl, fluoromethyl, and difluoromethyl.

The term “alkylene” refers to a diradical of a saturated straight or branched hydrocarbon. Preferably, the alkylene comprises from 1 to 12 (such as 1 to 10) carbon atoms, i.e., 1, 2, 3, 4, 5, 6, 7, 8, 9, 10, 11, or 12 carbon atoms (such as 1, 2, 3, 4, 5, 6, 7, 8, 9, or 10 carbon atoms), more preferably 1 to 8 carbon atoms, such as 1 to 6 or 1 to 4 carbon atoms. Exemplary alkylene groups include methylene, ethylene (i.e., 1,1-ethylene, 1,2-ethylene), propylene (i.e., 1,1-propylene, 1,2-propylene (—CH(CH₃)CH₂—), 2,2-propylene (—C(CH₃)₂—), and 1,3-propylene), the butylene isomers (e.g., 1,1-butylene, 1,2-butylene, 2,2-butylene, 1,3-butylene, 2,3-butylene (cis or trans or a mixture thereof), 1,4-butylene, 1,1-iso-butylene, 1,2-iso-butylene, and 1,3-iso-butylene), the pentylene isomers (e.g., 1,1-pentylene, 1,2-pentylene, 1,3-pentylene, 1,4-pentylene, 1,5-pentylene, 1,1-iso-pentylene, 1,1-sec-pentyl, 1,1-neo-pentyl), the hexylene isomers (e.g., 1,1-hexylene, 1,2-hexylene, 1,3-hexylene, 1,4-hexylene, 1,5-hexylene, 1,6-hexylene, and 1,1-isohexylene), the heptylene isomers (e.g., 1,1-heptylene, 1,2-heptylene, 1,3-heptylene, 1,4-heptylene, 1,5-heptylene, 1,6-heptylene, 1,7-heptylene, and 1,1-isoheptylene), the octylene isomers (e.g., 1,1-octylene, 1,2-octylene, 1,3-octylene, 1,4-octylene, 1,5-octylene, 1,6-octylene, 1,7-octylene, 1,8-octylene, and 1,1-isooctylene), and the like. The straight alkylene moieties having at least 3 carbon atoms and a free valence at each end can also be designated as a multiple of methylene (e.g., 1,4-butylene can also be called tetramethylene). Generally, instead of using the ending “ylene” for alkylene moieties as specified above, one can also use the ending “diyl” (e.g., 1,2-butylene can also be called butan-1,2-diyl). A “substituted alkylene” means that one or more (such as 1 to the maximum number of hydrogen atoms bound to an alkylene group, e.g., 1, 2, 3, 4, 5, 6, 7, 8, 9, or up to 10, such as between 1 to 5, 1 to 4, or 1 to 3, or 1 or 2) hydrogen atoms of the alkylene group are replaced with a substituent other than hydrogen (when more than one hydrogen atom is replaced the substituents may be the same or different). Preferably, the substituent other than hydrogen is a 1V level substituent, as specified herein.

The term “alkenyl” refers to a monoradical of an unsaturated straight or branched hydrocarbon having at least one carbon-carbon double bond. Generally, the maximal number of carbon-carbon double bonds in the alkenyl group can be equal to the integer which is calculated by dividing the number of carbon atoms in the alkenyl group by 2 and, if the number of carbon atoms in the alkenyl group is uneven, rounding the result of the division down to the next integer. For example, for an alkenyl group having 9 carbon atoms, the maximum number of carbon-carbon double bonds is 4. Preferably, the alkenyl group has 1 to 6 (such as 1 to 4), i.e., 1, 2, 3, 4, 5, or 6, carbon-carbon double bonds. Preferably, the alkenyl group comprises from 2 to 12 (such as 2 to 10) carbon atoms, i.e., 2, 3, 4, 5, 6, 7, 8, 9, 10, 11, or 12 carbon atoms (such as 2, 3, 4, 5, 6, 7, 8, 9, or 10 carbon atoms), more preferably 2 to 8 carbon atoms, such as 2 to 6 carbon atoms or 2 to 4 carbon atoms. Thus, in a preferred embodiment, the alkenyl group comprises from 2 to 12, abbreviated as C₂₋₁₂ alkenyl, (e.g., 2 to 10) carbon atoms and 1, 2, 3, 4, 5, or 6 (e.g., 1, 2, 3, 4, or 5) carbon-carbon double bonds, more preferably it comprises 2 to 8 carbon atoms and 1, 2, 3, or 4 carbon-carbon double bonds, such as 2 to 6 carbon atoms and 1, 2, or 3 carbon-carbon double bonds or 2 to 4 carbon atoms and 1 or 2 carbon-carbon double bonds. The carbon-carbon double bond(s) may be in cis (Z) or trans (E) configuration. Exemplary alkenyl groups include vinyl, 1-propenyl, 2-propenyl (i.e., allyl), 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, 11-dodecenyl, and the like. If an alkenyl group is attached to a nitrogen atom, the double bond cannot be alpha to the nitrogen atom. A “substituted alkenyl” means that one or more (such as 1 to the maximum number of hydrogen atoms bound to an alkenyl group, e.g., 1, 2, 3, 4, 5, 6, 7, 8, 9, or up to 10, such as between 1 to 5, 1 to 4, or 1 to 3, or 1 or 2) hydrogen atoms of the alkenyl group are replaced with a substituent other than hydrogen (when more than one hydrogen atom is replaced the substituents may be the same or different). Preferably, the substituent other than hydrogen is a 1t level substituent as specified herein.

The term “alkynyl” refers to a linear or branched monovalent hydrocarbon moiety having at least one carbon-carbon triple bond in which the total carbon atoms may be six to thirty, typically six to twenty, often six to eighteen. Alkynyl groups can optionally have one or more carbon carbon double bonds.

Generally, the maximal number of carbon-carbon triple bonds in the alkynyl group can be equal to the integer which is calculated by dividing the number of carbon atoms in the alkynyl group by 2 and, if the number of carbon atoms in the alkynyl group is uneven, rounding the result of the division down to the next integer. For example, for an alkynyl group having 9 carbon atoms, the maximum number of carbon-carbon triple bonds is 4. Preferably, the alkynyl group has 1 to 6 (such as 1 to 4), i.e., 1, 2, 3, 4, 5, or 6, more preferably 1 or 2 carbon-carbon triple bonds. Exemplary alkynyl groups include ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, 1-heptynyl, 2-heptynyl, 3-heptynyl, 4-heptynyl, 5-heptynyl, 6-heptynyl, 1-octynyl, 2-octynyl, 3-octynyl, 4-octynyl, 5-octynyl, 6-octynyl, 7-octynyl, 1-nonylyl, 2-nonynyl, 3-nonynyl, 4-nonynyl, 5-nonynyl, 6-nonynyl, 7-nonynyl, 8-nonynyl, 1-decynyl, 2-decynyl, 3-decynyl, 4-decynyl, 5-decynyl, 6-decynyl, 7-decynyl, 8-decynyl, 9-decynyl, and the like. If an alkynyl group is attached to a nitrogen atom, the triple bond cannot be alpha to the nitrogen atom. A “substituted alkynyl” means that one or more (such as 1 to the maximum number of hydrogen atoms bound to an alkynyl group, e.g., 1, 2, 3, 4, 5, 6, 7, 8, 9, or up to 10, such as between 1 to 5, 1 to 4, or 1 to 3, or 1 or 2) hydrogen atoms of the alkynyl group are replaced with a substituent other than hydrogen (when more than one hydrogen atom is replaced the substituents may be the same or different).

Preferably, the substituent other than hydrogen is a 1V level substituent as specified herein.

The term “alkenylene” refers to a diradical of an unsaturated straight or branched hydrocarbon having at least one carbon-carbon double bond. Generally, the maximal number of carbon-carbon double bonds in the alkenylene group can be equal to the integer which is calculated by dividing the number of carbon atoms in the alkenylene group by 2 and, if the number of carbon atoms in the alkenylene group is uneven, rounding the result of the division down to the next integer. For example, for an alkenylene group having 9 carbon atoms, the maximum number of carbon-carbon double bonds is 4. Preferably, the alkenylene group has 1 to 6 (such as 1 to 4), i.e., 1, 2, 3, 4, 5, or 6, carbon-carbon double bonds. Preferably, the alkenylene group comprises from 2 to 12 (such as 2 to 10) carbon atoms, i.e., 2, 3, 4, 5, 6, 7, 8, 9, 10, 11, or 12 carbon atoms (such as 2, 3, 4, 5, 6, 7, 8, 9, or 10 carbon atoms), more preferably 2 to 8 carbon atoms, such as 2 to 6 carbon atoms or 2 to 4 carbon atoms. Thus, in a preferred embodiment, the alkenylene group comprises from 2 to 12 (such as 2 to 10 carbon) atoms and 1, 2, 3, 4, 5, or 6 (such as 1, 2, 3, 4, or 5) carbon-carbon double bonds, more preferably it comprises 2 to 8 carbon atoms and 1, 2, 3, or 4 carbon-carbon double bonds, such as 2 to 6 carbon atoms and 1, 2, or 3 carbon-carbon double bonds or 2 to 4 carbon atoms and 1 or 2 carbon-carbon double bonds. The carbon-carbon double bond(s) may be in cis (Z) or trans (E) configuration. Exemplary alkenylene groups include ethen-1,2-diyl, vinylidene (also called ethenylidene), 1-propen-1,2-diyl, 1-propen-1,3-diyl, 1-propen-2,3-diyl, allylidene, 1-buten-1,2-diyl, 1-buten-1,3-diyl, 1-buten-1,4-diyl, 1-buten-2,3-diyl, 1-buten-2,4-diyl, 1-buten-3,4-diyl, 2-buten-1,2-diyl, 2-buten-1,3-diyl, 2-buten-1,4-diyl, 2-buten-2,3-diyl, 2-buten-2,4-diyl, 2-buten-3,4-diyl, and the like. If an alkenylene group is attached to a nitrogen atom, the double bond cannot be alpha to the nitrogen atom. A “substituted alkenylene” means that one or more (such as 1 to the maximum number of hydrogen atoms bound to an alkenylene group, e.g., 1, 2, 3, 4, 5, 6, 7, 8, 9, or up to 10, such as between 1 to 5, 1 to 4, or 1 to 3, or 1 or 2) hydrogen atoms of the alkenylene group are replaced with a substituent other than hydrogen (when more than one hydrogen atom is replaced the substituents may be the same or different). Preferably, the substituent other than hydrogen is a l^{st} level substituent as specified herein.

The term “alkynylene” refers to a diradical of an unsaturated straight or branched hydrocarbon having at least one carbon-carbon triple bond (e.g., it refers to a divalent version of the monoradical alkynyl defined above). Generally, the maximal number of carbon-carbon triple bonds in the alkynylene group can be equal to the integer which is calculated by dividing the number of carbon atoms in the alkynylene group by 2 and, if the number of carbon atoms in the alkynylene group is uneven, rounding the result of the division down to the next integer. For example, for an alkynylene group having 9 carbon atoms, the maximum number of carbon-carbon triple bonds is 4. Preferably, the alkynylene group has 1 to 7 (such as 1 to 4), i.e., 1, 2, 3, 4, 5, 6, or 7 (such as 1, 2, 3, or 4), more preferably 1 or 2 carbon-carbon triple bonds. Preferably, the alkynylene group comprises from 2 to 14 (such as 2 to 12 or 2 to 10) carbon atoms, i.e., 2, 3, 4, 5, 6, 7, 8, 9, 10, 11, 12, 13, or 14 carbon atoms (such as 2, 3, 4, 5, 6, 7, 8, 9, or 10 carbon atoms), more preferably 2 to 8 carbon atoms, such as 2 to 6 carbon atoms or 2 to 4 carbon atoms.

Thus, in a preferred embodiment, the alkynylene group comprises from 2 to 14 (such as 2 to 10) carbon atoms and 1, 2, 3, 4, 5, 6, or 7 (such as 1, 2, 3, 4, or 5 (preferably 1, 2, or 3)) carbon-carbon triple bonds, more preferably it comprises 2 to 8 carbon atoms and 1, 2, 3, or 4 (preferably 1 or 2) carbon-carbon triple bonds, such as 2 to 6 carbon atoms and 1, 2 or 3 carbon-carbon triple bonds or 2 to 4 carbon atoms and 1 or 2 carbon-carbon triple bonds. Exemplary alkynylene groups include ethyn-1,2-diyl, 1-propyn-1,3-diyl, 1-propyn-3,3-diyl, 1-butyn-1,3-diyl, 1-butyn-1,4-diyl, 1-butyn-3,4-diyl, 2-butyn-1,4-diyl and the like. If an alkynylene group is attached to a nitrogen atom, the triple bond cannot be alpha to the nitrogen atom. A “substituted alkynylene” means that one or more (such as 1 to the maximum number of hydrogen atoms bound to an alkynylene group, e.g., 1, 2, 3, 4, 5, 6, 7, 8, 9, or up to 10, such as between 1 to 5, 1 to 4, or 1 to 3, or 1 or 2) hydrogen atoms of the alkynylene group are replaced with a substituent other than hydrogen (when more than one hydrogen atom is replaced the substituents may be the same or different). Preferably, the substituent other than hydrogen is a 1^{st} level substituent as specified herein.

The term “cycloalkyl” represents cyclic non-aromatic versions of “alkyl” and “alkenyl” with preferably 3 to 14 carbon atoms, such as 3 to 12 or 3 to 10 carbon atoms, i.e., 3, 4, 5, 6, 7, 8, 9, 10, 11, 12, 13, or 14 carbon atoms (such as 3, 4, 5, 6, 7, 8, 9, or 10 carbon atoms), more preferably 3 to 7 carbon atoms.

Exemplary cycloalkyl groups include cyclopropyl, cyclopropenyl, cyclobutyl, cyclobutenyl, cyclopentyl, cyclopentenyl, cyclohexyl, cyclohexenyl, cycloheptyl, cycloheptenyl, cyclooctyl, cyclooctenyl, cyclononyl, cyclononenyl, cylcodecyl, cylcodecenyl, and adamantyl. The cycloalkyl group may consist of one ring (monocyclic), two rings (bicyclic), or more than two rings (polycyclic).

The term “cycloalkylene” represents cyclic non-aromatic versions of “alkylene” and is a geminal, vicinal or isolated diradical. In certain embodiments, the cycloalkylene (i) is monocyclic or polycyclic (such as bi- or tricyclic) and/or (ii) is 3- to 14-membered (i.e., 3-, 4-, 5-, 6-, 7-, 8-, 9-, 10-, 11-, 12-, 13-, or 14-membered, such as 3- to 12-membered or 3- to 10-membered). In one embodiment the cycloalkylene is a mono-, bi- or tricyclic 3- to 14-membered (i.e., 3-, 4-, 5-, 6-, 7-, 8-, 9-, 10-, 11-, 12-, 13-, or 14-membered, such as 3- to 12-membered or 3- to 10-membered)cycloalkylene. Generally, instead of using the ending “ylene” for cycloalkylene moieties as specified above, one can also use the ending “diyl” (e.g., 1,2-cyclopropylene can also be called cyclopropan-1,2-diyl) Exemplary cycloalkylene groups include cyclohexylene, cycloheptylene, cyclopropylene, cyclobutylene, cyclopentylene, cyclooctylene, bicyclo[3.2.1]octylene, bicyclo[3.2.2]nonylene, and adamantanylene (e.g., tricyclo[3.3.1.1^{3,7}]decan-2,2-diyl). A “substituted cycloalkylene” means that one or more (such as 1 to the maximum number of hydrogen atoms bound to an cycloalkylene group, e.g., 1, 2, 3, 4, 5, 6, 7, 8, 9, or up to 10, such as between 1 to 5, 1 to 4, or 1 to 3, or 1 or 2) hydrogen atoms of the alkylene group are replaced with a substituent other than hydrogen (when more than one hydrogen atom is replaced the substituents may be the same or different). Preferably, the substituent other than hydrogen is a 1t level substituent as specified herein.

The term “cycloalkenylene” represents cyclic non-aromatic versions of “alkenylene” and is a geminal, vicinal or isolated diradical. Generally, the maximal number of carbon-carbon double bonds in the cycloalkenylene group can be equal to the integer which is calculated by dividing the number of carbon atoms in the cycloalkenylene group by 2 and, if the number of carbon atoms in the cycloalkenylene group is uneven, rounding the result of the division down to the next integer. For example, for an cycloalkenylene group having 9 carbon atoms, the maximum number of carbon-carbon double bonds is 4. Preferably, the cycloalkenylene group has 1 to 6 (such as 1 to 4), i.e., 1, 2, 3, 4, 5, or 6, carbon-carbon double bonds. In certain embodiments, the cycloalkenylene (i) is monocyclic or polycyclic (such as bi- or tricyclic) and/or (ii) is 3- to 14-membered (i.e., 3-, 4-, 5-, 6-, 7-, 8-, 9-, 10-, 11-, 12-, 13-, or 14-membered, such as 3- to 12-membered or 3- to 10-membered). In one embodiment the cycloalkenylene is a mono-, bi- or tricyclic 3- to 14-membered (i.e., 3-, 4-, 5-, 6-, 7-, 8-, 9-, 10-, 11-, 12-, 13-, or 14-membered, such as 3- to 12-membered or 3- to 10-membered)cycloalkenylene. Exemplary cycloalkenylene groups include cyclohexenylene, cycloheptenylene, cyclopropenylene, cyclobutenylene, cyclopentenylene, and cyclooctenylene. A “substituted cycloalkenylene” means that one or more (such as 1 to the maximum number of hydrogen atoms bound to an cycloalkenylene group, e.g., 1, 2, 3, 4, 5, 6, 7, 8, 9, or up to 10, such as between 1 to 5, 1 to 4, or 1 to 3, or 1 or 2) hydrogen atoms of the cycloalkenylene group are replaced with a substituent other than hydrogen (when more than one hydrogen atom is replaced the substituents may be the same or different). Preferably, the substituent other than hydrogen is a 1St level substituent as specified herein.

The term “aryl” refers to a monoradical of an aromatic cyclic hydrocarbon. Preferably, the aryl group contains 3 to 14 (e.g., 5, 6, 7, 8, 9, or 10, such as 5, 6, or 10) carbon atoms which can be arranged in one ring (e.g., phenyl) or two or more condensed rings (e.g., naphthyl). Exemplary aryl groups include cyclopropenylium, cyclopentadienyl, phenyl, indenyl, naphthyl, azulenyl, fluorenyl, anthryl, and phenanthryl. Preferably, “aryl” refers to a monocyclic ring containing 6 carbon atoms or an aromatic bicyclic ring system containing 10 carbon atoms. Preferred examples are phenyl and naphthyl. Aryl does not encompass fullerenes. A “substituted aryl” means that one or more (such as 1 to the maximum number of hydrogen atoms bound to an aryl group, e.g., 1, 2, 3, 4, 5, 6, 7, 8, 9, or up to 10, such as between 1 to 5, 1 to 4, or 1 to 3, or 1 or 2) hydrogen atoms of the aryl group are replaced with a substituent other than hydrogen (when more than one hydrogen atom is replaced the substituents may be the same or different). Preferably, the substituent other than hydrogen is a 1 level substituent as specified herein.

The term “heteroaryl” means an aryl group as defined above in which one or more (e.g., 1, 2, 3, or 4) carbon atoms in the aryl group are replaced by heteroatoms (such as O, S, or N). Preferably, heteroaryl refers to a 3-8-membered (such as 5-6-membered) aromatic monocyclic ring, wherein one or more (e.g., 1, 2, or 3) carbon atoms are replaced by the same or different heteroatoms of O, N, or S. Alternatively, it means an aromatic bicyclic or tricyclic ring system, wherein 1, 2, 3, 4, or 5 carbon atoms are replaced with the same or different heteroatoms of O, N, or S. Preferably, in each ring of the heteroaryl group the maximum number of O atoms is 1, the maximum number of S atoms is 1, and the maximum total number of O and S atoms is 2. For example, 3- to 14-membered heteroaryl encompasses monocyclic heteroaryl (e.g., 3-, 5- or 6-membered), bicyclic heteroaryl (e.g., 9- or 10-membered), and tricyclic heteroaryl (e.g., 13- or 14-membered). Exemplary heteroaryl groups include furanyl, thienyl, oxazolyl, isoxazolyl, oxadiazolyl, pyrrolyl, imidazolyl, pyrazolyl, triazolyl, tetrazolyl, thiazolyl, isothiazolyl, thiadiazolyl, pyridyl, pyrimidinyl, pyrazinyl, triazinyl, benzofuranyl, indolyl, isoindolyl, benzothienyl, 1H-indazolyl, benzimidazolyl, benzoxazolyl, indoxazinyl, benzisoxazolyl, benzothiazolyl, benzisothiazolyl, benzotriazolyl, quinolinyl, isoquinolinyl, benzodiazinyl, quinoxalinyl, quinazolinyl, benzotriazinyl, pyridazinyl, phenoxazinyl, thiazolopyridinyl, pyrrolothiazolyl, phenothiazinyl, isobenzofuranyl, chromenyl, xanthenyl, pyrrolizinyl, indolizinyl, indazolyl, purinyl, quinolizinyl, phthalazinyl, naphthyridinyl, cinnolinyl, pteridinyl, carbazolyl, phenanthridinyl, acridinyl, perimidinyl, phenanthrolinyl, and phenazinyl. Exemplary 5- or 6-memered heteroaryl groups include furanyl, thienyl, oxazolyl, isoxazolyl, oxadiazolyl, pyrrolyl, imidazolyl (e.g., 2-imidazolyl), pyrazolyl, triazolyl, tetrazolyl, thiazolyl, isothiazolyl, thiadiazolyl, pyridyl (e.g., 4-pyridyl), pyrimidinyl, pyrazinyl, triazinyl, and pyridazinyl. A “substituted heteroaryl” means that one or more (such as 1 to the maximum number of hydrogen atoms bound to an heteroaryl group, e.g., 1, 2, 3, 4, 5, 6, 7, 8, 9, or up to 10, such as between 1 to 5, 1 to 4, or 1 to 3, or 1 or 2) hydrogen atoms of the heteroaryl group are replaced with a substituent other than hydrogen (when more than one hydrogen atom is replaced the substituents may be the same or different). Preferably, the substituent other than hydrogen is a 1t level substituent as specified herein.

The terms “heterocyclyl”, “heterocyclic”, and “heterocycloalkyl” (which are used interchangeably herein) mean a cycloalkyl group as defined above in which one or more (e.g., 1, 2, 3, or 4) carbon atoms in the cycloalkyl group are replaced by heteroatoms of O, N, Si, Se, P, or S, preferably O, S, or N. A heterocyclyl group has preferably 1 or 2 rings containing from 3 to 10, such as 3, 4, 5, 6, or 7, ring atoms. Preferably, in each ring of the heterocyclyl group the maximum number of O atoms is 1, the maximum number of S atoms is 1, and the maximum total number of O and S atoms is 2. The term “heterocyclyl” is also meant to encompass partially or completely hydrogenated forms (such as dihydro, tetrahydro or perhydro forms) of the above-mentioned heteroaryl groups. Exemplary heterocyclyl groups include morpholinyl, pyrrolidinyl, imidazolidinyl, 2,3-dihydro-1H-imidazolyl, pyrazolidinyl, piperidinyl (also called piperidyl), piperazinyl, azetidinyl, di- and tetrahydrofuranyl, di- and tetrahydrothienyl, di- and tetrahydropyranyl, urotropinyl, lactones, lactams, cyclic imides, and cyclic anhydrides. A “substituted heterocyclyl” means that one or more (such as 1 to the maximum number of hydrogen atoms bound to a heterocyclyl group, e.g., 1, 2, 3, 4, 5, 6, 7, 8, 9, or up to 10, such as between 1 to 5, 1 to 4, or 1 to 3, or 1 or 2) hydrogen atoms of the heterocyclyl group are replaced with a substituent other than hydrogen (when more than one hydrogen atom is replaced the substituents may be the same or different). Preferably, the substituent other than hydrogen is a 1t level substituent as specified herein.

The term “heterocycloalkylene” means a diradical version of the monoradical heterocycloalkyl as defined above. The heterocycloalkylene may be a geminal, vicinal or isolated diradical. In certain embodiments, the heterocycloalkylene may be unsaturated (e.g., heterocycloalkenylene (i.e., a heterocycloalkylene group heterocycloalkyl as defined herein containing at least one carbon-carbon double bond) or heterocycloalkynylene (i.e., a heterocycloalkylene group heterocycloalkyl as defined herein containing at least one carbon-carbon triple bond)) but cannot be aromatic. In certain embodiments, the heterocycloalkylene (i) is monocyclic or polycyclic (such as bi- or tricyclic) and/or (ii) is 3- to 14-membered (i.e., 3-, 4-, 5-, 6-, 7-, 8-, 9-, 10-, 11-, 12-, 13-, or 14-membered, such as 3- to 12-membered or 3- to 10-membered). In one embodiment, the heterocycloalkylene is a mono-, bi- or tricyclic 3- to 14-membered (i.e., 3-, 4-, 5-, 6-, 7-, 8-, 9-, 10-, 11-, 12-, 13-, or 14-membered, such as 3- to 12-membered or 3- to 10-membered) heterocycloalkylene. Generally, instead of using the ending “ylene” for the heterocycloalkylene moieties as specified above, one can also use the ending “diyl” (e.g., 1,3-imidazolidinylene can also be called imidazolidin-1,3-diyl). Exemplary cycloalkylene groups include pyrrolidinylene, imidazolidinylene, 2,3-dihydro-1H-imidazolylene, pyrazolidinylene, piperidinylene, piperazinylene, and azetidinylene. A “substituted heterocycloalkylene” means that one or more (such as 1 to the maximum number of hydrogen atoms bound to an heterocycloalkylene group, e.g., 1, 2, 3, 4, 5, 6, 7, 8, 9, or up to 10, such as between 1 to 5, 1 to 4, or 1 to 3, or 1 or 2) hydrogen atoms of the heterocycloalkylene group are replaced with a substituent other than hydrogen (when more than one hydrogen atom is replaced the substituents may be the same or different). Preferably, the substituent other than hydrogen is a 1t level substituent as specified herein.

The expression “partially hydrogenated form” of an unsaturated compound or group as used herein means that part of the unsaturation has been removed by formally adding hydrogen to the initially unsaturated compound or group without removing all unsaturated moieties. The phrase “completely hydrogenated form” of an unsaturated compound or group is used herein interchangeably with the term “perhydro” and means that all unsaturation has been removed by formally adding hydrogen to the initially unsaturated compound or group. For example, partially hydrogenated forms of a 5-membered heteroaryl group (containing 2 double bonds in the ring, such as furan) include dihydro forms of said 5-membered heteroaryl group (such as 2,3-dihydrofuran or 2,5-dihydrofuran), whereas the tetrahydro form of said 5-membered heteroaryl group (e.g., tetrahydrofuran, i.e., THF) is a completely hydrogenated (or perhydro) form of said 5-membered heteroaryl group. Likewise, for a 6-membered heteroaryl group having 3 double bonds in the ring (such as pyridyl), partially hydrogenated forms include di- and tetrahydro forms (such as di- and tetrahydropyridyl), whereas the hexahydro form (such as piperidinyl in case of the heteroaryl pyridyl) is the completely hydrogenated (or perhydro) derivative of said 6-membered heteroaryl group. Consequently, a hexahydro form of an aryl or heteroaryl can only be considered a partially hydrogenated form according to the present disclosure if the aryl or heteroaryl contains at least 4 unsaturated moieties consisting of double and triple bonds between ring atoms.

The term “aromatic” as used in the context of hydrocarbons means that the whole molecule has to be aromatic. For example, if a monocyclic aryl is hydrogenated (either partially or completely) the resulting hydrogenated cyclic structure is classified as cycloalkyl for the purposes of the present disclosure.

Likewise, if a bi- or polycyclic aryl (such as naphthyl) is hydrogenated the resulting hydrogenated bi- or polycyclic structure (such as 1,2-dihydronaphthyl) is classified as cycloalkyl for the purposes of the present disclosure (even if one ring, such as in 1,2-dihydronaphthyl, is still aromatic). A similar distinction is made within the present disclosure between heteroaryl and heterocyclyl. For example, indolinyl, i.e., a dihydro variant of indolyl, is classified as heterocyclyl for the purposes of the present disclosure, since only one ring of the bicyclic structure is aromatic and one of the ring atoms is a heteroatom.

The term “hydrocarbyl” as used herein relates to a monovalent organic group obtained by removing one H atom from a hydrocarbon molecule. In some embodiments, hydrocarbyl groups are non-cyclic, e.g., linear (straight) or branched. Typical examples of hydrocarbyl groups include alkyl, alkenyl, alkynyl, cycloalkyl, aryl groups, and combinations thereof (such as arylalkyl(aralkyl), etc.). Particular examples of hydrocarbyl groups are C1-6 alkyl, aryl, and aryl(C₁₋₆ alkyl). In some embodiments, the hydrocarbyl group is optionally substituted (e.g., with one or more 1V level substituents as defined herein), provided that the overall polarity of the hydrocarbon remains relatively nonpolar.

The term “aliphatic” refers to any organic group which is not aromatic and, in particular, encompasses any non-aromatic hydrocarbyl group defined herein and the diradical versions of such non-aromatic hydrocarbyl groups. In some embodiments, where the aliphatic group is a monoradical, the aliphatic group encompasses the alkyl, alkenyl, alkynyl, and cycloalkyl groups defined herein (wherein each of the alkyl, alkenyl, alkynyl, and cycloalkyl groups may be optionally substituted). In some embodiments, where the aliphatic group is a diradical, the aliphatic group encompasses the alkylene, alkenylene, alkynylene, and cycloalkylene groups defined herein (wherein each of the alkylene, alkenylene, alkynylene, and cycloalkylene groups may be optionally substituted).

The term “cycloaliphatic” refers to any organic group which is cyclic and which is not aromatic and, in particular, encompasses any non-aromatic cyclic hydrocarbyl group defined herein and the diradical versions of such non-aromatic cyclic hydrocarbyl groups. In some embodiments, where the cycloaliphatic group is a monoradical, the cycloaliphatic group encompasses the cycloalkyl groups defined herein (wherein the cycloalkyl groups may be optionally substituted). In some embodiments, where the cycloaliphatic group is a diradical, the cycloaliphatic group encompasses cycloalkylene groups defined herein (wherein the cycloalkylene groups may be optionally substituted).

The term “heteroaliphatic” means an aliphatic group as defined herein in which one or more (e.g., 1, 2, 3, or 4) carbon atoms in the aliphatic group are replaced by heteroatoms of O, N, Si, Se, P, or S, preferably O, S, or N. In some embodiments, where the heteroaliphatic group is a monoradical, the heteroaliphatic group encompasses the alkyl, alkenyl, alkynyl, and heterocyclic groups defined herein (each of which may be optionally substituted), wherein in each of the alkyl, alkenyl, and alkynyl groups one or more (e.g., 1, 2, 3, or 4) carbon atoms are replaced by heteroatoms of O, N, Si, Se, P, or S, preferably O, S, or N. In some embodiments, where the heteroaliphatic group is a diradical, the heteroaliphatic group encompasses the alkylene, alkenylene, alkynylene, and heterocycloalkylene groups defined herein (each of which may be optionally substituted), wherein in each of the alkylene, alkenylene, and alkynylene groups one or more (e.g., 1, 2, 3, or 4) carbon atoms are replaced by heteroatoms of O, N, Si, Se, P, or S, preferably O, S, or N.

Typical 1^{st} level substituents are preferably selected from the group consisting of C₁₋₃ alkyl, phenyl, halogen, —CF₃, —OH, —OCH₃, —SCH₃, —NH_{2-z}(CH₃)_{z}, —C(═O)OH, and —C(═O)OCH₃, wherein z is 0, 1, or 2 and C₁₋₃ alkyl is methyl, ethyl, propyl or isopropyl. Particularly preferred 1 level substituents are selected from the group consisting of methyl, ethyl, propyl, isopropyl, halogen (such as F, Cl, or Br), and —CF₃, such as halogen (e.g., F, Cl, or Br), and —CF₃.

The term “tertiary amine moiety” as used herein relates to a moiety containing a nitrogen atom which is substituted with three organic substituents (wherein the substituents may be the same or different from each other). In some embodiments, the organic substituents are selected from hydrocarbyl groups (such as alkyl groups, in particular C₁₋₆ alkyl groups) which are optionally substituted (e.g., with one or more 1^{st} level substituents as defined herein).

The term “filtrating” as used herein relates to any process that involves removal or separation of at least one component (such as permeable molecules like salts, small proteins, solvents etc.,) of a liquid composition based on the molecular size of the components contained in the composition. This separation may use micro-molecule permeable filters (e.g., for diafiltration or tangential flow filtration) or semipermeable membranes (e.g., for dialysis). Thus, examples of filtrating comprise dialyzing, tangential flow filtrating and diafiltrating.

A “monovalent” compound relates to a compound having only one functional group of interest. For example, a monovalent acid relates to a compound having only one acid group (such as one carboxyl (—COOH) group). A monovalent cation, for example, relates to a compound having only one cationic group, such as an alkaline cation (e.g., Na⁺, K⁺, Li⁺), an ammonium cation (NH₄⁺) or an organic compound having one primary, secondary or tertiary amine group (like the protonated form of triethylamine, trimethylamine, etc.) an organic compound having one quaternary amine group.

A “divalent” or “dibasic” compound relates to a compound having two functional groups of interest. For example, a dibasic organic acid has two carboxyl groups.

A “polyvalent” or “polybasic” compound relates to a compound having three or more functional groups of interest. For example, a polybasic organic acid has three or more acid carboxyl groups.

The expression “substantially free of X”, as used herein, means that a mixture (such as an aqueous phase of a composition or formulation described herein) is free of X in such manner as it is practically and realistically feasible. For example, if the mixture is substantially free of X, the amount of X in the mixture may be less than 1% by weight (e.g., less than 0.5% by weight, less than 0.4% by weight, less than 0.3% by weight, less than 0.2% by weight, less than 0.1% by weight, less than 0.09% by weight, less than 0.08% by weight, less than 0.07% by weight, less than 0.06% by weight, less than 0.05% by weight, less than 0.04% by weight, less than 0.03% by weight, less than 0.02% by weight, less than 0.01% by weight, less than 0.005% by weight, less than 0.001% by weight), based on the total weight of the mixture.

For example, “substantially free of a cryoprotectant” as used herein, means that a mixture (such as an aqueous phase of a composition or formulation described herein) is free of a cryoprotectant in such manner as it is practically and realistically feasible. For example, if the mixture is substantially free of a cryoprotectant, the amount of a cryoprotectant in the mixture may be less than 1% by weight (e.g., less than 0.5% by weight, less than 0.4% by weight, less than 0.3% by weight, less than 0.2% by weight, less than 0.1% by weight, less than 0.09% by weight, less than 0.08% by weight, less than 0.07% by weight, less than 0.06% by weight, less than 0.05% by weight, less than 0.04% by weight, less than 0.03% by weight, less than 0.02% by weight, less than 0.01% by weight, less than 0.005% by weight, less than 0.001% by weight), based on the total weight of the mixture. Similar considerations apply to other expressions comprising the phrase “substantially free of”, such as “substantially free of any chelating agent”, “substantially free of EDTA”, “substantially free of the cationically ionizable lipid”, “substantially free of the additional lipids”, “substantially free of lipids”, “substantially free of nucleic acid”, and “substantially free of one or more unwanted substances”.

The expression “nucleic acid integrity” means the percentage of the full-length (i.e., non-fragmented) nucleic acid to the total amount of nucleic acid (i.e., non-fragmented plus fragmented nucleic acid) contained in a sample. The nucleic acid integrity may be determined by chromatographically separating the nucleic acid (e.g., using capillary electrophoresis), determining the peak area of the main nucleic acid peak (i.e., the peak area of the full-length (i.e., non-fragmented) nucleic acid), determining the peak area of the total nucleic acid, and dividing the peak area of the main nucleic acid peak by the peak area of the total nucleic acid. Likewise, the expression “RNA integrity” means the percentage of the full-length (i.e., non-fragmented) RNA to the total amount of RNA (i.e., non-fragmented plus fragmented RNA) contained in a sample. The RNA integrity may be determined by chromatographically separating the RNA (e.g., using capillary electrophoresis), determining the peak area of the main RNA peak (i.e., the peak area of the full-length (i.e., non-fragmented) RNA), determining the peak area of the total RNA, and dividing the peak area of the main RNA peak by the peak area of the total RNA.

The term “tonicity agent” as used herein means a compound which is usually added to injectable preparations to prevent osmotic shock at the site of injection upon administration, and thereby reduce local irritation. Typical tonicity agents include salts (e.g., saline), sugars (e.g., glucose, sucrose, dextrose or trehalose) and sugar alcohols (e.g., glycerin, or mannitol). “Tonicity” preferably is a colligative property that depends primarily on the number of dissolved particles in solution. Hence, the amount of tonicity agent to be added depends on the specific formulation. Typically, osmolality of 0.280 to 0.320 osmol is considered iso-osmotic.

The term “cryoprotectant” relates to a substance that is added to a preparation (e.g., formulation or composition) in order to protect the active ingredients of the preparation during the freezing stages. According to the present disclosure, the term “peptide” comprises oligo- and polypeptides and refers to substances which comprise about two or more, about 3 or more, about 4 or more, about 6 or more, about 8 or more, about 10 or more, about 13 or more, about 16 or more, about 20 or more, and up to about 50, about 100 or about 150, consecutive amino acids linked to one another via peptide bonds. The term “polypeptide” refers to large peptides, in particular peptides having at least about 151 amino acids. “Peptides” and “polypeptides” are both protein molecules, although the terms “protein” and “polypeptide” are used herein usually as synonyms.

A “therapeutic polypeptide” has a positive or advantageous effect on a condition or disease state of a subject when provided to the subject in a therapeutically effective amount. In some embodiments, a therapeutic polypeptide has curative or palliative properties and may be administered to ameliorate, relieve, alleviate, reverse, delay onset of or lessen the severity of one or more symptoms of a disease or disorder. A therapeutic polypeptide may have prophylactic properties and may be used to delay the onset of a disease or to lessen the severity of such disease or pathological condition. The term “therapeutic polypeptide” includes entire polypeptides or peptides, and can also refer to therapeutically active fragments thereof. It can also include therapeutically active variants of a polypeptide. Examples of therapeutically active polypeptides include, but are not limited to, antigens for vaccination and immunostimulants such as cytokines. The terms “therapeutic polypeptide” and “pharmaceutically active peptide or polypeptide” are used interchangeable herein.

According to various embodiments of the present disclosure, a nucleic acid such as RNA (e.g., mRNA) encoding a peptide, polypeptide or protein is taken up by or introduced, i.e. transfected or transduced, into a cell which cell may be present in vitro or in a subject, resulting in expression of said peptide, polypeptide or protein. The cell may express the encoded peptide, polypeptide or protein intracellularly (e.g. in the cytoplasm and/or in the nucleus), may secrete the encoded peptide, polypeptide or protein, and/or may express it on the surface.

According to the present disclosure, terms such as “nucleic acid expressing” and “nucleic acid encoding” or similar terms are used interchangeably herein and with respect to a particular peptide, polypeptide or protein mean that the nucleic acid, if present in the appropriate environment, preferably within a cell, can be expressed to produce said peptide, polypeptide or protein.

The term “portion” refers to a fraction. With respect to a particular structure such as an amino acid sequence or protein the term “portion” thereof may designate a continuous or a discontinuous fraction of said structure.

The terms “part” and “fragment” are used interchangeably herein and refer to a continuous element. For example, a part of a structure such as an amino acid sequence or protein refers to a continuous element of said structure. When used in context of a composition, the term “part” means a portion of the composition. For example, a part of a composition may any portion from 0.1% to 99.9% (such as 0.1%, 0.5%, 1%, 5%, 10%, 50%, 90%, or 99%) of said composition. “Fragment”, with reference to an amino acid sequence (peptide, polypeptide or protein), relates to a part of an amino acid sequence, i.e. a sequence which represents the amino acid sequence shortened at the N-terminus and/or C-terminus. A fragment shortened at the C-terminus (N-terminal fragment) is obtainable, e.g., by translation of a truncated open reading frame that lacks the 3′-end of the open reading frame. A fragment shortened at the N-terminus (C-terminal fragment) is obtainable, e.g., by translation of a truncated open reading frame that lacks the 5′-end of the open reading frame, as long as the truncated open reading frame comprises a start codon that serves to initiate translation. A fragment of an amino acid sequence comprises, e.g., at least 50%, at least 60%, at least 70%, at least 80%, at least 90% of the amino acid residues from an amino acid sequence. A fragment of an amino acid sequence preferably comprises at least 6, in particular at least 8, at least 12, at least 15, at least 20, at least 30, at least 50, or at least 100 consecutive amino acids from an amino acid sequence. A fragment of an amino acid sequence comprises, e.g., a sequence of up to 8, in particular up to 10, up to 12, up to 15, up to 20, up to 30 or up to 55, consecutive amino acids of the amino acid sequence.

According to the present disclosure, a part or fragment of a peptide, polypeptide or protein preferably has at least one functional property of the peptide, polypeptide or protein from which it has been derived. Such functional properties comprise a pharmacological activity, the interaction with other peptides, polypeptides or proteins, an enzymatic activity, the interaction with antibodies, and the selective binding of nucleic acids. E.g., a pharmacological active fragment of a peptide, polypeptide or protein has at least one of the pharmacological activities of the peptide, polypeptide or protein from which the fragment has been derived. “Variant”, as used herein and with reference to an amino acid sequence (peptide, polypeptide, or protein), is meant an amino acid sequence that differs from a parent amino acid sequence by virtue of at least one amino acid (e.g., a different amino acid, or a modification of the same amino acid). The parent amino acid sequence may be a naturally occurring or wild type (WT) amino acid sequence, or may be a modified version of a wild type amino acid sequence. In some embodiments, the variant amino acid sequence has at least one amino acid difference as compared to the parent amino acid sequence, e.g., from 1 to about 20 amino acid differences, and preferably from 1 to about 10 or from 1 to about 5 amino acid differences compared to the parent.

By “wild type” or “WT” or “native” with respect to an amino acid sequence is meant an amino acid sequence that is found in nature, including allelic variations. A wild type amino acid sequence, peptide, polypeptide or protein has an amino acid sequence that has not been intentionally modified. Likewise by “wild type” or “WT” or “native” with respect to a nucleic acid sequence is meant a nucleic acid sequence that is found in nature, including allelic variations. For example, a wild type coding sequence is meant to be a coding sequence that is found in nature and that has not been intentionally modified.

A “coding sequence”, as sued herein means the portion of a nucleic acid (e.g., a gene's DNA or RNA) that codes for protein.

The expression “guanosine/cytosine (G/C) content” or “G/C content” means the percentage of bases in a DNA or RNA molecule that are either guanine (G) or cytosine (C). The G/C content may be given for a specific portion of DNA or RNA or for an entire genome. When the G/C content refers to a portion, it may denote the G/C content of an individual gene or portion of a gene (domain), a group of genes or gene clusters, a non-coding region, a coding sequence, or a synthetic oligonucleotide such as a primer.

For the purposes of the present disclosure, “variants” of an amino acid sequence (peptide, protein or polypeptide) comprise amino acid insertion variants, amino acid addition variants, amino acid deletion variants and/or amino acid substitution variants. The term “variant” includes all mutants, splice variants, post-translationally modified variants, conformations, isoforms, allelic variants, species variants, and species homologs, in particular those which are naturally occurring. The term “variant” includes, in particular, fragments of an amino acid sequence.

In some embodiments, the degree of similarity, preferably identity, between a given amino acid sequence and an amino acid sequence which is a variant of said given amino acid sequence will be at least about 60%, 70%, 80%, 81%, 82%, 83%, 84%, 85%, 86%, 87%, 88%, 89%, 90%, 91%, 92%, 93%, 94%, 95%, 96%, 97%, 98%, or 99%, optionally at least 80%, preferably at least 90%, most preferably at least 95% (such as 99%). The degree of similarity or identity is given preferably for an amino acid region which is at least about 10%, at least about 20%, at least about 30%, at least about 40%, at least about 50%, at least about 60%, at least about 70%, at least about 80%, at least about 90% or about 100% of the entire length of the reference amino acid sequence. For example, if the reference amino acid sequence consists of 200 amino acids, the degree of similarity or identity is given preferably for at least about 20, at least about 40, at least about 60, at least about 80, at least about 100, at least about 120, at least about 140, at least about 160, at least about 180, or about 200 amino acids, in some embodiments continuous amino acids. In some embodiments, the degree of similarity or identity is given for the entire length of the reference amino acid sequence.

“Sequence similarity” indicates the percentage of amino acids that either are identical or that represent conservative amino acid substitutions. “Sequence identity” between two amino acid sequences indicates the percentage of amino acids that are identical between the sequences. “Sequence identity” between two nucleic acid sequences indicates the percentage of nucleotides that are identical between the sequences.

The terms “% identical” and “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.). In some embodiments, percent identity of two sequences is determined using the BLASTN or BLASTP algorithm, as available on the United States National Center for Biotechnology Information (NCBI) website (e.g., at blast.ncbi.nlm.nih.gov/Blast.cgi?PAGE_TYPE=BlastSearch&BLAST_SPEC=blast2seq&LINK_LOC=align2seq). In some embodiments, the algorithm parameters used for BLASTN algorithm on the NCBI website include: (i) Expect Threshold set to 10; (ii) Word Size set to 28; (iii) Max matches in a query range set to 0; (iv) Match/Mismatch Scores set to 1, -2; (v) Gap Costs set to Linear; and (vi) the filter for low complexity regions being used. In some embodiments, the algorithm parameters used for BLASTP algorithm on the NCBI website include: (i) Expect Threshold set to 10; (ii) Word Size set to 3; (iii) Max matches in a query range set to 0; (iv) Matrix set to BLOSUM62; (v) Gap Costs set to Existence: 11 Extension: 1; and (vi) conditional compositional score matrix adjustment.

Percentage identity is obtained by determining the number of identical positions at which the sequences to be compared correspond, dividing this number by the number of positions compared (e.g., the number of positions in the reference sequence) and multiplying this result by 100.

In some embodiments, a fragment or variant of an amino acid sequence (peptide, polypeptide or protein) is preferably a “functional fragment” or “functional variant”. The term “functional fragment” or “functional variant” of an amino acid sequence relates to any fragment or variant exhibiting one or more functional properties identical or similar to those of the amino acid sequence from which it is derived, i.e., it is functionally equivalent. With respect to antigens or antigenic sequences, one particular function is one or more immunogenic activities displayed by the amino acid sequence from which the fragment or variant is derived. The term “functional fragment” or “functional variant”, as used herein, in particular refers to a variant molecule or sequence that comprises an amino acid sequence that is altered by one or more amino acids compared to the amino acid sequence of the parent molecule or sequence and that is still capable of fulfilling one or more of the functions of the parent molecule or sequence, e.g., inducing an immune response (immunogenic fragment). In one embodiment, the modifications in the amino acid sequence of the parent molecule or sequence do not significantly affect or alter the characteristics of the molecule or sequence. In different embodiments, the function of the functional fragment or functional variant may be reduced but still significantly present, e.g., immunogenicity of the functional variant may be at least 50%, at least 60%, at least 70%, at least 80%, or at least 90% of the parent molecule or sequence. However, in other embodiments, immunogenicity of the functional fragment or functional variant may be enhanced compared to the parent molecule or sequence.

An amino acid sequence (peptide, protein or polypeptide) “derived from” a designated amino acid sequence (peptide, protein or polypeptide) refers to the origin of the first amino acid sequence. In some embodiments, the amino acid sequence which is derived from a particular amino acid sequence has an amino acid sequence that is identical, essentially identical or homologous to that particular sequence or a fragment thereof. Amino acid sequences derived from a particular amino acid sequence may be variants of that particular sequence or a fragment thereof. For example, it will be understood by one of ordinary skill in the art that the antigens suitable for use herein may be altered such that they vary in sequence from the naturally occurring or native sequences from which they were derived, while retaining the desirable activity of the native sequences.

In some embodiments, “isolated” means altered or removed (e.g., purified) from the natural state or from an artificial composition, such as a composition from a production process. For example, a nucleic acid or a peptide naturally present in a living animal is not “isolated”, but the same nucleic acid or peptide partially or completely separated from the coexisting materials of its natural state is “isolated”. An isolated nucleic acid or protein can exist in substantially purified form, or can exist in a non-native environment such as, for example, a host cell. In some embodiments, the RNA (such as mRNA) used in the present disclosure is in substantially purified form. In some embodiments, a solution (preferably an aqueous solution) of RNA (such as mRNA) in substantially purified form contains a first buffer system.

The term “genetic modification” or simply “modification” includes the transfection of cells with nucleic acid. The term “transfection” relates to the introduction of nucleic acids, in particular RNA, into a cell.

For purposes of the present disclosure, the term “transfection” also includes the introduction of a nucleic acid into a cell or the uptake of a nucleic acid by such cell, wherein the cell may be present in a subject, e.g., a patient. Thus, according to the present disclosure, a cell for transfection of a nucleic acid described herein can be present in vitro (e.g., in cell culture) or in vivo, e.g., the cell can form part of an organ, a tissue and/or an organism of a patient.

An “antigen” according to the present disclosure covers any substance that will elicit an immune response and/or any substance against which an immune response or an immune mechanism such as a cellular response is directed. This also includes situations wherein the antigen is processed into antigen peptides and an immune response or an immune mechanism is directed against one or more antigen peptides, in particular if presented in the context of MHC molecules. In particular, an “antigen” relates to any substance, preferably a peptide or protein, that reacts specifically with antibodies or T-lymphocytes (T-cells). According to the present disclosure, the term “antigen” comprises any molecule which comprises at least one epitope, such as a T cell epitope. Preferably, an antigen in the context of the present disclosure is a molecule which, optionally after processing, induces an immune reaction, which is preferably specific for the antigen (including cells expressing the antigen). In one embodiment, an antigen is a disease-associated antigen, such as a tumor antigen, a viral antigen, or a bacterial antigen, or an epitope derived from such antigen.

The term “disease-associated antigen” is used in its broadest sense to refer to any antigen associated with a disease. A disease-associated antigen is a molecule which contains epitopes that will stimulate a host's immune system to make a cellular antigen-specific immune response and/or a huImoral antibody response against the disease. Disease-associated antigens include pathogen-associated antigens, i.e., antigens which are associated with infection by microbes, typically microbial antigens (such as bacterial or viral antigens), or antigens associated with cancer, typically tumors, such as tumor antigens.

In some embodiments, the antigen is a tumor antigen, i.e., a part of a tumor cell, in particular those which primarily occur intracellularly or as surface antigens of tumor cells. In another embodiment, the antigen is a pathogen-associated antigen, i.e., an antigen derived from a pathogen, e.g., from a virus, bacterium, unicellular organism, or parasite, for example a viral antigen such as viral ribonucleoprotein or coat protein. In particular, the antigen should be presented by MHC molecules which results in modulation, in particular activation of cells of the immune system, preferably CD4+ and CD8+ lymphocytes, in particular via the modulation of the activity of a T-cell receptor.

The term “tumor antigen” or “tumor-associated antigen” refers to a constituent of cancer cells which may be derived from the cytoplasm, the cell surface or the cell nucleus. In particular, it refers to those antigens which are produced intracellularly or as surface antigens on tumor cells. For example, tumor antigens include the carcinoembryonal antigen, α1-fetoprotein, isoferritin, and fetal sulphoglycoprotein, α2-H-ferroprotein and γ-fetoprotein, as well as various virus tumor antigens. According to some embodiments of the present disclosure, a tumor antigen comprises any antigen which is characteristic for tumors or cancers as well as for tumor or cancer cells with respect to type and/or expression level.

The term “viral antigen” refers to any viral component having antigenic properties, i.e., being able to provoke an immune response in an individual. The viral antigen may be a viral ribonucleoprotein or an envelope protein.

The term “bacterial antigen” refers to any bacterial component having antigenic properties, i.e. being able to provoke an immune response in an individual. The bacterial antigen may be derived from the cell wall or cytoplasm membrane of the bacterium.

Terms such as “epitope”, “fragment of an antigen”, “immunogenic peptide” and “antigen peptide” are used interchangeably herein and, e.g., may relate to an incomplete representation of an antigen which is, e.g., capable of eliciting an immune response against the antigen or a cell expressing or comprising and presenting the antigen. In some embodiments, the terms relate to an immunogenic portion of an antigen. Preferably, it is a portion of an antigen that is recognized (i.e., specifically bound) by a T cell receptor, in particular if presented in the context of MHC molecules. Certain preferred immunogenic portions bind to an MHC class I or class II molecule. The term “epitope” refers to a part or fragment of a molecule such as an antigen that is recognized by the immune system. For example, the epitope may be recognized by T cells, B cells or antibodies. An epitope of an antigen may include a continuous or discontinuous portion of the antigen and may be between about 5 and about 100, such as between about 5 and about 50, more preferably between about 8 and about 30, most preferably between about 8 and about 25 amino acids in length, for example, the epitope may be preferably 9, 10, 11, 12, 13, 14, 15, 16, 17, 18, 19, 20, 21, 22, 23, 24, or 25 amino acids in length. In some embodiments, an epitope is between about 10 and about 25 amino acids in length.

The peptide and protein antigen can be 2 to 100 amino acids, including for example, 5 amino acids, 10 amino acids, 15 amino acids, 20 amino acids, 25 amino acids, 30 amino acids, 35 amino acids, 40 amino acids, 45 amino acids, or 50 amino acids in length. In some embodiments, a peptide can be greater than 50 amino acids. In some embodiments, the peptide can be greater than 100 amino acids.

The peptide or protein antigen can be any peptide or protein that can induce or increase the ability of the immune system to develop antibodies and T cell responses to the peptide or protein.

In some embodiments, vaccine antigen, i.e., an antigen whose inoculation into a subject induces an immune response, is recognized by an immune effector cell. In some embodiments, the vaccine antigen if recognized by an immune effector cell is able to induce in the presence of appropriate co-stimulatory signals, stimulation, priming and/or expansion of the immune effector cell carrying an antigen receptor recognizing the vaccine antigen. In the context of the embodiments of the present disclosure, the vaccine antigen is preferably presented or present on the surface of a cell, preferably an antigen presenting cell.

In some embodiments, an antigen is presented by a diseased cell (such as tumor cell or an infected cell).

The terms “immune response” and “immune reaction” are used herein interchangeably in their conventional meaning and refer to an integrated bodily response to an antigen and may refer to a cellular immune response, a huImoral immune response, or both. According to the disclosure, the term “immune response to” or “immune response against” with respect to an agent such as an antigen, cell or tissue, relates to an immune response such as a cellular response directed against the agent. An immune response may comprise one or more reactions selected from the group consisting of developing antibodies against one or more antigens and expansion of antigen-specific T-lymphocytes, such as CD4+ and CD8+T-lymphocytes, e.g., CD8+T-lymphocytes, which may be detected in various proliferation or cytokine production tests in vitro.

The terms “inducing an immune response” and “eliciting an immune response” and similar terms in the context of the present disclosure refer to the induction of an immune response, such as the induction of a cellular immune response, a huImoral immune response, or both. The immune response may be protective/preventive/prophylactic and/or therapeutic. The immune response may be directed against any immunogen or antigen or antigen peptide, preferably against a tumor-associated antigen or a pathogen-associated antigen (e.g., an antigen of a virus (such as influenza virus (A, B, or C), CMV or RSV)). “Inducing” in this context may mean that there was no immune response against a particular antigen or pathogen before induction, but it may also mean that there was a certain level of immune response against a particular antigen or pathogen before induction and after induction said immune response is enhanced. Thus, “inducing the immune response” in this context also includes “enhancing the immune response”. In some embodiments, after inducing an immune response in an individual, said individual is protected from developing a disease such as an infectious disease or a cancerous disease or the disease condition is ameliorated by inducing an immune response.

The terms “vaccination” and “immunization” describe the process of treating an individual for therapeutic or prophylactic reasons and relate to the procedure of administering one or more immunogen(s) or antigen(s) or derivatives thereof, in particular in the form of RNA (especially mRNA) coding therefor, as described herein to an individual and stimulating an immune response against said one or more immunogen(s) or antigen(s) or cells characterized by presentation of said one or more immunogen(s) or antigen(s).

In the context of the present disclosure, the term “transcription” relates to a process, wherein the genetic code in a DNA sequence is transcribed into RNA (especially mRNA). Subsequently, the RNA (especially mRNA) may be translated into peptide, polypeptide or protein.

With respect to RNA, the term “expression” or “translation” relates to the process in the ribosomes of a cell by which a strand of mRNA directs the assembly of a sequence of amino acids to make a peptide or protein.

A medical preparation, in particular kit, described herein may comprise instructional material or instructions. As used herein, “instructional material” or “instructions” includes a publication, a recording, a diagram, or any other medium of expression which can be used to communicate the usefulness of the compositions and methods of the present disclosure. The instructional material of the kit of the present disclosure may, for example, be affixed to a container which contains the compositions of the present disclosure or be shipped together with a container which contains the compositions.

Alternatively, the instructional material may be shipped separately from the container with the intention that the instructional material and the compositions be used cooperatively by the recipient.

The term “optional” or “optionally” as used herein means that the subsequently described event, circumstance or condition may or may not occur, and that the description includes instances where said event, circumstance, or condition occurs and instances in which it does not occur.

In the present specification, a structural formula of a compound may represent a certain isomer of said compound. It is to be understood, however, that the present disclosure includes all isomers such as geometrical isomers, optical isomers based on an asymmetrical carbon, stereoisomers, tautomers and the like which occur structurally and isomer mixtures and is not limited to the description of the formula.

Furthermore, in the present specification, a structural formula of a compound may represent a specific salt and/or solvate of said compound. It is to be understood, however, that the present disclosure includes all salts (e.g., pharmaceutically acceptable salts) and solvates (e.g., hydrates) and is not limited to the description of the specific salt and/or solvate. “Isomers” are compounds having the same molecular formula but differ in structure (“structural isomers”) or in the geometrical (spatial) positioning of the functional groups and/or atoms (“stereoisomers”). “Enantiomers” are a pair of stereoisomers which are non-superimposable mirror-images of each other. A “racemic mixture” or “racemate” contains a pair of enantiomers in equal amounts and is denoted by the prefix (+). “Diastereomers” are stereoisomers which are non-superimposable and which are not mirror-images of each other. “Tautomers” are structural isomers of the same chemical substance that spontaneously and reversibly interconvert into each other, even when pure, due to the migration of individual atoms or groups of atoms; i.e., the tautomers are in a dynamic chemical equilibrium with each other. An example of tautomers are the isomers of the keto-enol-tautomerism. “Conformers” are stereoisomers that can be interconverted just by rotations about formally single bonds, and include —in particular —those leading to different 3-dimensional forms of (hetero)cyclic rings, such as chair, half-chair, boat, and twist-boat forms of cyclohexane.

The term “solvate” as used herein refers to an addition complex of a dissolved material in a solvent (such as an organic solvent (e.g., an aliphatic alcohol (such as methanol, ethanol, n-propanol, isopropanol), acetone, acetonitrile, ether, and the like), water or a mixture of two or more of these liquids), wherein the addition complex exists in the form of a crystal or mixed crystal. The amount of solvent contained in the addition complex may be stoichiometric or non-stoichiometric. A “hydrate” is a solvate wherein the solvent is water.

The term “average diameter” refers to the mean hydrodynamic diameter of particles as measured by dynamic light scattering (DLS) with data analysis using the so-called cumulant algorithm, which provides as results the so-called Z_{average} with the dimension of a length, and the polydispersity index (PDI), which is dimensionless (Koppel, D., J. Chem. Phys. 57, 1972, pp 4814-4820, ISO 13321). Here “average diameter”, “diameter” or “size” for particles is used synonymously with this value of the Z_{average}.

In some embodiments, the “polydispersity index” is calculated based on dynamic light scattering measurements by the so-called cumulant analysis as mentioned in the definition of the “average diameter”. Under certain prerequisites, it can be taken as a measure of the size distribution of an ensemble of nanoparticles.

The “radius of gyration” (abbreviated herein as R_{g}) of a particle about an axis of rotation is the radial distance of a point from the axis of rotation at which, if the whole mass of the particle is assumed to be concentrated, its moment of inertia about the given axis would be the same as with its actual distribution of mass. Mathematically, R_{g} is the root mean square distance of the particle's components from either its center of mass or a given axis. For example, for a macromolecule composed of n mass elements, of masses m, (i=1, 2, 3, . . . , n), located at fixed distances s, from the center of mass, R_{g} is the square-root of the mass average of sᵢ² over all mass elements and can be calculated as follows:

$${R}_{g} = {( {\sum }_{i = 1}^{n} {m}_{i} \cdot {s}_{i}^{2} / {\sum }_{i = 1}^{n} {m}_{i} )}^{1 / 2}$$

The radius of gyration can be determined or calculated experimentally, e.g., by using light scattering. In particular, for small scattering vectors 4 the structure function S is defined as follows:

$$S ( \vec{q} ) \approx N \cdot ( 1 - \frac{{q}^{2} \cdot {R}_{g}^{2}}{3} )$$

wherein N is the number of components (Guinier's law).

The “hydrodynamic radius” (which is sometimes called “Stokes radius” or “Stokes-Einstein radius”) of a particle is the radius of a hypothetical hard sphere that diffuses at the same rate as said particle. The hydrodynamic radius is related to the mobility of the particle, taking into account not only size but also solvent effects. For example, a smaller charged particle with stronger hydration may have a greater hydrodynamic radius than a larger charged particle with weaker hydration. This is because the smaller particle drags a greater number of water molecules with it as it moves through the solution. Since the actual dimensions of the particle in a solvent are not directly measurable, the hydrodynamic radius may be defined by the Stokes-Einstein equation:

$${R}_{h} = \frac{{k}_{B} \cdot T}{6 \cdot π \cdot η \cdot D}$$

wherein k_{B} is the Boltzmann constant; T is the temperature; q is the viscosity of the solvent; and D is the diffusion coefficient. The diffusion coefficient can be determined experimentally, e.g., by using dynamic light scattering (DLS). Thus, one procedure to determine the hydrodynamic radius of a particle or a population of particles (such as the hydrodynamic radius of particles such as LNPs contained in a formulation or composition as disclosed herein or the hydrodynamic radius of a particle peak obtained from subjecting such a formulation or composition to field-flow fractionation) is to measure the DLS signal of said particle or population of particles (such as DLS signal of particles such as LNPs contained in a formulation or composition as disclosed herein or the DLS signal of a particle peak obtained from subjecting such a formulation or composition to field-flow fractionation).

The term “aggregate” as used herein relates to a cluster of particles, wherein the particles are identical or very similar and adhere to each other in a non-covalently manner (e.g., via ionic interactions, H bridge interactions, dipole interactions, and/or van der Waals interactions).

The expression “light scattering” as used herein refers to the physical process where light is forced to deviate from a straight trajectory by one or more paths due to localized non-uniformities in the medium through which the light passes.

The term “UV” means ultraviolet and designates a band of the electromagnetic spectrum with a wavelength from 10 nm to 400 nm, i.e., shorter than that of visible light but longer than X-rays.

The expression “multi-angle light scattering” or “MALS” as used herein relates to a technique for measuring the light scattered by a sample into a plurality of angles. “Multi-angle” means in this respect that scattered light can be detected at different discrete angles as measured, for example, by a single detector moved over a range including the specific angles selected or an array of detectors fixed at specific angular locations. In one preferred embodiment, the light source used in MALS is a laser source (MALLS: multi-angle laser light scattering). Based on the MALS signal of a composition comprising particles and by using an appropriate formalism (e.g., Zimm plot, Berry plot, or Debye plot), it is possible to determine the radius of gyration (R_{g}) and, thus, the size of said particles. Preferably, the Zimm plot is a graphical presentation using the following equation:

$$\frac{{R}_{θ}}{{K}^{*} c} = {M}_{w} P ( θ ) - 2 {A}_{2} {cM}_{w}^{2} {P}^{2} ( θ )$$

wherein c is the mass concentration of the particles in the solvent (g/mL); A₂ is the second virial coefficient (mol·mL/g²); P(θ) is a form factor relating to the dependence of scattered light intensity on angle; R_{θ} is the excess Rayleigh ratio (cm⁻¹); and K* is an optical constant that is equal to 4π²η₀ (dn/dc)²λ₀⁻⁴N_{A}⁻¹, where η₀ is the refractive index of the solvent at the incident radiation (vacuum) wavelength, λ₀ is the incident radiation (vacuum) wavelength (nm), N_{A} is Avogadro's number (mol⁻¹), and dn dc is the differential refractive index increment (mL/g) (cf., e.g., Buchholz et al. (Electrophoresis 22 (2001), 4118-4128); B. H. Zimm (J. Chem. Phys. 13 (1945), 141; P. Debye (J. Appl. Phys. 15 (1944): 338; and W. Burchard (Anal. Chem. 75 (2003), 4279-4291). Preferably, the Berry plot is calculated the following term or the reciprocal thereof:

$$\sqrt{\frac{{R}_{θ}}{{K}^{*} c}}$$

wherein c, R_{θ} and K* are as defined above. Preferably, the Debye plot is calculated the following term or the reciprocal thereof:

$$\frac{{K}^{*} c}{{R}_{θ}}$$

wherein c, R_{θ} and K* are as defined above.

The expression “dynamic light scattering” or “DLS” as used herein refers to a technique to determine the size and size distribution profile of particles, in particular with respect to the hydrodynamic radius of the particles. A monochromatic light source, usually a laser, is shot through a polarizer and into a sample. The scattered light then goes through a second polarizer where it is detected and the resulting image is projected onto a screen. The particles in the solution are being hit with the light and diffract the light in all directions. The diffracted light from the particles can either interfere constructively (light regions) or destructively (dark regions). This process is repeated at short time intervals and the resulting set of speckle patterns are analyzed by an autocorrelator that compares the intensity of light at each spot over time.

The expression “static light scattering” or “SLS” as used herein refers to a technique to determine the size and size distribution profile of particles, in particular with respect to the radius of gyration of the particles, and/or the molar mass of particles. A high-intensity monochromatic light, usually a laser, is launched in a solution containing the particles. One or many detectors are used to measure the scattering intensity at one or many angles. The angular dependence is needed to obtain accurate measurements of both molar mass and size for all macromolecules of radius. Hence simultaneous measurements at several angles relative to the direction of incident light, known as multi-angle light scattering (MALS) or multi-angle laser light scattering (MALLS), is generally regarded as the standard implementation of static light scattering.

### Nucleic Acids

The term “nucleic acid” comprises deoxyribonucleic acid (DNA), ribonucleic acid (RNA), combinations thereof, and modified forms thereof. The term comprises genomic DNA, cDNA, mRNA, recombinantly produced and chemically synthesized molecules. A nucleic acid may be present as a single-stranded or double-stranded and linear or covalently circularly closed molecule. A nucleic acid can be isolated. The term “isolated nucleic acid” means, according to the present disclosure, that the nucleic acid (i) was amplified in vitro, for example via polymerase chain reaction (PCR) for DNA or in vitro transcription (using, e.g., an RNA polymerase) for RNA, (ii) was produced recombinantly by cloning, (iii) was purified, for example, by cleavage and separation by gel electrophoresis, or (iv) was synthesized, for example, by chemical synthesis. The nucleic acid may be mRNA, self-amplifying RNA (saRNA) or trans-amplifying RNA (taRNA), preferably mRNA. In some embodiments, the nucleic acid may be an RNA which comprises a coding region (such as mRNA) and an inhibitory region (such as miRNA).

The term “nucleoside” (abbreviated herein as “N”) relates to compounds which can be thought of as nucleotides without a phosphate group. While a nucleoside is a nucleobase linked to a sugar (e.g., ribose or deoxyribose), a nucleotide is composed of a nucleoside and one or more phosphate groups. Examples of nucleosides include cytidine, uridine, pseudouridine, adenosine, and guanosine.

The five standard nucleosides which usually make up naturally occurring nucleic acids are uridine, adenosine, thymidine, cytidine and guanosine. The five nucleosides are commonly abbreviated to their one letter codes U, A, T, C and G, respectively. However, thymidine is more commonly written as “dT” (“d” represents “deoxy”) as it contains a 2′-deoxyribofuranose moiety rather than the ribofuranose ring found in uridine. This is because thymidine is found in deoxyribonucleic acid (DNA) and not ribonucleic acid (RNA). Conversely, uridine is found in RNA and not DNA. The remaining three nucleosides may be found in both RNA and DNA. In RNA, they would be represented as A, C and G, whereas in DNA they would be represented as dA, dC and dG.

A modified purine (A or G) or pyrimidine (C, T, or U) base moiety is preferably modified by one or more alkyl groups, more preferably one or more C₁₋₄ alkyl groups, even more preferably one or more methyl groups. Particular examples of modified purine or pyrimidine base moieties include N⁷-alkyl-guanine, N⁶-alkyl-adenine, 5-alkyl-cytosine, 5-alkyl-uracil, and N(1)-alkyl-uracil, such as N⁷-C₁₋₄ alkyl-guanine, N⁶—C₁0.4 alkyl-adenine, 5-C₁₋₄ alkyl-cytosine, 5-C₁₋₄ alkyl-uracil, and N(1)-C₁₋₄ alkyl-uracil, preferably N⁷-methyl-guanine, N⁶-methyl-adenine, 5-methyl-cytosine, 5-methyl-uracil, and N(1)-methyl-uracil.

In some embodiments of all aspects of the disclosure, the nucleic acid is DNA.

Herein, the term “DNA” relates to a nucleic acid molecule which includes deoxyribonucleotide residues. In preferred embodiments, the DNA contains all or a majority of deoxyribonucleotide residues. As used herein, “deoxyribonucleotide” refers to a nucleotide which lacks a hydroxyl group at the 2′-position of a β-D-ribofuranosyl group. DNA encompasses without limitation, double stranded DNA, single stranded DNA, isolated DNA such as partially purified DNA, essentially pure DNA, synthetic DNA, recombinantly produced DNA, as well as modified DNA that differs from naturally occurring DNA by the addition, deletion, substitution and/or alteration of one or more nucleotides. Such alterations may refer to addition of non-nucleotide material to internal DNA nucleotides or to the end(s) of DNA. It is also contemplated herein that nucleotides in DNA may be non-standard nucleotides, such as chemically synthesized nucleotides or ribonucleotides. For the present disclosure, these altered DNAs are considered analogs of naturally-occurring DNA. A molecule contains “a majority of deoxyribonucleotide residues” if the content of deoxyribonucleotide residues in the molecule is more than 50% (such as at least 55%, at least 60%, at least 65%, at least 70%, at least 75%, at least 80%, at least 85%, at least 90%, at least 95%, at least 96%, at least 97%, at least 98%, at least 99%), based on the total number of nucleotide residues in the molecule. The total number of nucleotide residues in a molecule is the sum of all nucleotide residues (irrespective of whether the nucleotide residues are standard (i.e., naturally occurring) nucleotide residues or analogs thereof).

DNA may be recombinant DNA and may be obtained by cloning of a nucleic acid, in particular cDNA. The cDNA may be obtained by reverse transcription of RNA.

### RNA

In some embodiments of all aspects of the disclosure, the nucleic acid is RNA.

According to the present disclosure, the term “RNA” means a nucleic acid molecule which includes ribonucleotide residues. In preferred embodiments, the RNA contains all or a majority of ribonucleotide residues. As used herein, “ribonucleotide” refers to a nucleotide with a hydroxyl group at the 2′-position of a β-D-ribofuranosyl group. RNA encompasses without limitation, double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations may refer to addition of non-nucleotide material to internal RNA nucleotides or to the end(s) of RNA. It is also contemplated herein that nucleotides in RNA may be non-standard nucleotides, such as chemically synthesized nucleotides or deoxynucleotides. For the present disclosure, these altered/modified nucleotides (or modified nucleosides) can be referred to as analogs of naturally occurring nucleotides (nucleosides), and the corresponding RNAs containing such altered/modified nucleotides or nucleosides (i.e., altered/modified RNAs) can be referred to as analogs of naturally occurring RNAs. A molecule contains “a majority of ribonucleotide residues” if the content of ribonucleotide residues in the molecule is more than 50% (such as at least 55%, at least 60%, at least 65%, at least 70%, at least 75%, at least 80%, at least 85%, at least 90%, at least 95%, at least 96%, at least 97%, at least 98%, at least 99%), based on the total number of nucleotide residues in the molecule. The total number of nucleotide residues in a molecule is the sum of all nucleotide residues (irrespective of whether the nucleotide residues are standard (i.e., naturally occurring) nucleotide residues or analogs thereof).

“RNA” includes mRNA, tRNA, ribosomal RNA (rRNA), small nuclear RNA (snRNA), self-amplifying RNA (saRNA), trans-amplifying RNA (taRNA), single-stranded RNA (ssRNA), dsRNA, inhibitory RNA (such as antisense ssRNA, small interfering RNA (siRNA), or microRNA (miRNA)), activating RNA (such as small activating RNA) and immunostimulatory RNA (isRNA). In some embodiments, “RNA” refers to mRNA.

In a preferred embodiment, the RNA comprises an open reading frame (ORF) encoding a peptide, polypeptide or protein.

The term “in vitro transcription” or “IVT” as used herein means that the transcription (i.e., the generation of RNA) is conducted in a cell-free manner. I.e., IVT does not use living/cultured cells but rather the transcription machinery extracted from cells (e.g., cell lysates or the isolated components thereof, including an RNA polymerase (preferably T7, T3 or SP6 polymerase)).

mRNA

In some embodiments of all aspects of the disclosure, the nucleic acid is mRNA.

According to the present disclosure, the term “mRNA” means “messenger-RNA” and includes a “transcript” which may be generated by using a DNA template. Generally, mRNA encodes a peptide, polypeptide or protein. Typically, an mRNA comprises a 5′-UTR, a peptide/protein coding region, and a 3′-UTR. In the context of the present disclosure, mRNA is preferably generated by in vitro transcription (IVT) from a DNA template. As set forth above, the in vitro transcription methodology is known to the skilled person, and a variety of in vitro transcription kits is commercially available. mRNA is single-stranded but may contain self-complementary sequences that allow parts of the mRNA to fold and pair with itself to form double helices.

According to the present disclosure, “dsRNA” means double-stranded RNA and is RNA with two partially or completely complementary strands.

In preferred embodiments of the present disclosure, the mRNA relates to an RNA transcript which encodes a peptide, polypeptide or protein.

In some embodiments, the RNA which preferably encodes a peptide, polypeptide or protein has a length of at least 45 nucleotides (such as at least 60, at least 90, at least 100, at least 200, at least 300, at least 400, at least 500, at least 600, at least 700, at least 800, at least 900, at least 1,000, at least 1,500, at least 2,000, at least 2,500, at least 3,000, at least 3,500, at least 4,000, at least 4,500, at least 5,000, at least 6,000, at least 7,000, at least 8,000, at least 9,000 nucleotides), preferably up to 15,000, such as up to 14,000, up to 13,000, up to 12,000 nucleotides, up to 11,000 nucleotides or up to 10,000 nucleotides.

As established in the art, the RNA (such as mRNA) generally contains a 5′ untranslated region (5′-UTR), a peptide/polypeptide/protein coding region and a 3′ untranslated region (3′-UTR). In some embodiments, the RNA (such as mRNA) is produced by in vitro transcription or chemical synthesis. In one embodiment, the RNA (such as mRNA) is produced by in vitro transcription using a DNA template.

The in vitro transcription methodology is known to the skilled person; cf., e.g., Molecular Cloning: A Laboratory Manual, 2^{nd} Edition, J. Sambrook et al. eds., Cold Spring Harbor Laboratory Press, Cold Spring Harbor 1989. Furthermore, a variety of in vitro transcription kits is commercially available, e.g., from Thermo Fisher Scientific (such as TranscriptAid™ T7 kit, MEGAscript® T7 kit, MAXIscript®), New England BioLabs Inc. (such as HiScribe™ T7 kit, HiScribe™ T7 ARCA mRNA kit), Promega (such as RiboMAX™, HeLaScribe®, Riboprobe® systems), Jena Bioscience (such as SP6 or T7 transcription kits), and Epicentre (such as AmpliScribe™). For providing modified RNA (such as mRNA), correspondingly modified nucleotides, such as modified naturally occurring nucleotides, non-naturally occurring nucleotides and/or modified non-naturally occurring nucleotides, can be incorporated during synthesis (preferably in vitro transcription), or modifications can be effected in and/or added to the mRNA after transcription.

In some embodiments, RNA (such as mRNA) is in vitro transcribed RNA (IVT-RNA) and may be obtained by in vitro transcription of an appropriate DNA template. The promoter for controlling transcription can be any promoter for any RNA polymerase. Particular examples of RNA polymerases are the T7, T3, and SP6 RNA polymerases. Preferably, the in vitro transcription is controlled by a T7 or SP6 promoter. A DNA template for in vitro transcription may be obtained by cloning of a nucleic acid, in particular cDNA, and introducing it into an appropriate vector for in vitro transcription. The cDNA may be obtained by reverse transcription of RNA.

In some embodiments of the present disclosure, the RNA (such as mRNA) is “replicon RNA” (such as “replicon mRNA”) or simply a “replicon”, in particular “self-replicating RNA” (such as “self-replicating mRNA”) or “self-amplifying RNA” (or “self-amplifying mRNA”). In one particularly preferred embodiment, the replicon or self-replicating RNA (such as self-replicating mRNA) is derived from or comprises elements derived from an ssRNA virus, in particular a positive-stranded ssRNA virus such as an alphavirus. Alphaviruses are typical representatives of positive-stranded RNA viruses. Alphaviruses replicate in the cytoplasm of infected cells (for review of the alphaviral life cycle see José et al., Future Microbiol., 2009, vol. 4, pp. 837-856). The total genome length of many alphaviruses typically ranges between 11,000 and 12,000 nucleotides, and the genomic RNA typically has a 5′-cap, and a 3′ poly(A) tail. The genome of alphaviruses encodes non-structural proteins (involved in transcription, modification and replication of viral RNA and in protein modification) and structural proteins (forming the virus particle). There are typically two open reading frames (ORFs) in the genome. The four non-structural proteins (nsP1-nsP4) are typically encoded together by a first ORF beginning near the 5′ terminus of the genome, while alphavirus structural proteins are encoded together by a second ORF which is found downstream of the first ORF and extends near the 3′ terminus of the genome. Typically, the first ORF is larger than the second ORF, the ratio being roughly 2:1. In cells infected by an alphavirus, only the nucleic acid sequence encoding non-structural proteins is translated from the genomic RNA, while the genetic information encoding structural proteins is translatable from a subgenomic transcript, which is an RNA molecule that resembles eukaryotic messenger RNA (mRNA; Gould et al., 2010, Antiviral Res., vol. 87 pp. 111-124). Following infection, i.e. at early stages of the viral life cycle, the (+) stranded genomic RNA directly acts like a messenger RNA for the translation of the open reading frame encoding the non-structural poly-protein (nsP1234). Alphavirus-derived vectors have been proposed for delivery of foreign genetic information into target cells or target organisms. In simple approaches, the open reading frame encoding alphaviral structural proteins is replaced by an open reading frame encoding a protein of interest. Alphavirus-based trans-replication systems rely on alphavirus nucleotide sequence elements on two separate nucleic acid molecules: one nucleic acid molecule encodes a viral replicase, and the other nucleic acid molecule is capable of being replicated by said replicase in trans (hence the designation trans-replication system). Trans-replication requires the presence of both these nucleic acid molecules in a given host cell. The nucleic acid molecule capable of being replicated by the replicase in trans must comprise certain alphaviral sequence elements to allow recognition and RNA synthesis by the alphaviral replicase.

In some embodiments of the present disclosure, the RNA (such as mRNA) described herein (e.g., contained in the compositions of the present disclosure and/or used in the methods of the present disclosure) contains one or more modifications, e.g., in order to increase its stability and/or increase translation efficiency and/or decrease immunogenicity and/or decrease cytotoxicity. For example, in order to increase expression of the RNA (such as mRNA), it may be modified within the coding region, i.e., the sequence encoding the expressed peptide or protein, preferably without altering the sequence of the expressed peptide or protein. Such modifications are described, for example, in WO 2007/036366 and PCT/EP2019/056502, and include the following: a 5′-cap structure; an extension or truncation of the naturally occurring poly(A) tail; an alteration of the 5′- and/or 3′-untranslated regions (UTR) such as introduction of a UTR which is not related to the coding region of said RNA; the replacement of one or more naturally occurring nucleotides with synthetic nucleotides; and codon optimization (e.g., to alter, preferably increase, the G/C content of the RNA). The term “modification” in the context of modified mRNA according to the present disclosure preferably relates to any modification of an mRNA which is not naturally present in said RNA (such as mRNA).

In some embodiments, the RNA (such as mRNA) described herein comprises a 5′-cap structure. In one embodiment, the mRNA does not have uncapped 5′-triphosphates. In one embodiment, the RNA (such as mRNA) described herein may comprise a conventional 5′-cap and/or a 5′-cap analog. The term “conventional 5′-cap” refers to a cap structure found on the 5′-end of an mRNA molecule and generally consists of a guanosine 5′-triphosphate (Gppp) which is connected via its triphosphate moiety to the 5′-end of the next nucleotide of the mRNA (i.e., the guanosine is connected via a 5′ to 5′ triphosphate linkage to the rest of the mRNA). The guanosine may be methylated at position N⁷ (resulting in the cap structure m⁷ Gppp). The term “5′-cap analog” refers to a 5′-cap which is based on a conventional 5′-cap but which has been modified at either the 2′- or 3′-position of the m⁷ guanosine structure in order to avoid an integration of the 5′-cap analog in the reverse orientation (such 5′-cap analogs are also called anti-reverse cap analogs (ARCAs)). Particularly preferred 5′-cap analogs are those having one or more substitutions at the bridging and non-bridging oxygen in the phosphate bridge, such as phosphorothioate modified 5′-cap analogs at the β-phosphate (such as m₂^{7,2O} G(5′)ppSp(5′)G (referred to as beta-S-ARCA or β-S-ARCA)). Particularly preferred 5′-cap analogs are described in WO 2008/157688, WO 2011/015347, and WO 2019/175356. Providing an RNA (such as mRNA) with a 5′-cap structure as described herein may be achieved by in vitro transcription of a DNA template in presence of a corresponding 5′-cap compound, wherein said 5′-cap structure is co-transcriptionally incorporated into the generated RNA (such as mRNA) strand, or the RNA (such as mRNA) may be generated, for example, by in vitro transcription, and the 5′-cap structure may be attached to the mRNA post-transcriptionally using capping enzymes, for example, capping enzymes of vaccinia virus.

In some embodiments, the RNA (such as mRNA) comprises a cap0, cap1, or cap2, preferably cap1 or cap2. According to the present disclosure, the term “cap0” means the structure “m⁷ GpppN”, wherein N is any nucleoside bearing an OH moiety at position 2′. According to the present disclosure, the term “cap1” means the structure “m⁷GpppNm”, wherein Nm is any nucleoside bearing an OCH₃ moiety at position 2′. According to the present disclosure, the term “cap2” means the structure “m⁷ GpppNmNm”, wherein each Nm is independently any nucleoside bearing an OCH₃ moiety at position 2′.

As used herein, the term “poly-A tail” or “poly-A sequence” refers to an uninterrupted or interrupted sequence of adenylate residues which is typically located at the 3′-end of an RNA (such as mRNA) molecule. Poly-A tails or poly-A sequences are known to those of skill in the art and may follow the 3′-UTR in the RNAs (such as mRNAs) described herein. An uninterrupted poly-A tail is characterized by consecutive adenylate residues.

The poly-A tail may be of any length. In some embodiments, a poly-A tail comprises, essentially consists of, or consists of at least 20, at least 30, at least 40, at least 80, or at least 100 and up to 500, up to 400, up to 300, up to 200, or up to 150 A nucleotides, and, in particular, about 120 A nucleotides.

In some embodiments, a poly-A tail is attached during RNA transcription, e.g., during preparation of in vitro transcribed RNA, based on a DNA template comprising repeated dT nucleotides (deoxythymidylate) in the strand complementary to the coding strand. The DNA sequence encoding a poly-A tail (coding strand) is referred to as poly(A) cassette.

In some embodiments, the poly(A) cassette present in the coding strand of DNA essentially consists of dA nucleotides, but is interrupted by a random sequence of the four nucleotides (dA, dC, dG, and dT).

Such random sequence may be 5 to 50, 10 to 30, or 10 to 20 nucleotides in length. Such a cassette is disclosed in WO 2016/005324 A1, hereby incorporated by reference. Any poly(A) cassette disclosed in WO 2016/005324 A1 may be used in the present disclosure. Consequently, in some embodiments, the poly-A tail contained in an RNA (in particular, mRNA) molecule described herein essentially consists of A nucleotides, but is interrupted by a random sequence of the four nucleotides (A, C, G, U). Such random sequence may be 5 to 50, 10 to 30, or 10 to 20 nucleotides in length.

In some embodiments, no nucleotides other than A nucleotides flank a poly-A tail at its 3′-end, i.e., the poly-A tail is not masked or followed at its 3′-end by a nucleotide other than A.

In some embodiments, a poly-A tail may comprise at least 20, at least 30, at least 40, at least 80, or at least 100 and up to 500, up to 400, up to 300, up to 200, or up to 150 nucleotides. In some embodiments, the poly-A tail may essentially consist of at least 20, at least 30, at least 40, at least 80, or at least 100 and up to 500, up to 400, up to 300, up to 200, or up to 150 nucleotides. In some embodiments, the poly-A tail may consist of at least 20, at least 30, at least 40, at least 80, or at least 100 and up to 500, up to 400, up to 300, up to 200, or up to 150 nucleotides. In some embodiments, the poly-A tail comprises at least 100 nucleotides. In some embodiments, the poly-A tail comprises about 150 nucleotides. In some embodiments, the poly-A tail comprises about 120 nucleotides. In some embodiments, the poly-A tail comprises or consists of the nucleotide sequence of SEQ ID NO: 3. In some embodiments, the poly-A sequence has a nucleotide sequence having at least 99%, 98%, 97%, 96%, 95%, 90%, 85%, or 80% identity to the nucleotide sequence of SEQ ID NO: 3.

In some embodiments, RNA (such as mRNA) used in present disclosure comprises a 5′-UTR and/or a 3′-UTR. The term “untranslated region” or “UTR” relates to a region in a DNA molecule which is transcribed but is not translated into an amino acid sequence, or to the corresponding region in an RNA molecule, such as an mRNA molecule. An untranslated region (UTR) can be present 5′ (upstream) of an open reading frame (5′-UTR) and/or 3′ (downstream) of an open reading frame (3′-UTR). A 5′-UTR, if present, is located at the 5′-end, upstream of the start codon of a protein-encoding region. A 5′-UTR is downstream of the 5′-cap (if present), e.g., directly adjacent to the 5′-cap. A 3′-UTR, if present, is located at the 3′-end, downstream of the termination codon of a protein-encoding region, but the term “3′-UTR” does preferably not include the poly-A sequence. Thus, the 3′-UTR is upstream of the poly-A sequence (if present), e.g., directly adjacent to the poly-A sequence. Incorporation of a 3′-UTR into the 3′-non translated region of an RNA (preferably mRNA) molecule can result in an enhancement in translation efficiency.

In some embodiments, the RNA (such as mRNA) used in present disclosure comprises a 5′-UTR comprising the nucleotide sequence of SEQ ID NO: 1, or a nucleotide sequence having at least 99%, 98%, 97%, 96%, 95%, 90%, 85%, or 80% identity to the nucleotide sequence of SEQ ID NO: 1. In some embodiments, the RNA (such as mRNA) used in present disclosure comprises a 3′-UTR comprising the nucleotide sequence of SEQ ID NO: 2, or a nucleotide sequence having at least 99%, 98%, 97%, 96%, 95%, 90%, 85%, or 80% identity to the nucleotide sequence of SEQ ID NO: 2.

The RNA (such as mRNA) described herein may have modified ribonucleotides in order to increase its stability and/or decrease immunogenicity and/or decrease cytotoxicity. For example, in some embodiments, uridine in the RNA (such as mRNA) described herein is replaced (partially or completely, preferably completely) by a modified nucleoside. In some embodiments, the modified nucleoside is a modified uridine.

In some embodiments, the modified uridine replacing uridine is selected from the group consisting of pseudouridine (ψ), Ni-methyl-pseudouridine (mlψ), 5-methyl-uridine (m5U), and combinations thereof.

An RNA (preferably mRNA) which is modified by pseudouridine (replacing partially or completely, preferably completely, uridine) is referred to herein as “β-modified”, whereas the term “m 1W-modified” means that the RNA (preferably mRNA) contains N(1)-methylpseudouridine (replacing partially or completely, preferably completely, uridine). Furthermore, the term “m5U-modified” means that the RNA (preferably mRNA) contains 5-methyluridine (replacing partially or completely, preferably completely, uridine). Such T- or m1T- or m5U-modified RNAs usually exhibit decreased immunogenicity compared to their unmodified forms and, thus, are preferred in applications where the induction of an immune response is to be avoided or minimized. In some embodiments, the RNA (preferably mRNA) contains N(1)-methylpseudouridine replacing completely uridine.

The codons of the RNA (preferably mRNA) described in the present disclosure may further be optimized, e.g., to increase the G/C content of the RNA and/or to replace codons which are rare in the cell (or subject) in which the peptide or protein of interest is to be expressed by codons which are synonymous frequent codons in said cell (or subject). In some embodiments, the amino acid sequence encoded by the RNA described in the present disclosure is encoded by a coding sequence which is codon-optimized and/or the G/C content of which is increased compared to wild type coding sequence.

This also includes embodiments, wherein one or more sequence regions of the coding sequence are codon-optimized and/or increased in the G/C content compared to the corresponding sequence regions of the wild type coding sequence. In one embodiment, the codon-optimization and/or the increase in the G/C content preferably does not change the sequence of the encoded amino acid sequence.

The term “codon-optimized” refers to the alteration of codons in the coding region of a nucleic acid molecule to reflect the typical codon usage of a host organism without preferably altering the amino acid sequence encoded by the nucleic acid molecule. Within the context of the present disclosure, coding regions are preferably codon-optimized for optimal expression in a subject to be treated using the RNA (preferably mRNA) described herein. Codon-optimization is based on the finding that the translation efficiency is also determined by a different frequency in the occurrence of tRNAs in cells. Thus, the sequence of RNA (preferably mRNA) may be modified such that codons for which frequently occurring tRNAs are available are inserted in place of “rare codons”.

In some embodiments, the guanosine/cytosine (G/C) content of the coding region of the RNA (preferably mRNA) described herein is increased compared to the G/C content of the corresponding coding sequence of the wild type RNA, wherein the amino acid sequence encoded by the RNA (preferably mRNA) is preferably not modified compared to the amino acid sequence encoded by the wild type RNA. This modification of the RNA sequence is based on the fact that the sequence of any RNA region to be translated is important for efficient translation of that RNA (preferably mRNA). Sequences having an increased G (guanosine)/C (cytosine) content are more stable than sequences having an increased A (adenosine)/U (uracil) content. In respect to the fact that several codons code for one and the same amino acid (so-called degeneration of the genetic code), the most favorable codons for the stability can be determined (so-called alternative codon usage). Depending on the amino acid to be encoded by the RNA (preferably mRNA), there are various possibilities for modification of the RNA sequence, compared to its wild type sequence. In particular, codons which contain A and/or U nucleotides can be modified by substituting these codons by other codons, which code for the same amino acids but contain no A and/or U or contain a lower content of A and/or U nucleotides.

In various embodiments, the G/C content of the coding region of the RNA (in particular, mRNA) described herein is increased by at least 10%, at least 20%, at least 30%, at least 40%, at least 50%, at least 55%, or even more compared to the G/C content of the coding region of the wild type RNA.

A combination of the above described modifications, i.e., incorporation of a 5′-cap structure, incorporation of a poly-A sequence, unmasking of a poly-A sequence, alteration of the 5′- and/or 3′-UTR (such as incorporation of one or more 3′-UTRs), replacing one or more naturally occurring nucleotides with synthetic nucleotides (e.g., 5-methylcytidine for cytidine and/or pseudouridine (T) or N(1)-methylpseudouridine (m1ψ) or 5-methyluridine (m5U) for uridine), and codon optimization, has a synergistic influence on the stability of RNA (preferably mRNA) and increase in translation efficiency.

Thus, in some embodiments, the RNA (preferably mRNA) described in the present disclosure, in particular an RNA (preferably mRNA) encoding an antigen or epitope for inducing an immune response disclosed herein, contains a combination of at least two, at least three, at least four or all five of the above-mentioned modifications, i.e., (i) incorporation of a 5′-cap structure; (ii) incorporation of a poly-A sequence, unmasking of a poly-A sequence; (iii) alteration of the 5′- and/or 3′-UTR (such as incorporation of one or more 3′-UTRs); (iv) replacing one or more naturally occurring nucleotides with synthetic nucleotides (e.g., 5-methylcytidine for cytidine and/or pseudouridine (ψ) or N(1)-methylpseudouridine (m1ψ) or 5-methyluridine (m5U) for uridine); and (v) codon optimization. In some embodiments, the RNA (preferably mRNA) described in the present disclosure comprises a cap1 or cap2, preferably a cap1 structure. In some embodiments, the poly-A sequence comprises at least 100 nucleotides. In some embodiments, the poly-A sequence comprises or consists of the nucleotide sequence of SEQ ID NO: 3. In some embodiments, the a 5′-UTR comprises the nucleotide sequence of SEQ ID NO: 1, or a nucleotide sequence having at least 99%, 98%, 97%, 96%, 95%, 90%, 85%, or 80% identity to the nucleotide sequence of SEQ ID NO: 1. In some embodiments, the 3′-UTR comprising the nucleotide sequence of SEQ ID NO: 2, or a nucleotide sequence having at least 99%, 98%, 97%, 96%, 95%, 90%, 85%, or 80% identity to the nucleotide sequence of SEQ ID NO: 2.

In some embodiments, after administration of the RNA (in particular, mRNA) compositions described herein, at least a portion of the RNA is delivered to a target cell or target organ. In some embodiments, at least a portion of the RNA is delivered to the cytosol of the target cell. In some embodiments, the RNA is RNA (preferably mRNA) encoding a peptide or protein and the RNA is translated by the target cell to produce the peptide or protein. In some embodiments, the target cell is a cell in the liver. In some embodiments, the target cell is a muscle cell. In some embodiments, the target cell is an endothelial cell. In some embodiments, the target cell is a tumor cell or a cell in the tumor microenvironment. In some embodiments, the target cell is a blood cell. In some embodiments, the target cell is a cell in the lymph nodes. In some embodiments, the target cell is a cell in the lung. In some embodiments, the target cell is a cell in the skin. In some embodiments, the target cell is a spleen cell. In some embodiments, the target cell is an antigen presenting cell such as a professional antigen presenting cell in the spleen. In some embodiments, the target cell is a dendritic cell in the spleen. In some embodiments, the target cell is a T cell. In some embodiments, the target cell is a B cell. In some embodiments, the target cell is a NK cell. In some embodiments, the target cell is a monocyte. Thus, nucleic acid (such as RNA) particle (such as RNA LNP) compositions described herein may be used for delivering nucleic acid (such as RNA, preferably mRNA) to such target cell. Accordingly, the present disclosure also relates to a method for delivering nucleic acid (such as RNA, preferably mRNA) to a target cell in a subject comprising the administration of the nucleic acid (such as RNA, preferably mRNA) compositions described herein to the subject. In some embodiments, the RNA is delivered to the cytosol of the target cell. In some embodiments, the RNA is RNA (preferably mRNA) encoding a peptide or protein and the RNA is translated by the target cell to produce the peptide or protein.

### Inhibitory RNA

In some embodiments of all aspects of the disclosure, the nucleic acid is an inhibitory RNA, such as an siRNA or a miRNA.

The term “inhibitory RNA” as used herein means RNA which selectively hybridizes to and/or is specific for a target mRNA, thereby inhibiting (e.g., reducing) transcription and/or translation thereof. Inhibitory RNA includes RNA molecules having sequences in the antisense orientation relative to the target mRNA. Suitable inhibitory oligonucleotides typically vary in length from five to several hundred nucleotides, more typically about 20 to 70 nucleotides in length or shorter, even more typically about 10 to 30 nucleotides in length. Examples of inhibitory RNA include antisense RNA, ribozyme, iRNA, siRNA and miRNA. In some embodiments of all aspects of the disclosure, the inhibitory RNA is siRNA.

The term “antisense RNA” as used herein refers to an RNA which hybridizes under physiological conditions to DNA comprising a particular gene or to mRNA of said gene, thereby inhibiting transcription of said gene and/or translation of said mRNA. An antisense transcript of a nucleic acid or of a part thereof may form a duplex with naturally occurring mRNA and thus prevent accumulation of or translation of the mRNA. Another possibility is the use of ribozymes for inactivating a nucleic acid. The antisense RNA may hybridize with an N-terminal or 5′ upstream site such as a translation initiation site, transcription initiation site or promoter site. In some embodiments, the antisense RNA may hybridize with a 3′-untranslated region or mRNA splicing site.

The size of the antisense RNA may vary from 15 nucleotides to 15,000, preferably 20 to 12,000, in particular 100 to 10,000, 150 to 8,000, 200 to 7,000, 250 to 6,000, 300 to 5,000 nucleotides, such as 15 to 2,000, 20 to 1,000, 25 to 800, 30 to 600, 35 to 500, 40 to 400, 45 to 300, 50 to 250, 55 to 200, 60 to 150, or 65 to 100 nucleotides.

An antisense RNA can be targeted to any stretch of approximately 19 to 25 contiguous nucleotides in any of the target mRNA sequences (the “target sequence”). Generally, a target sequence on the target mRNA can be selected from a given cDNA sequence corresponding to the target mRNA, preferably beginning 50 to 100 nt downstream (i.e., in the 3′-direction) from the start codon. The target sequence can, however, be located in the 5′- or 3′-untranslated regions, or in the region nearby the start codon.

By “small interfering RNA” or “siRNA” as used herein is meant an RNA molecule, preferably greater than 10 nucleotides in length, more preferably greater than 15 nucleotides in length, and most preferably 18, 19, 20, 21, 22, 23, 24, 25, 26, 27, 28, 29, or 30 nucleotides in length that is capable of binding specifically to a portion of a target mRNA. This binding induces a process, in which said portion of the target mRNA is cut or degraded and thereby the gene expression of said target mRNA inhibited. A range of 19 to 25 nucleotides is the most preferred size for siRNAs. Although, in principle, the sense and antisense strands of siRNAs can comprise two complementary, single-stranded RNA molecules, the siRNAs, according to the present disclosure, comprise a single molecule in which two complementary portions are base-paired and are covalently linked by a single-stranded “hairpin” area. That is, the sense region and antisense region can be covalently connected via a linker molecule. The linker molecule can be a polynucleotide or non-nucleotide linker, but is preferably a polynucleotide linker. Without wishing to be bound by any theory, it is believed that the hairpin area of the siRNA molecule is cleaved intracellularly by the “Dicer” protein (or its equivalent) to form an siRNA of two individual base-paired RNA molecules.

As used herein, “target mRNA” refers to an RNA molecule that is a target for downregulation. In some embodiments, the target mRNA comprises an ORF encoding a pharmaceutically active peptide or polypeptide as specified herein. In some embodiments, the pharmaceutically active peptide or polypeptide is one whose expression (in particular increased expression, e.g., compared to the expression in a healthy subject) is associated with a disease. In some embodiments, the target mRNA comprises an ORF encoding a pharmaceutically active peptide or polypeptide whose expression (in particular increased expression, e.g., compared to the expression in a healthy subject) is associated with cancer.

According to the present disclosure, siRNA can be targeted to any stretch of approximately 19 to 25 contiguous nucleotides in any of the target mRNA sequences (the “target sequence”). Techniques for selecting target sequences for siRNA are given, for example, in Tuschl T. et al., “The siRNA User Guide”, revised Oct. 11, 2002, the entire disclosure of which is herein incorporated by reference. “The siRNA User Guide” is available on the world wide web at a website maintained by Dr. Thomas Tuschl, Laboratory of RNA Molecular Biology, Rockefeller University, New York, USA, and can be found by accessing the website of the Rockefeller University and searching with the keyword “siRNA”. Further guidance with respect to the selection of target sequences and/or the design of siRNA can be found on the webpages of Protocol Online (www.protocol-online.com) using the keyword “siRNA”. Thus, in some embodiments, the sense strand of the siRNA used in the present disclosure comprises a nucleotide sequence substantially identical to any contiguous stretch of about 19 to about 25 nucleotides in the target mRNA.

The term “miRNA” (microRNA) as used herein relates to non-coding RNAs which have a length of 21 to 25 (such as 21 to 23, preferably 22) nucleotides and which induce degradation and/or prevent translation of target mRNAs. miRNAs are typically found in plants, animals and some viruses, wherein they are encoded by eukaryotic nuclear DNA in plants and animals and by viral DNA (in viruses whose genome is based on DNA), respectively. miRNAs are post-transcriptional regulators that bind to complementary sequences on target messenger RNA transcripts (mRNAs), usually resulting in translational repression or target degradation and gene silencing.

Antisense RNA, siRNA or miRNA can be obtained using a number of techniques known to those of skill in the art. Preferably, antisense RNA, siRNA or miRNA is transcribed from recombinant circular or linear DNA plasmids using any suitable promoter.

### Pharmaceutically Active Peptides or Polypeptides

“Encoding” refers to the inherent property of specific sequences of nucleotides in a polynucleotide, such as a gene, a cDNA, or an RNA (preferably mRNA), to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of RNA (preferably mRNA) corresponding to that gene produces the protein in a cell or other biological system. Both the coding strand, the nucleotide sequence of which is identical to the RNA sequence and is usually provided in sequence listings, and the non-coding strand, used as the template for transcription of a gene or cDNA, can be referred to as encoding the protein or other product of that gene or cDNA.

In some embodiments, RNA (preferably mRNA) described in the present disclosure comprises a nucleic acid sequence (e.g., an ORF) encoding one or more polypeptides, e.g., a peptide or protein, preferably a pharmaceutically active peptide or polypeptide.

In some embodiments, RNA (preferably mRNA) described in the present disclosure comprises a nucleic acid sequence (e.g., an ORF) encoding a peptide or polypeptide, preferably a pharmaceutically active peptide or polypeptide, and is capable of expressing said peptide or polypeptide, in particular if transferred into a cell or subject. Thus, in some embodiments, the RNA (preferably mRNA) described in the present disclosure contains a coding region (ORF) encoding a peptide or polypeptide, preferably encoding a pharmaceutically active peptide or polypeptide. In this respect, an “open reading frame” or “ORF” is a continuous stretch of codons beginning with a start codon and ending with a stop codon.

Such RNA (preferably mRNA) encoding a pharmaceutically active peptide or polypeptide is also referred to herein as “pharmaceutically active RNA” (or “pharmaceutically active mRNA”). In some embodiments, RNA (preferably mRNA) described in the present disclosure comprises a nucleic acid sequence encoding more than one peptide or polypeptide, e.g., two, three, four or more peptides or polypeptides.

According to the present disclosure, the term “pharmaceutically active peptide or polypeptide” means a peptide or polypeptide that can be used in the treatment of an individual where the expression of the peptide or polypeptide would be of benefit, e.g., in ameliorating the symptoms of a disease or disorder.

Preferably, a pharmaceutically active peptide or polypeptide has curative or palliative properties and may be administered to ameliorate, relieve, alleviate, reverse, delay onset of or lessen the severity of one or more symptoms of a disease or disorder. In some embodiments, a pharmaceutically active peptide or polypeptide has a positive or advantageous effect on the condition or disease state of an individual when administered to the individual in a therapeutically effective amount. A pharmaceutically active peptide or polypeptide may have prophylactic properties and may be used to delay the onset of a disease or disorder or to lessen the severity of such disease or disorder. The term “pharmaceutically active peptide or polypeptide” includes entire peptides or polypeptides, and can also refer to pharmaceutically active fragments thereof. It can also include pharmaceutically active analogs of a peptide or polypeptide. The terms “pharmaceutically active peptide or polypeptide” and “therapeutic polypeptide” are used interchangeable herein.

Specific examples of pharmaceutically active peptides and polypeptides include, but are not limited to, immunostimulants, e.g., cytokines, hormones, adhesion molecules, immunoglobulins, immunologically active compounds, growth factors, protease inhibitors, enzymes, receptors, apoptosis regulators, transcription factors, tumor suppressor proteins, structural proteins, reprogramming factors, genomic engineering proteins, and blood proteins. In some embodiments, the pharmaceutically active peptide and polypeptide includes a replacement protein.

An “immunostimulant” is any substance that stimulates the immune system by inducing activation or increasing activity of any of the immune system's components, in particular immune effector cells. The immunostimulant may be pro-inflammatory (e.g., when treating infections or cancer), or anti-inflammatory (e.g., when treating autoimmune diseases).

In some embodiments, the pharmaceutically active peptide or polypeptide is a cytokine or a variant thereof. Examples of cytokines include interferons, such as interferon-alpha (IFN-α) or interferon-gamma (IFN-γ), interleukins, such as IL2, IL7, IL12, IL15 and IL23, colony stimulating factors, such as M-CSF and GM-CSF, and tumor necrosis factor.

The term “cytokines” relates to proteins which have a molecular weight of about 5 to 60 kDa (such as about 5 to 20 kDa) and which participate in cell signaling (e.g., paracrine, endocrine, and/or autocrine signaling). In particular, when released, cytokines exert an effect on the behavior of cells around the place of their release. Examples of cytokines include lymphokines, interleukins, chemokines, interferons, and tumor necrosis factors (TNFs). According to the present disclosure, cytokines do not include hormones or growth factors. Cytokines differ from hormones in that (i) they usually act at much more variable concentrations than hormones and (ii) generally are made by a broad range of cells (nearly all nucleated cells can produce cytokines). Interferons are usually characterized by antiviral, antiproliferative and immunomodulatory activities. Interferons are proteins that alter and regulate the transcription of genes within a cell by binding to interferon receptors on the regulated cell's surface, thereby preventing viral replication within the cells. The interferons can be grouped into two types. IFN-gamma is the sole type II interferon; all others are type I interferons. Particular examples of cytokines include erythropoietin (EPO), colony stimulating factor (CSF), granulocyte colony stimulating factor (G-CSF), granulocyte-macrophage colony stimulating factor (GM-CSF), tumor necrosis factor (TNF), bone morphogenetic protein (BMP), interferon alfa (IFNα), interferon beta (IFNβ), interferon gamma (INFγ), interleukin 2 (IL-2), interleukin 4 (IL-4), interleukin 10 (IL-10), interleukin 11 (IL-11), interleukin 12 (IL-12), and interleukin 21 (IL-21).

According to the disclosure, a cytokine may be a naturally occurring cytokine or a functional fragment or variant thereof. A cytokine may be human cytokine and may be derived from any vertebrate, especially any mammal. One particularly preferred cytokine is interferon-α.

In some embodiments, a pharmaceutically active peptide or polypeptide comprises a replacement protein. In these embodiments, the present disclosure provides a method for treatment of a subject having a disorder requiring protein replacement (e.g., protein deficiency disorders) comprising administering to the subject RNA as described herein encoding a replacement protein. The term “protein replacement” refers to the introduction of a protein (including functional variants thereof) into a subject having a deficiency in such protein. The term also refers to the introduction of a protein into a subject otherwise requiring or benefiting from providing a protein, e.g., suffering from protein insufficiency.

The term “disorder characterized by a protein deficiency” refers to any disorder that presents with a pathology caused by absent or insufficient amounts of a protein. This term encompasses protein folding disorders, i.e., conformational disorders, that result in a biologically inactive protein product. Protein insufficiency can be involved in infectious diseases, immunosuppression, organ failure, glandular problems, radiation illness, nutritional deficiency, poisoning, or other environmental or external insults.

The term “hormones” relates to a class of signaling molecules produced by glands, wherein signaling usually includes the following steps: (i) synthesis of a hormone in a particular tissue; (ii) storage and secretion; (iii) transport of the hormone to its target; (iv) binding of the hormone by a receptor; (v) relay and amplification of the signal; and (vi) breakdown of the hormone. Hormones differ from cytokines in that (1) hormones usually act in less variable concentrations and (2) generally are made by specific kinds of cells. In some embodiments, a “hormone” is a peptide or protein hormone, such as insulin, vasopressin, prolactin, adrenocorticotropic hormone (ACTH), thyroid hormone, growth hormones (such as human grown hormone or bovine somatotropin), oxytocin, atrial-natriuretic peptide (ANP), glucagon, somatostatin, cholecystokinin, gastrin, and leptins.

The term “adhesion molecules” relates to proteins which are located on the surface of a cell and which are involved in binding of the cell with other cells or with the extracellular matrix (ECM). Adhesion molecules are typically transmembrane receptors and can be classified as calcium-independent (e.g., integrins, immunoglobulin superfamily, lymphocyte homing receptors) and calcium-dependent (cadherins and selectins). Particular examples of adhesion molecules are integrins, lymphocyte homing receptors, selectins (e.g., β-selectin), and addressins.

Integrins are also involved in signal transduction. In particular, upon ligand binding, integrins modulate cell signaling pathways, e.g., pathways of transmembrane protein kinases such as receptor tyrosine kinases (RTK). Such regulation can lead to cellular growth, division, survival, or differentiation or to apoptosis. Particular examples of integrins include: α₁β₁, α₂, β₁, α₃β₁, α₄β₁, α₅β₁, α₆β₁, α₇β₁, α_{L}β₂, α_{M}β₂, α_{IIb}β₃, α_{V}β₁, α_{V}β₃, α_{V}β₅, αᵥβ6, α_{V}β₈, and α₆β₄.

The term “immunoglobulins” or “immunoglobulin superfamily” refers to molecules which are involved in the recognition, binding, and/or adhesion processes of cells. Molecules belonging to this superfamily share the feature that they contain a region known as immunoglobulin domain or fold. Members of the immunoglobulin superfamily include antibodies (e.g., IgG), T cell receptors (TCRs), major histocompatibility complex (MHC) molecules, co-receptors (e.g., CD4, CD8, CD19), antigen receptor accessory molecules (e.g., CD-3γ, CD3-δ, CD-3ε, CD79a, CD79b), co-stimulatory or inhibitory molecules (e.g., CD28, CD80, CD86), and other.

In some embodiments, RNA (in particular, mRNA) described in the present disclosure comprises a nucleic acid sequence encoding a peptide or polypeptide comprising an epitope for inducing an immune response against an antigen in a subject. The “peptide or polypeptide comprising an epitope for inducing an immune response against an antigen in a subject” is also designated herein as “vaccine antigen”, “peptide and protein antigen” or simply “antigen”.

In some embodiments, the RNA encoding the vaccine antigen is expressed in cells of the subject to provide the vaccine antigen. In some embodiments, expression of the vaccine antigen is at the cell surface. In some embodiments, the vaccine antigen is presented in the context of MHC. In some embodiments, the RNA encoding the vaccine antigen is transiently expressed in cells of the subject. In some embodiments, the RNA encoding the vaccine antigen is administered systemically. In some embodiments, after systemic administration of the RNA encoding the vaccine antigen, expression of the RNA encoding the vaccine antigen in spleen occurs. In some embodiments, after systemic administration of the RNA encoding the vaccine antigen, expression of the RNA encoding the vaccine antigen in antigen presenting cells, preferably professional antigen presenting cells occurs. In some embodiments, the antigen presenting cells are selected from the group consisting of dendritic cells, macrophages and B cells. In some embodiments, after systemic administration of the RNA encoding the vaccine antigen, no or essentially no expression of the RNA encoding the vaccine antigen in lung and/or liver occurs. In some embodiments, after systemic administration of the RNA encoding the vaccine antigen, expression of the RNA encoding the vaccine antigen in spleen is at least 5-fold the amount of expression in lung.

The vaccine antigen comprises an epitope for inducing an immune response against an antigen in a subject. Accordingly, the vaccine antigen comprises an antigenic sequence for inducing an immune response against an antigen in a subject. Such antigenic sequence may correspond to a target antigen or disease-associated antigen, e.g., a protein of an infectious agent (e.g., viral or bacterial antigen) or tumor antigen, or may correspond to an immunogenic variant thereof, or an immunogenic fragment of the target antigen or disease-associated antigen or the immunogenic variant thereof. Thus, the antigenic sequence may comprise at least an epitope of a target antigen or disease-associated antigen or an immunogenic variant thereof.

In some embodiments, a secretory sequence, e.g., a sequence comprising the amino acid sequence of SEQ ID NO: 4, may be fused to the N-terminus of the antigenic peptide or polypeptide.

In some embodiments, an amino acid sequence enhancing antigen processing and/or presentation comprises the amino acid sequence of SEQ ID NO: 5, an amino acid sequence having at least 99%, 98%, 97%, 96%, 95%, 90%, 85%, or 80% identity to the amino acid sequence of SEQ ID NO: 5, or a functional fragment of the amino acid sequence of SEQ ID NO: 5, or the amino acid sequence having at least 99%, 98%, 97%, 96%, 95%, 90%, 85%, or 80% identity to the amino acid sequence of SEQ ID NO: 5. In some embodiments, an amino acid sequence enhancing antigen processing and/or presentation comprises the amino acid sequence of SEQ ID NO: 5.

Accordingly, in some embodiments, the RNA described herein comprises at least one coding region encoding an antigenic peptide or polypeptide and an amino acid sequence enhancing antigen processing and/or presentation, said amino acid sequence enhancing antigen processing and/or presentation preferably being fused to the antigenic peptide or polypeptide, more preferably to the C-terminus of the antigenic peptide or polypeptide as described herein.

In the following, embodiments of vaccine RNAs are described, wherein certain terms used when describing elements thereof have the following meanings:
cap: 5′-cap structure selected from the group consisting of m₂^{7,2O}G(5′)ppSp(5′)G (in particular its D1 diastereomer), m₂^{7,3O}G(5′)ppp(5′)G, and m₂^{7,3′-O}Gppp(m₁^{2′-O})ApG.
hAg-Kozak: 5′-UTR sequence of the human alpha-globin mRNA with an optimized ‘Kozak sequence’ to increase translational efficiency.
sec/MITD: Fusion-protein tags derived from the sequence encoding the human MHC class I complex (HLA-B51, haplotype A2, B27/B51, Cw2/Cw3), which have been shown to improve antigen processing and presentation. Sec corresponds to the 78 bp fragment coding for the secretory signal peptide, which guides translocation of the nascent polypeptide chain into the endoplasmatic reticulum. MITD corresponds to the transmembrane and cytoplasmic domain of the MHC class I molecule, also called MHC class I trafficking domain.
Antigen: Sequences encoding the respective vaccine antigen/epitope.
Glycine-serine linker (GS): Sequences coding for short peptide linkers predominantly consisting of the amino acids glycine (G) and serine (S), as commonly used for fusion proteins.
P2P16: Sequence coding for tetanus toxoid-derived helper epitopes to break immunological tolerance.
FI element: The 3′-UTR is a combination of two sequence elements derived from the “amino terminal enhancer of split” (AES) mRNA (called F) and the mitochondrial encoded 12S ribosomal RNA (called I). These were identified by an ex vivo selection process for sequences that confer RNA stability and augment total protein expression.
A30L70: A poly(A)-tail measuring 110 nucleotides in length, consisting of a stretch of 30 adenosine residues, followed by a 10 nucleotide linker sequence and another 70 adenosine residues designed to enhance RNA stability and translational efficiency in dendritic cells.

In some embodiments, vaccine RNA described herein has one of the following structures:

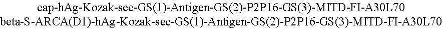

In some embodiments, vaccine antigen described herein has the structure:

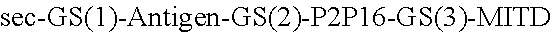

In some embodiments, hAg-Kozak comprises the nucleotide sequence of SEQ ID NO: 1. In some embodiments, see comprises the amino acid sequence of SEQ ID NO: 4. In some embodiments, P2P16 comprises the amino acid sequence of SEQ ID NO: 6. In some embodiments, MITD comprises the amino acid sequence of SEQ ID NO: 5. In some embodiments, GS(1) comprises the amino acid sequence of SEQ ID NO: 7. In some embodiments, GS(2) comprises the amino acid sequence of SEQ ID NO: 8. In some embodiments, GS(3) comprises the amino acid sequence of SEQ ID NO: 8. In some embodiments, FI comprises the nucleotide sequence of SEQ ID NO: 2. In some embodiments, A30L70 comprises the nucleotide sequence of SEQ ID NO: 3.

In some embodiments, the sequence encoding the vaccine antigen/epitope comprises a modified nucleoside replacing (partially or completely, preferably completely) uridine, wherein the modified nucleoside is selected from the group consisting of pseudouridine (ψ), N1-methyl-pseudouridine (mlψ), and 5-methyl-uridine.

In some embodiments, the sequence encoding the vaccine antigen/epitope is codon-optimized.

In some embodiments, the G/C content of the sequence encoding the vaccine antigen/epitope is increased compared to the wild type coding sequence.

Thus, in some embodiments, the pharmaceutically active peptide or polypeptide is (i) a cytokine, preferably selected from the group consisting of erythropoietin (EPO), interleukin 4 (IL-2), and interleukin 10 (IL-11), more preferably EPO; (ii) an adhesion molecule, in particular an integrin; (iii) an immunoglobulin, in particular an antibody; (iv) an immunologically active compound, in particular an antigen, such as a viral or bacterial antigen, e.g., an antigen of SARS-CoV-2; (v) a hormone, in particular vasopressin, insulin or growth hormone; (vi) a growth factor, in particular VEGFA; (vii) a protease inhibitor, in particular alpha 1-antitrypsin; (viii) an enzyme, preferably selected from the group consisting of herpes simplex virus type 1 thymidine kinase (HSV1-TK), hexosaminidase, phenylalanine hydroxylase, pseudocholinesterase, pancreatic enzymes, and lactase; (ix) a receptor, in particular growth factor receptors; (x) an apoptosis regulator, in particular BAX; (xi) a transcription factor, in particular FOXP3; (xii) a tumor suppressor protein, in particular p53; (xiii) a structural protein, in particular surfactant protein B; (xiv) a reprogramming factor, e.g., selected from the group consisting of OCT4, SOX2, c-MYC, KLF4, LIN28 and NANOG; (xv) a genomic engineering protein, in particular clustered regularly spaced short palindromic repeat-CRISPR-associated protein 9 (CRISPR-Cas9); and (xvi) a blood protein, in particular fibrinogen.

In some embodiments, a pharmaceutically active peptide or polypeptide comprises one or more antigens or one or more epitopes, i.e., administration of the peptide or polypeptide to a subject elicits an immune response against the one or more antigens or one or more epitopes in a subject which may be therapeutic or partially or fully protective.

In certain embodiments, the RNA (preferably mRNA) encodes at least one epitope, e.g., at least two epitopes, at least three epitopes, at least four epitopes, at least five epitopes, at least six epitopes, at least seven epitopes, at least eight epitopes, at least nine epitopes, or at least ten epitopes.

In certain embodiments, the target antigen is a tumor antigen and the antigenic sequence (e.g., an epitope) is derived from the tumor antigen. The tumor antigen may be a “standard” antigen, which is generally known to be expressed in various cancers. The tumor antigen may also be a “neo-antigen”, which is specific to an individual's tumor and has not been previously recognized by the immune system.

A neo-antigen or neo-epitope may result from one or more cancer-specific mutations in the genome of cancer cells resulting in amino acid changes. If the tumor antigen is a neo-antigen, the vaccine antigen preferably comprises an epitope or a fragment of said neo-antigen comprising one or more amino acid changes.

Examples of tumor antigens include, without limitation, p53, ART-4, BAGE, beta-catenin/m, Bcr-abL CAMEL, CAP-1, CASP-8, CDC27/m, CDK4/m, CEA, the cell surface proteins of the claudin family, such as CLAUDIN-6, CLAUDIN-18.2 and CLAUDIN-12, c-MYC, CT, Cyp-B, DAM, ELF2M, ETV6-AML1, G250, GAGE, GnT-V, Gap 100, HAGE, HER-2/neu, HPV-E7, HPV-E6, HAST-2, hTERT (or hTRT), LAGE, LDLR/FUT, MAGE-A, preferably MAGE-A1, MAGE-A2, MAGE-A3, MAGE-A4, MAGE-A5, MAGE-A6, MAGE-A7, MAGE-A8, MAGE-A9, MAGE-A10, MAGE-A11, or MAGE-A12, MAGE-B, MAGE-C, MART-1/Melan-A, MC1R, Myosin/m, MUC1, MUM-1, MUM-2, MUM-3, NA88-A, NF1, NY-ESO-1, NY-BR-1, p190 minor BCR-abL, Pml/RARa, PRAME, proteinase 3, PSA, PSM, RAGE, RU1 or RU2, SAGE, SART-1 or SART-3, SCGB3A2, SCP1, SCP2, SCP3, SSX, SURVIVIN, TEL/AML1, TPI/m, TRP-1, TRP-2, TRP-2/INT2, TPTE, WT, and WT-1.

Cancer mutations vary with each individual. Thus, cancer mutations that encode novel epitopes (neo-epitopes) represent attractive targets in the development of vaccine compositions and immunotherapies. The efficacy of tumor immunotherapy relies on the selection of cancer-specific antigens and epitopes capable of inducing a potent immune response within a host. RNA can be used to deliver patient-specific tumor epitopes to a patient. Dendritic cells (DCs) residing in the spleen represent antigen-presenting cells of particular interest for RNA expression of immunogenic epitopes or antigens such as tumor epitopes. The use of multiple epitopes has been shown to promote therapeutic efficacy in tumor vaccine compositions. Rapid sequencing of the tumor mutanome may provide multiple epitopes for individualized vaccines which can be encoded by RNA (in particular mRNA) described herein, e.g., as a single polypeptide wherein the epitopes are optionally separated by linkers. In certain embodiments of the present disclosure, the RNA (in particular mRNA) encodes at least one epitope, at least two epitopes, at least three epitopes, at least four epitopes, at least five epitopes, at least six epitopes, at least seven epitopes, at least eight epitopes, at least nine epitopes, or at least ten epitopes. Exemplary embodiments include RNA (in particular, mRNA) that encodes at least five epitopes (termed a “pentatope”) and RNA (in particular, mRNA) that encodes at least ten epitopes (termed a “decatope”).

In certain embodiments, the epitope is derived from a pathogen-associated antigen. In some embodiments, the pharmaceutically active polypeptide and/or the antigen or epitope is derived from or is a protein of a pathogen, an immunogenic variant of the protein, or an immunogenic fragment of the protein or the immunogenic variant thereof.

In some embodiments, the pathogen is selected from viruses, bacteria, fungi, parasites, and other microorganisms.

Exemplary viruses include, but are not limited to, are severe acute respiratory syndrome coronavirus (SARS-CoV), such as SARS-CoV2, human immunodeficiency virus (HIV), Epstein-Barr virus (EBV), cytomegalovirus (CMV) (e.g., CMV5), human herpesviruses (HHV) (e.g., HHV6, 7 or 8), herpes simplex viruses (HSV), bovine herpes virus (BHV) (e.g., BHV4), equine herpes virus (EHV) (e.g., EHV2), human T-CeJl leukemia viruses (HTLV)5, Varicella-Zoster virus (VZV), measles virus, papovaviruses (JC and BK), hepatitis viruses (e.g., HBV or HCV), myxoma virus, adenoviruses, rhinoviruses, enteroviruses, parvoviruses, polyoma virus, influenza viruses, papillomaviruses (such as human papillomavirus (HPV)), poxviruses such as vaccinia virus, and molluscum contagiosum virus (MCV), lyssaviruses, rotaviruses, noroviruses, rubella viruses, and mumps viruses. Exemplary diseases caused by viral infection include, but are not limited to, SARS, acquired immune deficiency syndrome (AIDS), measles, chicken pox, cytomegalovirus infections, genital herpes, hepatitis (such as hepatitis B or C), influenza (flu, such as human flu, swine flu, dog flu, horse flu, and avian flu), HPV infection, shingles, rabies, common cold, gastroenteritis, rubella, and mumps.

Exemplary bacteria include, but are not limited to, Campylobacter (such as Campylobacter jejuni), Enterobacter species, Enterococcus faecium, Enterococcus faecalis, Escherichia coli (e.g., F. coli O157:H7), Group A streptococci, Haemophilus influenzae, Helicobacterpylori, listeria, Mycobacterium tuberculosis, Pseudomonas aeruginosa, S. pneumoniae, Salmonella, Shigella, Staphylococcus aureus, Staphylococcus epidermidis, Borrelia and Rickettsia, Chlamydiaceae, Neisseria gonorrhoeae, Bordetella pertussis, Clostridium tetani, Neisseria meningitidis, Streptococcus (such as Streptococcus pneumoniae or Streptococcus pyogenes), and Treponema pallidum. Exemplary diseases caused by bacterial infection include, but are not limited to, anthrax, cholera, diphtheria, foodborne illnesses, leprosy, meningitis, peptic ulcer disease, pneumonia, sepsis, septic shock, tetanus, tuberculosis, typhoid fever, urinary tract infection, Lyme disease, Rocky Mountain spotted fever, chlamydia, gonorrhea, pertussis, tetanus, meningitis, scarlet fever, and syphilis.

Exemplary parasites include, but are not limited to, Plasmodium, Trypanosoma, Leishmania, Trichomonas, Dientamoeba, Giardia, Entamoeba histolytica, Naegleria, Isospora, Toxoplasma, Sarcocystis, Rhinosporidium seeberi, and Balantidium. Exemplary diseases caused by parasite infection include, but are not limited to, malaria, trypanosomiasis, Chagas disease, leishmaniasis, trichomoniasis, dientamoebiasis, giardiasis, amebic dysentery, coccidiosis, toxoplasmosis, sarcocystosis, rhinosporidiosis, and balantidiasis.

In some embodiments, the pathogen is an infectious pathogen, in particular a pathogen causing an infectious disease, such as a viral disease, a bacterial disease, or a parasitic disease. In some embodiments, the pathogen is a virus, bacterium, or parasite. Thus, in these embodiments, the RNA (in particular mRNA) and/or compositions described herein can be used to prevent and/or treat an infectious disease caused by said pathogen.

In certain embodiments, the epitope is derived from a viral antigen.

In some embodiments, the antigen or epitope is derived from a coronavirus protein, an immunogenic variant thereof, or an immunogenic fragment of the coronavirus protein or the immunogenic variant thereof. Thus, in some embodiments, the mRNA used in the present disclosure encodes an amino acid sequence comprising a coronavirus protein, an immunogenic variant thereof, or an immunogenic fragment of the coronavirus protein or the immunogenic variant thereof.

In some embodiments, the antigen or epitope is derived from a coronavirus S protein, an immunogenic variant thereof, or an immunogenic fragment of the coronavirus S protein or the immunogenic variant thereof. Thus, in some embodiments, the RNA (in particular, mRNA) described in the present disclosure encodes an amino acid sequence comprising a coronavirus S protein, an immunogenic variant thereof, or an immunogenic fragment of the coronavirus S protein or the immunogenic variant thereof. In some embodiments, the coronavirus is MERS-CoV. In some embodiments, the coronavirus is SARS-CoV. In some embodiments, the coronavirus is SARS-CoV-2.

### Particles

Nucleic acids such as RNA, in particular mRNA, described herein may be present in particles comprising (i) the nucleic acid and (ii) at least one cationic or cationically ionizable compound. In some embodiments, the particles comprise (i) the nucleic acid (such as RNA, in particular mRNA); (ii) at least one cationic or cationically ionizable compound as disclosed herein; (iii) a steroid as disclosed herein; (iv) a neutral lipid as disclosed herein; and (v) optionally a polymer-conjugated lipid as disclosed herein.

Different types of RNA containing particles have been described previously to be suitable for delivery of RNA in particulate form (cf., e.g., Kaczmarek, J. C. et al., 2017, Genome Medicine 9, 60). For non-viral RNA delivery vehicles, nanoparticle encapsulation of RNA physically protects RNA from degradation and, depending on the specific chemistry, can aid in cellular uptake and endosomal escape.

Electrostatic interactions between positively charged molecules such as polymers and lipids and negatively charged nucleic acid are involved in particle formation. This results in complexation and spontaneous formation of nucleic acid particles.

In the context of the present disclosure, the term “particle” relates to a structured entity formed by molecules or molecule complexes, in particular particle forming compounds. In some embodiments, the particle contains an envelope (e.g., one or more layers or lamellas) made of one or more types of amphiphilic substances (e.g., amphiphilic lipids, amphiphilic polymers, and/or amphiphilic proteins/polypeptides). In this context, the expression “amphiphilic substance” means that the substance possesses both hydrophilic and lipophilic properties. The envelope may also comprise additional substances (e.g., additional lipids and/or additional polymers) which do not have to be amphiphilic. Thus, the particle may be a monolamellar or multilamellar structure, wherein the substances constituting the one or more layers or lamellas comprise one or more types of amphiphilic substances (in particular selected from the group consisting of amphiphilic lipids, amphiphilic polymers, and/or amphiphilic proteins/polypeptides) optionally in combination with additional substances (e.g., additional lipids and/or additional polymers) which do not have to be amphiphilic. In some embodiments, the term “particle” relates to a micro- or nano-sized structure, such as a micro- or nano-sized compact structure. In this respect, the term “micro-sized” means that all three external dimensions of the particle are in the microscale, i.e., between 1 and 5 μm. According to the present disclosure, the term “particle” includes lipoplex particles (LPXs), lipid nanoparticles (LNPs), and liposomes.

A “nucleic acid particle” can be used to deliver nucleic acid (such as RNA, in particular mRNA) to a target site of interest (e.g., cell, tissue, organ, and the like). A nucleic acid particle may be formed from at least one cationic or cationically ionizable lipid or lipid-like material, at least one cationic polymer such as protamine, or a mixture thereof and nucleic acid. Nucleic acid particles include lipid nanoparticle (LNP)-based, lipoplex (LPX)-based, and/or liposome-based formulations.

Without intending to be bound by any theory, it is believed that the cationic or cationically ionizable lipid combine together with the nucleic acid to form aggregates, and this aggregation results in colloidally stable particles. In some embodiments, particles comprise a cationic or cationically ionizable amphiphilic lipid, and nucleic acid (such as RNA, especially mRNA) as described herein. In some embodiments, particles comprise or consist of a cationic/cationically ionizable lipid (in particular, a cationically ionizable lipid of formula (X) disclosed herein; a cationically ionizable lipid having one of the structures A to G disclosed herein; or a cationically ionizable lipid of formula (XI) disclosed herein); and additional lipids such as a neutral lipid (such as a phospholipid), and a steroid (such as cholesterol), and combinations thereof; and optionally a polymer-conjugated lipid.

In some embodiments, the nucleic acid (such as RNA) particles disclosed herein comprise (i) nucleic acid (such as RNA, especially mRNA) as described herein; (ii) an amphiphilic lipid, in particular cationic or cationically ionizable amphiphilic lipid (such as a cationically ionizable lipid of formula (X) disclosed herein; a cationically ionizable lipid having one of the structures A to G disclosed herein; a cationically ionizable lipid of formula (XI) disclosed herein; or a cationic lipid as disclosed herein); (iii) a steroid (such as cholesterol), a neutral lipid (such as a phospholipid); and (v) optionally a polymer-conjugated lipid. In some embodiments, the steroid is cholesterol; and the neutral lipid is selected from the group consisting of DSPC, DOPC, and DOPE. In some embodiments, the particles are dispersed in an aqueous phase, wherein the aqueous phase comprises a buffer system comprising histidine or HEPES as buffering substance. In some embodiments, the steroid is cholesterol; the neutral lipid is selected from the group consisting of DSPC, DOPC, and DOPE; and the particles are dispersed in an aqueous phase, wherein the aqueous phase comprises a buffer system comprising histidine or HEPES as buffering substance. In some embodiments, the steroid is cholesterol; the neutral lipid is selected from the group consisting of DSPC, DOPC, and DOPE; and the particles are dispersed in an aqueous phase, wherein the aqueous phase comprises a buffer system comprising histidine as buffering substance. In some embodiments, the steroid is cholesterol; the neutral lipid is selected from the group consisting of DSPC, DOPC, and DOPE; and the particles are dispersed in an aqueous phase, wherein the aqueous phase comprises a buffer system comprising HEPES as buffering substance.

In some embodiments, in the nucleic acid particles (such as RNA particles) described herein the nucleic acid (such as RNA, in particular, mRNA) is bound by cationically ionizable lipid (in particular a cationically ionizable lipid of formula (X) disclosed herein; a cationically ionizable lipid having one of the structures A to G disclosed herein; or a cationically ionizable lipid of formula (XI) disclosed herein) that, in the case of LNPs, occupies the central core of the LNPs. In some embodiments, phospholipids form the surface of the particles (such as LNPs), along with, e.g., polymer-conjugated lipids. In some embodiments, the particles are substantially free of a PEG lipid having at least 30 consecutive ethylene glycol repeating units. In some embodiments, the surface comprises a bilayer. In some embodiments, cholesterol and cationically ionizable lipid (in particular a cationically ionizable lipid of formula (X) disclosed herein; a cationically ionizable lipid having one of the structures A to G disclosed herein; or a cationically ionizable lipid of formula (XI) disclosed herein) in charged and uncharged forms can be distributed throughout the particles such as LNPs.

In general, a lipoplex (LPX) is obtainable from mixing two aqueous phases, namely a phase comprising nucleic acid (in particular RNA) and a phase comprising a dispersion of lipids. In some embodiments, the lipid phase comprises liposomes.

In some embodiments, liposomes are self-closed unilamellar or multilamellar vesicular particles wherein the lamellae comprise lipid bilayers and the encapsulated lumen comprises an aqueous phase.

A prerequisite for using liposomes for nanoparticle formation is that the lipids in the mixture as required are able to form lamellar (bilayer) phases in the applied aqueous environment.

In some embodiments, liposomes comprise unilamellar or multilamellar phospholipid bilayers enclosing an aqueous core (also referred to herein as an aqueous lumen). They may be prepared from materials possessing polar head (hydrophilic) groups and nonpolar tail (hydrophobic) groups. In some embodiments, cationic lipids employed in formulating liposomes designed for the delivery of nucleic acids are amphiphilic in nature and consist of a positively charged (cationic)amine head group linked to a hydrocarbon chain or cholesterol derivative via glycerol.

In some embodiments, lipoplexes are multilamellar liposome-based formulations that form upon electrostatic interaction of cationic liposomes with nucleic acids (such as RNAs). In some embodiments, formed lipoplexes possess distinct internal arrangements of molecules that arise due to the transformation from liposomal structure into compact nucleic acid-lipoplexes (such as RNA-lipoplexes).

In some embodiments, an LPX particle comprises an amphiphilic lipid, in particular cationic or cationically ionizable amphiphilic lipid, and nucleic acid (such as RNA, especially mRNA) as described herein. In some embodiments, electrostatic interactions between positively charged liposomes (made from one or more amphiphilic lipids, in particular cationic or cationically ionizable amphiphilic lipids) and negatively charged nucleic acid (such as RNA, especially mRNA) results in complexation and spontaneous formation of nucleic acid lipoplex particles. Positively charged liposomes may be generally synthesized using a cationic or cationically ionizable amphiphilic lipid, such as a cationically ionizable lipid of formula (I), DOTMA and/or DODMA, and additional lipids, such as DSPC or DOPC. In some embodiments, a nucleic acid (such as RNA, especially mRNA) lipoplex particle is a nanoparticle.

In general, a lipid nanoparticle (LNP) is obtainable from direct mixing of nucleic acid (such as RNA) in an aqueous phase with lipids in a phase comprising an organic solvent, such as ethanol. For LNPs, lipids or lipid mixtures used for particle formation, typically do not form lamellar (bilayer) phases in water. In some embodiments, the lipids comprise a cationically ionizable lipid (in particular a cationically ionizable lipid of formula (X) disclosed herein; a cationically ionizable lipid having one of the structures A to G disclosed herein; or a cationically ionizable lipid of formula (XI) disclosed herein), a steroid as disclosed herein (such as cholesterol), and a neutral lipid as disclosed herein (such as a phospholipid).

In some embodiments, particles described herein comprise a cationically ionizable lipid as disclosed herein (in particular a cationically ionizable lipid of formula (X) disclosed herein; a cationically ionizable lipid having one of the structures A to G disclosed herein; or a cationically ionizable lipid of formula (XI) disclosed herein), a steroid as disclosed herein (such as cholesterol), a neutral lipid as disclosed herein (such as a phospholipid), and optionally a polymer-conjugated lipid.

In some embodiments, nucleic acid particles (especially RNA particles such as RNA LNPs (e.g., mRNA particles such as mRNA LNPs)) comprise more than one type of nucleic acid molecules, where the molecular parameters of the nucleic acid molecules may be similar or different from each other, like with respect to molar mass or fundamental structural elements such as molecular architecture, capping, coding regions or other features,

In some embodiments, nucleic acid (such as RNA, e.g., mRNA) described herein may be noncovalently associated with a particle as described herein. In some embodiments, the nucleic acid (such as RNA, especially mRNA) may be adhered to the outer surface of the particle (surface nuclei acid (such as surface RNA, especially surface mRNA)) and/or may be contained in the particle (encapsulated nucleic acid (such as surface RNA, especially encapsulated mRNA)).

As used in the present disclosure, “nanoparticle” refers to a particle comprising nucleic acid (especially RNA such as mRNA) as described herein and at least one cationic lipid, wherein all three external dimensions of the particle are in the nanoscale, i.e., at least about 1 nm and below about 1000 nm (preferably, between 10 and 990 nm, such as between 15 and 900 nm, between 20 and 800 nm, between 30 and 700 nm, between 40 and 600 nm, or between 50 and 500 nm). Preferably, the longest and shortest axes do not differ significantly. Preferably, the size of a particle is its diameter.

Nucleic acid particles described herein (especially RNA particles, such as mRNA particles) may exhibit a polydispersity index (PDI) less than about 0.5, less than about 0.4, less than about 0.3, less than about 0.2, less than about 0.1, or less than about 0.05. By way of example, the nucleic acid particles can exhibit a polydispersity index in a range of about 0.01 to about 0.4 or about 0.1 to about 0.3.

In the context of the present disclosure, the term “lipoplex particle” relates to a particle that contains an amphiphilic lipid, in particular a cationic lipid, and nucleic acid (especially RNA such as mRNA) as described herein. Electrostatic interactions between positively charged liposomes (made from one or more amphiphilic lipids, in particular cationic amphiphilic lipids) and negatively charged nucleic acid (especially RNA such as mRNA) results in complexation and spontaneous formation of nucleic acid lipoplex particles. Positively charged liposomes may be generally synthesized using a cationic amphiphilic lipid, such as DOTMA, and additional lipids, such as DSPC or DOPC. In one embodiment, a nucleic acid (especially RNA such as mRNA) lipoplex particle is a nanoparticle.

The term “nucleic acid containing particle” relates to a particle as described herein to which nucleic acid (especially RNA such as mRNA) is bound. In this respect, the nucleic acid (especially RNA such as mRNA) may be adhered to the outer surface of the particle (surface nucleic acid (especially surface RNA such as surface mRNA)) and/or may be contained in the particle (encapsulated nucleic acid (especially encapsulated RNA such as encapsulated mRNA)).

In one embodiment, the particles contained in the compositions of the present disclosure and/or utilized in the methods and uses of the present disclosure have a size (preferably a diameter, i.e., double the radius such as double the radius of gyration (R_{g}) value or double the hydrodynamic radius) in the range of about 10 to about 2000 nm, such as at least about 15 nm (preferably at least about 20 n, at least about 25 nm, at least about 30 nm, at least about 35 n, at least about 40 n, at least about 45 nm, at least about 50 n, at least about 55 n, at least about 60 nm, at least about 65 n, at least about 70 n, at least about 75 n, at least about 80 n, at least about 85 n, at least about 90 n, at least about 95 n, or at least about 100 nm) and/or at most 1900 nm (preferably at most about 1900 n, at most about 1800 n, at most about 1700 n, at most about 1600 n, at most about 1500 n, at most about 1400 n, at most about 1300 nm, at most about 1200 nm, at most about 1100 n, at most about 1000 n, at most about 950 n, at most about 900 n, at most about 850 n, at most about 800 nm, at most about 750 n, at most about 700 nm, at most about 650 n, at most about 600 n, at most about 550 nm, or at most about 500 n), preferably in the range of about 20 to about 1500 n, such as about 30 to about 1200 n, about 40 to about 1100 nm, about 50 to about 1000 n, about 60 to about 900 n, about 70 to 800 nm, about 80 to 700 n, about 90 to 600 n, or about 50 to 500 nm or about 100 to 500 nm, such as in the range of 10 to 1000 nm, 15 to 500 n, 20 to 450 n, 25 to 400 n, 30 to 350 n, 40 to 300 n, 50 to 250 n, 60 to 200 nm, or 70 to 150 nm.

In some embodiments, the particles (e.g., LNPs and LPXs) described herein have an average diameter that in some embodiments ranges from about 50 nm to about 1000 nm, from about 50 nm to about 800 n, from about 50 nm to about 700 nm, from about 50 nm to about 600 n, from about 50 nm to about 500 nm, from about 50 nm to about 450 n, from about 50 nm to about 400 nm, from about 50 nm to about 350 nm, from about 50 nm to about 300 n, from about 50 nm to about 250 nm, from about 50 nm to about 200 n, from about 100 nm to about 1000 nm, from about 100 nm to about 800 nm, from about 100 nm to about 700 nm, from about 100 nm to about 600 n, from about 100 nm to about 500 n, from about 100 nm to about 450 n, from about 100 nm to about 400 n, from about 100 nm to about 350 n, from about 100 nm to about 300 n, from about 100 nm to about 250 n, from about 100 nm to about 200 n, from about 150 nm to about 1000 nm, from about 150 nm to about 800 n, from about 150 nm to about 700 n, from about 150 nm to about 600 nm, from about 150 nm to about 500 nm, from about 150 nm to about 450 nm, from about 150 nm to about 400 nm, from about 150 nm to about 350 nm, from about 150 nm to about 300 nm, from about 150 nm to about 250 nm, from about 150 nm to about 200 nm, from about 200 nm to about 1000 nm, from about 200 nm to about 800 nm, from about 200 nm to about 700 nm, from about 200 nm to about 600 nm, from about 200 nm to about 500 nm, from about 200 nm to about 450 nm, from about 200 nm to about 400 nm, from about 200 nm to about 350 nm, from about 200 nm to about 300 nm, or from about 200 nm to about 250 nm. With respect to nucleic acid particles (such as RNA lipid particles, especially RNA LNPs such as mRNA LNPs), the N/P ratio or N/P value gives the ratio of the nitrogen groups (in particular positively-chargeable polymer amine (N=nitrogen) groups) in the lipid to the number of negatively-charged phosphate (P) groups in the nucleic acid. It is correlated to the charge ratio, as the nitrogen atoms (depending on the pH) are usually positively charged and the phosphate groups are negatively charged.

The N/P ratio, where a charge equilibrium exists, depends on the pH. Lipid formulations are frequently formed at N/P ratios larger than four up to twelve, because positively charged nanoparticles are considered favorable for transfection. In that case, RNA is considered to be completely bound to nanoparticles.

Nucleic acid particles (especially RNA particles such as mRNA particles) described herein can be prepared using a wide range of methods that may involve obtaining a colloid from at least one cationic or cationically ionizable lipid and/or at least one cationic polymer and mixing the colloid with nucleic acid to obtain nucleic acid particles.

The term “colloid” as used herein relates to a type of homogeneous mixture in which dispersed particles do not settle out. The insoluble particles in the mixture are microscopic, with particle sizes between 1 and 1000 nanometers. The mixture may be termed a colloid or a colloidal suspension. Sometimes the term “colloid” only refers to the particles in the mixture and not the entire suspension.

For the preparation of colloids comprising at least one cationic or cationically ionizable lipid methods are applicable herein that are conventionally used for preparing liposomal vesicles and are appropriately adapted. The most commonly used methods for preparing liposomal vesicles share the following fundamental stages: (i) lipids dissolution in organic solvents, (ii) drying of the resultant solution, and (iii) hydration of dried lipid (using various aqueous media).

In the film hydration method, lipids are firstly dissolved in a suitable organic solvent, and dried down to yield a thin film at the bottom of the flask. The obtained lipid film is hydrated using an appropriate aqueous medium to produce a liposomal dispersion. Furthermore, an additional downsizing step may be included.

Reverse phase evaporation is an alternative method to the film hydration for preparing liposomal vesicles that involves formation of a water-in-oil emulsion between an aqueous phase and an organic phase containing lipids. A brief sonication of this mixture is required for system homogenization. The removal of the organic phase under reduced pressure yields a milky gel that turns subsequently into a liposomal suspension.

The term “ethanol injection technique” refers to a process, in which an ethanol solution comprising lipids is rapidly injected into an aqueous solution through a needle. This action disperses the lipids throughout the solution and promotes lipid structure formation, for example lipid vesicle formation such as liposome formation. Generally, the nucleic acid (especially RNA such as mRNA) lipoplex particles described herein are obtainable by adding nucleic acid (especially RNA such as mRNA) to a colloidal liposome dispersion. Using the ethanol injection technique, such colloidal liposome dispersion is, in one embodiment, formed as follows: an ethanol solution comprising lipids, such as cationically ionizable lipids (like a cationically ionizable lipid of formula (X) disclosed herein; a cationically ionizable lipid having one of the structures A to G disclosed herein; a cationically ionizable lipid of formula (XI) disclosed herein; DOTMA and/or DODMA) and additional lipids (such as a polymer-conjugated lipid (e.g., a polyethylene glycol (PEG) lipid; or a polysarcosine-lipid conjugate or a conjugate of polysarcosine and a lipid-like material); a neutral lipid (such as a phospholipid); a steroid (such as cholesterol); and combinations), is injected into an aqueous solution under stirring. In some embodiments, the nucleic acid (especially RNA such as mRNA) lipoplex particles described herein are obtainable without a step of extrusion.

The term “extruding” or “extrusion” refers to the creation of particles having a fixed, cross-sectional profile. In particular, it refers to the downsizing of a particle, whereby the particle is forced through filters with defined pores.

Other methods having organic solvent free characteristics may also be used according to the present disclosure for preparing a colloid.

LNPs typically comprise four components: cationically ionizable or cationic lipids; neutral lipids such as phospholipids; a steroid such as cholesterol; and a polymer-conjugated lipid (which is sometimes called “stealth lipid” or “sterically stabilizing lipid”), such as a PEG lipid or a polysarcosine-conjugated lipid. Each component is responsible for payload protection, and enables effective intracellular delivery.

However, due to the disadvantages of commonly utilized PEG lipids, i.e., PEG lipids having at least 30 consecutive ethylene glycol repeating units (which are the most common polymer-conjugated lipids), LNPs of the present disclosure preferably are substantially free of PEG lipids having at least 30 consecutive ethylene glycol repeating units.

Different types of nucleic acid containing particles have been described previously to be suitable for delivery of nucleic acid in particulate form (cf., e.g., Kaczmarek, J. C. et al., 2017, Genome Medicine 9, 60). For non-viral nucleic acid delivery vehicles, nanoparticle encapsulation of nucleic acid physically protects nucleic acid from degradation and, depending on the specific chemistry, can aid in cellular uptake and endosomal escape.

In some embodiments, the LNPs comprising nucleic acid (such as RNA) and at least one cationically ionizable lipid described herein are prepared by (a) providing (e.g., preparing) a nucleic acid solution containing water and a first buffer system; (b) providing (e.g., preparing) an organic (e.g. ethanolic) solution comprising the cationically ionizable lipid, the steroid, and the neutral lipid, and optionally one or more additional lipids (e.g., a polymer-conjugated lipid); (c) and mixing the nucleic acid solution provided (e.g., prepared) under (a) with the organic (e.g., ethanolic) solution provided (e.g., prepared) under (b), thereby preparing a first intermediate formulation comprising the LNPs dispersed in a first aqueous phase comprising the first buffer system; (d) filtrating (e.g., dialyzing, tangential flow filtrating, or diafiltrating) and/or diluting the first intermediate formulation prepared under (c) using a final aqueous buffer solution comprising the final buffer system, thereby preparing the formulation comprising LNPs dispersed in a final aqueous phase comprising the final buffer system. After step (d) one or more steps selected from diluting and filtrating, such as dialyzing, tangential flow filtrating or diafiltrating, can follow. In some embodiments, the first buffer system differs from the final buffer system. In alternative embodiments, the first buffer system and the final buffer system are the same. In some embodiments, in particular those where the first aqueous phase is highly similar to or substantially corresponds to the final aqueous phase or where the first aqueous phase can be transformed into the final aqueous phase simply by diluting the first aqueous phase with a suitable dilution solution (e.g., the final aqueous buffer solution), the filtrating the first intermediate formulation prepared under (c) using a final aqueous buffer solution comprising the final buffer system may be replaced by diluting the first intermediate formulation prepared under (c) using a dilution solution (e.g., the final aqueous buffer solution comprising the final buffer system).

In some embodiments, the LNPs comprising nucleic acid (such as RNA) and at least one cationically ionizable lipid described herein are prepared by (a′) providing (e.g., preparing) liposomes or a colloidal preparation of the cationically ionizable lipid and, if present, one or more additional lipids in an aqueous phase; (b′) providing (e.g., preparing) a nucleic acid (such as RNA) solution containing water and a buffering system; and (c′) mixing the liposomes or colloidal preparation provided (e.g., prepared) under (a′) with the nucleic acid (such as RNA) solution provided (e.g., prepared) under (b′). After step (c′) one or more steps selected from diluting and filtrating, such as dialyzing, tangential flow filtrating, or diafiltrating, can follow.

The present disclosure describes compositions which comprise nucleic acid (such as RNA, especially mRNA), and at least one cationically ionizable lipid which associates with the nucleic acid (such as RNA) to form nucleic acid particles. The nucleic acid particles may comprise nucleic acid (such as RNA) which is complexed in different forms by non-covalent interactions to the particle. The particles described herein are not viral particles, in particular infectious viral particles, i.e., they are not able to virally infect cells.

Suitable cationically ionizable lipids are those that form nucleic acid particles and are included by the term “particle forming components” or “particle forming agents”. The term “particle forming components” or “particle forming agents” relates to any components which associate with nucleic acid to form nucleic acid particles. Such components include any component which can be part of nucleic acid particles.

In some embodiments, nucleic acid (such as RNA) particles (especially mRNAparticles) comprise more than one type of nucleic acid (such as RNA) molecules, where the molecular parameters of the nucleic acid molecules may be similar or different from each other, like with respect to molar mass or fundamental structural elements such as molecular architecture, capping, coding regions or other features.

In particulate formulation, it is possible that each nucleic acid (such as RNA) species is separately formulated as an individual particulate formulation. In that case, each individual particulate formulation will comprise one nucleic acid (such as RNA) species. The individual particulate formulations may be present as separate entities, e.g. in separate containers. Such formulations are obtainable by providing each nucleic acid (such as RNA) species separately (typically each in the form of a nucleic acid-containing solution) together with a particle-forming agent, thereby allowing the formation of particles.

Respective particles will contain exclusively the specific nucleic acid (such as RNA) species that is being provided when the particles are formed (individual particulate formulations). In some embodiments, a composition such as a pharmaceutical composition comprises more than one individual particle formulation. Respective pharmaceutical compositions are referred to as mixed particulate formulations. Mixed particulate formulations according to the present disclosure are obtainable by forming, separately, individual particulate formulations, followed by a step of mixing of the individual particulate formulations. By the step of mixing, a formulation comprising a mixed population of nucleic acid-containing particles is obtainable. Individual particulate populations may be together in one container, comprising a mixed population of individual particulate formulations. Alternatively, it is possible that all nucleic acid (such as RNA) species of the pharmaceutical composition are formulated together as a combined particulate formulation. Such formulations are obtainable by providing a combined formulation (typically combined solution) of all nucleic acid (such as RNA) species together with a particle-forming agent, thereby allowing the formation of particles. As opposed to a mixed particulate formulation, a combined particulate formulation will typically comprise particles which comprise more than one nucleic acid (such as RNA) species. In a combined particulate composition different nucleic acid (such as RNA) species are typically present together in a single particle.

### Lipids

The terms “lipid” and “lipid-like material” are broadly defined herein as molecules which comprise one or more hydrophobic moieties or groups and optionally also one or more hydrophilic moieties or groups. Molecules comprising hydrophobic moieties and hydrophilic moieties are also frequently denoted as amphiphiles. Lipids are usually insoluble or poorly soluble in water, but soluble in many organic solvents. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). The hydrophilic groups may comprise polar and/or charged groups and include carbohydrates, phosphate, carboxylic, sulfate, amino (e.g., tertiary amino), sulfhydryl, nitro, hydroxyl, and other like groups.

As used herein, the term “hydrophobic” refers to any a molecule, moiety or group which is substantially immiscible or insoluble in aqueous solution. The term hydrophobic group includes hydrocarbons having at least 6 carbon atoms. The monovalent radical of a hydrocarbon is referred to as hydrocarbyl herein. The hydrophobic group can have functional groups (e.g., ether, ester, halide, etc.) and atoms other than carbon and hydrogen as long as the group satisfies the condition of being substantially immiscible or insoluble in aqueous solution.

The term “hydrocarbon” includes non-cyclic, e.g., linear (straight) or branched, hydrocarbyl groups, such as alkyl, alkenyl, or alkynyl as defined herein. It should be appreciated that one or more of the hydrogen atoms in alkyl, alkenyl, or alkynyl may be substituted with other atoms, e.g., halogen, oxygen or sulfur. Unless stated otherwise, hydrocarbon groups can also include a cyclic (alkyl, alkenyl or alkynyl) group or an aryl group, provided that the overall polarity of the hydrocarbon remains relatively nonpolar.

As used herein, the term “amphiphilic” refers to a molecule having both a polar portion and a non-polar portion. Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt. For purposes of the disclosure, the amphiphilic compound can be, but is not limited to, one or a plurality of natural or non-natural lipids and lipid-like compounds.

The term “lipid-like material”, “lipid-like compound” or “lipid-like molecule” relates to substances that structurally and/or functionally relate to lipids but may not be considered as lipids in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term refers to molecules, which comprise hydrophilic and hydrophobic moieties with different structural organization, which may or may not be similar to that of lipids. Examples of lipid-like compounds capable of spontaneous integration into cell membranes include functional lipid constructs such as synthetic function-spacer-lipid constructs (FSL), synthetic function-spacer-sterol constructs (FSS) as well as artificial amphipathic molecules. Lipids comprising two long alkyl chains and a polar head group are generally cylindrical. The area occupied by the two alkyl chains is similar to the area occupied by the polar head group. Such lipids have low solubility as monomers and tend to aggregate into planar bilayers that are water insoluble. Traditional surfactant monomers comprising only one linear alkyl chain and a hydrophilic head group are generally cone shaped. The hydrophilic head group tends to occupy more molecular space than the linear alkyl chain. In some embodiments, surfactants tend to aggregate into spherical or elliptoid micelles that are water soluble. While lipids also have the same general structure as surfactants—a polar hydrophilic head group and a nonpolar hydrophobic tail —lipids differ from surfactants in the shape of the monomers, in the type of aggregates formed in solution, and in the concentration range required for aggregation. As used herein, the term “lipid” is to be construed to cover both lipids and lipid-like materials unless otherwise indicated herein or clearly contradicted by context.

Specific examples of amphiphilic compounds that may be included in an amphiphilic layer include, but are not limited to, phospholipids, aminolipids and sphingolipids.

In certain embodiments, the amphiphilic compound is a lipid. The term “lipid” refers to a group of organic compounds that are characterized by being insoluble in water, but soluble in many organic solvents. Generally, lipids may be divided into eight categories: fatty acids, glycerolipids, glycerophospholipids, sphingolipids, saccharolipids, polyketides (derived from condensation of ketoacyl subunits), sterol lipids and prenol lipids (derived from condensation of isoprene subunits). Although the term “lipid” is sometimes used as a synonym for fats, fats are a subgroup of lipids called triglycerides. Lipids also encompass molecules such as fatty acids and their derivatives (including tri-, di-, monoglycerides, and phospholipids), as well as steroids, i.e., sterol-containing metabolites such as cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

Fatty acids, or fatty acid residues are a diverse group of molecules made of a hydrocarbon chain that terminates with a carboxylic acid group; this arrangement confers the molecule with a polar, hydrophilic end, and a nonpolar, hydrophobic end that is insoluble in water. The carbon chain, typically between four and 24 carbons long, may be saturated or unsaturated, and may be attached to functional groups containing oxygen, halogens, nitrogen, and sulfur. If a fatty acid contains a double bond, there is the possibility of either a cis or trans geometric isomerism, which significantly affects the molecule's configuration. Cis-double bonds cause the fatty acid chain to bend, an effect that is compounded with more double bonds in the chain. Other major lipid classes in the fatty acid category are the fatty esters and fatty amides.

Glycerolipids are composed of mono-, di-, and tri-substituted glycerols, the best-known being the fatty acid triesters of glycerol, called triglycerides. The word “triacylglycerol” is sometimes used synonymously with “triglyceride”. In these compounds, the three hydroxyl groups of glycerol are each esterified, typically by different fatty acids. Additional subclasses of glycerolipids are represented by glycosylglycerols, which are characterized by the presence of one or more sugar residues attached to glycerol via a glycosidic linkage.

Glycerophospholipids are amphipathic molecules (containing both hydrophobic and hydrophilic regions) that contain a glycerol core linked to two fatty acid-derived “tails” by ester linkages and to one “head” group by a phosphate ester linkage. Examples of glycerophospholipids, usually referred to as phospholipids (though sphingomyelins are also classified as phospholipids) are phosphatidylcholine (also known as PC, GPCho or lecithin), phosphatidylethanolamine (PE or GPEtn) and phosphatidylserine (PS or GPSer).

Sphingolipids are a complex family of compounds that share a common structural feature, a sphingoid base backbone. The major sphingoid base in mammals is commonly referred to as sphingosine. Ceramides (N-acyl-sphingoid bases) are a major subclass of sphingoid base derivatives with an amide-linked fatty acid. The fatty acids are typically saturated or mono-unsaturated with chain lengths from 16 to 26 carbon atoms. The major phosphosphingolipids of mammals are sphingomyelins (ceramide phosphocholines), whereas insects contain mainly ceramide phosphoethanolamines and fungi have phytoceramide phosphoinositols and mannose-containing headgroups. The glycosphingolipids are a diverse family of molecules composed of one or more sugar residues linked via a glycosidic bond to the sphingoid base. Examples of these are the simple and complex glycosphingolipids such as cerebrosides and gangliosides.

Sterol lipids, such as cholesterol and its derivatives, or tocopherol and its derivatives, are an important component of membrane lipids, along with the glycerophospholipids and sphingomyelins.

Saccharolipids describe compounds in which fatty acids are linked directly to a sugar backbone, forming structures that are compatible with membrane bilayers. In the saccharolipids, a monosaccharide substitutes for the glycerol backbone present in glycerolipids and glycerophospholipids. The most familiar saccharolipids are the acylated glucosamine precursors of the Lipid A component of the lipopolysaccharides in Gram-negative bacteria. Typical lipid A molecules are disaccharides of glucosamine, which are derivatized with as many as seven fatty-acyl chains. The minimal lipopolysaccharide required for growth in E. coli is Kdo2-Lipid A, a hexa-acylated disaccharide of glucosamine that is glycosylated with two 3-deoxy-D-manno-octulosonic acid (Kdo) residues.

Polyketides are synthesized by polymerization of acetyl and propionyl subunits by classic enzymes as well as iterative and multimodular enzymes that share mechanistic features with the fatty acid synthases. They comprise a large number of secondary metabolites and natural products from animal, plant, bacterial, fungal and marine sources, and have great structural diversity. Many polyketides are cyclic molecules whose backbones are often further modified by glycosylation, methylation, hydroxylation, oxidation, or other processes.

### Cationic and Cationically Ionizable Lipids

The nucleic acid (such as RNA) compositions described herein and the nucleic acid particles (especially RNA LNPs) described herein comprise at least one cationic or cationically ionizable lipid as particle forming agent. Cationic and cationically ionizable lipids contemplated for use herein include any cationic/cationically ionizable lipids or lipid-like materials which are able to electrostatically bind nucleic acid. In one embodiment, cationic/cationically ionizable lipids contemplated for use herein can be associated with nucleic acid, e.g. by forming complexes with the nucleic acid or forming vesicles in which the nucleic acid is enclosed or encapsulated.

As used herein, a “cationic lipid” or “cationic lipid-like material” refers to a lipid or lipid-like material having a net positive charge. Cationic lipids or lipid-like materials bind negatively charged nucleic acid by electrostatic interaction. Generally, cationic lipids possess a lipophilic moiety, such as a sterol, an acyl chain, a diacyl or more acyl chains, and the head group of the lipid typically carries the positive charge.

In certain embodiments, a cationic lipid or lipid-like material has a net positive charge only at certain pH, in particular acidic pH, while it has preferably no net positive charge, preferably has no charge, i.e., it is neutral, at a different, preferably higher pH such as physiological pH. This ionizable behavior is thought to enhance efficacy through helping with endosomal escape and reducing toxicity as compared with particles that remain cationic at physiological pH.

As used herein, a “cationically ionizable lipid” refers to a lipid or lipid-like material which has a net positive charge or is neutral, i.e., a lipid which is not permanently cationic. Thus, depending on the pH of the composition in which the cationically ionizable lipid is solved, the cationically ionizable lipid is either positively charged or neutral.

In one embodiment, the cationically ionizable lipid comprises a head group which includes at least one nitrogen atom (N) which is positive charged or capable of being protonated, preferably under physiological conditions. In some embodiments, the cationically ionizable lipid comprises a head group which includes at least one tertiary amine moiety.

Examples of cationic or cationically ionizable lipids include, but are not limited to N,N-dimethyl-2,3-dioleyloxypropylamine (DODMA), 1,2-di-O-octadecenyl-3-trimethylammonium propane (DOTMA), 3-(N-(N′,N′-dimethylaminoethane)-carbamoyl)cholesterol (DC-Chol), dimethyldioctadecylammonium (DDAB); 1,2-dioleoyl-3-trimethylammonium propane (DOTAP); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-l-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis, cis-9,12-oc-tadecadienoxy)propane (CLinDMA), 2-[5′-(cholest-5-en-3-beta-oxy)-3′-oxapentoxy)-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-dilinoleoyloxy-N,N-dimethylpropylamine (DLinDAP), 1,2-N,N′-Dilinoleylcarbamyl-3-dimethylaminopropane (DLincarbDAP), 1,2-dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), N-(2-Hydroxyethyl)-N,N-dimethyl-2,3-bis(tetradecyloxy)-1-propanaminium bromide (DMRIE), (+)-N-(3-aminopropyl)-N,N-dimethyl-2,3-bis(cis-9-tetradecenyloxy)-1-propanaminium bromide (GAP-DMORIE), (+)-N-(3-aminopropyl)-N,N-dimethyl-2,3-bis(dodecyloxy)-1-propanaminium bromide (GAP-DLRIE), (+)-N-(3-aminopropyl)-N,N-dimethyl-2,3-bis(tetradecyloxy)-1-propanaminium bromide (GAP-DMRIE), N-(2-aminoethyl)-N,N-dimethyl-2,3-bis(tetradecyloxy)-1-propanaminium bromide (PAE-DMRIE), N-(4-carboxybenzyl)-N,N-dimethyl-2,3-bis(oleoyloxy)propan-1-aminium (DOBAQ), 2-({8-[(3p)-cholest-5-en-3-yloxy]octyl}oxy)-N,N-dimethyl-3-[(9Z,12Z)-octadeca-9,12-dien-1-yloxy]propan-1-amine (octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-amonium bromide (DLRIE), N-(2-aminoethyl)-N,N-dimethyl-2,3-bis(tetradecyloxy)propan-1-aminium bromide (DMORIE), di((Z)-non-2-en-1-yl) 8,8′-((((2(dimethylamino)ethyl)thio)carbonyl)azanediyl)-dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), Di((Z)-non-2-en-1-yl)-9-((4-(dimethylaminobutanoyl)-oxy)heptadecanedioate (L319), N-dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethyl-amino)propionamide (lipidoid 98N₁₂-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200), and the following structures (XV-1) to (XV-6):

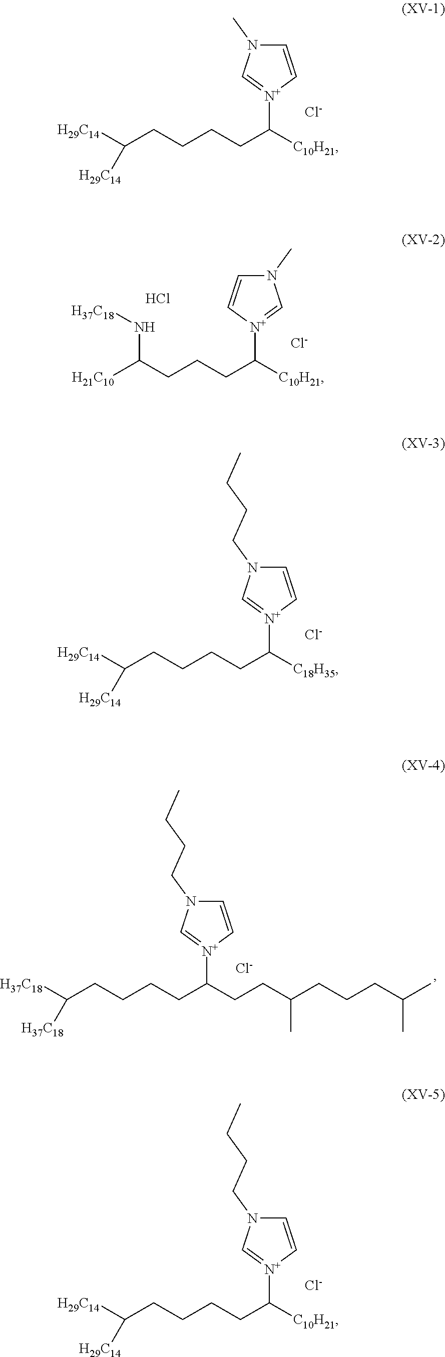

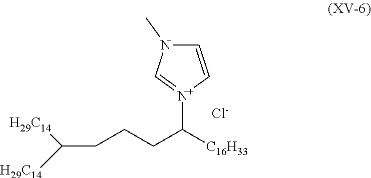

Preferred are DODMA, DOTMA, DOTAP, DODAC, and DOSPA. In specific embodiments, the cationic or cationically ionizable lipid is DODMA.

DOTMA is a cationic lipid with a quaternary amine headgroup. The structure of DOTMA may be represented as follows:

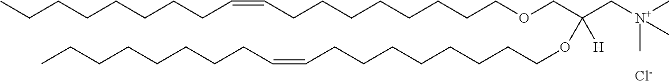 [SMILES: CCCCCCCC/C=C\CCCCCCCCOCC(C[N+](C)(C)C)OCCCCCCCC/C=C\CCCCCCCC ; [Cl-]]

DODMA is a cationically ionizable lipid with a tertiary amine headgroup. The structure of DODMA may be represented as follows:

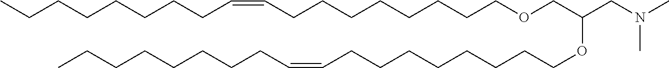 [SMILES: CCCCCCCC/C=C\CCCCCCCCOCC(CN(C)C)OCCCCCCCC/C=C\CCCCCCCC]

In certain embodiments, the composition comprises a cationically ionizable lipid (in particular, when the composition comprises lipid nanoparticles (LNPs)).

In certain embodiments, the composition comprises a cationic lipid (in particular, when the composition comprises lipoplexes (LPXs)).

Examples of cationically ionizable lipids are disclosed, for example, in WO 2016/176330 and WO 2018/078053. In some embodiments, the cationically ionizable lipid has the structure of Formula (X):

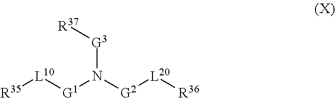

or a pharmaceutically acceptable salt, tautomer, or stereoisomer thereof, wherein:
one of L¹⁰ and L²⁰ is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹⁰ and L²⁰ is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₂₋₁₂ alkenylene;
G³ is C₁₋₂₄ alkylene, C₂₋₂₄ alkenylene, C₃₋₈ cycloalkylene, or C₃₋ₛ cycloalkenylene;
R^{a} is H or C1-12 alkyl;
R³⁵ and R³⁶ are each independently C₆₋₂₄ alkyl or C₆₋₂₄ alkenyl;
R³⁷ is H, OR⁵⁰, CN, —C(═O)OR⁴⁰, —OC(═O)R⁴⁰ or NR⁵⁰C(═O)R⁴⁰;
R⁴⁰ is C₁₋₁₂ alkyl;
R⁵⁰ is H or C₁₋₆ alkyl; and
x is 0, 1 or 2.

In some of the foregoing embodiments of Formula (X), the lipid has one of the following structures (XA) or (XB):

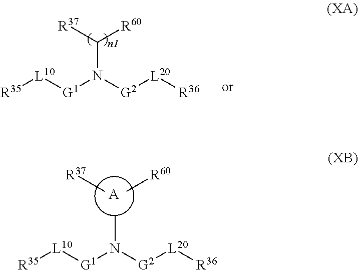

wherein:
A is a 3 to 8-membered cycloalkyl or cycloalkylene group;
R⁶⁰ is, at each occurrence, independently H, OH or C₁-C₂₄ alkyl;
n1 is an integer ranging from 1 to 15.

In some of the foregoing embodiments of Formula (X), the lipid has structure (XA), and in other embodiments, the lipid has structure (XB).

In other embodiments of Formula (X), the lipid has one of the following structures (XC) or (XD):

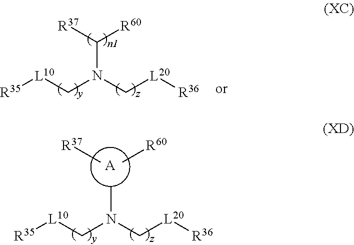

wherein y and z are each independently integers ranging from 1 to 12.

In any of the foregoing embodiments of Formula (X), one of L¹⁰ and L²⁰ is —O(C═O)—. For example, in some embodiments each of L¹⁰ and L²⁰ are —O(C═O)—. In some different embodiments of any of the foregoing, L¹⁰ and L²⁰ are each independently —(C═O)O— or —O(C═O)—. For example, in some embodiments each of L¹⁰ and L²⁰ is —(C═O)O—.

In some different embodiments of Formula (X), the lipid has one of the following structures (XE) or (XF):

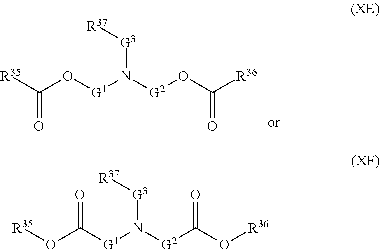

In some of the foregoing embodiments of Formula (X), the lipid has one of the following structures (XG), (XH), (XJ), or (XK):

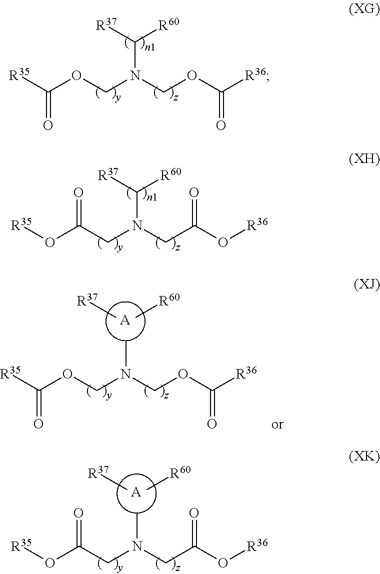

In some of the foregoing embodiments of Formula (X), n1 is an integer ranging from 2 to 12, for example from 2 to 8 or from 2 to 4. For example, in some embodiments, n1 is 3, 4, 5 or 6. In some embodiments, n1 is 3. In some embodiments, n1 is 4. In some embodiments, n1 is 5. In some embodiments, n1 is 6.

In some other of the foregoing embodiments of Formula (X), y and z are each independently an integer ranging from 2 to 10. For example, in some embodiments, y and z are each independently an integer ranging from 4 to 9 or from 4 to 6.

In some of the foregoing embodiments of Formula (X), R⁶⁰ is H. In other of the foregoing embodiments, R⁶⁰ is C₁-C₂₄ alkyl. In other embodiments, R⁶⁰ is OH.

In some embodiments of Formula (X), G3 is unsubstituted. In other embodiments, G3 is substituted. In various different embodiments, GU is linear C₁-C₂₄ alkylene or linear C₂-C₂₄ alkenylene.

In some other foregoing embodiments of Formula (X), R³⁵ or R³⁶, or both, is C₆-C₂₄ alkenyl. For example, in some embodiments, R³⁵ and R³⁶ each, independently have the following structure:

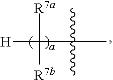

wherein:
R^{7a} and R^{7b} are, at each occurrence, independently H or C₁-C₁₂ alkyl; and a is an integer from 2 to 12,
wherein R^{7a}, R^{7b} and a are each selected such that R³⁵ and R³⁶ each independently comprise from 6 to 20 carbon atoms. For example, in some embodiments a is an integer ranging from 5 to 9 or from 8 to 12.

In some of the foregoing embodiments of Formula (X), at least one occurrence of R^{7a} is H. For example, in some embodiments, R^{7a} is H at each occurrence. In other different embodiments of the foregoing, at least one occurrence of R^{7b} is C₁-C₅ alkyl. For example, in some embodiments, C₁-C₅ alkyl is methyl, ethyl, n-propyl, iso-propyl, n-butyl, iso-butyl, tert-butyl, n-hexyl or n-octyl.

In different embodiments of Formula (X), R³⁵ or R³⁶, or both, has one of the following structures:

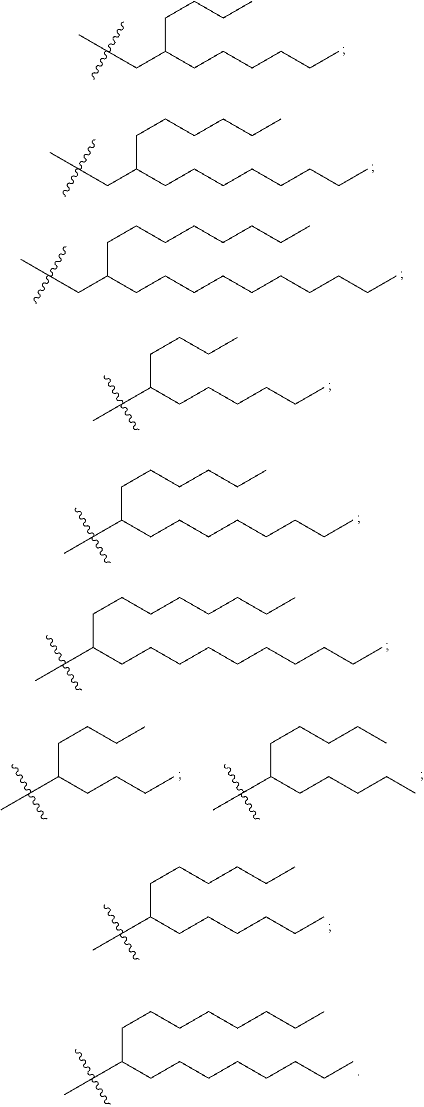

In some of the foregoing embodiments of Formula (X), R³² is OH, CN, —C(═O)OR⁴⁰, —OC(═O)R⁴¹ or —NHC(═O)R⁴¹. In some embodiments, R⁴¹ is methyl or ethyl.

In various different embodiments, the cationically ionizable lipid of Formula (X) has one of the structures set forth below.

Representative Compounds of Formula (X) 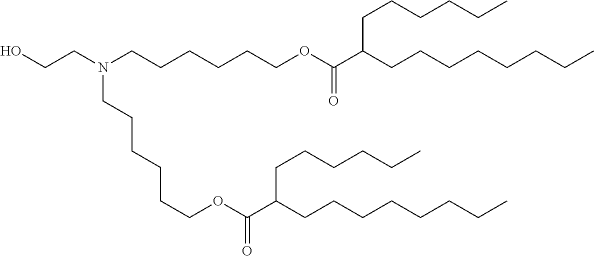 [SMILES: CCCCCCCCC(CCCCCC)C(=O)OCCCCCCN(CCO)CCCCCCOC(=O)C(CCCCCC)CCCCCCCC]
X-1 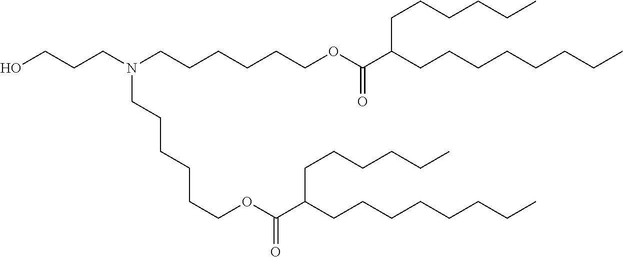 [SMILES: CCCCCCCCC(CCCCCC)C(=O)OCCCCCCN(CCCO)CCCCCCOC(=O)C(CCCCCC)CCCCCCCC]
X-2 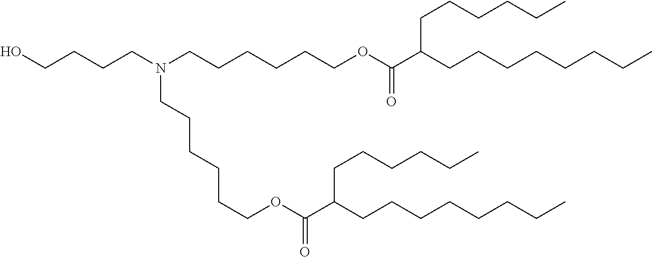 [SMILES: CCCCCCCCC(CCCCCC)C(=O)OCCCCCCN(CCCCO)CCCCCCOC(=O)C(CCCCCC)CCCCCCCC]
X-3 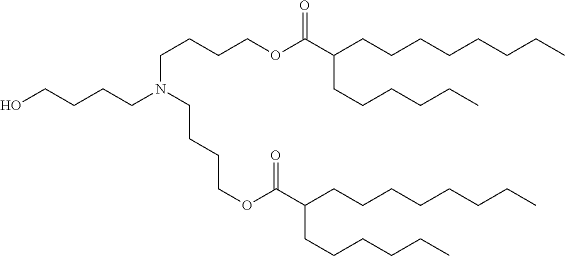 [SMILES: CCCCCCCCC(CCCCCC)C(=O)OCCCCN(CCCCO)CCCCOC(=O)C(CCCCCC)CCCCCCCC]
X-4 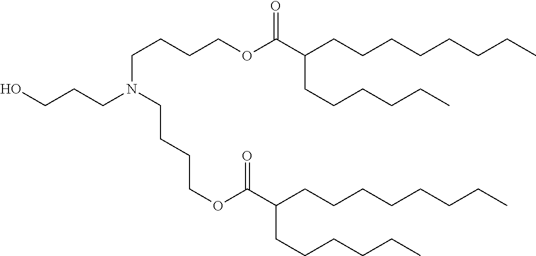 [SMILES: CCCCCCCCC(CCCCCC)C(=O)OCCCCN(CCCO)CCCCOC(=O)C(CCCCCC)CCCCCCCC]
X-5 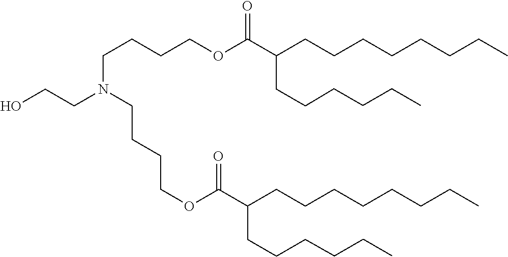 [SMILES: CCCCCCCCC(CCCCCC)C(=O)OCCCCN(CCO)CCCCOC(=O)C(CCCCCC)CCCCCCCC]
X-6 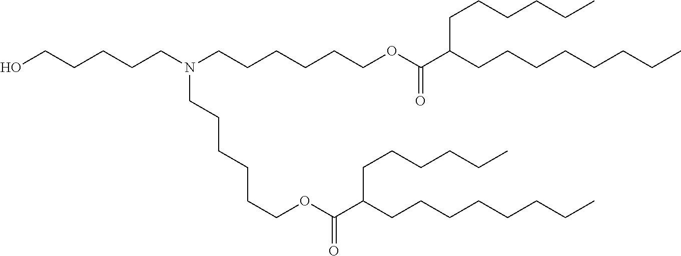 [SMILES: CCCCCCCCC(CCCCCC)C(=O)OCCCCCCN(CCCCCO)CCCCCCOC(=O)C(CCCCCC)CCCCCCCC]
X-7 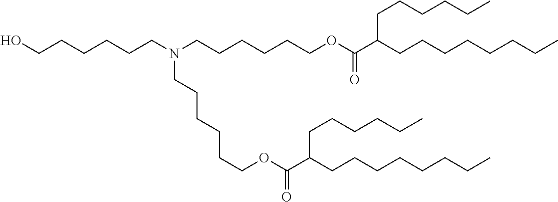 [SMILES: CCCCCCCCC(CCCCCC)C(=O)OCCCCCCN(CCCCCCO)CCCCCCOC(=O)C(CCCCCC)CCCCCCCC]
X-8 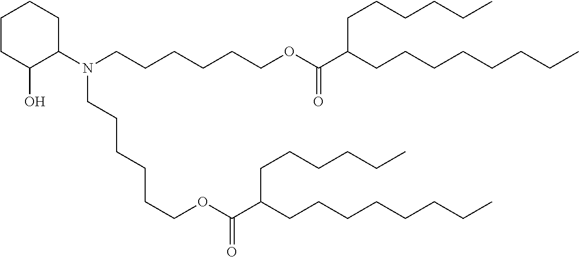 [SMILES: CCCCCCCCC(CCCCCC)C(=O)OCCCCCCN(CCCCCCOC(=O)C(CCCCCC)CCCCCCCC)C1CCCCC1O]
X-9 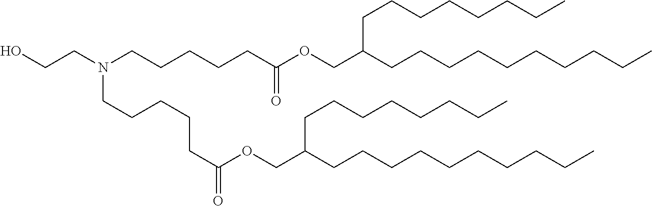 [SMILES: CCCCCCCCCCC(CCCCCCCC)COC(=O)CCCCCN(CCO)CCCCCC(=O)OCC(CCCCCCCC)CCCCCCCCCC]
X-10 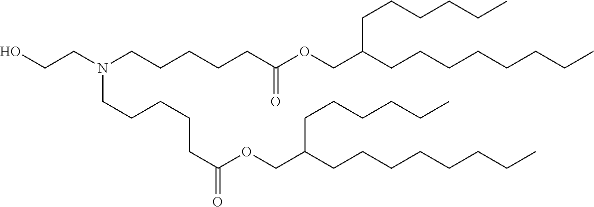 [SMILES: CCCCCCCCC(CCCCCC)COC(=O)CCCCCN(CCO)CCCCCC(=O)OCC(CCCCCC)CCCCCCCC]
X-11 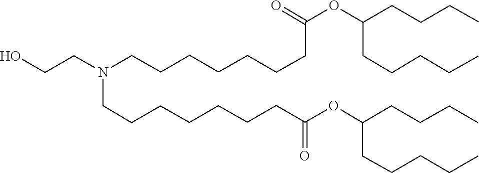 [SMILES: CCCCCC(CCCC)OC(=O)CCCCCCCN(CCO)CCCCCCCC(=O)OC(CCCC)CCCCC]
X-12 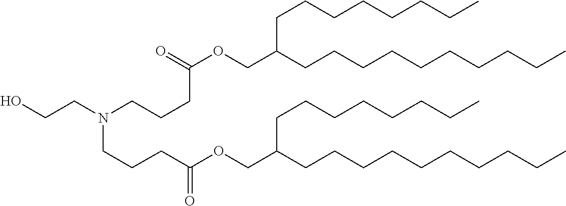 [SMILES: CCCCCCCCCCC(CCCCCCCC)COC(=O)CCCN(CCO)CCCC(=O)OCC(CCCCCCCC)CCCCCCCCCC]
X-13 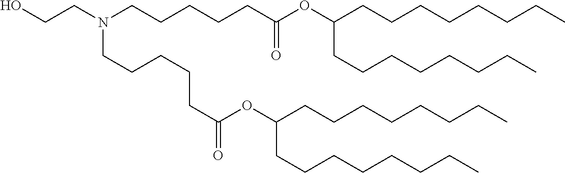 [SMILES: CCCCCCCCC(CCCCCCCC)OC(=O)CCCCCN(CCO)CCCCCC(=O)OC(CCCCCCCC)CCCCCCCC]
X-14 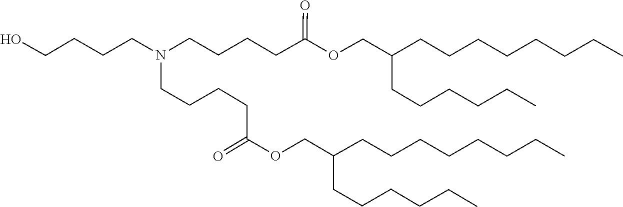 [SMILES: CCCCCCCCC(CCCCCC)COC(=O)CCCCN(CCCCO)CCCCC(=O)OCC(CCCCCC)CCCCCCCC]
X-15 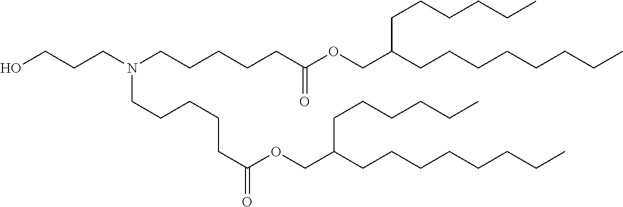 [SMILES: CCCCCCCCC(CCCCCC)COC(=O)CCCCCN(CCCO)CCCCCC(=O)OCC(CCCCCC)CCCCCCCC]
X-16 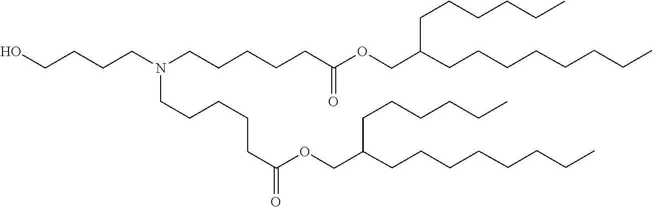 [SMILES: CCCCCCCCC(CCCCCC)COC(=O)CCCCCN(CCCCO)CCCCCC(=O)OCC(CCCCCC)CCCCCCCC]
X-17 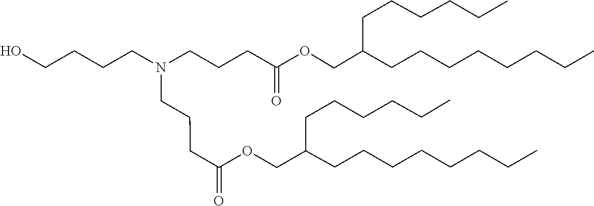 [SMILES: CCCCCCCCC(CCCCCC)COC(=O)CCCN(CCCCO)CCCC(=O)OCC(CCCCCC)CCCCCCCC]
X-18 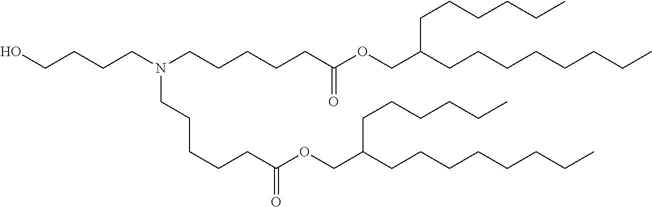 [SMILES: CCCCCCCCC(CCCCCC)COC(=O)CCCCCN(CCCCO)CCCCCC(=O)OCC(CCCCCC)CCCCCCCC]
X-19 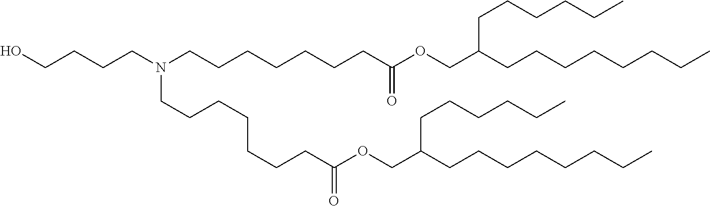 [SMILES: CCCCCCCCC(CCCCCC)COC(=O)CCCCCCCN(CCCCO)CCCCCCCC(=O)OCC(CCCCCC)CCCCCCCC]
X-20 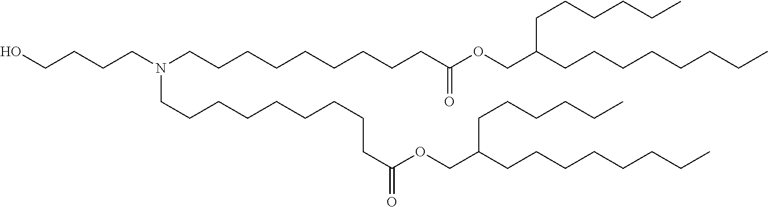 [SMILES: CCCCCCCCC(CCCCCC)COC(=O)CCCCCCCCCN(CCCCO)CCCCCCCCCC(=O)OCC(CCCCCC)CCCCCCCC]
X-21 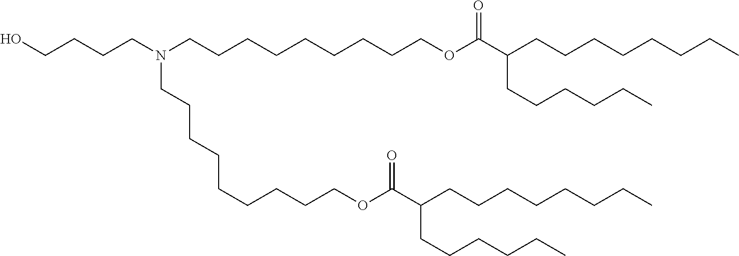 [SMILES: CCCCCCCCC(CCCCCC)C(=O)OCCCCCCCCCN(CCCCO)CCCCCCCCCOC(=O)C(CCCCCC)CCCCCCCC]
X-22 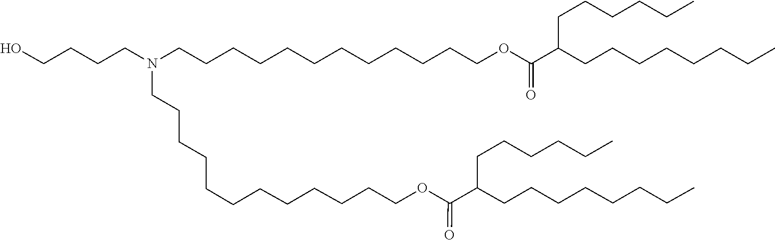 [SMILES: CCCCCCCCC(CCCCCC)C(=O)OCCCCCCCCCCCCN(CCCCO)CCCCCCCCCCCCOC(=O)C(CCCCCC)CCCCCCCC]
X-23 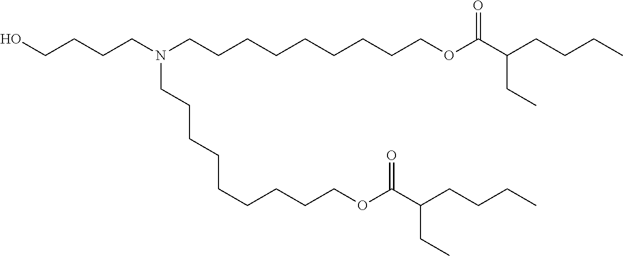 [SMILES: CCCCC(CC)C(=O)OCCCCCCCCCN(CCCCO)CCCCCCCCCOC(=O)C(CC)CCCC]
X-24 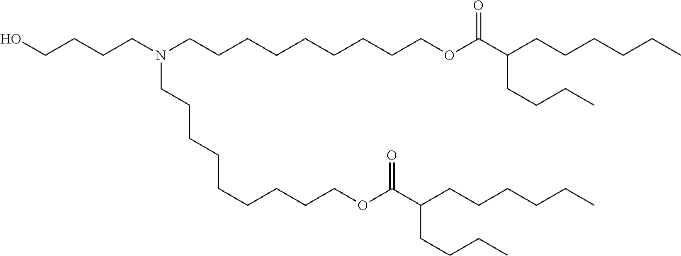 [SMILES: CCCCCCC(CCCC)C(=O)OCCCCCCCCCN(CCCCO)CCCCCCCCCOC(=O)C(CCCC)CCCCCC]
X-25 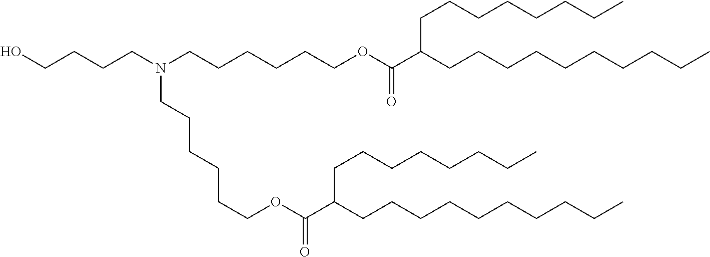 [SMILES: CCCCCCCCCCC(CCCCCCCC)C(=O)OCCCCCCN(CCCCO)CCCCCCOC(=O)C(CCCCCCCC)CCCCCCCCCC]
X-26 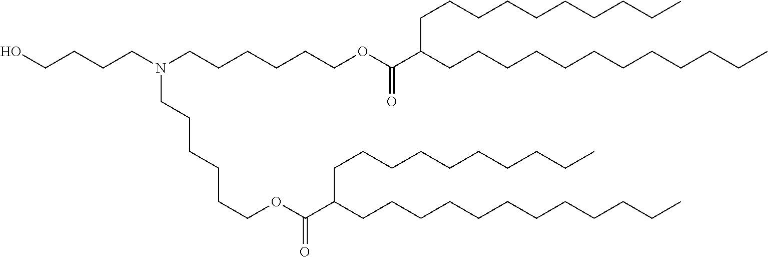 [SMILES: CCCCCCCCCCCCC(CCCCCCCCCC)C(=O)OCCCCCCN(CCCCO)CCCCCCOC(=O)C(CCCCCCCCCC)CCCCCCCCCCCC]
X-27 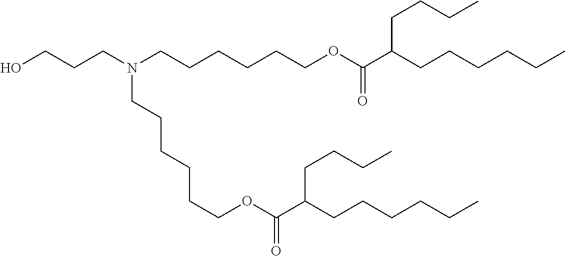 [SMILES: CCCCCCC(CCCC)C(=O)OCCCCCCN(CCCO)CCCCCCOC(=O)C(CCCC)CCCCCC]
X-28 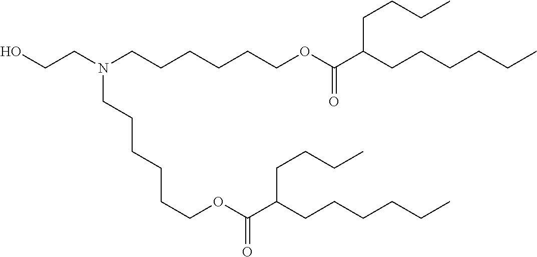 [SMILES: CCCCCCC(CCCC)C(=O)OCCCCCCN(CCO)CCCCCCOC(=O)C(CCCC)CCCCCC]
X-29 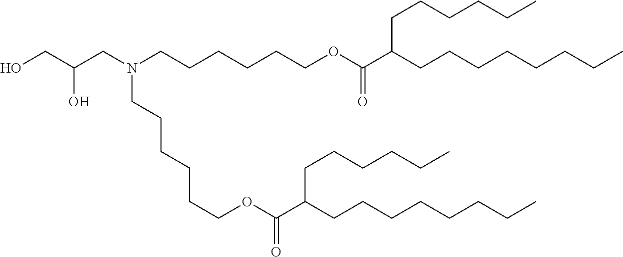 [SMILES: CCCCCCCCC(CCCCCC)C(=O)OCCCCCCN(CCCCCCOC(=O)C(CCCCCC)CCCCCCCC)CC(O)CO]
X-30 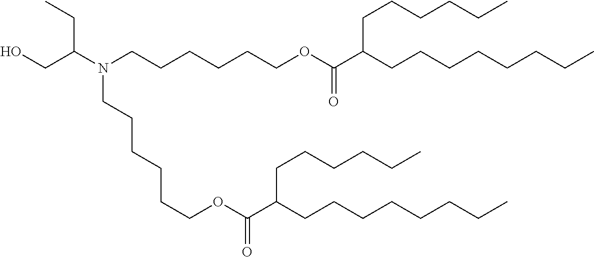 [SMILES: CCCCCCCCC(CCCCCC)C(=O)OCCCCCCN(CCCCCCOC(=O)C(CCCCCC)CCCCCCCC)C(CC)CO]
X-31 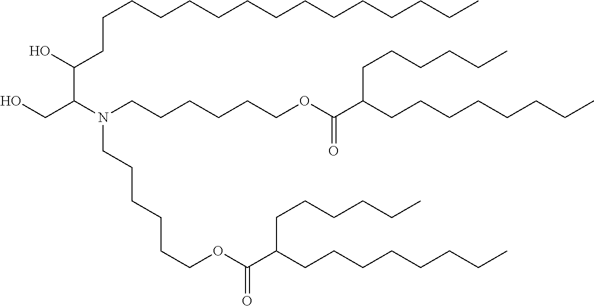 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC]
X-32 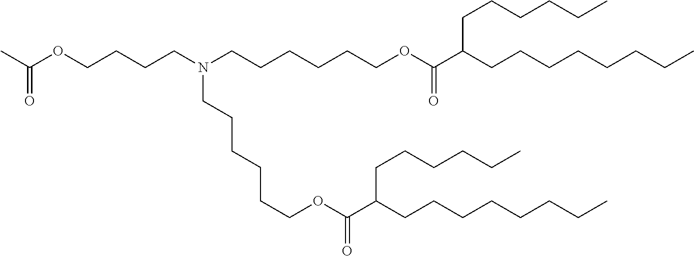 [SMILES: CCCCCCCCC(CCCCCC)C(=O)OCCCCCCN(CCCCCCOC(=O)C(CCCCCC)CCCCCCCC)CCCCOC(C)=O]
X-33 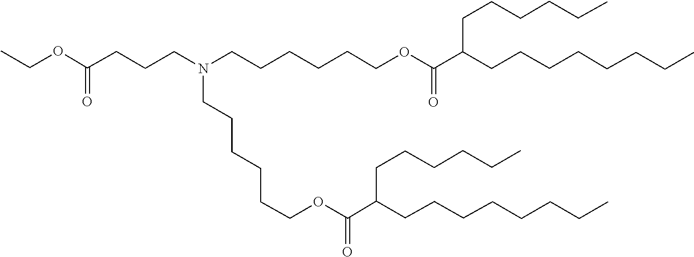 [SMILES: CCCCCCCCC(CCCCCC)C(=O)OCCCCCCN(CCCCCCOC(=O)C(CCCCCC)CCCCCCCC)CCCC(=O)OCC]
X-34 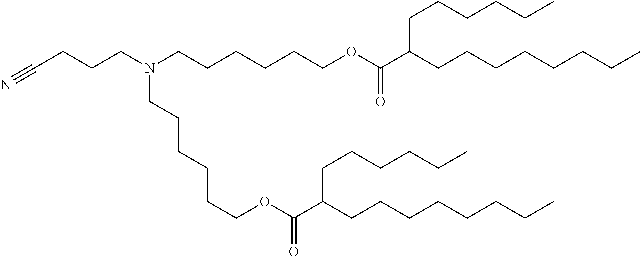 [SMILES: CCCCCCCCC(CCCCCC)C(=O)OCCCCCCN(CCCC#N)CCCCCCOC(=O)C(CCCCCC)CCCCCCCC]
X-35 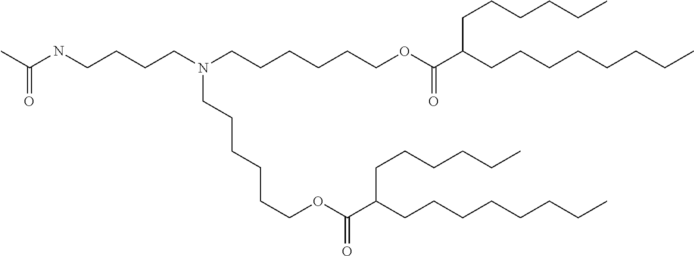 [SMILES: CCCCCCCCC(CCCCCC)C(=O)OCCCCCCN(CCCCCCOC(=O)C(CCCCCC)CCCCCCCC)CCCCNC(C)=O]
X-36 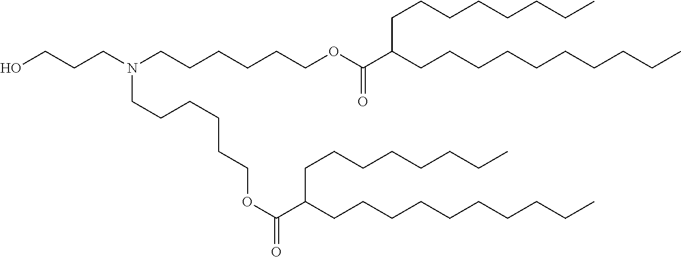 [SMILES: CCCCCCCCCCC(CCCCCCCC)C(=O)OCCCCCCN(CCCO)CCCCCCOC(=O)C(CCCCCCCC)CCCCCCCCCC]
X-37 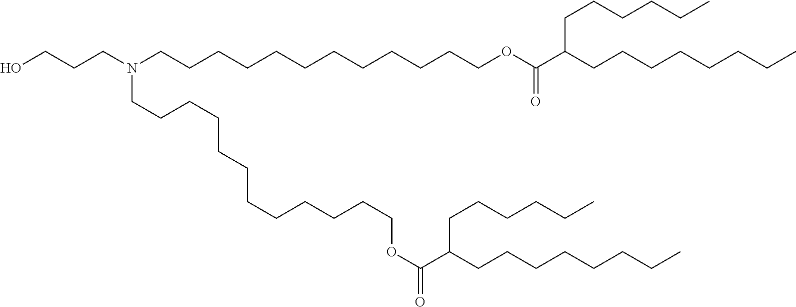 [SMILES: CCCCCCCCC(CCCCCC)C(=O)OCCCCCCCCCCCCN(CCCO)CCCCCCCCCCCCOC(=O)C(CCCCCC)CCCCCCCC]
X-38 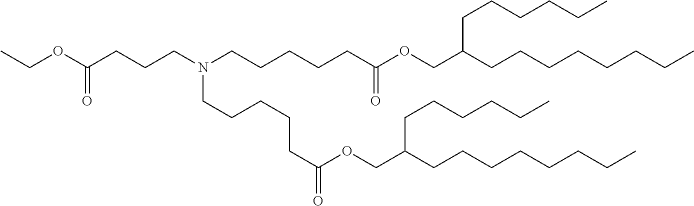 [SMILES: CCCCCCCCC(CCCCCC)COC(=O)CCCCCN(CCCCCC(=O)OCC(CCCCCC)CCCCCCCC)CCCC(=O)OCC]
X-39 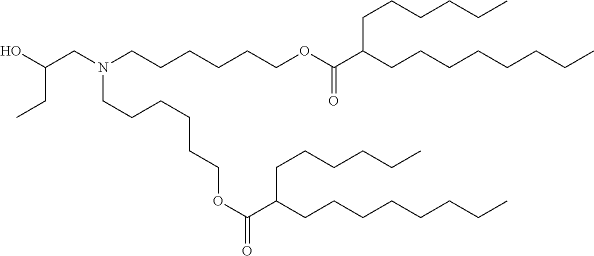 [SMILES: CCCCCCCCC(CCCCCC)C(=O)OCCCCCCN(CCCCCCOC(=O)C(CCCCCC)CCCCCCCC)CC(O)CC]
X-40 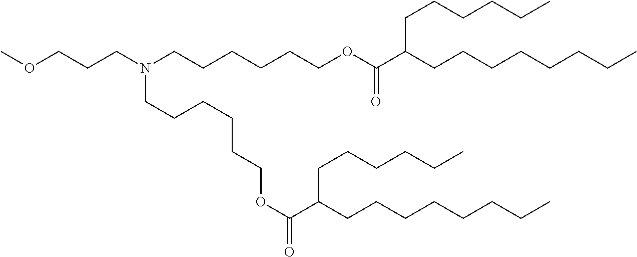 [SMILES: CCCCCCCCC(CCCCCC)C(=O)OCCCCCCN(CCCCCCOC(=O)C(CCCCCC)CCCCCCCC)CCCOC]
X-41 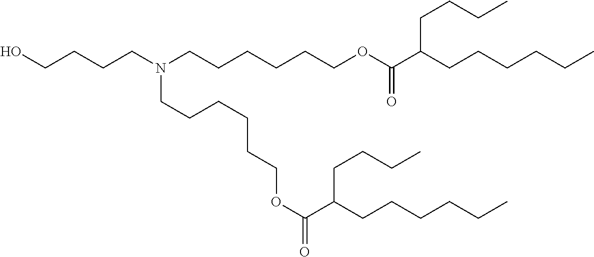 [SMILES: CCCCCCC(CCCC)C(=O)OCCCCCCN(CCCCO)CCCCCCOC(=O)C(CCCC)CCCCCC]
X-42 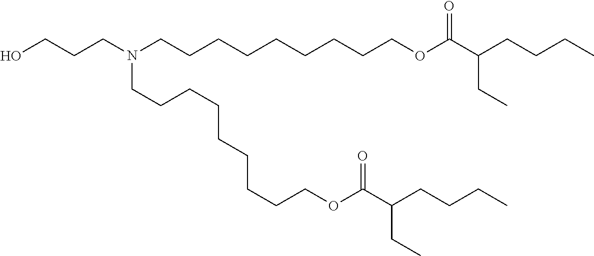 [SMILES: CCCCC(CC)C(=O)OCCCCCCCCCN(CCCO)CCCCCCCCCOC(=O)C(CC)CCCC]
X-43 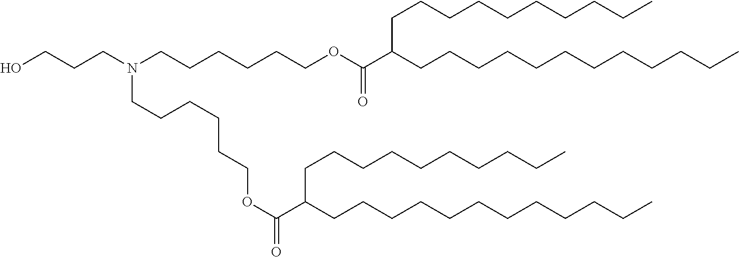 [SMILES: CCCCCCCCCCCCC(CCCCCCCCCC)C(=O)OCCCCCCN(CCCO)CCCCCCOC(=O)C(CCCCCCCCCC)CCCCCCCCCCCC]
X-44 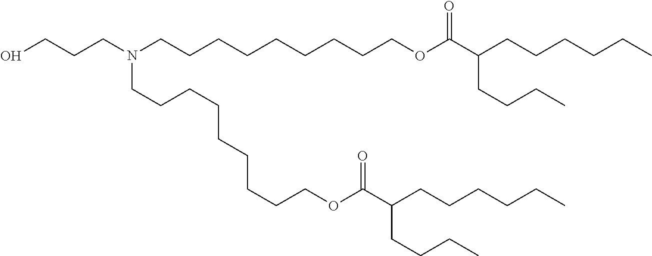 [SMILES: CCCCCCC(CCCC)C(=O)OCCCCCCCCCN(CCCO)CCCCCCCCCOC(=O)C(CCCC)CCCCCC]
X-45 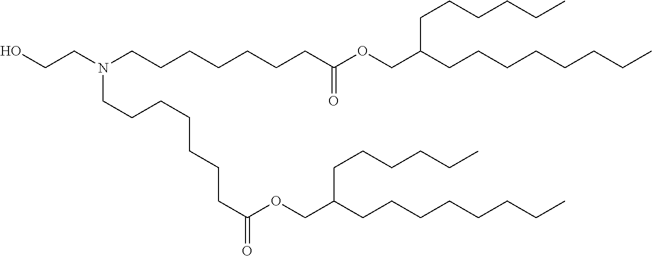 [SMILES: CCCCCCCCC(CCCCCC)COC(=O)CCCCCCCN(CCO)CCCCCCCC(=O)OCC(CCCCCC)CCCCCCCC]
X-46 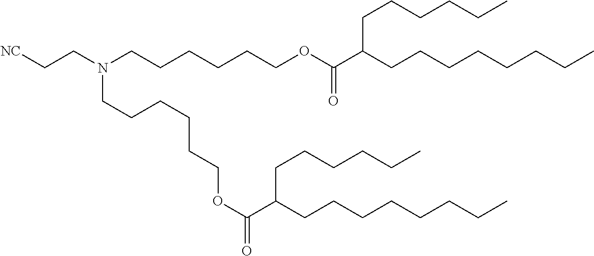 [SMILES: CCCCCCCCC(CCCCCC)C(=O)OCCCCCCN(CCC#N)CCCCCCOC(=O)C(CCCCCC)CCCCCCCC]
X-47 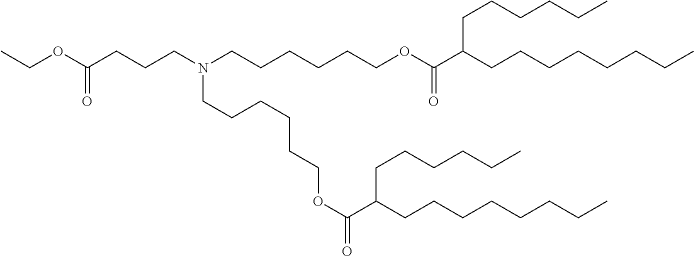 [SMILES: CCCCCCCCC(CCCCCC)C(=O)OCCCCCCN(CCCCCCOC(=O)C(CCCCCC)CCCCCCCC)CCCC(=O)OCC]
X-48 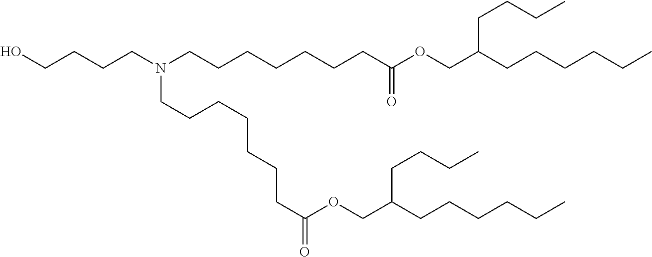 [SMILES: CCCCCCC(CCCC)COC(=O)CCCCCCCN(CCCCO)CCCCCCCC(=O)OCC(CCCC)CCCCCC]
X-49

In various different embodiments, the cationically ionizable lipid has one of the structures set forth in the table below.

| No. | Structure |
|---|---|
| A | 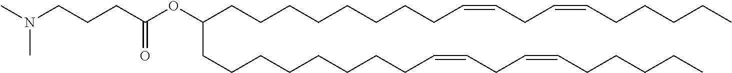 [SMILES: CCCCC/C=C\C/C=C\CCCCCCCCC(CCCCCCCC/C=C\C/C=C\CCCCC)OC(=O)CCCN(C)C] |
| B | 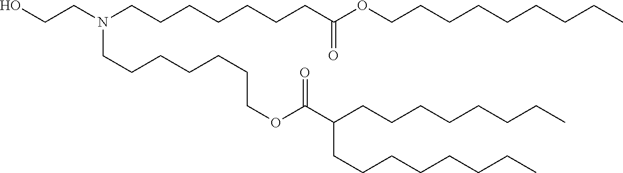 [SMILES: CCCCCCCCCOC(=O)CCCCCCCN(CCO)CCCCCCCOC(=O)C(CCCCCCCC)CCCCCCCC] |
| C | 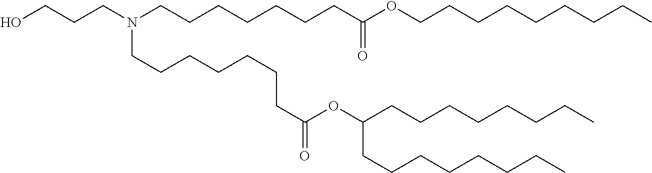 [SMILES: CCCCCCCCCOC(=O)CCCCCCCN(CCCO)CCCCCCCC(=O)OC(CCCCCCCC)CCCCCCCC] |
| D | 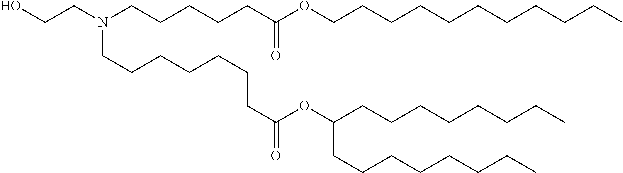 [SMILES: CCCCCCCCCCCOC(=O)CCCCCN(CCO)CCCCCCCC(=O)OC(CCCCCCCC)CCCCCCCC] |
| E | 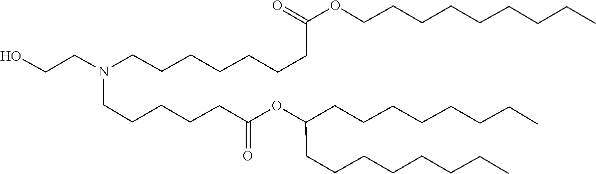 [SMILES: CCCCCCCCCOC(=O)CCCCCCCN(CCO)CCCCCC(=O)OC(CCCCCCCC)CCCCCCCC] |
| F | 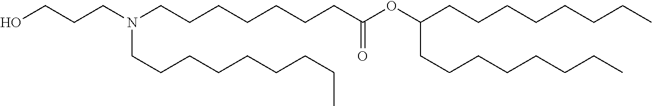 [SMILES: CCCCCCCCCN(CCCO)CCCCCCCC(=O)OC(CCCCCCCC)CCCCCCCC] |
| G | 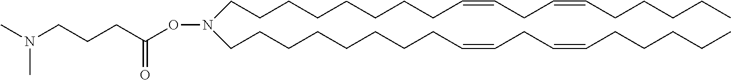 [SMILES: CCCCC/C=C\C/C=C\CCCCCCCCN(CCCCCCCC/C=C\C/C=C\CCCCC)OC(=O)CCCN(C)C] |

In some embodiments, the cationically ionizable lipid has the structure of Formula (XI):

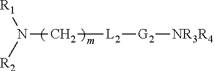

wherein
each of R₁ and R₂ is independently R₅ or —G₁—L₁—R₆, wherein at least one of R₁ and R₂ is —G₁—L₁—R₆;
each of R₃ and R₄ is independently selected from the group consisting of C₁₋₆ alkyl, C₂₋₆ alkenyl, aryl, and C₃₋₁₀ cycloalkyl;
each of R₅ and R₆ is independently a non-cyclic hydrocarbyl group having at least 10 carbon atoms;
each of G₁ and G₂ is independently unsubstituted C₁₋₁₂ alkylene or C₂₋₁₂ alkenylene;
each of L₁ and L₂ is independently selected from the group consisting of —O(C═O)—, —(C═O)O—, —C(═O)—, —O—, —S(O)ₓ—, —S—S—, —C(═O)S—, —SC(═O)—, —NRaC(═O)—, —C(═O)NR^{a}—, —NRaC(═O)NR^{a}—, —OC(═O)NR^{a}— and —NRaC(═O)O—;
R^{a} is H or C₁₋₁₂ alkyl;
m is 0, 1, 2, 3, or 4; and
x is 0, 1 or 2.

In some of the foregoing embodiments of Formula (XI), G₁ is independently unsubstituted C₁-C₁₂ alkylene or unsubstituted C₂₋₁₂ alkenylene, e.g., unsubstituted, straight C₁₋₁₂ alkylene or unsubstituted, straight C₂₋₁₂ alkenylene. In some embodiments, each G₁ is independently unsubstituted C₆₋₁₂ alkylene or unsubstituted C₆₋₁₂ alkenylene, e.g., unsubstituted, straight C₆₋₁₂ alkylene or unsubstituted, straight C₆₋₁₂ alkenylene. In some embodiments, each G₁ is independently unsubstituted C₈₋₁₂ alkylene or unsubstituted C₈₋₁₂ alkenylene, e.g., unsubstituted, straight C₈₋₁₂ alkylene or unsubstituted, straight C₈₋₁₂ alkenylene. In some embodiments, each G₁ is independently unsubstituted C₆₋₁₀ alkylene or unsubstituted C₆₋₁₀ alkenylene, e.g., unsubstituted, straight C₆₋₁₀ alkylene or unsubstituted, straight C₆₋₁₀ alkenylene. In some embodiments, each G₁ is independently unsubstituted alkylene having 8, 9 or 10 carbon atoms, e.g., unsubstituted, straight alkylene having 8, 9 or 10 carbon atoms. In some embodiments, where R₁ and R₂ are both independently —G₁—L₁—R₆, G₁ for R₁ may be different from G₁ for R₂. In some of these embodiments, for example, G₁ for R₁ is unsubstituted, straight C₁₋₁₂ alkylene and G₁ for R₂ is unsubstituted, straight C₂₋₁₂ alkenylene; or G₁ for R₁ is an unsubstituted, straight C₁₋₁₂ alkylene group and G₁ for R₂ is a different unsubstituted, straight C₁₋₁₂ alkylene group. In some embodiments, where R₁ and R₂ are both independently —G₁—L₁—R₆, G₁ for R₁ may be identical to G₁ for R₂. In some of these embodiments, for example, each G₁ is the same unsubstituted, straight C₈₋₁₂ alkylene, such as unsubstituted, straight C₈₋₁₀ alkylene, or each G₁ is the same unsubstituted, straight C₆₋₁₂ alkenylene.

In some of the foregoing embodiments of Formula (XI), each L₁ is independently selected from the group consisting of —O(C═O)—, —(C═O)O—, —C(═O)S—, —SC(═O)—, —NRaC(═O)—, and —C(═O)NR^{a}—. In some embodiments, R^{a} of L₁ is H or C₁₋₁₂ alkyl. In some embodiments, R^{a} of L₁ is H or C₁₋₆ alkyl, e.g., H or C₁₋₃ alkyl. In some embodiments, R^{a} of L₁ is H, methyl, or ethyl. In some embodiments, each L₁ is independently selected from the group consisting of —O(C═O)—, —(C═O)O—, —C(═O)S—, and —SC(═O)—. In some embodiments, each L₁ is independently —O(C═O)— or —(C═O)O—. In some embodiments, where R₁ and R₂ are both independently —G₁—L₁—R₆, L₁ for R₁ may be different from L₁ for R₂. In some of these embodiments, for example, L₁ for R₁ is one moiety selected from the group consisting of —O(C═O)—, —(C═O)O—, —C(═O)S—, —SC(═O)—, —NRaC(═O)—, and —C(═O)NR^{a}— (e.g., L₁ for R₁ is —O(C═O)—), and L₁ for R₂ is a different moiety selected from the group consisting of —O(C═O)—, —(C═O)O—, —C(═O)S—, —SC(═O)—, —NRaC(═O)—, and —C(═O)NR^{a}— (e.g., L₁ for R₂ is —(C═O)O—). In some embodiments, where R₁ and R₂ are both independently —G₁—L₁—R₆, L₁ for R₁ may be identical to L₁ for R₂. In some of these embodiments, for example, each L₁ is the same moiety selected from the group consisting of —O(C═O)—, —(C═O)O—, —C(═O)S—, —SC(═O)—, —NRaC(═O)—, and —C(═O)NR^{a}—, e.g., each L₁ is —O(C═O)— or each L₁ is —(C═O)O—.

In some of the foregoing embodiments of Formula (XI), each R₆ is independently a non-cyclic hydrocarbyl group having at least 10 carbon atoms, e.g., a straight hydrocarbyl group having at least 10 carbon atoms. In some embodiments, each R₆ has independently at most 30 carbon atoms, such as at most 28, at most 26, at most 24, at most 22, or at most 20 carbon atoms. In some embodiments, each R₆ is independently a non-cyclic hydrocarbyl group having 10 to 30 carbon atoms (such as 10 to 28, 10 to 26, 10 to 24, 10 to 22, or 10 to 20 carbon atoms), e.g., a straight hydrocarbyl group having 10 to 30 carbon atoms (such as 10 to 28, 10 to 26, 10 to 24, 10 to 22, or 10 to 20 carbon atoms). In some embodiments, each R₆ is attached to L₁ via an internal carbon atom of R₆. In some embodiments, each R₆ has independently at most 30 carbon atoms (such as at most 28, at most 26, at most 24, at most 22, or at most 20 carbon atoms), and each R₆ is attached to Li via an internal carbon atom of R₆. In some embodiments, each R₆ is independently a non-cyclic hydrocarbyl group having at least 10 carbon atoms, e.g., a straight hydrocarbyl group having at least 10 carbon atoms, and each R₆ is attached to L₁ via an internal carbon atom of R₆. In some embodiments, each R₆ is independently a non-cyclic hydrocarbyl group having 10 to 30 carbon atoms (such as 10 to 28, 10 to 26, 10 to 24, 10 to 22, or 10 to 20 carbon atoms), e.g., a straight hydrocarbyl group having 10 to 30 carbon atoms (such as 10 to 28, 10 to 26, 10 to 24, 10 to 22, or 10 to 20 carbon atoms), and each R₆ is attached to Li via an internal carbon atom of R₆. In some embodiments, the hydrocarbyl group of R₆ is an alkyl or alkenyl group, e.g., a C₁₀₋₃₀ alkyl or alkenyl group. Thus, in some embodiments, each R₆ is independently a non-cyclic alkyl group having at least 10 carbon atoms or a non-cyclic alkenyl group having at least 10 carbon atoms, e.g., a straight alkyl group having at least 10 carbon atoms or a straight alkenyl group having at least 10 carbon atoms.

In some embodiments, each R₆ is independently a non-cyclic alkyl group having 10 to 30 carbon atoms (such as 10 to 28, 10 to 26, 10 to 24, 10 to 22, or 10 to 20 carbon atoms) or a non-cyclic alkenyl group having 10 to 30 carbon atoms (such as 10 to 28, 10 to 26, 10 to 24, 10 to 22, or 10 to 20 carbon atoms), e.g., a straight alkyl group having 10 to 30 carbon atoms (such as 10 to 28, 10 to 26, 10 to 24, 10 to 22, or 10 to 20 carbon atoms) or a straight alkenyl group having 10 to 30 carbon atoms (such as 10 to 28, 10 to 26, 10 to 24, 10 to 22, or 10 to 20 carbon atoms). In some embodiments, each R₆ is independently a non-cyclic alkyl group having 11 to 19 carbon atoms (such as 11, 13, 15, 17, or 17 carbon atoms), e.g., a straight alkyl group having 11 to 19 carbon atoms (such as 11, 13, 15, 17, or 17 carbon atoms). In some embodiments, each R₆ is independently a non-cyclic alkyl group having 10 to 30 carbon atoms (such as 10 to 28, 10 to 26, 10 to 24, 10 to 22, or 10 to 20 carbon atoms) or a non-cyclic alkenyl group having 10 to 30 carbon atoms (such as 10 to 28, 10 to 26, 10 to 24, 10 to 22, or 10 to 20 carbon atoms), e.g., a straight alkyl group having 10 to 30 carbon atoms (such as 10 to 28, 10 to 26, 10 to 24, 10 to 22, or 10 to 20 carbon atoms) or a straight alkenyl group having 10 to 30 carbon atoms (such as 10 to 28, 10 to 26, 10 to 24, 10 to 22, or 10 to 20 carbon atoms), and each R₆ is attached to L₁ via an internal carbon atom of R₆. In some embodiments, each R₆ is independently a non-cyclic alkyl group having 11 to 19 carbon atoms (such as 11, 13, 15, 17, or 17 carbon atoms), e.g., a straight alkyl group having 11 to 19 carbon atoms (such as 11, 13, 15, 17, or 17 carbon atoms), and each R₆ is attached to L₁ via an internal carbon atom of R₆. The expression “internal carbon atom” means that the carbon atom of R₆ by which R₆ is attached to L₁ is directly bonded to at least 2 other carbon atoms of R₆. For example, for the following C₁₁ alkyl group, each carbon atom at any one of positions 2, 3, 4, 5, and 7 qualifies as “internal carbon atom” according to the present disclosure, whereas the carbon atoms at positions 1, 6, 8, 9, 10, and 11 do not.

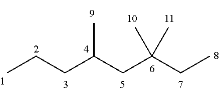

Consequently, R₆ being a C₁ alkyl group attached to L₁ via an internal carbon of R₆ includes the following groups:

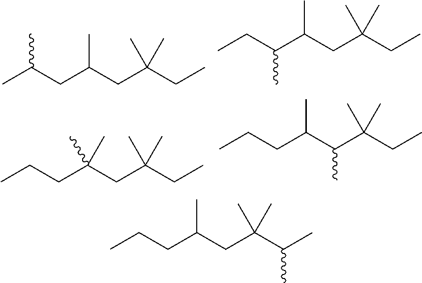

wherein [symbol] we represents the bond by which R₆ is bound to L₁. Furthermore, for a straight alkyl group, e.g., a straight C₁ alkyl group, each carbon atom except for the first and last carbon atoms of the straight alkyl group (i.e., except the carbon atoms at positions 1 and 11 of the straight C₁ alkyl group) qualifies as “internal carbon atom”. Thus, in some embodiments, R₆ being a straight alkyl group having p carbon atoms and being attached to L₁ via an internal carbon atom of R₆ means that R₆ is attached to L₁ via a carbon atom of R₆ at any one of positions 2 to (p-1) (thereby excluding the terminal C atoms at positions 1 and p). In some embodiments, where R₆ is a straight alkyl group having p′ carbon atoms (wherein p′ is an even number) and being attached to L₁ via an internal carbon atom of R₆, R₆ is attached to L₁ via a carbon at any one of positions (p′/2-1), (p′/2), and (p′/2+1) of R₆ (e.g., if p′ is 10, R₆ is attached to L₁ via a carbon atom at any one of positions 4, 5, and 6 of R₆). In some embodiments, where R₆ is a straight alkyl group having p″ carbon atoms (wherein p″ is an uneven number) and being attached to L₁ via an internal carbon atom of R₆, R₆ is attached to L₁ via a carbon atom at any one of positions (p″-1)/2 and (p″+1)/2 of R₆ (e.g., if p″ is 11, R₆ is attached to L₁ via a carbon at any one of positions 5 and 6 of R₆). Generally, it is to be understood that if both R₁ and R₂ are —G₁—L₁—R₆ and each R₆ is attached to L₁ via an internal carbon atom of R₆, R₆ of R₁ is attached to L₁ of R₁ (and not to L₁ of R₂) via an internal carbon atom of R₆ of R₁ and R₆ of R₂ is attached to L₁ of R₂ (and not to L₁ of R₁) via an internal carbon atom of R₆ of R₂. In some embodiments, each R₆ is independently selected from the group consisting of:

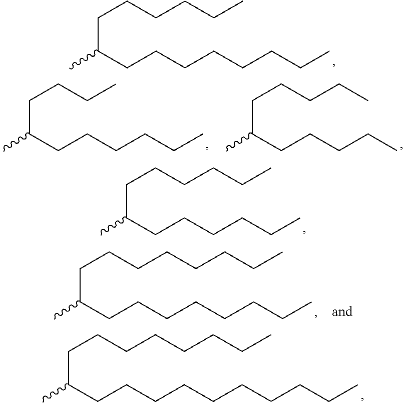

wherein [symbol] represents the bond by which R₆ is bound to L₁. In some embodiments, where R₁ and R₂ are both independently —G₁—L₁—R₆, R₆ for R₁ is different from R₆ for R₂. In some of these embodiments, for example, R₆ for R₁ may be a non-cyclic, preferably straight, hydrocarbyl group having at least 10 carbon atoms (e.g., R₆ for R₁ is

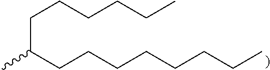 [SMILES: CCCCCCCCC(C)CCCCCC]

and R₆ for R₂ may be a different non-cyclic, preferably straight, hydrocarbyl group having at least 10 carbon atoms (e.g., R₆ for R₂ is

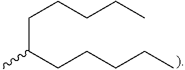

In some embodiments, where R₁ and R₂ are both independently —G₁—L₁—R₆, R₆ for R₁ is identical to R₆ for R₂. In some of these embodiments, for example, each R₆ is the same non-cyclic, preferably straight, hydrocarbyl group having at least 10 carbon atoms (e.g., each R₆ is

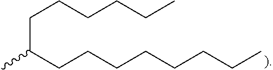 [SMILES: CCCCCCCCC(C)CCCCCC]

In some of the foregoing embodiments of Formula (XI), R₅ is a non-cyclic hydrocarbyl group having at least 10 carbon atoms, e.g., a straight hydrocarbyl group having at least 10 carbon atoms. In some embodiments, R₅ is a non-cyclic hydrocarbyl group having at least 12 carbon atoms, such as at least 14, at least 16, or at least 18 carbon atoms, e.g., a straight hydrocarbyl group having at least 12, at least 14, at least 16, or at least 18 carbon atoms. In some embodiments, R₅ has at most 30 carbon atoms, such as at most 28, at most 26, at most 24, at most 22, or at most 20 carbon atoms. In some embodiments, R₅ is a non-cyclic hydrocarbyl group, e.g., a straight hydrocarbyl group, wherein each hydrocarbyl group has 10 to 30 carbon atoms (such as 10 to 28, 10 to 26, 10 to 24, 10 to 22, 10 to 20 carbon atoms, or 12 to 30, 12 to 28, 12 to 26, 12 to 24, 12 to 22, 12 to 20 carbon atoms, or 14 to 30, 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms). In some embodiments, the hydrocarbyl group of R₅ is an alkyl or alkenyl group, e.g., a C₁₀₋₃₀ alkyl or alkenyl group. Thus, in some embodiments, R₅ is a non-cyclic alkyl group having at least 10 carbon atoms (such as at least 12, at least 14, at least 16, or at least 18 carbon atoms) or a non-cyclic alkenyl group having at least 10 carbon atoms (such as at least 12, at least 14, at least 16, or at least 18 carbon atoms), e.g., a straight alkyl group having at least 10 carbon atoms (such as at least 12, at least 14, at least 16, or at least 18 carbon atoms) or a straight alkenyl group having at least 10 carbon atoms (such as at least 12, at least 14, at least 16, or at least 18 carbon atoms). In some embodiments, R₅ is a non-cyclic alkyl group or a non-cyclic alkenyl group, e.g., a straight alkyl group or a straight alkenyl group, wherein each of the alkyl and alkenyl groups has independently 10 to 30 carbon atoms (such as 10 to 28, 10 to 26, 10 to 24, 10 to 22, 10 to 20 carbon atoms, or 12 to 30, 12 to 28, 12 to 26, 12 to 24, 12 to 22, 12 to 20 carbon atoms, or 14 to 30, 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms). In some embodiments, the alkenyl group has at least 2 carbon-carbon double bonds, e.g., 2 or 3 carbon-carbon double bonds, such as 2 carbon-carbon double bonds. In some embodiments, the alkenyl group has at least 1 carbon-carbon double bond in cis configuration, e.g., 1, 2 or 3, such as 2, carbon-carbon double bonds in cis configuration. Thus, in some embodiments, R₅ is a non-cyclic alkyl group or a non-cyclic alkenyl group, e.g., a straight alkyl group or a straight alkenyl group, wherein each of the alkyl and alkenyl groups has independently 10 to 30 carbon atoms (such as 10 to 28, 10 to 26, 10 to 24, 10 to 22, 10 to 20 carbon atoms, or 12 to 30, 12 to 28, 12 to 26, 12 to 24, 12 to 22, 12 to 20 carbon atoms, or 14 to 30, 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has at least 2 carbon-carbon double bonds, e.g., 2 or 3 carbon-carbon double bonds. In some embodiments, R₅ is a non-cyclic alkyl group or a non-cyclic alkenyl group, e.g., a straight alkyl group or a straight alkenyl group, wherein each of the alkyl and alkenyl groups has independently 10 to 30 carbon atoms (such as 10 to 28, 10 to 26, 10 to 24, 10 to 22, 10 to 20 carbon atoms, or 12 to 30, 12 to 28, 12 to 26, 12 to 24, 12 to 22, 12 to 20 carbon atoms, or 14 to 30, 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has at least 1 carbon-carbon double bond, such as 1, 2, or 3 carbon-carbon double bonds, in cis configuration. In some embodiments, R₅ has the following structure:

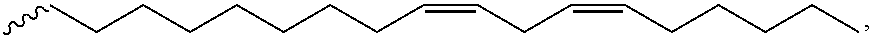

wherein represents the bond by which R⁵ is bound to the remainder of the compound.

[symbol]

In some of the foregoing embodiments of Formula (XI), L₂ is selected from the group consisting of —O(C═O)—, —(C═O)O—, —C(═O)—, —S—S—, —C(═O)S—, —SC(═O)—, —NRaC(═O)—, —C(═O)NR^{a}—, —NRaC(═O)NR^{a}—, —OC(═O)NR^{a}— and —NRaC(═O)O—. In some embodiments, L₂ is selected from the group consisting of —O(C═O)—, —(C═O)O—, —C(═O)—, —C(═O)S—, —SC(═O)—, —NRaC(═O)—, and —C(═O)NR^{a}—. In some embodiments, R^{a} of L₂ is H or C₁₋₁₂ alkyl. In some embodiments, R^{a} of L₂ is H or C₁₋₆ alkyl, e.g., H or C₁₋₃ alkyl. In some embodiments, R^{a} of L₂ is H, methyl, or ethyl. In some embodiments, L₂ is selected from the group consisting of —O(C═O)—, —(C═O)O—, —C(═O)S—, and —SC(═O)—. In some embodiments, L₂ is —O(C═O)— or —(C═O)O—.

In some of the foregoing embodiments of Formula (XI), G₂ is unsubstituted C₁₋₁₂ alkylene or unsubstituted C₂₋₁₂ alkenylene, e.g., unsubstituted, straight C₁₋₁₂ alkylene or unsubstituted, straight C₂₋₁₂ alkenylene. In some embodiments, G₂ is unsubstituted C₂₋₁₀ alkylene or unsubstituted C₂—10 alkenylene, e.g., unsubstituted, straight C₂₋₁₀ alkylene or unsubstituted, straight C₂₋₁₀ alkenylene. In some embodiments, G₂ is unsubstituted C₂₋₆ alkylene or unsubstituted C₂₋₆ alkenylene, e.g., unsubstituted, straight C₂₋₆ alkylene or unsubstituted, straight C₂₋₆ alkenylene. In some embodiments, G₂ is unsubstituted C₂₋₄ alkylene or unsubstituted C₂₋₄ alkenylene, e.g., unsubstituted, straight C2-4 alkylene or unsubstituted, straight C₂₋₄ alkenylene. In some embodiments, G₂ is ethylene or trimethylene.

In some of the foregoing embodiments of Formula (XI), each of R₃ and R₄ is independently C₁₋₆ alkyl or C₂₋₆ alkenyl. In some embodiments, each of R₃ and R₄ is independently C₁₋₄ alkyl or C₂₋₄ alkenyl. In some embodiments, each of R₃ and R₄ is independently C₁₋₃ alkyl. In some embodiments, each of R₃ and R₄ is independently methyl or ethyl. In some embodiments, each of R₃ and R₄ is methyl.

In some of the foregoing embodiments of Formula (XI), m is 0, 1, 2 or 3. In some embodiments, m is 0 or 2. In some embodiments, m is 0. In some embodiments, m is 2.

In some of the foregoing embodiments of Formula (XI), the cationically ionizable lipid has the structure of Formula (XIIa) or (XIIb):

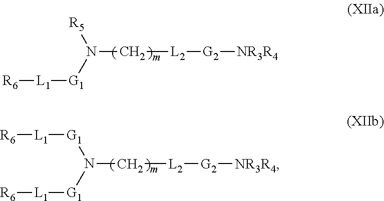

wherein
each of R₃ and R₄ is independently C₁-C₆ alkyl or C₂₋₆ alkenyl; R₅ is a straight hydrocarbyl group having at least 14 carbon atoms (such as at least 16 carbon atoms), wherein the hydrocarbyl group preferably has at least 2 carbon-carbon double bonds;
each R₆ is independently a straight hydrocarbyl group (e.g., a straight alkyl group) having at least 10 carbon atoms and/or each R₆ is attached to L₁ via an internal carbon atom of R₆, preferably each R₆ is independently a straight hydrocarbyl group (e.g., a straight alkyl group) having at least 10 carbon atoms and each R₆ is attached to L₁ via an internal carbon atom of R₆;
each G₁ is independently unsubstituted, straight C₄₋₁₂ alkylene or C₄₋₁₂ alkenylene, e.g., unsubstituted, straight C₆₋₁₂ alkylene or C₆₋₁₂ alkenylene, such as unsubstituted, straight C₈₋₁₂ alkylene or unsubstituted, straight C₈₋₁₂ alkenylene;
G₂ is unsubstituted C₂-C₁₀ alkylene or C₂₋₁₀ alkenylene, preferably unsubstituted C₂-C₆ alkylene or C2-6 alkenylene;
each of L₁ and L₂ is independently —O(C═O)— or —(C═O)O—; and
m is 0, 1, 2 or 3, preferably 0 or 2.

In some of the foregoing embodiments of Formula (XIIa), R₅ has at most 30 carbon atoms, such as at most 28, at most 26, at most 24, at most 22, or at most 20 carbon atoms. In some embodiments of formulas (XIIa), R₅ is a straight hydrocarbyl group having 14 to 30 carbon atoms (such as 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms). In some embodiments of formula (XIIa), R₅ is a straight alkyl or alkenyl group having 14 to 30 carbon atoms (such as 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms). In some embodiments of formula (XIIa), the alkenyl group has at least 2 carbon-carbon double bonds, e.g., 2 or 3 carbon-carbon double bonds, such as 2 carbon-carbon double bonds. In some embodiments, the alkenyl group has at least 1 carbon-carbon double bond in cis configuration, e.g., 1, 2 or 3, such as 2, carbon-carbon double bonds in cis configuration. Thus, in some embodiments of formula (XIIa), R₅ is a straight alkyl group or a straight alkenyl group, wherein each of the alkyl and alkenyl groups has independently 14 to 30 carbon atoms (such as 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has at least 2 carbon-carbon double bonds, e.g., 2 or 3 carbon-carbon double bonds. In some embodiments of formula (XIIa), R₅ is a straight alkyl group or a straight alkenyl group, wherein each of the alkyl and alkenyl groups has independently 14 to 30 carbon atoms (such as 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has at least 1 carbon-carbon double bond, such as 1, 2, or 3 carbon-carbon double bonds, in cis configuration. In some embodiments of formula (XIIa), R₅ is a straight alkyl group or a straight alkenyl group, wherein each of the alkyl and alkenyl groups has independently 14 to 30 carbon atoms (such as 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has 2 or 3 carbon-carbon double bonds, wherein at least 1 carbon-carbon double bond, such as 1, 2, or 3 carbon-carbon double bonds, is in cis configuration. In some embodiments of formula (XIIa), R⁵ has the following structure:

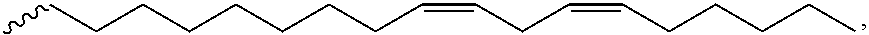

wherein [symbol] represents the bond by which R₅ is bound to the remainder of the compound. In some embodiments of formula (XIIa), R₆ has at most 30 carbon atoms, such as at most 28, at most 26, at most 24, at most 22, or at most 20 carbon atoms. In some embodiments of formula (XIIa), R₆ is a non-cyclic hydrocarbyl group (e.g., a non-cyclic alkyl group) having 10 to 30 carbon atoms (such as 10 to 28, 10 to 26, 10 to 24, 10 to 22, or 10 to 20 carbon atoms), e.g., a straight hydrocarbyl group (e.g., a straight alkyl group) having 10 to 30 carbon atoms (such as 10 to 28, 10 to 26, 10 to 24, 10 to 22, or 10 to 20 carbon atoms). In some embodiments of formula (XIIa), R₆ is a straight hydrocarbyl group (e.g., a straight alkyl group) having at least 10 carbon atoms and R⁶ is attached to L₁ via an internal carbon atom of R₆. In some embodiments of formula (XIIa), R₆ is a non-cyclic hydrocarbyl group (e.g., a non-cyclic alkyl group) having 10 to 30 carbon atoms (such as 10 to 28, 10 to 26, 10 to 24, 10 to 22, or 10 to 20 carbon atoms), e.g., a straight hydrocarbyl group (e.g., a straight alkyl group) having 10 to 30 carbon atoms (such as 10 to 28, 10 to 26, 10 to 24, 10 to 22, or 10 to 20 carbon atoms), and R₆ is attached to L₁ via an internal carbon atom of R₆. In some embodiments of formula (XIIa), G₁ is independently unsubstituted, straight C₄₋₁₂ alkylene or C₄₋₁₂ alkenylene, e.g., unsubstituted, straight C6-12 alkylene or C₆₋₁₂ alkenylene. In some embodiments of formula (XIIa), R₅ is a straight hydrocarbyl group, e.g., a straight alkenyl group, having at least 14 carbon atoms (such as 14 to 30 carbon atoms) and 2 or 3 carbon-carbon double bonds; R₆ is a straight hydrocarbyl group (e.g., a straight alkyl group) having at least 10 carbon atoms (e.g., having 10 to 30 carbon atoms) and R₆ is attached to L₁ via an internal carbon atom of R₆; and G₁ is independently unsubstituted, straight C₄₋₁₂ alkylene or C₄₋₁₂ alkenylene, e.g., unsubstituted, straight C₆₋₁₂ alkylene or C₆₋₁₂ alkenylene.

In some of the foregoing embodiments of Formula (XIIb), each R₆ has independently at most 30 carbon atoms, such as at most 28, at most 26, at most 24, at most 22, or at most 20 carbon atoms. In some embodiments of formula (XIIb), each R₆ is independently a straight hydrocarbyl group (e.g., a straight alkyl group) having 10 to 30 carbon atoms (such as 10 to 28, 10 to 26, 10 to 24, 10 to 22, or 10 to 20 carbon atoms, or 11 to 19 carbon atoms, such as 11, 13, 15, 17, or 17 carbon atoms). In some embodiments of formula (XIIb), each R₆ is independently a straight hydrocarbyl group (e.g., a straight alkyl group) having 10 to 30 carbon atoms (such as 10 to 28, 10 to 26, 10 to 24, 10 to 22, or 10 to 20 carbon atoms, or 11 to 19 carbon atoms, such as 11, 13, 15, 17, or 17 carbon atoms) and each R₆ is attached to L₁ via an internal carbon atom of R₆. In some embodiments of formula (XIIb), each R₆ is independently selected from the group consisting of:

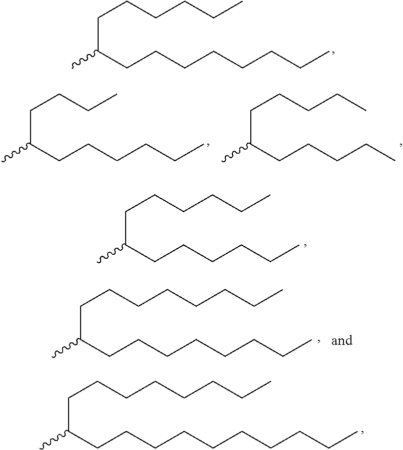

wherein [symbol] represents the bond by which R₆ is bound to L₁. In some embodiments of formula (XIIb), each G₁ is independently unsubstituted, straight C₆₋₁₂ alkylene or C₆₋₁₂ alkenylene. In some embodiments of formula (XIIb), each G₁ is independently unsubstituted, straight C₈₋₁₂ alkylene or C₈₋₁₂ alkenylene. In some embodiments of formula (XIIb), each R₆ is independently a straight hydrocarbyl group (e.g., a straight alkyl group) having at least 10 carbon atoms (such as 10 to 28, 10 to 26, 10 to 24, 10 to 22, or 10 to 20 carbon atoms, or 11 to 19 carbon atoms, such as 11, 13, 15, 17, or 17 carbon atoms) and is attached to L₁ via an internal carbon atom of R₆; and each G₁ is independently unsubstituted, straight C₈₋₁₂ alkylene or C₈₋₁₂ alkenylene.

In some of the foregoing embodiments of Formula (XI), the cationically ionizable lipid has the structure of Formula (XIIIa) or (XIIIb):

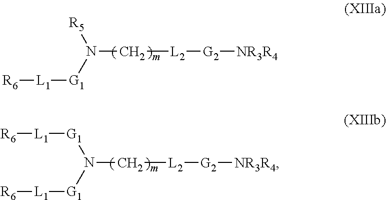

wherein
each of R₃ and R₄ is independently C₁₋₄ alkyl or C₂₋₄ alkenyl, more preferably C₁₋₃ alkyl, such as methyl or ethyl;
R₅ is a straight alkyl or alkenyl group having at least 16 carbon atoms, wherein the alkenyl group preferably has at least 2 carbon-carbon double bonds;
each R₆ is independently a straight hydrocarbyl group having at least 10 carbon atoms, wherein R₆ is attached to L₁ via an internal carbon atom of R₆;
each G₁ is independently unsubstituted, straight C₆₋₁₂ alkylene or unsubstituted, straight C₆₋₁₂ alkenylene, e.g., unsubstituted, straight C₈₋₁₂ alkylene or unsubstituted, straight C₈₋₁₂ alkenylene, such as unsubstituted, straight C₈₋₁₀ alkylene or unsubstituted, straight C₈₋₁₀ alkenylene, such as unsubstituted, straight C₈ alkylene;
G₂ is unsubstituted C₂₋₆ alkylene or C₂₋₆ alkenylene, preferably unsubstituted C₂₋₄ alkylene or C₂₋₄ alkenylene, such as ethylene or trimethylene;
each of L₁ and L₂ is independently —O(C═O)— or —(C═O)O—; and
m is 0, 1, 2 or 3, preferably 0 or 2.

In some of the foregoing embodiments of Formula (XIIIa), R₅ has at most 30 carbon atoms, such as at most 28, at most 26, at most 24, at most 22, or at most 20 carbon atoms. In some embodiments of formulas (XIIIa), R₅ is a straight alkyl or alkenyl group having 16 to 30 carbon atoms (such as 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms). In some embodiments of formula (XIIIa), the alkenyl group has at least 2 carbon-carbon double bonds, e.g., 2 or 3 carbon-carbon double bonds, such as 2 carbon-carbon double bonds.

In some embodiments, the alkenyl group has at least 1 carbon-carbon double bond in cis configuration, e.g., 1, 2 or 3, such as 2, carbon-carbon double bonds in cis configuration. Thus, in some embodiments of formula (XIIIa), R₅ is a straight alkyl group or a straight alkenyl group, wherein each of the alkyl and alkenyl groups has independently 16 to 30 carbon atoms (such as 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has at least 2 carbon-carbon double bonds, e.g., 2 or 3 carbon-carbon double bonds.

In some embodiments of formula (XIIIa), R₅ is a straight alkyl group or a straight alkenyl group, wherein each of the alkyl and alkenyl groups has independently 16 to 30 carbon atoms (such as 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has at least 1 carbon-carbon double bond, such as 1, 2, or 3 carbon-carbon double bonds, in cis configuration. In some embodiments of formula (XIIIa), R₅ is a straight alkyl group or a straight alkenyl group, wherein each of the alkyl and alkenyl groups has independently 16 to 30 carbon atoms (such as 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has 2 or 3 carbon-carbon double bonds, wherein at least 1 carbon-carbon double bond, such as 1, 2, or 3 carbon-carbon double bonds, is in cis configuration. In some embodiments of formula (XIIIa), R₅ has the following structure:

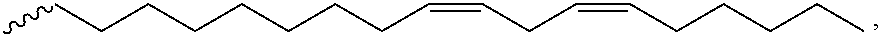

wherein [symbol] represents the bond by which R₅ is bound to the remainder of the compound. In some embodiments of formula (XIIIa), R₆ has at most 30 carbon atoms, such as at most 28, at most 26, at most 24, at most 22, or at most 20 carbon atoms. In some embodiments of formula (XIIIa), R₆ is a straight hydrocarbyl group (e.g., a straight alkyl group) having 10 to 30 carbon atoms (such as 10 to 28, 10 to 26, 10 to 24, 10 to 22, or 10 to 20 carbon atoms) and R₆ is attached to L₁ via an internal carbon atom of R₆. In some embodiments of formula (XIIIa), R₆ is a straight alkyl group having 10 to 30 carbon atoms (such as 10 to 28, 10 to 26, 10 to 24, 10 to 22, or 10 to 20 carbon atoms) and R₆ is attached to L₁ via an internal carbon atom of R₆. In some embodiments of formula (XIIIa), G₁ is independently unsubstituted, straight C₄₋₁₂ alkylene or C₄₋₁₂ alkenylene, e.g., unsubstituted, straight C6-12 alkylene or C6-12 alkenylene. In some embodiments of formula (XIIIa), R₅ is a straight hydrocarbyl group, e.g., a straight alkenyl group, having at least 16 carbon atoms (such as 16 to 30 carbon atoms) and 2 or 3 carbon-carbon double bonds; R₆ is a straight hydrocarbyl group (e.g., a straight alkyl group) having at least 10 carbon atoms (e.g., having 10 to 30 carbon atoms) and R₆ is attached to L₁ via an internal carbon atom of R₆; and G₁ is independently unsubstituted, straight C₄₋₁₂ alkylene or C₄₋₁₂ alkenylene, e.g., unsubstituted, straight C₆₋₁₂ alkylene or C₆₋₁₂ alkenylene.

In some of the foregoing embodiments of Formula (XIIIb), each R₆ has independently at most 30 carbon atoms, such as at most 28, at most 26, at most 24, at most 22, or at most 20 carbon atoms. In some embodiments of formula (XIIIb), each R₆ is independently a straight hydrocarbyl group (e.g., a straight alkyl group) having 10 to 30 carbon atoms (such as 10 to 28, 10 to 26, 10 to 24, 10 to 22, or 10 to 20 carbon atoms, or 11 to 19 carbon atoms, such as 11, 13, 15, 17, or 17 carbon atoms) and each R₆ is attached to L₁ via an internal carbon atom of R₆. In some embodiments of formula (XIIIb), each R₆ is attached to L₁ via an internal carbon atom of R₆ and is independently selected from the group consisting of;

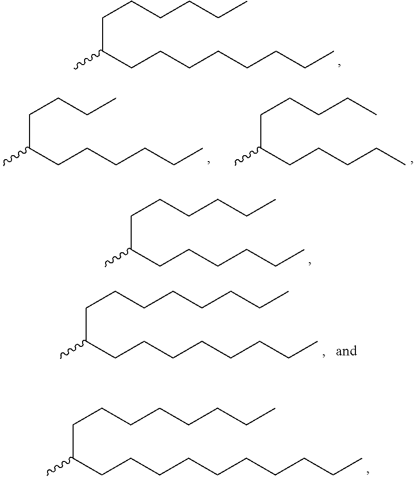

wherein [symbol] represents the bond by which R₆ is bound to L₁. In some embodiments of formula (XIIIb), each G₁ is independently unsubstituted, straight C₈₋₁₂ alkylene or C₈₋₁₂ alkenylene, e.g., unsubstituted, straight C₈₋₁₀ alkylene or C₈₋₁₀ alkenylene. In some embodiments of formula (XIIIb), each R₆ is independently a straight hydrocarbyl group (e.g., a straight alkyl group) having at least 10 carbon atoms (such as 10 to 28, 10 to 26, 10 to 24, 10 to 22, or 10 to 20 carbon atoms, or 11 to 19 carbon atoms, such as 11, 13, 15, 17, or 17 carbon atoms) and is attached to L₁ via an internal carbon atom of R₆; and each G₁ is independently unsubstituted, straight C₈₋₁₂ alkylene or C₈₋₁₂ alkenylene, e.g., unsubstituted, straight C₈₋₁₀ alkylene or C₈₋₁₀ alkenylene.

In some of the foregoing embodiments of Formula (XI), the cationically ionizable lipid has one of the following formulas (XIV-1), (XIV-2), and (XIV-3):

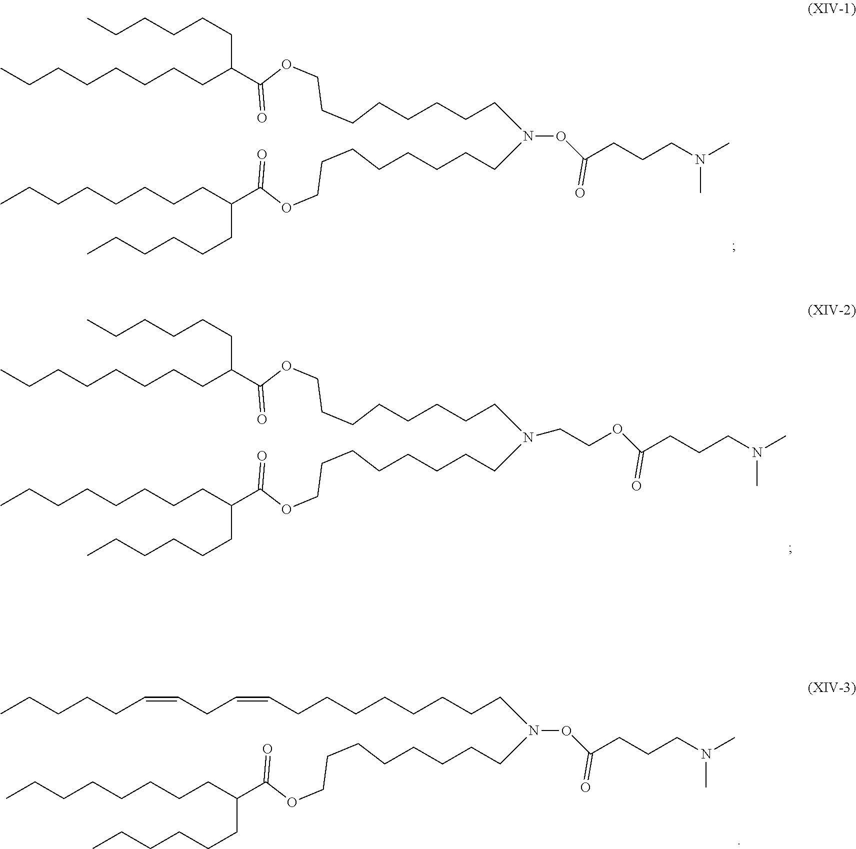

In some embodiments, the cationically ionizable lipid is (6Z, 16Z)-12-((Z)-dec-4-en-1-yl)docosa-6,16 dien-11-yl 5-(dimethylamino)pentanoate (3D-P-DMA). The structure of 3D-P-DMA may be represented as follows:

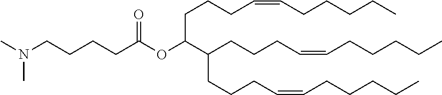 [SMILES: CCCCC/C=C\CCCC(CCC/C=C\CCCCC)C(CCC/C=C\CCCCC)OC(=O)CCCCN(C)C]

In some embodiments, the cationically ionizable lipid is a thiolipid compound represented by formula (TL-I):

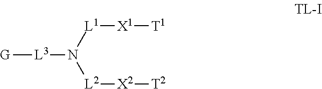

or a pharmaceutically acceptable salt thereof, wherein:
each of L¹ and L² is independently an optionally substituted C₁-C₃₀ aliphatic group (e.g., an optionally substituted C₁-C₃₀ alkylene, C₂-C₃₀ alkenylene, C₂-C₃₀ alkynylene, or C₃-C₃₀ cycloalkylene group); L³ is a bond, optionally substituted C₁-C₁₀ aliphatic group (e.g., an optionally substituted C₁-C₁₀ alkylene, C₂-C₁₀ alkenylene, C₂-C₁₀ alkynylene, or C₃-C₁₀ cycloalkylene group), or optionally substituted 2- to 10-membered heteroaliphatic group (e.g., an optionally substituted 2- to 10-membered alkylene, alkenylene, alkynylene, or heterocycloalkylene group, wherein in each of the optionally substituted 2- to 10-membered alkylene, alkenylene, and alkynylene groups one or more (e.g., 1, 2, 3, or 4) carbon atoms are replaced by heteroatoms of O, N, Si, Se, P, or S, preferably O, S, or N); each of X¹ and X² is independently selected from a bond, —OC(O)—, —C(O)O—, —S(O)₂N(R¹)—, —N(R¹)S(O)₂—, —S(O)—, —S(O)₂—, —S(O)₂C(R¹)₂—, —OC(S)C(R¹)₂—, —C(R¹)₂C(S)O—, and —S—, wherein one or both of X¹ or X² is selected from —S(O)₂N(R¹)—, —N(R¹)S(O)₂, —S(O)—, —S(O)₂—, —S(O)₂C(R¹)₂—, —OC(S)C(R¹)₂—, —C(R¹)₂C(S)O—, and —S—;
each R⁷ is independently optionally substituted C₁-C₂₀ aliphatic group (e.g., an optionally substituted C₁-C₂₀ alkyl, C₂-C₂₀ alkenyl, C₂-C₂₀ alkynyl, or C₃-C₂₀ cycloalkyl group) or H;
each of T¹ and T² is independently an optionally substituted C₃-C₃₀ aliphatic group (e.g., an optionally substituted C₃-C₃₀ alkyl, C₃-C₃₀ alkenyl, C₃-C₃₀ alkynyl, or C₃-C₃₀ cycloalkyl group); G is —N(R²)C(S)N(R²)₂, —N⁺(R³)₃, —OH, —N(R²)₂, —N(R⁵)C(O)R³, —N(R⁵)S(O)₂R₃, —N(R⁵)C(O)N(R³)₂, —CH(N—R²), or —R⁴;
each R² is independently selected from the group consisting of H, optionally substituted C₁-C₆ aliphatic group (e.g., an optionally substituted C₁-C₆ alkyl, C₂-C₆ alkenyl, C₂-C₆ alkynyl, or C₃-C₆ cycloalkyl group) or OR³; or two instances of R² come together with the atoms to which they are attached to form an optionally substituted 4- to 12-membered heterocycle ring or an optionally substituted 4- to 12-membered heteroaryl ring;
each R³ is independently selected from the group consisting of H and optionally substituted C₁-C₁₀ aliphatic group (e.g., an optionally substituted C₁-C₁₀ alkyl, C₂-C₁₀ alkenyl, C₂-C₁₀ alkynyl, or C₃-C₁₀ cycloalkyl group); R⁴ is optionally substituted 4- to 12-membered heterocycle, optionally substituted 4- to 12 membered heteroaryl, C₆-C₁₂ aryl substituted with one or more of —(CH₂)₀₋₆—OH or —(CH₂)₀₋₆—N(R⁵)₂, or C₃-C₁₂ cycloaliphatic (e.g., C₃-C₁₂ alkyl, C₃-C₁₂ alkenyl, C₃-C₁₂ alkynyl, or C₃-C₁₂ cycloalkyl) substituted with one or more of oxo, —(CH₂)₀₋₆—OH, or —(CH₂)₀₋₆—N(R⁵)₂; and each R⁵ is independently selected from H and optionally substituted C₁-C₆ aliphatic group (e.g., an optionally substituted C₁-C₆ alkyl, C₂-C₆ alkenyl, C₂-C₆ alkynyl, or C₃-C₆ cycloalkyl group).

In some embodiments of formula (TL-I), L¹ and L² are each independently —(CH₂)₆₋₁₀—.

In some embodiments of formula (TL-I), X¹ and X² are each independently selected from —S(O)₂N(R¹)—, —N(R¹)S(O)₂, —S(O)—, —S(O)₂—, —S(O)₂C(R¹)₂—, —OC(S)C(R¹)₂—, —C(R¹)₂C(S)O—, and —S—.

In some embodiments of formula (TL-J), X¹ and X² are each —S(O)₂N(R¹)—, where each R⁷ is independently C₁-C₁₀ aliphatic (e.g., C₁-C₁₀ alkyl, C₂-C₁₀ alkenyl, C₂-C₁₀ alkynyl, or C₃-C₁₀ cycloalkyl group, preferably C₁-C₁₀ alkyl).

In some embodiments of formula (TL-J), each of T¹ and T² is independently selected from optionally substituted C₃-C₂₀ alkyl.

In some embodiments of formula (TL-I), each of T¹ and T² is independently selected from:

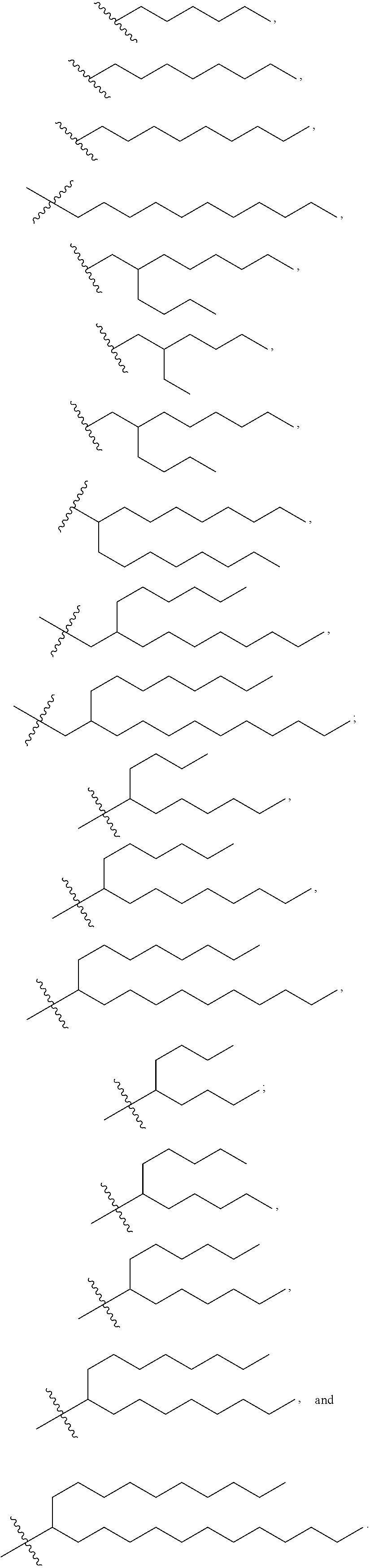

In some embodiments of formula (TL-I), G is —N(R²)C(S)N(R²)₂ or —N(R⁵)S(O)₂R₃.

In some embodiments of formula (TL-I), G is —N(H)C(S)N(R²)₂, where each R² is selected from optionally substituted C₁-C₆ aliphatic group (e.g., an optionally substituted C₁-C₆ alkyl, C₂-C₆ alkenyl, C₂-C₆ alkynyl, or C₃-C₆ cycloalkyl group, preferably optionally substituted C₁-C₆ alkyl) and —OH.

In some embodiments of formula (TL-I), G is —OH.

In some embodiments of formula (TL-I), G is selected from:

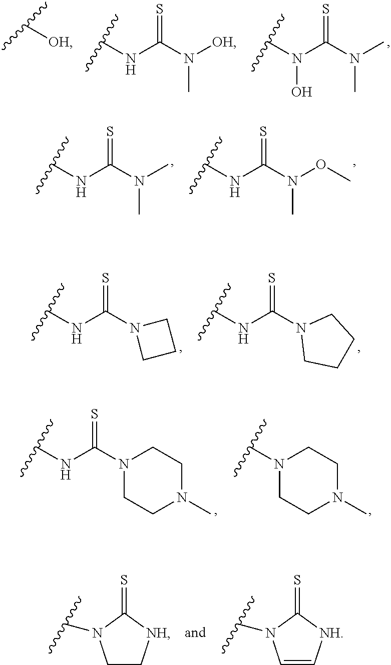

In some embodiments of formula (TL-I), -L³-G is selected from:

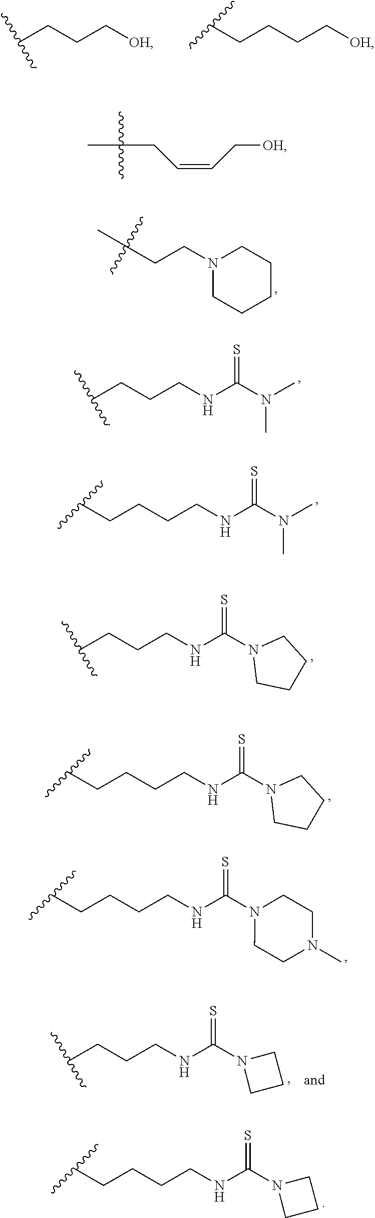

In some embodiments of formula (TL-I), the compound is represented by Formula (TL-IIa):

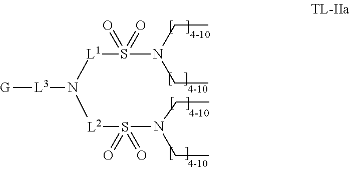

or a pharmaceutically acceptable salt thereof.

In some embodiments of formula (TL-I), the compound is represented by Formula (TL-IIc):

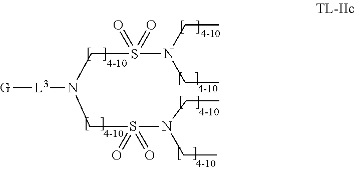

or a pharmaceutically acceptable salt thereof.

In some embodiments of formula (TL-I), the compound is represented by Formula (TL-IIIb):

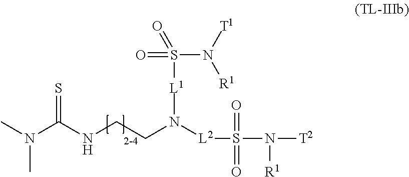

or a pharmaceutically acceptable salt thereof.

In some embodiments of formula (TL-I), the compound is represented by Formula (TL-IIIe):

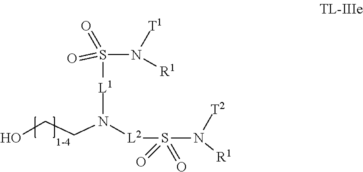

or a pharmaceutically acceptable salt thereof.

In some embodiments of formula (TL-I), the compound is 7,7′-((4-hydroxybutyl)azanediyl)bis(N-hexyl-N-octylheptane-1-sulfonamide) having the following structure:

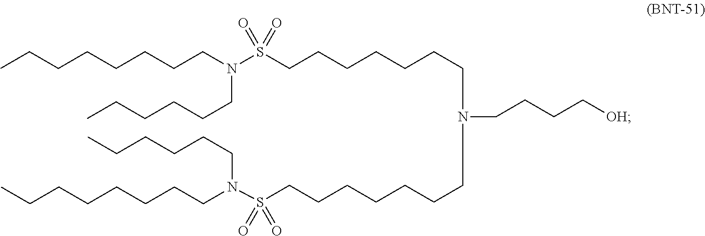 [SMILES: CCCCCCCCN(CCCCCC)S(=O)(=O)CCCCCCCN(CCCCO)CCCCCCCS(=O)(=O)N(CCCCCC)CCCCCCCC]

or a pharmaceutically acceptable salt thereof.

In some embodiments of formula (TL-I), the compound is 7,7′-((4-(3,3-dimethylthioureido)butyl)azanediyl)bis(N-hexyl-N-octylheptane-1-sulfonamide) having the following structure:

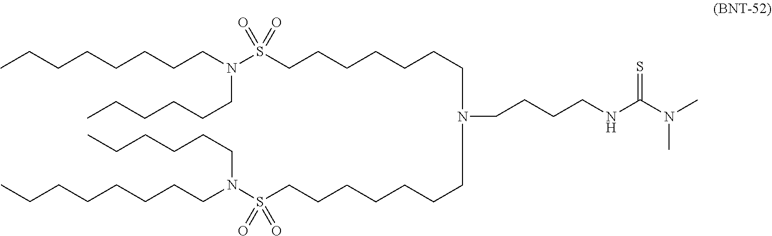 [SMILES: CCCCCCCCN(CCCCCC)S(=O)(=O)CCCCCCCN(CCCCCCCS(=O)(=O)N(CCCCCC)CCCCCCCC)CCCCNC(=S)N(C)C]

or a pharmaceutically acceptable salt thereof.

In some embodiments of formula (TL-I), the compound is a compound having the following structure:

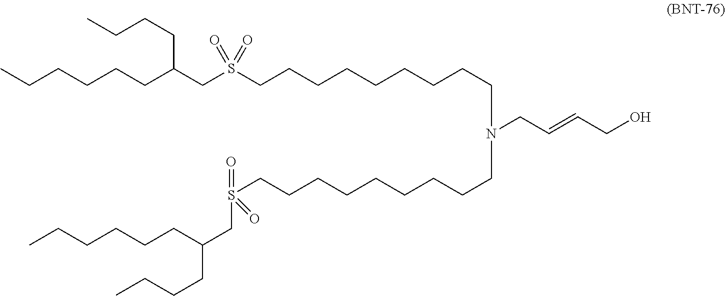 [SMILES: CCCCCCC(CCCC)CS(=O)(=O)CCCCCCCCCN(C/C=C/CO)CCCCCCCCCS(=O)(=O)CC(CCCC)CCCCCC]

or a pharmaceutically acceptable salt thereof.

Thiolipid compounds of formula (TL-J) can be prepared according to PCT/EP2023/071270, the contents of which are incorporated herein by reference.

In some embodiments, the cationically ionizable lipid is a compound represented by formula (AXL-J):

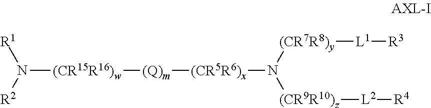

or a pharmaceutically acceptable salt thereof, wherein:
each R¹ and R² is independently selected from the group consisting of H, an optionally substituted C₁₋₁₂alkyl, optionally substituted C₂₋₁₂ alkenyl, optionally substituted C₂₋₁₂ alkynyl, optionally substituted C₃₋₆ cycloalkyl, optionally substituted C₄₋₆ heterocycloalkyl, optionally substituted C₄₋₆ alkylcycloalkyl, optionally substituted C₄₋₆ aryl, optionally substituted C₃₋₆ heteroaryl, optionally substituted C₄₋₈ aryloxy, optionally substituted C₇₋₁₀ arylalkyl; optionally substituted C₅₋₁₀ heteroarylalkyl group, optionally substituted amine; or R¹ and R² can together form an optionally substituted cycloalkyl or heterocycloalkyl ring;
each R³ and R⁴ is independently selected from the group consisting of optionally substituted C₁₋₂₂ alkyl, optionally substituted C₂₋₂₂ alkenyl, and optionally substituted C₂₋₂₂ alkynyl;
each R⁵, R⁶, R⁷, R⁸, R⁹, R¹⁰, R¹⁵, and R¹⁶ is independently selected from the group consisting of H, OH, halogen, phenyl, benzyl, optionally substituted C₁₋₂₂ alkyl, optionally substituted C₂₋₂₂ alkenyl, optionally substituted C₂₋₂₂ alkynyl;
each of w, y, and z is independently an integer from 0-10;
each Q is independently an atom selected from O, NR¹, NR¹NR¹, S, or a disulfide bond;
m is 0 or 1; and
each of L¹ and L² is independently selected from the group consisting of —C(═O)—; —OC(═O)—; —OC(═O)O—; —C(═O)O—; —C(═O)O(CR⁶R⁷)ₘ—; —NH—C(═O)—; —C(═O)NH—; —SO—; —SO₂—; —SO₃—; —NSO₂—; —SO₂N—; —NH(C₁₋₈ alkyl)-; —N(C₁₋₈ alkyl)₂—; —NH(C₆ aryl)-; —N(C₆ aryl)₂—; —NHC(═O)NH—; —NHC(═O)O—; —OC(═O)NH—; —NHC(═O)NR¹—; —NHC(═O)O—; —OC(═O)NR¹—; —C(═O)R¹—; —CO(C₁₋₈ alkyl)-; —CO(C₆ aryl)-; —CO₂(C₁₋₈ alkyl)-; —CO₂(C₆ aryl)-; —SO₂(C₁₋₈ alkyl)-; and —SO₂(C₆ aryl)-.

In some embodiments of formula (AXL-I), the compound is selected from the group consisting of: di(heptadecan-9-yl) 3,3′-((2-(4-methylpiperazin-1-yl)ethyl)azanediyl)dipropionate having the following structure:

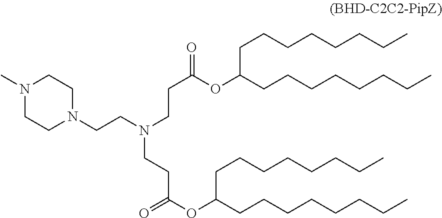 [SMILES: CCCCCCCCC(CCCCCCCC)OC(=O)CCN(CCC(=O)OC(CCCCCCCC)CCCCCCCC)CCN1CCN(C)CC1]

or a pharmaceutically acceptable salt thereof;
bis(2-octyldodecyl) 3,3′-((2-(1-methylpyrrolidin-2-yl)ethyl)azanediyl)dipropionate having the following structure: (BODD-C2C2-1Me-Pyr)

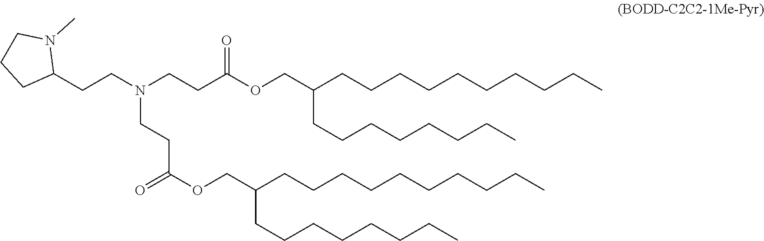 [SMILES: CCCCCCCCCCC(CCCCCCCC)COC(=O)CCN(CCC(=O)OCC(CCCCCCCC)CCCCCCCCCC)CCC1CCCN1C]

or a pharmaceutically acceptable salt thereof;
bis(2-octyldodecyl) 3,3′-((2-(pyrrolidin-1-yl)ethyl)azanediyl)dipropionate (BODD-C2C2-Pyr) or a pharmaceutically acceptable salt thereof;
bis(2-octyldodecyl) 3,3′-(((1-methylpiperidin-3-yl)methyl)azanediyl)dipropionate (BODD-C2C1-1Me-3PipD) or a pharmaceutically acceptable salt thereof;
bis(2-octyldodecyl) 3,3′-(((1-methylpiperidin-4-yl)methyl)azanediyl)dipropionate (BODD-C2C1-1Me-PipD) or a pharmaceutically acceptable salt thereof;
bis(2-octyldodecyl) 3,3′-((2-(dimethylamino)ethyl)azanediyl)dipropionate (BODD-C2C2-DMA) or a pharmaceutically acceptable salt thereof;
bis(2-octyldodecyl) 3,3′-((4-(4-methylpiperazin-1-yl)butyl)azanediyl)dipropionate (BODD-C2C4-PipZ) or a pharmaceutically acceptable salt thereof;
bis(2-octyldodecyl) 3,3′-((4-(pyrrolidin-1-yl)butyl)azanediyl)dipropionate (BODD-C2C4-Pyr) or a pharmaceutically acceptable salt thereof;
bis(2-hexyldecyl) 3,3′-((4-(4-methylpiperazin-1-yl)butyl)azanediyl)dipropionate (BHD-C2C4-PipZ) or

a pharmaceutically acceptable salt thereof;

or a mixture of any thereof.

In various different embodiments, the cationically ionizable lipid is selected from the group consisting of N,N-dimethyl-2,3-dioleyloxypropylamine (DODMA), 1,2-dioleoyl-3-dimethylammonium-propane (DODAP), heptatriaconta-6,9,28,31-tetraen-19-yl-4-(dimethylamino)butanoate (DLin-MC3-DMA), and 4-((di((9Z,12Z)-octadeca-9,12-dien-1-yl)amino)oxy)-N,N-dimethyl-4-oxobutan-1-amine (DPL-14).

Further examples of cationically ionizable lipids include, but are not limited to, 3-(N-(N′,N′-dimethylaminoethane)-carbamoyl)cholesterol (DC-Chol), 1,2-dioleoyl-3-dimethylammonium-propane (DODAP); 1,2-diacyloxy-3-dimethylammonium propanes; 1,2-dialkyloxy-3-dimethylammonium propanes, 1,2-distearyloxy-N,N-dimethyl-3-aminopropane (DSDMA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), 2-({8-[(3f)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVL5), di((Z)-non-2-en-1-yl) 8,8′-((((2(dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), di((Z)-non-2-en-1-yl)-9-((4-(dimethylaminobutanoyl)oxy)heptadecanedioate (L319), N-dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino)propionamide (lipidoid 98N12-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200).

In certain embodiments, the cationically ionizable lipid has the structure X-3.

In some embodiments, the cationically ionizable lipid for use herein is or comprises DPL-14. As used herein, “DPL-14” is a lipid comprising the following general formula:

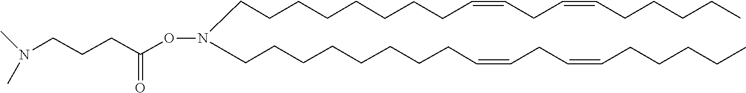

In certain embodiments, the cationic or cationically ionizable lipid comprises [(4-hydroxybutyl)azanediyl]di(hexane-6,1-diyl)bis(2-hexyldecanoate) (ALC-315); 1,2-dioleoyloxy-3-dimethylaminopropane (DODMA); 2,2-dilinoleyl-4-dimethylaminoethyl-[1,3]-dioxolane (DLin-KC2-DMA); heptatriaconta-6,9,28,31-tetraen-19-yl-4-(dimethylamino)butanoate (D-Lin-MC3-DMA); 1,2-dilinoleyloxy-N,N-dimethylaminopropane (DLin-DMA); di((Z)-non-2-en-1-yl)-9-((4-(dimethylaminobutanoyl)oxy)heptadecanedioate (L319); bis-(2-butyloctyl) 10-(N-(3-(dimethylamino)propyl)nonanamido)-nonadecanedioate (A9); (heptadecan-9-yl 8-{(2-hydroxyethyl)[6-oxo-6-(undecyloxy)octyl]amino}-octanoate) (L5); heptadecan-9-yl 8-{(2-hydroxyethyl)[6-oxo-6-(undecyloxy)hexyl]amino}-octanoate) (SM-102); O-[N-{(9Z,12Z)-octadeca-9,12-dien-1-yl)}-N-{7-pentadecylcarbonyloxyoctyl}-amino]4-(dimethylamino)butanoate (HY501); 2-(di-((9Z,12Z)-octadeca-9,12-dien-1-yl)amino)ethyl 4-(dimethylamino)butanoate (EA-2); 4-((di-((9Z,12Z)-octadeca-9,12-dien-1-yl)amino)oxy)-N,N-dimethyl-4-oxobutan-4-amine (HYAM-2); ((2-(4-(dimethylamino)butanoyl)oxy)ethyl)azanediylbis(octane 8,1-diyl)bis(2-hexyldecanoate) (EA-405); (2-(4-(dimethylamino)butanoyl)oxy)azanediylbis(octane 8,1-diyl)bis(2-hexyldecanoate) (HY-405); palmitoyl-oleoyl-nor-arginine (PONA); guanidino-di[(heptadecyl)methyl]carboxylic acid (GUADACA); 4-methylpyridinium-di(heptadecyl)methylcarboxylic acid (MPDACA); 1,2-dioleoyl-3 trimethylammonium propane (DOTAP); 1,2-dioleoyl-3-dimethylammomium propane (DODAP); or 1,2-di-O-octadecenyl-3-trimethylammonium propane (DOTMA); or a mixture of any thereof.

In certain embodiments, the cationic or cationically ionizable lipid is selected from the group consisting of [(4-hydroxybutyl)azanediyl]di(hexane-6,1-diyl)bis(2-hexyldecanoate) (ALC-315); 1,2-dioleoyloxy-3-dimethylaminopropane (DODMA); 2,2-dilinoleyl-4-dimethylaminoethyl-[1,3]-dioxolane (DLin-KC2-DMA); heptatriaconta-6,9,28,31-tetraen-19-yl-4-(dimethylamino)butanoate (D-Lin-MC3-DMA); 1,2-dilinoleyloxy-N,N-dimethylaminopropane (DLin-DMA); di((Z)-non-2-en-1-yl)-9-((4-(dimethylaminobutanoyl)oxy)heptadecanedioate (L319); bis-(2-butyloctyl) 10-(N-(3-(dimethylamino)propyl)nonanamido)-nonadecanedioate (A9); (heptadecan-9-yl 8-{(2-hydroxyethyl)[6-oxo-6-(undecyloxy)octyl]amino}-octanoate) (L5); heptadecan-9-yl 8-{(2-hydroxyethyl)[6-oxo-6-(undecyloxy)hexyl]amino}-octanoate) (SM-102); O-[N-{(9Z,12Z)-octadeca-9,12-dien-1-yl)}-N-{7-pentadecylcarbonyloxyoctyl}-amino]4-(dimethylamino)butanoate (HY501); 2-(di-((9Z,12Z)-octadeca-9,12-dien-1-yl)amino)ethyl 4-(dimethylamino)butanoate (EA-2); 4-((di-((9Z,12Z)-octadeca-9,12-dien-1-yl)amino)oxy)-N,N-dimethyl-4-oxobutan-4-amine (HYAM-2); ((2-(4-(dimethylamino)butanoyl)oxy)ethyl)azanediylbis(octane 8,1-diyl)bis(2-hexyldecanoate) (EA-405); (2-(4-(dimethylamino)butanoyl)oxy)azanediylbis(octane 8,1-diyl)bis(2-hexyldecanoate) (HY-405); palmitoyl-oleoyl-nor-arginine (PONA); guanidino-di[(heptadecyl)methyl]carboxylic acid (GUADACA); 4-methylpyridinium-di(heptadecyl)methylcarboxylic acid (MPDACA); 1,2-dioleoyl-3 trimethylammonium propane (DOTAP); 1,2-dioleoyl-3-dimethylammomium propane (DODAP); 1,2-di-O-octadecenyl-3-trimethylammonium propane (DOTMA); and a mixture of any thereof.

In some embodiments, the cationically ionizable lipid is selected from the group consisting of 7,7′-((4-hydroxybutyl)azanediyl)bis(N-hexyl-N-octylheptane-1-sulfonamide) (BNT-51); 7,7′-((4-(3,3-dimethylthioureido)butyl)azanediyl)bis(N-hexyl-N-octylheptane-1-sulfonamide) (BNT-52), BNT-76; [(4-hydroxybutyl)azanediyl]di(hexane-6,1-diyl)bis(2-hexyldecanoate) (ALC-315); 1,2-dioleoyloxy-3-dimethylaminopropane (DODMA); 2,2-dilinoleyl-4-dimethylaminoethyl-[1,3]-dioxolane (DLin-KC2-DMA); heptatriaconta-6,9,28,31-tetraen-19-yl-4-(dimethylamino)butanoate (D-Lin-MC3-DMA); 1,2-dilinoleyloxy-N,N-dimethylaminopropane (DLin-DMA); di((Z)-non-2-en-1-yl)-9-((4-(dimethylaminobutanoyl)oxy)heptadecanedioate (L319); bis-(2-butyloctyl) 10-(N-(3-(dimethylamino)propyl)nonanamido)-nonadecanedioate (A9); (heptadecan-9-yl 8-{(2-hydroxyethyl)[6-oxo-6-(undecyloxy)octyl]amino}-octanoate) (L5); heptadecan-9-yl 8-{(2-hydroxyethyl)[6-oxo-6-(undecyloxy)hexyl]amino}-octanoate) (SM-102); O-[N-{(9Z,12Z)-octadeca-9,12-dien-1-yl)}-N-{7-pentadecylcarbonyloxyoctyl}-amino]4-(dimethylamino)butanoate (HY501); 2-(di-((9Z,12Z)-octadeca-9,12-dien-1-yl)amino)ethyl 4-(dimethylamino)butanoate (EA-2); 4-((di-((9Z,12Z)-octadeca-9,12-dien-1-yl)amino)oxy)-N,N-dimethyl-4-oxobutan-4-amine (HYAM-2); ((2-(4-(dimethylamino)butanoyl)oxy)ethyl)azanediylbis(octane 8,1-diyl)bis(2-hexyldecanoate) (EA-405); (2-(4-(dimethylamino)butanoyl)oxy)azanediylbis(octane 8,1-diyl)bis(2-hexyldecanoate) (HY-405); di(heptadecan-9-yl) 3,3′-((2-(4-methylpiperazin-1-yl)ethyl)azanediyl)dipropionate (BHD-C2C2-PipZ); bis(2-octyldodecyl) 3,3′-((2-(1-methylpyrrolidin-2-yl)ethyl)azanediyl)dipropionate (BODD-C2C2-1Me-Pyr); bis(2-octyldodecyl) 3,3′-((2-(pyrrolidin-1-yl)ethyl)azanediyl)dipropionate (BODD-C2C2-Pyr); bis(2-octyldodecyl) 3,3′-(((1-methylpiperidin-3-yl)methyl)azanediyl)dipropionate (BODD-C2C1-1Me-3PipD); bis(2-octyldodecyl) 3,3′-(((1-methylpiperidin-4-yl)methyl)azanediyl)dipropionate (BODD-C2C1-1Me-PipD); bis(2-octyldodecyl) 3,3′-((2-(dimethylamino)ethyl)azanediyl)dipropionate (BODD-C2C2-DMA); bis(2-octyldodecyl) 3,3′-((4-(4-methylpiperazin-1-yl)butyl)azanediyl)dipropionate (BODD-C2C4-PipZ); bis(2-octyldodecyl) 3,3′-((4-(pyrrolidin-1-yl)butyl)azanediyl)dipropionate (BODD-C2C4-Pyr); bis(2-hexyldecyl) 3,3′-((4-(4-methylpiperazin-1-yl)butyl)azanediyl)dipropionate (BHD-C2C4-PipZ); and a mixture of any thereof.

In some embodiments, the cationically ionizable lipid is selected from the group consisting of 7,7′-((4-hydroxybutyl)azanediyl)-bis(N-hexyl-N-octylheptane-1-sulfonamide) (BNT-51); 7,7′-((4-(3,3-dimethylthioureido)butyl)azanediyl)bis(N-hexyl-N-octylheptane-1-sulfonamide) (BNT-52); BNT-76; [(4-hydroxybutyl)azanediyl]di(hexane-6,1-diyl)bis(2-hexyldecanoate) (ALC-0315); 1,2-dioleoyloxy-3-dimethylaminopropane (DODMA); 2,2-dilinoleyl-4-dimethylaminoethyl-[1,3]-dioxolane (DLin-KC2-DMA); heptatriaconta-6,9,28,31-tetraen-19-yl-4-(dimethylamino)butanoate (D-Lin-MC3-DMA); 1,2-dilinoleyloxy-N,N-dimethylaminopropane (DLin-DMA); di((Z)-non-2-en-1-yl)-9-((4-(dimethylaminobutanoyl)oxy)heptadecanedioate (L319); bis-(2-butyloctyl) 10-(N-(3-(dimethylamino)propyl)nonanamido)-nonadecanedioate (A9); (heptadecan-9-yl 8-{(2-hydroxyethyl)[6-oxo-6-(undecyloxy)octyl]amino}-octanoate) (L5); heptadecan-9-yl 8-{(2-hydroxyethyl)[6-oxo-6-(undecyloxy)hexyl]amino}-octanoate) (SM-102); O-[N-{(9Z,12Z)-octadeca-9,12-dien-1-yl)}-N-{7-pentadecylcarbonyloxyoctyl}-amino]4-(dimethylamino)butanoate (HY501); ((2-(4-(dimethylamino)butanoyl)oxy)ethyl)azanediylbis(octane 8,1-diyl)bis(2-hexyldecanoate) (EA-405); (2-(4-(dimethylamino)butanoyl)oxy)azanediylbis(octane 8,1-diyl)bis(2-hexyldecanoate) (HY-405); di(heptadecan-9-yl) 3,3′-((2-(4-methylpiperazin-1-yl)ethyl)azanediyl)dipropionate (BHD-C2C2-PipZ); bis(2-octyldodecyl) 3,3′-((2-(1-methylpyrrolidin-2-yl)ethyl)azanediyl)dipropionate (BODD-C2C2-1Me-Pyr); bis(2-octyldodecyl) 3,3′-((2-(pyrrolidin-1-yl)ethyl)azanediyl)dipropionate (BODD-C2C2-Pyr); bis(2-octyldodecyl) 3,3′-(((1-methylpiperidin-3-yl)methyl)azanediyl)dipropionate (BODD-C2C1-1Me-3PipD); bis(2-octyldodecyl) 3,3′-(((1-methylpiperidin-4-yl)methyl)azanediyl)dipropionate (BODD-C2C1-1Me-PipD); bis(2-octyldodecyl) 3,3′-((2-(dimethylamino)ethyl)azanediyl)dipropionate (BODD-C2C2-DMA); bis(2-octyldodecyl) 3,3′-((4-(4-methylpiperazin-1-yl)butyl)azanediyl)dipropionate (BODD-C2C4-PipZ); bis(2-octyldodecyl) 3,3′-((4-(pyrrolidin-1-yl)butyl)azanediyl)dipropionate (BODD-C2C4-Pyr); bis(2-hexyldecyl) 3,3′-((4-(4-methylpiperazin-1-yl)butyl)azanediyl)dipropionate (BHD-C2C4-PipZ); and a mixture of any thereof.

It is to be understood that any reference to a cationically ionizable lipid disclosed herein also includes the salts (in particular pharmaceutically acceptable salts), tautomers, stereoisomers, solvates (e.g., hydrates), and isotopically labeled forms thereof.

In some embodiments, the cationic or cationically ionizable lipid may comprise from about 10 mol % to about 100 mol %, about 20 mol % to about 100 mol %, about 30 mol % to about 100 mol %, about 40 mol % to about 100 mol %, or about 50 mol % to about 100 mol % of the total lipid present in the composition/particle. In some embodiments, the cationic or cationically ionizable lipid comprises from about 40 mol % to about 75 mol %, preferably from about 40 mol % to about 70 mol %, more preferably from about 45 mol % to about 65 mol %, of the total lipid present in the composition/particles.

In some embodiments, wherein the nucleic acid compositions/particles (in particular the RNA compositions/particles) described herein comprise a cationic or cationically ionizable lipid and one or more additional lipids, the cationic or cationically ionizable lipid comprises from about 10 mol % to about 80 mol %, from about 20 mol % to about 80 mol %, from about 20 mol % to about 75 mol %, from about 25 mol % to about 70 mol %, from about 25 mol % to about 65 mol %, from about 25 mol % to about 65 mol %, from about 25 mol % to about 60 mol %, from about 25 mol % to about 55 mol %, from about 30 mol % to about 50 mol %, from about 35 mol % to about 50 mol %, or from about 40 mol % to about 50 mol % of the total lipid present in the composition/particles.

In some embodiments, the N/P ratio is at least about 2. In some embodiments, the N/P ratio ranges from about 2 to about 20, such as from about 2 to about 12, from about 4 to about 12, from about 4 to about 10, from about 4 to about 8, or from about 5 to about 7. In some embodiments, the N/P ratio is about 6.

### Additional Lipids

In some embodiments, the nucleic acid (such as RNA) compositions described herein may also comprise lipids or lipid-like materials other than cationic/cationically ionizable lipids, i.e., non-cationic lipids or lipid-like materials (including non-cationic lipids or lipid-like materials). Collectively, anionic and neutral lipids or lipid-like materials are referred to herein as non-cationic lipids or lipid-like materials. In some embodiments, optimizing the formulation of nucleic acid particles by addition of other hydrophobic moieties, such as cholesterol and lipids, in addition to a cationic/cationically ionizable lipid may enhance composition and/or particle stability and efficacy of nucleic acid (such as RNA) delivery.

One or more additional lipids may be incorporated which may or may not affect the overall charge of the nucleic acid particles. In certain embodiments, the one or more additional lipids are a non-cationic lipid or lipid-like material. The non-cationic lipid may comprise, e.g., one or more anionic lipids and/or neutral lipids. As used herein, an “anionic lipid” refers to any lipid that is negatively charged at a selected pH. As used herein, a “neutral lipid” refers to any of a number of lipid species that exist either in an uncharged or neutral zwitterionic form at a selected pH.

In certain embodiments, the nucleic acid (such as RNA) compositions (especially the mRNA compositions) described herein comprise a cationic/cationically ionizable lipid and one or more additional lipids.

In some embodiments, the one or more additional lipids comprised in the nucleic acid (such as RNA) compositions (especially in the mRNA compositions) described herein comprise one or more of the following: neutral lipids, steroids, and combinations thereof. In some embodiments, the one or more additional lipids comprised in the nucleic acid (such as RNA) compositions (especially in the mRNA compositions) described herein comprise: a neutral lipid (in particular a phospholipid), a steroid (in particular cholesterol), and optionally a polymer-conjugated lipid.

### Neutral Lipids

In some embodiments, the one or more additional lipids comprise a neutral lipid which is preferably a phospholipid. In some embodiments, the phospholipid is selected from the group consisting of phosphatidylcholines, phosphatidylethanolamines, phosphatidylglycerols, phosphatidic acids, phosphatidylserines and sphingomyelins. Specific phospholipids that can be used include, but are not limited to, phosphatidylcholines, phosphatidylethanolamines, phosphatidylglycerols, phosphatidic acids, phosphatidylserines or sphingomyelin. Such phospholipids include in particular diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC) and phosphatidylethanolamines, in particular diacylphosphatidylethanolamines, such as dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), diphytanoyl-phosphatidylethanolamine (DPyPE), 1,2-di-(9Z-octadecenoyl)-sn-glycero-3-phosphocholine (DOPG), 1,2-dipalmitoyl-sn-glycero-3-phospho-(1′-rac-glycerol) (DPPG), 1-palmitoyl-2-oleoyl-sn-glycero-3-phosphoethanolamine (POPE), N-palmitoyl-D-erythro-sphingosylphosphorylcholine (SM), and further phosphatidylethanolamine lipids with different hydrophobic chains. In some embodiments, the neutral lipid is selected from the group consisting of DSPC, DOPC, and DOPE. In some embodiments, the neutral lipid is DSPC or DOPC. In some embodiments, the neutral lipid is DSPC. In some embodiments, the neutral lipid is DOPC. In some embodiments, the neutral lipid is DOPE.

Thus, in some embodiments, the nucleic acid (such as RNA) compositions (especially the mRNA compositions) described herein comprise a cationically ionizable lipid and a phospholipid. In some embodiments, the nucleic acid (such as RNA) compositions (especially the mRNA compositions) described herein comprise a cationically ionizable lipid and a phospholipid selected from the group consisting of DSPC, DOPC, and DOPE. In some embodiments, the nucleic acid (such as RNA) compositions (especially the mRNA compositions) described herein comprise a cationically ionizable lipid and DSPC. In some embodiments, the nucleic acid (such as RNA) compositions (especially the mRNA compositions) described herein comprise a cationically ionizable lipid and DOPC. In some embodiments, the nucleic acid (such as RNA) compositions (especially the mRNA compositions) described herein comprise a cationically ionizable lipid and DOPE.

In some embodiments, the neutral lipid is present in the nucleic acid (such as RNA) compositions (in particular the mRNA compositions) described herein in a concentration ranging from 5 to 40 mol percent. In some embodiments, the neutral lipid is present in a concentration of from about 5 mol % to about 20 mol %, such as from about 5 mol % to about 15 mol %, of the total lipids present in the nucleic acid (such as RNA) composition (especially the mRNA composition) described herein.

### Steroid

In some embodiments, the steroid is cholesterol. Thus, in some embodiments, the nucleic acid (such as RNA) compositions (especially the mRNA compositions) described herein comprise a cationically ionizable lipid and cholesterol.

In some embodiments, the steroid is present in the nucleic acid (such as RNA) compositions (in particular the mRNA compositions) described herein in a concentration ranging from 10 to 65 mol percent. In some embodiments, the steroid is present in a concentration from about 20 mol % to about 60 mol %, such as from about 30 mol % to about 50 mol %, of the total lipids present in the compositions (especially the mRNA compositions) described herein.

In certain preferred embodiments, the nucleic acid (such as RNA) compositions (especially the mRNA compositions) described herein comprise a phospholipid and cholesterol, preferably in the concentrations given above. In some embodiments, the nucleic acid (such as RNA) compositions (especially the mRNA compositions) described herein comprise a phospholipid selected from the group consisting of DSPC, DOPC, and DOPE, and cholesterol, preferably in the concentrations given above.

In some embodiments, the nucleic acid (such as RNA) compositions (especially the mRNA compositions) described herein comprise DSPC and cholesterol, preferably in the concentrations given above. In some embodiments, the nucleic acid (such as RNA) compositions (especially the mRNA compositions) described herein comprise DOPC and cholesterol, preferably in the concentrations given above. In some embodiments, the nucleic acid (such as RNA) compositions (especially the mRNA compositions) described herein comprise DOPE and cholesterol, preferably in the concentrations given above.

In some embodiments, the combined concentration of the neutral lipid (in particular, one or more phospholipids) and steroid (in particular, cholesterol) may comprise from about 0 mol % to about 70 mol %, such as from about 2 mol % to about 60 mol %, from about 5 mol % to about 55 mol %, from about 5 mol % to about 50 mol %, from about 5 mol % to about 40 mol %, or from about 20 mol % to about 40 mol % of the total lipids present in the nucleic acid compositions (such as DNA or RNA compositions, especially the mRNA compositions) described herein.

### Polymer-Conjugated Lipids

A polymer-conjugated lipid is typically a molecule comprising a lipid portion and a polymer portion conjugated thereto.

A “polymer,” as used herein, is given its ordinary meaning, i.e., a molecular structure comprising one or more repeat units (monomers), connected by covalent bonds. The repeat units can all be identical, or in some cases, there can be more than one type of repeat unit present within the polymer. In some cases, the polymer is biologically derived, i.e., a biopolymer such as a protein. In some cases, additional moieties can also be present in the polymer, for example targeting moieties. If more than one type of repeat unit is present within the polymer, then the polymer is said to be a “copolymer.” The repeat units forming the copolymer can be arranged in any fashion. For example, the repeat units can be arranged in a random order, in an alternating order, or as a “block” copolymer, i.e., comprising one or more regions each comprising a first repeat unit (e.g., a first block), and one or more regions each comprising a second repeat unit (e.g., a second block), etc. Block copolymers can have two (a diblock copolymer), three (a triblock copolymer), or more numbers of distinct blocks.

In some embodiments, a polymer-conjugated lipid is designed to sterically stabilize a lipid particle by forming a protective hydrophilic layer that shields the hydrophobic lipid layer. In some embodiments, a polymer-conjugated lipid can reduce its association with serum proteins and/or the resulting uptake by the reticuloendothelial system when such lipid particles are administered in vivo.

In some embodiments, the polymer-conjugated lipid comprises from about 0.1 mol % to about 10 mol %, such as from about 0.5 mol % to about 5 mol %, such as from about 1 mol % to about 5 mol % or from about 1 mol % to about 4.5 mol % of the total lipids present in the nucleic acid compositions (such as DNA or RNA, mRNA compositions) described herein.

In some embodiments, the nucleic acid compositions (such as DNA or RNA compositions, especially mRNA compositions) described herein may comprise a pegylated lipid.

However, due to the disadvantages of PEG lipids, in particular of those PEG lipids having at least 30 consecutive ethylene glycol repeating units (e.g., potential association with accelerated blood clearance (ABC) phenomenon induced by anti-PEG antibodies), in some embodiments, the nucleic acid compositions (such as DNA or RNA compositions, especially mRNA compositions) described herein are substantially free of a pegylated lipid having at least 30 consecutive ethylene glycol repeating units (but may comprise a polymer-conjugated other than PEG lipids having at least 30 consecutive ethylene glycol repeating units). In some embodiments, the nucleic acid compositions (such as DNA or RNA compositions, especially mRNA compositions) described herein do not contain a pegylated lipid having at least 30 consecutive ethylene glycol repeating units (but may comprise a polymer-conjugated other than PEG lipids having at least 30 consecutive ethylene glycol repeating units).

Thus, in some embodiments the nucleic acid compositions (such as DNA or RNA compositions, especially mRNA compositions) described herein may comprise a polymer-conjugated lipid selected from the group consisting of: poly(sarcosine) (pSar)-conjugated lipids, poly(oxazoline) (POX)-conjugated lipids; poly(oxazine) (POZ)-conjugated lipids; POX/POZ-conjugated lipids; poly(vinyl pyrrolidone) (PVP)-conjugated lipids; poly(N-(2-hydroxypropyl)-methacrylamide) (pHPMA)-conjugated lipids; poly(dehydroalanine) (pDha)-conjugated lipids; oligo ethylene glycol (OEG)-conjugated lipids (such as poly(aminoethoxy ethoxy acetic acid) (pAEEA)-conjugated lipids and poly(2-methylaminoethoxy ethoxy acetic acid) (pmAEEA)-conjugated lipids), and PEG-conjugated lipids.

In some embodiments the nucleic acid compositions (such as DNA or RNA compositions, especially mRNA compositions) described herein may comprise a polymer-conjugated lipid that is not a pegylated lipid. In these embodiments, it is preferred that the polymer-conjugated lipid that is not a pegylated lipid is selected from the group consisting of: pSar-conjugated lipids; POX-conjugated lipids; POZ-conjugated lipids; POX/POZ-conjugated lipids; PVP-conjugated lipids; pHPMA-conjugated lipids; pDha-conjugated lipids; and OEG-conjugated lipids (in particular, pAEEA-conjugated lipids, and pmAEEA-conjugated lipids). In preferred embodiments, the polymer-conjugated lipid is a sarcosinylated lipid or a pAEEA- or pmAEAA-conjugated lipid.

In some embodiments, the nucleic acid compositions (such as DNA or RNA compositions, especially mRNA compositions) described herein may comprise a sarcosinylated lipid. In some embodiments, the nucleic acid compositions (such as DNA or RNA compositions, especially mRNA compositions) described herein may comprise an OEG-conjugated lipid (in particular a pAEEA-conjugated lipid). In some embodiments, the nucleic acid compositions (such as DNA or RNA compositions, especially mRNA compositions) described herein comprise a sarcosinylated lipid or an OEG-conjugated lipid (in particular a pAEEA-conjugated lipid), and are substantially free of a pegylated lipid having at least 30 consecutive ethylene glycol repeating units.

### PEG-Conjugated Lipids

In some embodiments, the nucleic acid compositions (such as DNA or RNA compositions, especially mRNA compositions) described herein comprise a cationic/cationically ionizable lipid as described herein and a PEG-conjugated lipid. A PEG-conjugated lipid is also referred to herein as pegylated lipid or PEG-lipid. The term “pegylated lipid” refers to a molecule comprising both a lipid portion and a polyethylene glycol portion, wherein the polyethylene glycol portion preferably comprises at least 30 consecutive ethylene glycol repeating units. Pegylated lipids are known in the art.

In some embodiments, the nucleic acid compositions (such as DNA or RNA compositions, especially mRNA compositions) described herein may further comprise a neutral lipid (e.g., a phospholipid, cholesterol or a derivative thereof) or a combination of neutral lipids (e.g., a phospholipid, and cholesterol or a derivative thereof). In some embodiments, the nucleic acid compositions (such as DNA or RNA compositions, especially mRNA compositions) described herein comprise a cationic/cationically ionizable lipid as described herein, a PEG-conjugated lipid, a neutral lipid (e.g., a phospholipid), and cholesterol or a derivative thereof. In some embodiments, the phospholipid is selected from the group consisting of DSPC, DOPC, and DOPE. In some embodiments, the cationic/cationically ionizable lipid is a cationically ionizable lipid, such as that of formula (X) (e.g. a cationically ionizable lipid of formula (X-3) or (X-45)), or that of formula (TL-I) (e.g., a cationically ionizable lipid of formula BNT-51, BNT-52, or BNT-76), or that of formula (AXL-I) (e.g., a cationically ionizable lipid of formula BHD-C2C2-PipZ, BODD-C2C2-1Me-Pyr, BODD-C2C2-Pyr, BODD-C2C1-1Me-3PipD, BODD-C2C1-1Me-PipD, BODD-C2C2-DMA, BODD-C2C4-PipZ, or BODD-C2C4-Pyr, BHD-C2C4-PipZ). In some embodiments, the cationic/cationically ionizable lipid is a cationically ionizable lipid of formula (XI) (such as a cationically ionizable lipid of formula (XIV-1), (XIV-2), or (XIV-3)). In some embodiments, the cationic/cationically ionizable lipid is DPL14, EA-2, or 3D-β-DMA. In some embodiments, the cationic/cationically ionizable lipid comprises [(4-hydroxybutyl)azanediyl]di(hexane-6,1-diyl)bis(2-hexyldecanoate) (ALC-315); 1,2-dioleoyloxy-3-dimethylaminopropane (DODMA); 2,2-dilinoleyl-4-dimethylaminoethyl-[1,3]-dioxolane (DLin-KC2-DMA); heptatriaconta-6,9,28,31-tetraen-19-yl-4-(dimethylamino)butanoate (D-Lin-MC3-DMA); 1,2-dilinoleyloxy-N,N-dimethylaminopropane (DLin-DMA); di((Z)-non-2-en-1-yl)-9-((4-(dimethylaminobutanoyl)oxy)heptadecanedioate (L319); bis-(2-butyloctyl) 10-(N-(3-(dimethylamino)propyl)nonanamido)-nonadecanedioate (A9); (heptadecan-9-yl 8-{(2-hydroxyethyl)[6-oxo-6-(undecyloxy)octyl]amino}-octanoate) (L5); heptadecan-9-yl 8-{(2-hydroxyethyl)[6-oxo-6-(undecyloxy)hexyl]amino}-octanoate) (SM-102); O-[N-{(9Z,12Z)-octadeca-9,12-dien-1-yl)}-N-{7-pentadecylcarbonyloxyoctyl}-amino]4-(dimethylamino)butanoate (HY501); 2-(di-((9Z,12Z)-octadeca-9,12-dien-1-yl)amino)ethyl 4-(dimethylamino)butanoate (EA-2); 4-((di-((9Z,12Z)-octadeca-9,12-dien-1-yl)amino)oxy)-N,N-dimethyl-4-oxobutan-4-amine (HYAM-2); ((2-(4-(dimethylamino)butanoyl)oxy)ethyl)azanediylbis(octane 8,1-diyl)bis(2-hexyldecanoate) (EA-405); (2-(4-(dimethylamino)butanoyl)oxy)azanediylbis(octane 8,1-diyl)bis(2-hexyldecanoate) (HY-405); palmitoyl-oleoyl-nor-arginine (PONA); guanidino-di[(heptadecyl)methyl]carboxylic acid (GUADACA); 4-methylpyridinium-di(heptadecyl)methylcarboxylic acid (MPDACA); 1,2-dioleoyl-3 trimethylammonium propane (DOTAP); 1,2-dioleoyl-3-dimethylammomium propane (DODAP); or 1,2-di-O-octadecenyl-3-trimethylammonium propane (DOTMA); or a mixture of any thereof. In some embodiments, the cationic/cationically ionizable lipid is BNT-51, BNT-52, BNT-76, BHD-C2C2-PipZ, BODD-C2C2-1Me-Pyr, BODD-C2C2-Pyr, BODD-C2C1-1Me-3PipD, BODD-C2C1-1Me-PipD, BODD-C2C2-DMA, BODD-C2C4-PipZ, BODD-C2C4-Pyr, or BHD-C2C4-PipZ, or a mixture of any thereof.

In some embodiments, the PEG-conjugated lipid is a lipid having the structure of the following general formula (XVI):

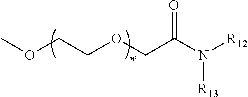

or a pharmaceutically acceptable salt, tautomer or stereoisomer thereof, wherein: each of R₁₂ and R₁₃ is each independently a straight or branched, alkyl or alkenyl chain containing from 10 to 30 carbon atoms, wherein the alkyl/alkenyl chain is optionally interrupted by one or more ester bonds; and w has a mean value ranging from 30 to 60.

In some embodiments of formula (XVI), each of R₁₂ and R₁₃ is independently a straight alkyl chain containing from 10 to 18 carbon atoms, preferably from 12 to 16 carbon atoms.

In some embodiments of formula (XVI), R₁₂ and R₁₃ are identical. In some embodiments, each of R₁₂ and R₁₃ is a straight alkyl chain containing 12 carbon atoms. In some embodiments, each of R₁₂ and R₁₃ is a straight alkyl chain containing 14 carbon atoms. In some embodiments, each of R₁₂ and R₁₃ is a straight alkyl chain containing 16 carbon atoms.

In some embodiments of formula (XVI), R₁₂ and R₁₃ are different. In some embodiments, one of R₁₂ and R₁₃ is a straight alkyl chain containing 12 carbon atoms and the other of R₁₂ and R₁₃ is a straight alkyl chain containing 14 carbon atoms.

In some embodiments of formula (XVI), w has a mean value ranging from 40 to 50, such as a mean value of 45.

In some embodiments of formula (XVI), w is within a range such that the PEG portion of the pegylated lipid of formula (XVI) has an average molecular weight of from about 400 to about 6000 g/mol, such as from about 1000 to about 5000 g/mol, from about 1500 to about 4000 g/mol, or from about 2000 to about 3000 g/mol.

Various PEG-conjugated lipids are known in the art and include, but are not limited to pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(o-methoxy(polyethoxy)ethyl)-butanedioate (PEG-S-DMG), a pegylated ceramide (PEG-cer), or a PEG dialkoxypropylcarbamate such as o-methoxy(polyethoxy)ethyl-N-(2,3-di(tetradecanoxy)propyl)carbamate or 2,3-di(tetradecanoxy)-propyl-N-(o methoxy(polyethoxy)ethyl)carbamate, and the like.

In some embodiments, the PEG-conjugated lipid is or comprises 2-[(polyethylene glycol)-2000]-N,N-ditetradecylacetamide. In some embodiments, the pegylated lipid has the following structure:

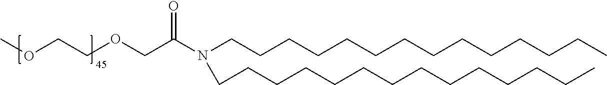

Thus, in some embodiments, the nucleic acid compositions (such as DNA or RNA compositions, especially mRNA compositions) described herein may comprise a cationic/cationically ionizable lipid as described herein (e.g., a cationically ionizable lipid, such as that of formula (X-3), BNT-51, BNT-52, BNT-76, BHD-C2C2-PipZ, BODD-C2C2-1Me-Pyr, BODD-C2C2-Pyr, BODD-C2C1-1Me-3PipD, BODD-C2C1-1Me-PipD, BODD-C2C2-DMA, BODD-C2C4-PipZ, BODD-C2C4-Pyr, or BHD-C2C4-PipZ), 2-[(polyethylene glycol)-2000]-N,N-ditetradecylacetamide, a neutral lipid (e.g., a phospholipid like DSPC), and a steroid (e.g., cholesterol).

In some embodiments, the PEG-conjugated lipid is DMG-PEG 2000, e.g., having the following structure:

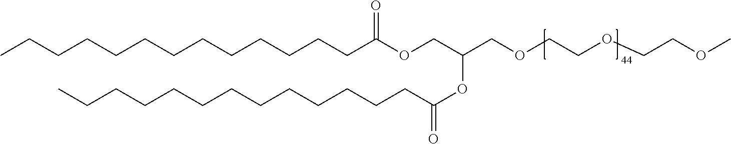

Thus, in some embodiments, the nucleic acid compositions (such as DNA or RNA compositions, especially mRNA compositions) described herein may comprise a cationic/cationically ionizable lipid as described herein (e.g., a cationically ionizable lipid, such as that of formula (X-3), BNT-51, BNT-52, BNT-76, BHD-C2C2-PipZ, BODD-C2C2-1Me-Pyr, BODD-C2C2-Pyr, BODD-C2C1-1Me-3PipD, BODD-C2C1-1Me-PipD, BODD-C2C2-DMA, BODD-C2C4-PipZ, BODD-C2C4-Pyr, or BHD-C2C4-PipZ), DMG-PEG 2000, a neutral lipid (e.g., a phospholipid like DSPC), and a steroid (e.g., cholesterol).

In some embodiments, the PEG-conjugated lipid has the following structure:

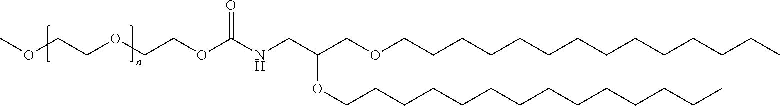

wherein n has a mean value ranging from 30 to 60, such as about 50. In one embodiment, the PEG-conjugated lipid (pegylated lipid) is PEG₂₀₀₀-C-DMA which preferably refers to 3-N-[(ω-methoxy poly(ethylene glycol)2000)carbamoyl]-1,2-dimyristyloxy-propylamine (MPEG-(2 kDa)-C-DMA) or methoxy-polyethylene glycol-2,3-bis(tetradecyloxy)propylcarbamate (2000). PEG₂₀₀₀-C-DMA may be selected in order to provide optimum delivery of RNA to the liver. It has been found that by modulating the alkyl chain length of the PEG lipid anchor, the pharmacology of encapsulated nucleic acid can be controlled in a predictable manner. In a vial, i.e., prior to administration, the particles retain a full complement of PEG₂₀₀₀-C-DMA. In the blood compartment, i.e., after administration, PEG₂₀₀₀-C-DMA dissociates from the RNA particle over time, revealing a more fusogenic particle that is more readily taken up by cells, ultimately leading to release of the RNA payload. Thus, in some embodiments, the nucleic acid compositions (such as DNA or RNA compositions, especially mRNA compositions) described herein may comprise a cationic/cationically ionizable lipid as described herein (e.g., a cationically ionizable lipid, such as that of formula (X-3), BNT-51, BNT-52, BNT-76, BHD-C2C2-PipZ, BODD-C2C2-1Me-Pyr, BODD-C2C2-Pyr, BODD-C2C1-1Me-3PipD, BODD-C2C1-1Me-PipD, BODD-C2C2-DMA, BODD-C2C4-PipZ, BODD-C2C4-Pyr, or BHD-C2C4-PipZ), PEG₂₀₀₀-C-DMA, a neutral lipid (e.g., a phospholipid like DSPC), and a steroid (e.g., cholesterol).

In some embodiments, the PEG-conjugated lipid is or comprises DSPE-PEG, DOPE-PEG, DPPE-PEG, and DMPE-PEG. Thus, in some embodiments, the nucleic acid compositions (such as DNA or RNA compositions, especially mRNA compositions) described herein may comprise a cationic/cationically ionizable lipid as described herein (e.g., a cationically ionizable lipid, such as that of formula (X-3), BNT-51, BNT-52, BNT-76, BHD-C2C2-PipZ, BODD-C2C2-1Me-Pyr, BODD-C2C2-Pyr, BODD-C2C1-1Me-3PipD, BODD-C2C1-1Me-PipD, BODD-C2C2-DMA, BODD-C2C4-PipZ, or BODD-C2C4-Pyr, BHD-C2C4-PipZ); one or more PEG-conjugated lipids selected from the group consisting of comprises DSPE-PEG, DOPE-PEG, DPPE-PEG, and DMPE-PEG (wherein the PEG moiety preferably is PEG₂₀₀₀); a neutral lipid (e.g., a phospholipid like DSPC); and a steroid (e.g., cholesterol).

In some embodiments, the PEG-conjugated lipid is or comprises one or more PEG-conjugated lipids or pegylated lipids as described in WO 2017/075531 and WO 2018/081480, the entire contents of each of which are incorporated herein by reference for the purposes described herein. Thus, in some embodiments, the nucleic acid compositions (such as DNA or RNA compositions, especially mRNA compositions) described herein may comprise a cationic/cationically ionizable lipid as described herein (e.g., a cationically ionizable lipid, such as that of formula (X-3), BNT-51, BNT-52, BNT-76, BHD-C2C2-PipZ, BODD-C2C2-1Me-Pyr, BODD-C2C2-Pyr, BODD-C2C1-1Me-3PipD, BODD-C2C1-1Me-PipD, BODD-C2C2-DMA, BODD-C2C4-PipZ, BODD-C2C4-Pyr, or BHD-C2C4-PipZ); one or more PEG-conjugated lipids or pegylated lipids as described in WO 2017/075531 and WO 2018/081480; a neutral lipid (e.g., a phospholipid like DSPC); and a steroid (e.g., cholesterol).

In some embodiments, the pegylated lipid comprises from about 1 mol % to about 10 mol %, such as from about 1 mol % to about 5 mol %, or from about 1 mol % to about 2.5 mol % of the total lipids present in the nucleic acid compositions (such as DNA or RNA, mRNA compositions) described herein.

In some embodiments, the nucleic acid compositions (such as DNA or RNA compositions, especially mRNA compositions) described herein may comprise a cationic/cationically ionizable lipid as described herein (e.g., a cationically ionizable lipid, such as that of formula (X)), a pegylated lipid as described herein, a phospholipid as described herein, and cholesterol, wherein the cationic/cationically ionizable lipid comprises from about 40 mol % to about 50 mol % of the total lipid present in the composition, the pegylated lipid comprises from about 1 mol % to about 5 mol % (such as from about 1 mol % to about 2.5 mol %) of the total lipid present in the composition, the phospholipid comprises from about 5 mol % to about 15 mol % of the total lipid present in the composition, and the cholesterol comprises from about 30 mol % to about 50 mol % of the total lipid present in the composition. In some embodiments, the pegylated lipid is selected from the group consisting of 2-[(polyethylene glycol)-2000]-N,N-ditetradecylacetamide, DMG-PEG 2000, and PEG₂₀₀₀-C-DMA.

In some embodiments of the nucleic acid compositions (such as DNA or RNA compositions, especially mRNA compositions) described herein which comprise a pegylated lipid as described herein, the N/P ratio is at least about 2. In some embodiments, the N/P value ranges from about 2 to about 20, such as from about 2 to about 12, from about 4 to about 12, from about 4 to about 10, from about 4 to about 8, or from about 5 to about 7. In some embodiments, the N/P ratio is about 6.

### Sarcosinylated Lipids

In some embodiments, the nucleic acid compositions (such as DNA or RNA compositions, especially mRNA compositions) described herein comprise a cationic/cationically ionizable lipid as described herein and a sarcosinylated lipid (pSAR-conjugated lipid). A polysarcosine-conjugated lipid is also referred to herein as sarcosinylated lipid or pSar-lipid. The term “sarcosinylated lipid” refers to a molecule comprising both a lipid portion and a polysarcosine (poly(N-methylglycine) portion.

In some embodiments, the nucleic acid compositions (such as DNA or RNA compositions, especially mRNA compositions) described herein may further comprise a neutral lipid (e.g., a phospholipid, cholesterol or a derivative thereof) or a combination of neutral lipids (e.g., a phospholipid, and cholesterol or a derivative thereof). In some embodiments, the nucleic acid compositions (such as DNA or RNA compositions, especially mRNA compositions) described herein comprise a cationic/cationically ionizable lipid as described herein, a sarcosinylated lipid, a neutral lipid (e.g., a phospholipid), and cholesterol or a derivative thereof. In some embodiments, the phospholipid is selected from the group consisting of DSPC, DOPC, and DOPE. In some embodiments, the cationic/cationically ionizable lipid is a cationically ionizable lipid, such as that of formula (X), e.g., a cationically ionizable lipid of formula (X-3) or (X-45). In some embodiments, the cationic/cationically ionizable lipid is a cationically ionizable lipid, such as that of formula (XI), e.g., a cationically ionizable lipid of formula (XIV-1), (XIV-2), or (XIV-3), or that of formula (TL-I), e.g., a cationically ionizable lipid of formula BNT-51, BNT-52, or BNT-76, or that of formula (AXL-I), e.g., a cationically ionizable lipid of formula BHD-C2C2-PipZ, BODD-C2C2-1Me-Pyr, BODD-C2C2-Pyr, BODD-C2C1-1Me-3PipD, BODD-C2C1-1Me-PipD, BODD-C2C2-DMA, BODD-C2C4-PipZ, BODD-C2C4-Pyr, or BHD-C2C4-PipZ. In some embodiments, the cationic/cationically ionizable lipid is DPL14, EA-2, or 3D-P-DMA. In some embodiments, the cationic/cationically ionizable lipid comprises [(4-hydroxybutyl)azanediyl]di(hexane-6,1-diyl)bis(2-hexyldecanoate) (ALC-315); 1,2-dioleoyloxy-3-dimethylaminopropane (DODMA); 2,2-dilinoleyl-4-dimethylaminoethyl-[1,3]-dioxolane (DLin-KC2-DMA); heptatriaconta-6,9,28,31-tetraen-19-yl-4-(dimethylamino)butanoate (D-Lin-MC3-DMA); 1,2-dilinoleyloxy-N,N-dimethylaminopropane (DLin-DMA); di((Z)-non-2-en-1-yl)-9-((4-(dimethylaminobutanoyl)oxy)heptadecanedioate (L319); bis-(2-butyloctyl) 10-(N-(3-(dimethylamino)propyl)nonanamido)-nonadecanedioate (A9); (heptadecan-9-yl 8-{(2-hydroxyethyl)[6-oxo-6-(undecyloxy)octyl]amino}-octanoate) (L5); heptadecan-9-yl 8-{(2-hydroxyethyl)[6-oxo-6-(undecyloxy)hexyl]amino}-octanoate) (SM-102); O-[N-{(9Z,12Z)-octadeca-9,12-dien-1-yl)}-N-{7-pentadecylcarbonyloxyoctyl}-amino]4-(dimethylamino)butanoate (HY501); 2-(di-((9Z,12Z)-octadeca-9,12-dien-1-yl)amino)ethyl 4-(dimethylamino)butanoate (EA-2); 4-((di-((9Z,12Z)-octadeca-9,12-dien-1-yl)amino)oxy)-N,N-dimethyl-4-oxobutan-4-amine (HYAM-2); ((2-(4-(dimethylamino)butanoyl)oxy)ethyl)azanediylbis(octane 8,1-diyl)bis(2-hexyldecanoate) (EA-405); (2-(4-(dimethylamino)butanoyl)oxy)azanediylbis(octane 8,1-diyl)bis(2-hexyldecanoate) (HY-405); palmitoyl-oleoyl-nor-arginine (PONA); guanidino-di[(heptadecyl)methyl]carboxylic acid (GUADACA); 4-methylpyridinium-di(heptadecyl)methylcarboxylic acid (MPDACA); 1,2-dioleoyl-3 trimethylammonium propane (DOTAP); 1,2-dioleoyl-3-dimethylammomium propane (DODAP); or 1,2-di-O-octadecenyl-3-trimethylammonium propane (DOTMA); or a mixture of any thereof.

In some embodiments of the nucleic acid compositions (such as DNA or RNA compositions, especially mRNA compositions) described herein which comprise a sarcosinylated lipid, said compositions are substantially free of a pegylated lipid having at least 30 consecutive ethylene glycol repeating units (or do not contain a pegylated lipid having at least 30 consecutive ethylene glycol repeating units).

In some embodiments, the sarcosinylated lipid comprises between 2 and 200 sarcosine units, such as between 5 and 100 sarcosine units, between 10 and 50 sarcosine units, between 15 and 40 sarcosine units, e.g., about 23 sarcosine units.

In some embodiments, the sarcosinylated lipid comprises the structure of the following general formula (XVII):

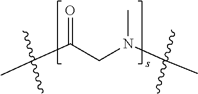

wherein s is the number of sarcosine units.

In some embodiments, the sarcosinylated lipid comprises the structure of the following general formula (XVIII):

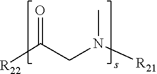

wherein one of R₂₁ and R₂₂ comprises a hydrophobic group and the other is H, a hydrophilic group or a functional group optionally comprising a targeting moiety; and x is the number of sarcosine units.

In some embodiments of formula (XVIII), R₂₁ is H, a hydrophilic group or a functional group optionally comprising a targeting moiety; and R₂₂ comprises one or two straight alkyl or alkenyl groups each having at least 12 carbon atoms, such as at least 14 carbon atoms. In some embodiments, each of the straight alkyl and alkenyl groups has at most 30 carbon atoms, such as at most 28, at most 26, at most 24, at most 22, at most 20, or at most 18 carbon atoms. In some embodiments, R₂₂ comprises one or two straight alkyl or alkenyl groups each having 12 to 30 carbon atoms (such as 12 to 28 carbon atoms, 12 to 26 carbon atoms, 12 to 24 carbon atoms, 12 to 22 carbon atoms, 12 to 20 carbon atoms, or 12 to 18 carbon atoms).

In some embodiments, the sarcosinylated lipid has the structure of the following general formula (IXX):

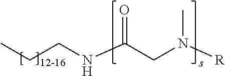

wherein R is H, a hydrophilic group or a functional group optionally comprising a targeting moiety; and s is the number of sarcosine units.

In some embodiments, the sarcosinylated lipid has the structure of the following formula (IXX-1):

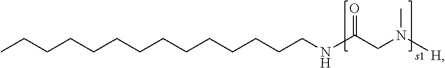

wherein s1 is 23. The sarcosinylated lipid of formula (IXX-1) is also referred to herein as “C14pSar23”.
Oxazolinylated and/or Oxazinylated Lipids

In some embodiments, the nucleic acid compositions (such as DNA or RNA compositions, especially mRNA compositions) described herein comprise a cationic/cationically ionizable lipid as described herein and an oxazolinylated and/or oxazinylated lipid (POX and/or POZ-conjugated lipid). The term “oxazolinylated lipid” or “POX-lipid” refers to a molecule comprising both a lipid portion and a polyoxazoline portion. The term “oxazinylated lipid” or “POZ-lipid” refers to a molecule comprising both a lipid portion and a polyoxazine portion. The term “oxazolinylated/oxazinylated lipid” or “POX/POZ-lipid” or “POXZ-lipid” refers to a molecule comprising both a lipid portion and a portion of a copolymer of polyoxazoline and polyoxazine.

In some embodiments, the nucleic acid compositions (such as DNA or RNA compositions, especially mRNA compositions) described herein may further comprise a neutral lipid (e.g., a phospholipid, cholesterol or a derivative thereof) or a combination of neutral lipids (e.g., a phospholipid, and cholesterol or a derivative thereof). In some embodiments, the nucleic acid compositions (such as DNA or RNA compositions, especially mRNA compositions) described herein comprise a cationic/cationically ionizable lipid as described herein, an oxazolinylated and/or oxazinylated lipid, a neutral lipid (e.g., a phospholipid), and cholesterol or a derivative thereof. In some embodiments, the phospholipid is selected from the group consisting of DSPC, DOPC, and DOPE. In some embodiments, the cationic/cationically ionizable lipid is a cationically ionizable lipid, such as that of formula (X), e.g. a cationically ionizable lipid of formula (X-3) or (X-45). In some embodiments, the cationic/cationically ionizable lipid is a cationically ionizable lipid of formula (XI) (such as a cationically ionizable lipid of formula (XIV-1), (XIV-2), or (XIV-3)), or that of formula (TL-J) (such as a cationically ionizable lipid of formula BNT-51, BNT-52, or BNT-76), or that of formula (AXL-J) (such as a cationically ionizable lipid of formula BHD-C2C2-PipZ, BODD-C2C2-1Me-Pyr, BODD-C2C2-Pyr, BODD-C2C1-1Me-3PipD, BODD-C2C1-1Me-PipD, BODD-C2C2-DMA, BODD-C2C4-PipZ, BODD-C2C4-Pyr, or BHD-C2C4-PipZ). In some embodiments, the cationic/cationically ionizable lipid is DPL14, EA-2, or 3D-P-DMA. In some embodiments, the cationic/cationically ionizable lipid comprises [(4-hydroxybutyl)azanediyl]di(hexane-6,1-diyl)bis(2-hexyldecanoate) (ALC-315); 1,2-dioleoyloxy-3-dimethylaminopropane (DODMA); 2,2-dilinoleyl-4-dimethylaminoethyl-[1,3]-dioxolane (DLin-KC2-DMA); heptatriaconta-6,9,28,31-tetraen-19-yl-4-(dimethylamino)butanoate (D-Lin-MC3-DMA); 1,2-dilinoleyloxy-N,N-dimethylaminopropane (DLin-DMA); di((Z)-non-2-en-1-yl)-9-((4-(dimethylaminobutanoyl)oxy)heptadecanedioate (L319); bis-(2-butyloctyl) 10-(N-(3-(dimethylamino)propyl)nonanamido)-nonadecanedioate (A9); (heptadecan-9-yl 8-{(2-hydroxyethyl)[6-oxo-6-(undecyloxy)octyl]amino}-octanoate) (L5); heptadecan-9-yl 8-{(2-hydroxyethyl)[6-oxo-6-(undecyloxy)hexyl]amino}-octanoate) (SM-102); O-[N-{(9Z,12Z)-octadeca-9,12-dien-1-yl)}-N-{7-pentadecylcarbonyloxyoctyl}-amino]4-(dimethylamino)butanoate (HY501); 2-(di-((9Z,12Z)-octadeca-9,12-dien-1-yl)amino)ethyl 4-(dimethylamino)butanoate (EA-2); 4-((di-((9Z,12Z)-octadeca-9,12-dien-1-yl)amino)oxy)-N,N-dimethyl-4-oxobutan-4-amine (HYAM-2); ((2-(4-(dimethylamino)butanoyl)oxy)ethyl)azanediylbis(octane 8,1-diyl)bis(2-hexyldecanoate) (EA-405); (2-(4-(dimethylamino)butanoyl)oxy)azanediylbis(octane 8,1-diyl)bis(2-hexyldecanoate) (HY-405); palmitoyl-oleoyl-nor-arginine (PONA); guanidino-di[(heptadecyl)methyl]carboxylic acid (GUADACA); 4-methylpyridinium-di(heptadecyl)methylcarboxylic acid (MPDACA); 1,2-dioleoyl-3 trimethylammonium propane (DOTAP); 1,2-dioleoyl-3-dimethylammomium propane (DODAP); or 1,2-di-O-octadecenyl-3-trimethylammonium propane (DOTMA); or a mixture of any thereof.

In some embodiments of the nucleic acid compositions (such as DNA or RNA compositions, especially mRNA compositions) described herein which comprise an oxazolinylated and/or oxazinylated lipid, said compositions are substantially free of a pegylated lipid having at least 30 consecutive ethylene glycol repeating units (or do not contain a pegylated lipid having at least 30 consecutive ethylene glycol repeating units).

In some embodiments, the POX and/or POZ polymer in the oxazolinylated and/or oxazinylated lipid comprises the following general formula (XX):

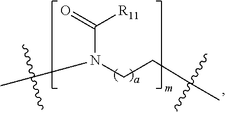

wherein a is an integer between 1 and 2; R₁₁ is alkyl, in particular C₁₋₃ alkyl, such as methyl, ethyl, iso-propyl, or n-propyl, and is independently selected for each repeating unit; and m refers to the number of POX and/or POZ repeating units.

In some embodiments of the oxazolinylated and/or oxazinylated lipid, the POX and/or POZ polymer is a polymer of POX and comprises repeating units of the following general formula (XXa):

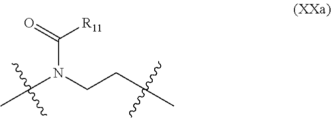

In some embodiments of the oxazolinylated and/or oxazinylated lipid, the POX and/or POZ polymer is a polymer of POZ and comprises repeating units of the following general formula (XXb):

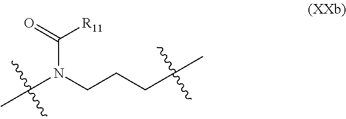

In any of the above embodiments of formulas (XX), (XXa), and (XXb), m (i.e., the number of repeating units of formula (XXa) or formula (XXb) in the polymer) preferably is between 2 and 190, such as between 2 and 180, between 2 and 170, between 2 and 160, between 2 and 150, between 2 and 140, between 2 and 130, between 2 and 120, between 2 and 110, between 2 and 100, between 2 and 90, between 2 and 80, between 2 and 70, between 5 and 200, between 5 and 190, between 5 and 180, between 5 and 170, between 5 and 160, between 5 and 150, between 5 and 140, between 5 and 130, between 5 and 120, between 5 and 110, between 5 and 100, between 5 and 90, between 5 and 80, between 5 and 70, between 10 and 200, between 10 and 190, between 10 and 180, between 10 and 170, between 10 and 160, between 10 and 150, between 10 and 140, between 10 and 130, between 10 and 120, between 10 and 110, between 10 and 100, between 10 and 90, between 10 and 80, or between 10 and 70. In certain embodiments of any of the above embodiments of formulas (XX), (XXa), and (XXb), m is 2 to 180, such as 4 to 160, 6 to 140, 8 to 120 or 10 to 100, e.g., 20 to 80, 30 to 70, or 40 to 50. In some embodiments of any of the above embodiments of formulas (XX), (XXa), and (XXb), m is 20 to 25. In some embodiments of any of the above embodiments of formulas (XX), (XXa), and (XXb), m is 45 to 50.

In some embodiments of the oxazolinylated and/or oxazinylated lipid, the POX and/or POZ polymer is a copolymer comprising repeating units of the following general formulas (XXa) and (XXb):

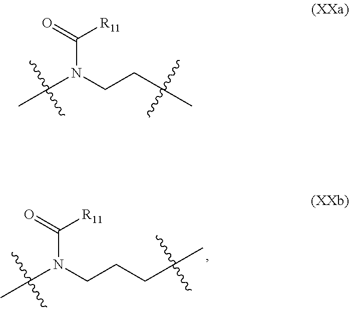

wherein the number of repeating units of formula (XXa) m the copolymer is to 199; the number of repeating units of formula (XXb) in the copolymer is 1 to 199; and the sum of the number of repeating units of formula (XXa) and the number of repeating units of formula (XXb) in the copolymer is 2 to 200.

In some embodiments of the oxazolinylated and/or oxazinylated lipid, the number of repeating units of formula (XXa) in the copolymer is 1 to 179, such as 1 to 159, 1 to 139, 1 to 119 or 1 to 99; the number of repeating units of formula (XXb) in the copolymer is 1 to 179, such as 1 to 159, 1 to 139, 1 to 119 or 1 to 99; and the sum of the number of repeating units of formula (XXa) and the number of repeating units of formula (XXb) in the copolymer is 2 to 180, such as 4 to 160, 6 to 140, 8 to 120 or 10 to 100, e.g., 20 to 80, 30 to 70, or 40 to 50.

In some of the above embodiments of formulas (XX), (XXa), and (XXb), R₁₁ at each occurrence (i.e., in each repeating unit) may be the same alkyl group (e.g., R₁₁ may be methyl in each repeating unit). In some alternative embodiments of formulas (XX), (XXa), and (XXb), R₁₁ in at least one repeating unit differs from R₁₁ in another repeating unit (e.g., for at least one repeating unit R₁₁ is one specific alkyl (such as ethyl), and for at least one different repeating unit R₁₁ is a different specific alkyl (such as methyl)). For example, each R₁₁ may be selected from two different alkyl groups (such as methyl and ethyl) and not all R₁₁ are the same alkyl.

In any of the above embodiments of formulas (XX), (XXa), and (XXb), R₁₁ preferably is methyl or ethyl, more preferably methyl. Thus, in some embodiments of formulas (XX), (XXa), and (XXb), each R₁₁ is methyl or each R₁₁ is ethyl. In some alternative embodiments of formulas (XX), (XXa), and (XXb), R₁₁ is independently selected from methyl and ethyl for each repeating unit, wherein in at least one repeating unit R₁₁ is methyl, and in at least one repeating unit R₁₁ is ethyl.

In some embodiments, the oxazolinylated and/or oxazinylated lipid has one of the following structures:

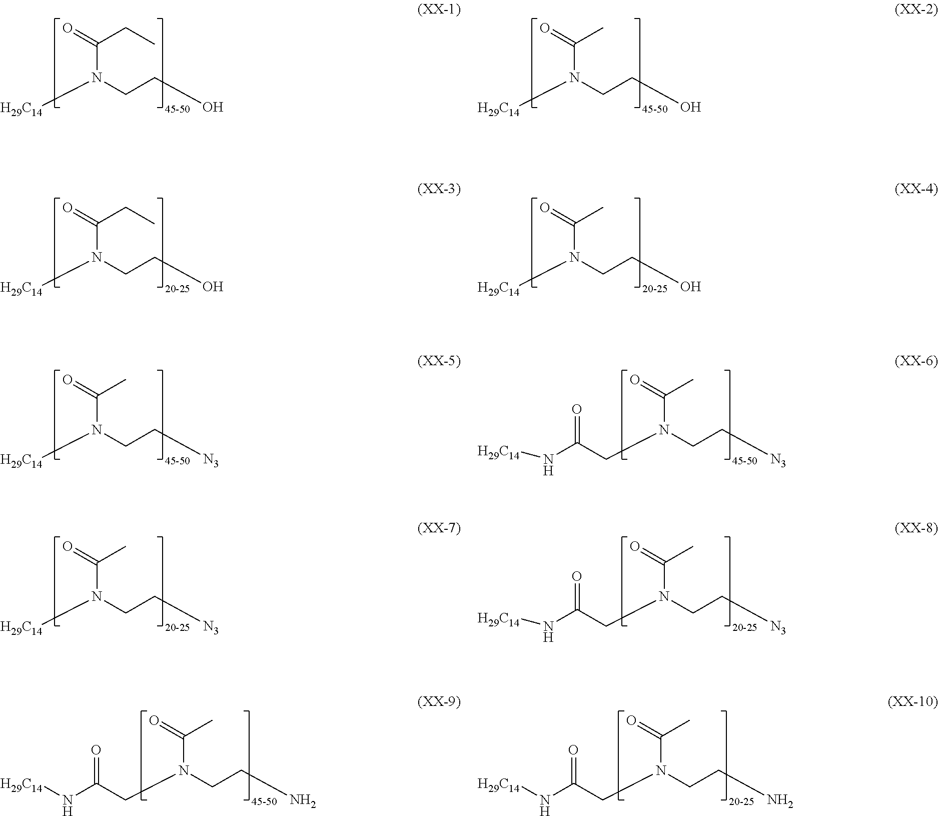

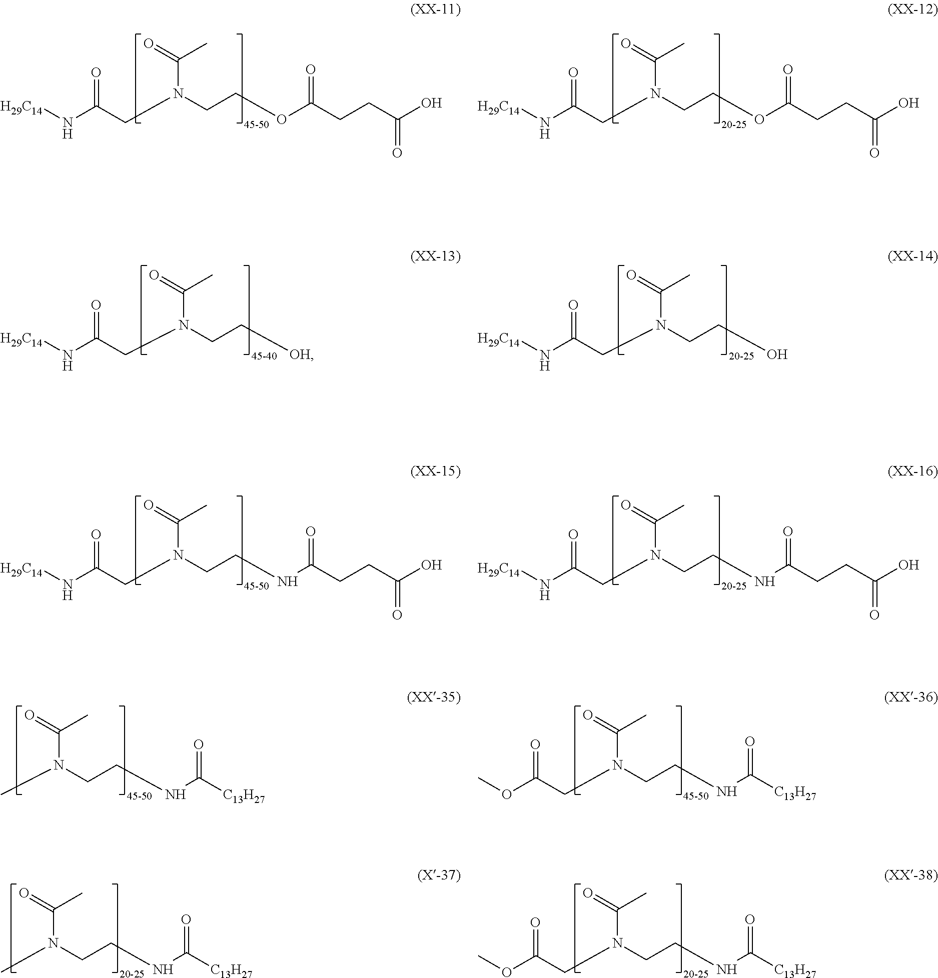

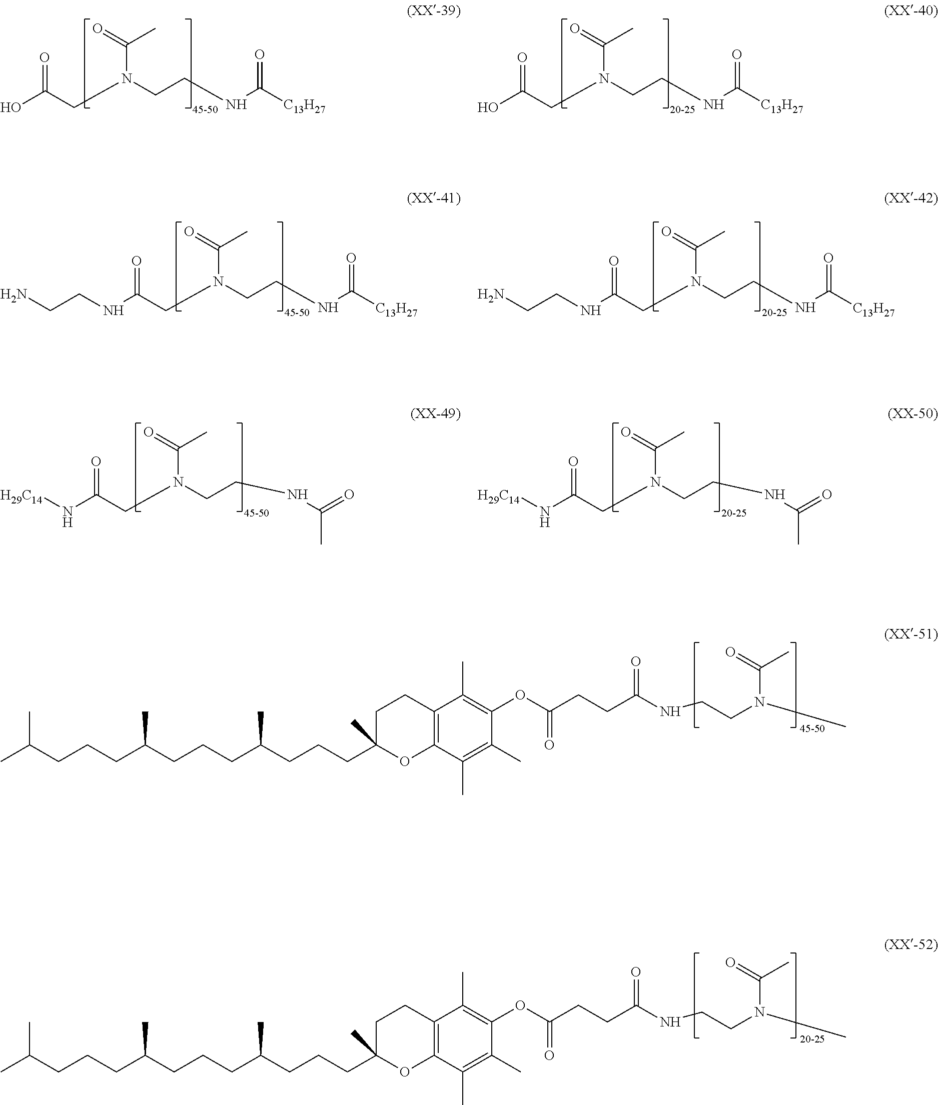

wherein in each case C₁₄H₂₉ refers to the moiety —(CH₂)₁₃CH₃, and in each case C₁₃H₂₇ refers to the moiety —(CH₂)₁₂CH₃.
Poly(vinyl pyrrolidone) (PVP)-conjugated lipids

In some embodiments, the nucleic acid compositions (such as DNA or RNA compositions, especially mRNA compositions) described herein comprise a cationic/cationically ionizable lipid as described herein and a poly(vinyl pyrrolidone) (PVP)-conjugated lipid. The term “poly(vinyl pyrrolidone)” or “PVP” means a polymer having a vinyl pyrrolidine repeating unit, i.e. the repeating unit shown below.

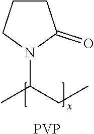

wherein x refers to the number of repeating units.

In some embodiments, the nucleic acid compositions (such as DNA or RNA compositions, especially mRNA compositions) described herein may further comprise a neutral lipid (e.g., a phospholipid, cholesterol or a derivative thereof) or a combination of neutral lipids (e.g., a phospholipid, and cholesterol or a derivative thereof). In some embodiments, the nucleic acid compositions (such as DNA or RNA compositions, especially mRNA compositions) described herein comprise a cationic/cationically ionizable lipid as described herein, a PVP-conjugated lipid, a neutral lipid (e.g., a phospholipid), and cholesterol or a derivative thereof. In some embodiments, the phospholipid is selected from the group consisting of DSPC, DOPC, and DOPE. In some embodiments, the cationic/cationically ionizable lipid is a cationically ionizable lipid, such as that of formula (X), e.g. a cationically ionizable lipid of formula (X-3) or (X-45). In some embodiments, the cationic/cationically ionizable lipid is a cationically ionizable lipid of formula (XI) (such as a cationically ionizable lipid of formula (XIV-1), (XIV-2), or (XIV-3)), or that of formula (TL-J) (such as a cationically ionizable lipid of formula BNT-51, BNT-52, or BNT-76), or that of formula (AXL-J) (such as a cationically ionizable lipid of formula BHD-C2C2-PipZ, BODD-C2C2-1Me-Pyr, BODD-C2C2-Pyr, BODD-C2C1-1Me-3PipD, BODD-C2C1-1Me-PipD, BODD-C2C2-DMA, BODD-C2C4-PipZ, BODD-C2C4-Pyr, or BHD-C2C4-PipZ). In some embodiments, the cationic/cationically ionizable lipid is DPL14, EA-2, or 3D-P-DMA. In some embodiments, the cationic/cationically ionizable lipid comprises [(4-hydroxybutyl)azanediyl]di(hexane-6,1-diyl)bis(2-hexyldecanoate) (ALC-315); 1,2-dioleoyloxy-3-dimethylaminopropane (DODMA); 2,2-dilinoleyl-4-dimethylaminoethyl-[1,3]-dioxolane (DLin-KC2-DMA); heptatriaconta-6,9,28,31-tetraen-19-yl-4-(dimethylamino)butanoate (D-Lin-MC3-DMA); 1,2-dilinoleyloxy-N,N-dimethylaminopropane (DLin-DMA); di((Z)-non-2-en-1-yl)-9-((4-(dimethylaminobutanoyl)oxy)heptadecanedioate (L319); bis-(2-butyloctyl) 10-(N-(3-(dimethylamino)propyl)nonanamido)-nonadecanedioate (A9); (heptadecan-9-yl 8-{(2-hydroxyethyl)[6-oxo-6-(undecyloxy)octyl]amino}-octanoate) (L5); heptadecan-9-yl 8-{(2-hydroxyethyl)[6-oxo-6-(undecyloxy)hexyl]amino}-octanoate) (SM-102); O-[N-{(9Z,12Z)-octadeca-9,12-dien-1-yl)}-N-{7-pentadecylcarbonyloxyoctyl}-amino]4-(dimethylamino)butanoate (HY501); 2-(di-((9Z,12Z)-octadeca-9,12-dien-1-yl)amino)ethyl 4-(dimethylamino)butanoate (EA-2); 4-((di-((9Z,12Z)-octadeca-9,12-dien-1-yl)amino)oxy)-N,N-dimethyl-4-oxobutan-4-amine (HYAM-2); ((2-(4-(dimethylamino)butanoyl)oxy)ethyl)azanediylbis(octane 8,1-diyl)bis(2-hexyldecanoate) (EA-405); (2-(4-(dimethylamino)butanoyl)oxy)azanediylbis(octane 8,1-diyl)bis(2-hexyldecanoate) (HY-405); palmitoyl-oleoyl-nor-arginine (PONA); guanidino-di[(heptadecyl)methyl]carboxylic acid (GUADACA); 4-methylpyridinium-di(heptadecyl)methylcarboxylic acid (MPDACA); 1,2-dioleoyl-3 trimethylammonium propane (DOTAP); 1,2-dioleoyl-3-dimethylammomium propane (DODAP); or 1,2-di-O-octadecenyl-3-trimethylammonium propane (DOTMA); or a mixture of any thereof.

In some embodiments of the nucleic acid compositions (such as DNA or RNA compositions, especially mRNA compositions) described herein which comprise a PVP-conjugated lipid, said compositions are substantially free of a pegylated lipid having at least 30 consecutive ethylene glycol repeating units (or do not contain a pegylated lipid having at least 30 consecutive ethylene glycol repeating units).

Poly(N-(2-Hydroxypropyl)Methacrylamide) (pHPMA)-Conjugated Lipids

In some embodiments, the nucleic acid compositions (such as DNA or RNA compositions, especially mRNA compositions) described herein comprise a cationic/cationically ionizable lipid as described herein and a poly(N-(2-hydroxypropyl)methacrylamide) (pHPMA)-conjugated lipid. The term “poly(N-(2-hydroxypropyl)-methacrylamide” or “pHPMA” means a polymer having the repeating unit shown below.

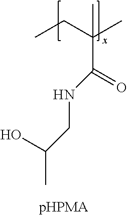

wherein x refers to the number of repeating units.

In some embodiments, the nucleic acid compositions (such as DNA or RNA compositions, especially mRNA compositions) described herein may further comprise a neutral lipid (e.g., a phospholipid, cholesterol or a derivative thereof) or a combination of neutral lipids (e.g., a phospholipid, and cholesterol or a derivative thereof). In some embodiments, the nucleic acid compositions (such as DNA or RNA compositions, especially mRNA compositions) described herein comprise a cationic/cationically ionizable lipid as described herein, a pHPMA-conjugated lipid, a neutral lipid (e.g., a phospholipid), and cholesterol or a derivative thereof. In some embodiments, the phospholipid is selected from the group consisting of DSPC, DOPC, and DOPE. In some embodiments, the cationic/cationically ionizable lipid is a cationically ionizable lipid, such as that of formula (X), e.g. a cationically ionizable lipid of formula (X-3) or (X-45). In some embodiments, the cationic/cationically ionizable lipid is a cationically ionizable lipid of formula (XI) (such as a cationically ionizable lipid of formula (XIV-1), (XIV-2), or (XIV-3)), or that of formula (TL-J) (such as a cationically ionizable lipid of formula BNT-51, BNT-52, or BNT-76), or that of formula (AXL-J) (such as a cationically ionizable lipid of formula BHD-C2C2-PipZ, BODD-C2C2-1Me-Pyr, BODD-C2C2-Pyr, BODD-C2C1-1Me-3PipD, BODD-C2C1-1Me-PipD, BODD-C2C2-DMA, BODD-C2C4-PipZ, BODD-C2C4-Pyr, or BHD-C2C4-PipZ). In some embodiments, the cationic/cationically ionizable lipid is DPL14, EA-2, or 3D-P-DMA. In some embodiments, the cationic/cationically ionizable lipid comprises [(4-hydroxybutyl)azanediyl]di(hexane-6,1-diyl)bis(2-hexyldecanoate) (ALC-315); 1,2-dioleoyloxy-3-dimethylaminopropane (DODMA); 2,2-dilinoleyl-4-dimethylaminoethyl-[1,3]-dioxolane (DLin-KC2-DMA); heptatriaconta-6,9,28,31-tetraen-19-yl-4-(dimethylamino)butanoate (D-Lin-MC3-DMA); 1,2-dilinoleyloxy-N,N-dimethylaminopropane (DLin-DMA); di((Z)-non-2-en-1-yl)-9-((4-(dimethylaminobutanoyl)oxy)heptadecanedioate (L319); bis-(2-butyloctyl) 10-(N-(3-(dimethylamino)propyl)nonanamido)-nonadecanedioate (A9); (heptadecan-9-yl 8-{(2-hydroxyethyl)[6-oxo-6-(undecyloxy)octyl]amino}-octanoate) (L5); heptadecan-9-yl 8-{(2-hydroxyethyl)[6-oxo-6-(undecyloxy)hexyl]amino}-octanoate) (SM-102); O-[N-{(9Z,12Z)-octadeca-9,12-dien-1-yl)}-N-{7-pentadecylcarbonyloxyoctyl}-amino]4-(dimethylamino)butanoate (HY501); 2-(di-((9Z,12Z)-octadeca-9,12-dien-1-yl)amino)ethyl 4-(dimethylamino)butanoate (EA-2); 4-((di-((9Z,12Z)-octadeca-9,12-dien-1-yl)amino)oxy)-N,N-dimethyl-4-oxobutan-4-amine (HYAM-2); ((2-(4-(dimethylamino)butanoyl)oxy)ethyl)azanediylbis(octane 8,1-diyl)bis(2-hexyldecanoate) (EA-405); (2-(4-(dimethylamino)butanoyl)oxy)azanediylbis(octane 8,1-diyl)bis(2-hexyldecanoate) (HY-405); palmitoyl-oleoyl-nor-arginine (PONA); guanidino-di[(heptadecyl)methyl]carboxylic acid (GUADACA); 4-methylpyridinium-di(heptadecyl)methylcarboxylic acid (MPDACA); 1,2-dioleoyl-3 trimethylammonium propane (DOTAP); 1,2-dioleoyl-3-dimethylammomium propane (DODAP); or 1,2-di-O-octadecenyl-3-trimethylammonium propane (DOTMA); or a mixture of any thereof.

In some embodiments of the nucleic acid compositions (such as DNA or RNA compositions, especially mRNA compositions) described herein which comprise a pHPMA-conjugated lipid, said compositions are substantially free of a pegylated lipid having at least 30 consecutive ethylene glycol repeating units (or do not contain a pegylated lipid having at least 30 consecutive ethylene glycol repeating units).

Poly(Dehydroalanine) (pDha)-Conjugated Lipids

In some embodiments, the nucleic acid compositions (such as DNA or RNA compositions, especially mRNA compositions) described herein comprise a cationic/cationically ionizable lipid as described herein and a poly(dehydroalanine) (pDha)-conjugated lipid. The term “pDha” means a polymer having the repeating unit shown below:

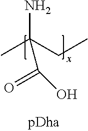

wherein x refers to the number of repeating units.

In some embodiments, the nucleic acid compositions (such as DNA or RNA compositions, especially mRNA compositions) described herein may further comprise a neutral lipid (e.g., a phospholipid, cholesterol or a derivative thereof) or a combination of neutral lipids (e.g., a phospholipid, and cholesterol or a derivative thereof). In some embodiments, the nucleic acid compositions (such as DNA or RNA compositions, especially mRNA compositions) described herein comprise a cationic/cationically ionizable lipid as described herein, a pDha-conjugated lipid, a neutral lipid (e.g., a phospholipid), and cholesterol or a derivative thereof. In some embodiments, the phospholipid is selected from the group consisting of DSPC, DOPC, and DOPE. In some embodiments, the cationic/cationically ionizable lipid is a cationically ionizable lipid, such as that of formula (X), e.g. a cationically ionizable lipid of formula (X-3) or (X-45). In some embodiments, the cationic/cationically ionizable lipid is a cationically ionizable lipid of formula (XI) (such as a cationically ionizable lipid of formula (XIV-1), (XIV-2), or (XIV-3)), or that of formula (TL-J) (such as a cationically ionizable lipid of formula BNT-51, BNT-52, or BNT-76), or that of formula (AXL-J) (such as a cationically ionizable lipid of formula BHD-C2C2-PipZ, BODD-C2C2-1Me-Pyr, BODD-C2C2-Pyr, BODD-C2C1-1Me-3PipD, BODD-C2C1-1Me-PipD, BODD-C2C2-DMA, BODD-C2C4-PipZ, BODD-C2C4-Pyr, or BHD-C2C4-PipZ). In some embodiments, the cationic/cationically ionizable lipid is DPL14, EA-2, or 3D-P-DMA. In some embodiments, the cationic/cationically ionizable lipid comprises [(4-hydroxybutyl)azanediyl]di(hexane-6,1-diyl)bis(2-hexyldecanoate) (ALC-315); 1,2-dioleoyloxy-3-dimethylaminopropane (DODMA); 2,2-dilinoleyl-4-dimethylaminoethyl-[1,3]-dioxolane (DLin-KC2-DMA); heptatriaconta-6,9,28,31-tetraen-19-yl-4-(dimethylamino)butanoate (D-Lin-MC3-DMA); 1,2-dilinoleyloxy-N,N-dimethylaminopropane (DLin-DMA); di((Z)-non-2-en-1-yl)-9-((4-(dimethylaminobutanoyl)oxy)heptadecanedioate (L319); bis-(2-butyloctyl) 10-(N-(3-(dimethylamino)propyl)nonanamido)-nonadecanedioate (A9); (heptadecan-9-yl 8-{(2-hydroxyethyl)[6-oxo-6-(undecyloxy)octyl]amino}-octanoate) (L5); heptadecan-9-yl 8-{(2-hydroxyethyl)[6-oxo-6-(undecyloxy)hexyl]amino}-octanoate) (SM-102); O-[N-{(9Z,12Z)-octadeca-9,12-dien-1-yl)}-N-{7-pentadecylcarbonyloxyoctyl}-amino]4-(dimethylamino)butanoate (HY501); 2-(di-((9Z,12Z)-octadeca-9,12-dien-1-yl)amino)ethyl 4-(dimethylamino)butanoate (EA-2); 4-((di-((9Z,12Z)-octadeca-9,12-dien-1-yl)amino)oxy)-N,N-dimethyl-4-oxobutan-4-amine (HYAM-2); ((2-(4-(dimethylamino)butanoyl)oxy)ethyl)azanediylbis(octane 8,1-diyl)bis(2-hexyldecanoate) (EA-405); (2-(4-(dimethylamino)butanoyl)oxy)azanediylbis(octane 8,1-diyl)bis(2-hexyldecanoate) (HY-405); palmitoyl-oleoyl-nor-arginine (PONA); guanidino-di[(heptadecyl)methyl]carboxylic acid (GUADACA); 4-methylpyridinium-di(heptadecyl)methylcarboxylic acid (MPDACA); 1,2-dioleoyl-3 trimethylammonium propane (DOTAP); 1,2-dioleoyl-3-dimethylammomium propane (DODAP); or 1,2-di-O-octadecenyl-3-trimethylammonium propane (DOTMA); or a mixture of any thereof.

In some embodiments of the nucleic acid compositions (such as DNA or RNA compositions, especially mRNA compositions) described herein which comprise a pDha-conjugated lipid, said compositions are substantially free of a pegylated lipid having at least 30 consecutive ethylene glycol repeating units (or do not contain a pegylated lipid having at least 30 consecutive ethylene glycol repeating units).

### Oligo Ethylene Glycol (OEG)-Conjugated Lipids

In some embodiments, the nucleic acid compositions (such as DNA or RNA compositions, especially mRNA compositions) described herein comprise a cationic/cationically ionizable lipid as described herein and an oligoethylene glycol (OEG)-conjugated lipid. The term “OEG-conjugated lipid” refers to a molecule comprising both a lipid portion and a portion of a polymer comprising oligoethylene glycol stretches separated by amide, optionally substituted thioamide or ester groups, wherein each of the oligoethylene glycol stretches comprises at most 24 consecutive ethylene glycol repeating units.

In some embodiments, the nucleic acid compositions (such as DNA or RNA compositions, especially mRNA compositions) described herein may further comprise a neutral lipid (e.g., a phospholipid, cholesterol or a derivative thereof) or a combination of neutral lipids (e.g., a phospholipid, and cholesterol or a derivative thereof). In some embodiments, the nucleic acid compositions (such as DNA or RNA compositions, especially mRNA compositions) described herein comprise a cationic/cationically ionizable lipid as described herein, an OEG-conjugated lipid, a neutral lipid (e.g., a phospholipid), and cholesterol or a derivative thereof. In some embodiments, the phospholipid is selected from the group consisting of DSPC, DOPC, and DOPE. In some embodiments, the cationic/cationically ionizable lipid is a cationically ionizable lipid, such as that of formula (X), e.g. a cationically ionizable lipid of formula (X-3) or (X-45). In some embodiments, the cationic/cationically ionizable lipid is a cationically ionizable lipid of formula (XI) (such as a cationically ionizable lipid of formula (XIV-1), (XIV-2), or (XIV-3)), or that of formula (TL-J) (such as a cationically ionizable lipid of formula BNT-51, BNT-52, or BNT-76), or that of formula (AXL-J) (such as a cationically ionizable lipid of formula BHD-C2C2-PipZ, BODD-C2C2-1Me-Pyr, BODD-C2C2-Pyr, BODD-C2C1-1Me-3PipD, BODD-C2C1-1Me-PipD, BODD-C2C2-DMA, BODD-C2C4-PipZ, BODD-C2C4-Pyr, or BHD-C2C4-PipZ). In some embodiments, the cationic/cationically ionizable lipid is DPL14, EA-2, or 3D-P-DMA. In some embodiments, the cationic/cationically ionizable lipid comprises [(4-hydroxybutyl)azanediyl]di(hexane-6,1-diyl)bis(2-hexyldecanoate) (ALC-315); 1,2-dioleoyloxy-3-dimethylaminopropane (DODMA); 2,2-dilinoleyl-4-dimethylaminoethyl-[1,3]-dioxolane (DLin-KC2-DMA); heptatriaconta-6,9,28,31-tetraen-19-yl-4-(dimethylamino)butanoate (D-Lin-MC3-DMA); 1,2-dilinoleyloxy-N,N-dimethylaminopropane (DLin-DMA); di((Z)-non-2-en-1-yl)-9-((4-(dimethylaminobutanoyl)oxy)heptadecanedioate (L319); bis-(2-butyloctyl) 10-(N-(3-(dimethylamino)propyl)nonanamido)-nonadecanedioate (A9); (heptadecan-9-yl 8-{(2-hydroxyethyl)[6-oxo-6-(undecyloxy)octyl]amino}-octanoate) (L5); heptadecan-9-yl 8-{(2-hydroxyethyl)[6-oxo-6-(undecyloxy)hexyl]amino}-octanoate) (SM-102); O-[N-{(9Z,12Z)-octadeca-9,12-dien-1-yl)}-N-{7-pentadecylcarbonyloxyoctyl}-amino]4-(dimethylamino)butanoate (HY501); 2-(di-((9Z,12Z)-octadeca-9,12-dien-1-yl)amino)ethyl 4-(dimethylamino)butanoate (EA-2); 4-((di-((9Z,12Z)-octadeca-9,12-dien-1-yl)amino)oxy)-N,N-dimethyl-4-oxobutan-4-amine (HYAM-2); ((2-(4-(dimethylamino)butanoyl)oxy)ethyl)azanediylbis(octane 8,1-diyl)bis(2-hexyldecanoate) (EA-405); (2-(4-(dimethylamino)butanoyl)oxy)azanediylbis(octane 8,1-diyl)bis(2-hexyldecanoate) (HY-405); palmitoyl-oleoyl-nor-arginine (PONA); guanidino-di[(heptadecyl)methyl]carboxylic acid (GUADACA); 4-methylpyridinium-di(heptadecyl)methylcarboxylic acid (MPDACA); 1,2-dioleoyl-3 trimethylammonium propane (DOTAP); 1,2-dioleoyl-3-dimethylammomium propane (DODAP); or 1,2-di-O-octadecenyl-3-trimethylammonium propane (DOTMA); or a mixture of any thereof.

In some embodiments of the nucleic acid compositions (such as DNA or RNA compositions, especially mRNA compositions) described herein which comprise an OEG-conjugated lipid, said compositions are substantially free of a pegylated lipid having at least 30 consecutive ethylene glycol repeating units (or do not contain a pegylated lipid having at least 30 consecutive ethylene glycol repeating units).

In some embodiments, the OEG-conjugated lipid is a polymer-conjugated compound comprising (a) a polymer which comprises the following general formula (XXI) and b) one or more hydrophobic chains:

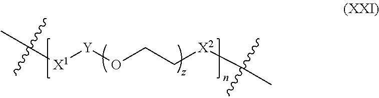

wherein
X² and X¹ taken together are optionally substituted amide, optionally substituted thioamide or ester;
Y is —CH₂—, —(CH₂)₂—, or —(CH₂)₃—;
z is 2 to 24; and
n is 1 to 100.

In some embodiments, the polymer in the OEG-conjugated lipid comprises the following general formula (XXII)

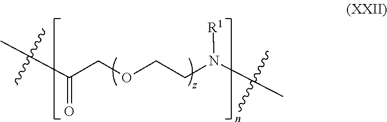

wherein R¹ is hydrogen or C₁₋₈alkyl. In some embodiments of formula (XXII), R¹ is hydrogen or methyl.

For example, R¹ may be hydrogen. In some embodiments, R¹ at each occurrence (i.e., in each repeating unit) is the same (e.g., R¹ may be H or methyl in each repeating unit). In some embodiments, R¹ in at least one repeating unit differs from R¹ in another repeating unit (e.g., for at least one repeating unit R¹ is one specific alkyl (such as H), and for at least one different repeating unit R¹ is a different specific alkyl (such as methyl)).

In some embodiments (in particular with respect to any one of formulas (XXI) and (XXII)), z is 2 to 20, such as 2 to 15, 2 to 10, or 2 to 7. In some embodiments, z is 2 to 7. In some embodiments, z is 2 to 5.

In some embodiments, z is 2 or 3. In some embodiments, z is 2.

In some embodiments, the polymer in the OEG-conjugated lipid comprises the following general formula (XXIII):

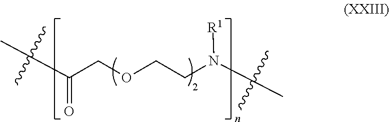

wherein R¹ is hydrogen or C₁₋₈ alkyl. In some embodiments of formula (XXIII), R¹ is hydrogen or methyl. For example, R¹ may be hydrogen. In some embodiments, R¹ at each occurrence (i.e., in each repeating unit) is the same (e.g., R¹ may be H or methyl in each repeating unit). In some embodiments, R¹ in at least one repeating unit differs from R¹ in another repeating unit (e.g., for at least one repeating unit R¹ is one specific alkyl (such as H), and for at least one different repeating unit R¹ is a different specific alkyl (such as methyl)).

In some embodiments, the polymer in the OEG-conjugated lipid comprises poly(aminoethyl-ethylene glycol acetyl) (pAEEA) having the following general formula (XXIV):

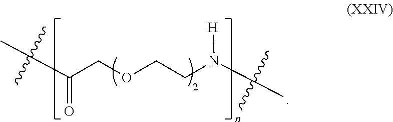

Thus, in these embodiments, the OEG-conjugated lipid comprises the structure of formula (XXIV) and may be designated as pAEEA-conjugated lipid.

In some embodiments, the polymer in the OEG-conjugated lipid comprises poly(methylaminoethyl-ethylene glycol acetyl) (pmAEEA) having the following general formula (XXIVa):

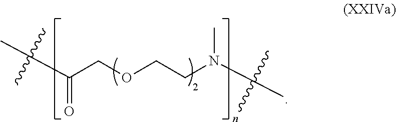

Thus, in these embodiments, the OEG-conjugated lipid comprises the structure of formula (XXIVa) and may be designated as pmAEEA-conjugated lipid.

In some embodiments (in particular with respect to any one of formulas (XXI), (XXII), (XXIII), (XXIV), and (XXIVa)), n is 5 to 50, such as 5 to 45, 5 to 40, 5 to 35 or 5 to 30. In some embodiments, n is 5 to 25, such as 6 to 20 or 6 to 15. In some embodiments, n is 7 to 14, preferably 8, 10, or 14. In some embodiments, n is 14. In some embodiments, n is 10. In some embodiments, n is 8.

In some embodiments, the OEG-conjugated lipid has one of the following structures:

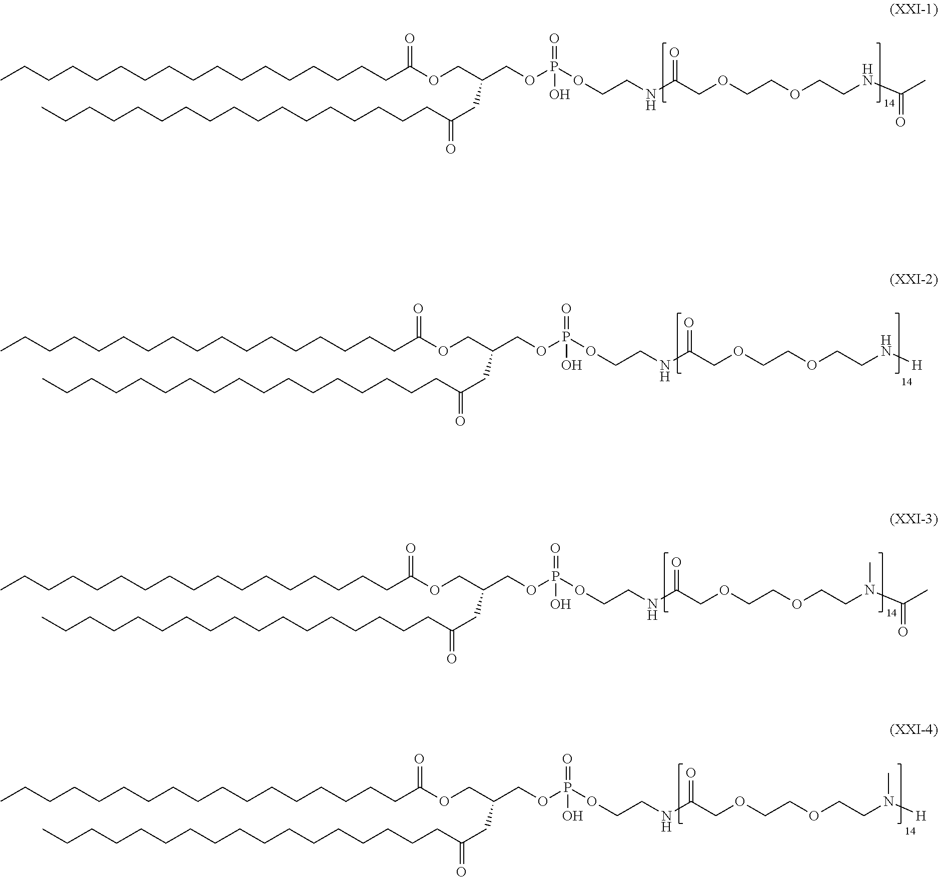

In some embodiments of the nucleic acid compositions (such as DNA or RNA compositions, especially mRNA compositions) described above, which comprise a polymer-conjugated lipid that is not a pegylated lipid, the polymer-conjugated lipid (such as preferably a sarcosinylated lipid or pAEEA-conjugated lipid) comprises from about 0.1 mol % to about 5 mol %, preferably from about 0.5 mol % to about 4.5 mol %, more preferably from about 1 mol % to about 4 mol %, such as from about 3 to about 4 mol %, of the total lipid present in the nucleic acid compositions (such as DNA or RNA compositions, especially mRNA compositions) described herein.

In some embodiments, the nucleic acid compositions (such as DNA or RNA compositions, especially mRNA compositions) described herein may comprise a cationic/cationically ionizable lipid as described herein (e.g., a cationically ionizable lipid, such as that of formula (X)), a polymer-conjugated lipid (such as preferably a sarcosinylated lipid or pAEEA-conjugated lipid), a phospholipid, and cholesterol, wherein the cationic/cationically ionizable lipid comprises from about 40 mol % to about 50 mol % of the total lipid present in the composition, the polymer-conjugated lipid (such as preferably a sarcosinylated lipid or pAEEA-conjugated lipid) comprises from about 1 mol % to about 4.5 mol % (such as from about 3 mol % to about 4 mol %) of the total lipid present in the composition, the phospholipid comprises from about 5 mol % to about 15 mol % of the total lipid present in the composition, and the cholesterol comprises from about 30 mol % to about 50 mol % of the total lipid present in the composition.

In some embodiments of the nucleic acid compositions (such as DNA or RNA compositions, especially mRNA compositions) described herein which comprise a polymer-conjugated lipid (such as preferably a sarcosinylated lipid or pAEEA-conjugated lipid as described herein), the N/P ratio is at least about 2. In some embodiments, the N/P value ranges from about 2 to about 20, such as from about 2 to about 12, from about 4 to about 12, from about 4 to about 10, from about 4 to about 8, or from about 5 to about 7. In some embodiments, the N/P ratio is about 6.

### Embodiments of Nucleic Acid (Such as RNA) Compositions

In some embodiments, the one or more additional lipids comprise one of the following components: (1) a neutral lipid; (2) a steroid; or (3) a mixture of a neutral lipid and a steroid; preferably each in the concentration given above. In some embodiments, the one or more additional lipids comprise (3) a mixture of a neutral lipid and a steroid (and preferably do not comprise a PEG-lipid having at least 30 consecutive ethylene glycol repeating units). In some embodiments, the one or more additional lipids comprise one of the following components: (1) a phospholipid; (2) cholesterol; (3) a mixture of a phospholipid and cholesterol; preferably each in the concentration given above. In some embodiments, the one or more additional lipids comprise (3) a mixture a phospholipid and cholesterol (and preferably do not comprise a PEG-lipid having at least 30 consecutive ethylene glycol repeating units). In some embodiments, the aqueous phase of the nucleic acid (such as RNA) compositions (especially the mRNA compositions) described herein further comprises a chelating agent, and/or the buffer system comprises histidine or HEPES.

Thus, in preferred embodiments, the nucleic acid (such as RNA) compositions (especially the mRNA compositions) described herein comprise (ii) a cationic or cationically ionizable lipid; (iii) a steroid; (iv) a neutral lipid (in particular, a phospholipid); (v) optionally a polymer-conjugated lipid (preferably not a PEG-lipid having at least 30 consecutive ethylene glycol repeating units); and (vi) a chelating agent, such as EDTA, preferably each in the concentration given above. In some embodiments, the steroid is cholesterol; and the neutral lipid is selected from the group consisting of DSPC, DOPC, and DOPE, preferably the neutral lipid is DOPE. In some embodiments, the buffer system comprises histidine or HEPES.

Thus, in some preferred embodiments, the nucleic acid (such as RNA) compositions (especially the mRNA compositions) described herein comprise (ii) a cationic or cationically ionizable lipid (preferably a cationically ionizable lipid); (iii) cholesterol; (iv) a phospholipid selected from the group consisting of DSPC, DOPC, and DOPE, preferably the neutral lipid is DOPE; (v) optionally a polymer-conjugated lipid (preferably not a PEG-lipid having at least 30 consecutive ethylene glycol repeating units; optionally a polysarcosine- or pAEEA-conjugated lipid); and (vi) histidine as part of the buffer system, preferably each in the concentration given above. Optionally the nucleic acid compositions may further comprise a chelating agent, such as EDTA.

Thus, in some other preferred embodiments, the nucleic acid (such as RNA) compositions (especially the mRNA compositions) described herein comprise (ii) a cationic or cationically ionizable lipid (preferably a cationically ionizable lipid); (iii) cholesterol; (iv) a phospholipid selected from the group consisting of DSPC, DOPC, and DOPE, preferably the neutral lipid is DOPE; (v) optionally a polymer-conjugated lipid (preferably not a PEG-lipid having at least 30 consecutive ethylene glycol repeating units; optionally a polysarcosine- or pAEEA-conjugated lipid); and (vi) HEPES as part of the buffer system, preferably each in the concentration given above. In some particularly preferred embodiments, these nucleic acid compositions comprising HEPES may further comprise a chelating agent, such as EDTA.

In each of the above preferred embodiments, the N/P ratio preferably is at least about 2. In some embodiments, the N/P value ranges from about 2 to about 20, such as from about 2 to about 12, from about 4 to about 12, from about 4 to about 10, from about 4 to about 8, or from about 5 to about 7. In some embodiments, the N/P ratio is about 6.

In each of the above preferred embodiments, the pH of the aqueous phase preferably is below 5.5 and/or above 4.0. In some embodiments, the pH of the aqueous phase is at least 4.0 and below 5.5, such as at least 4.1 and below 5.5, at least 4.2 and below 5.5, at least 4.3 and below 5.5, at least 4.4 and below 5.5, or at least 4.5 and below 5.5, such as at least 5.0 and below 5.5. In some embodiments, the pH of the aqueous phase is at least 4.0 and at most 5.4, such as at least 4.0 and at most 5.3, at least 4.0 and at most 5.2, at least 4.0 and at most 5.1, at least 4.0 and at most 5.0. In some embodiments, the pH of the aqueous phase is at least 4.1 and at most 5.4, at least 4.2 and at most 5.3, at least 4.3 and at most 5.2, at least 4.4 and at most 5.1, or at least 4.5 and at most 5.0. In some embodiments, the pH of the aqueous phase is at least 4.6 and at most 5.4, such as at least 4.7 and at most 5.3, at least 4.8 and at most 5.2, at least 4.9 and at most 5.1, e.g., about 5.0. The pH of the aqueous phase may be from about 4.0 to about 5.2, from about 4.5 to about 5.2, from about 4.8 to about 5.2, or from about 5.0 to about 5.2. In some embodiments, the pH of the aqueous phase is between about 4.0 and about 5.0, optionally between about 4.5 and 5.0. In some embodiments, the pH of the aqueous phase is at least 4.5 and below 5.5, optionally at least 4.5 and below 5.2. In some embodiments, the pH of the aqueous phase is at least 5.0 and below 5.5, e.g., at least 5.1 and below 5.5, at least 5.1 and at most 5.4, at least 5.2 and below 5.5, or at least 5.2 and at most 5.4.

In some embodiments, the pH of the aqueous phase is about 5.3.

In some embodiments, the nucleic acid (such as RNA) is present in the nucleic acid compositions (such as RNA compositions) described herein in a concentration from about 0.1 mg/l to about 500 mg/l, such as from about 0.5 mg/l to about 400 mg/l, from about 1 mg/l to about 300 mg/l, from about 2 mg/l to about 200 mg/l, from about 3 mg/l to about 150 mg/l, or from about 5 mg/l to about 100 mg/l.

Compositions Comprising Nucleic Acid (Such as RNA, Preferably mRNA) Particles

The nucleic acid (such as RNA) compositions described herein comprise nucleic acid particles (such as RNA particles, like RNA LNPs or RNA LPXs), preferably a plurality of nucleic acid particles (such as a plurality of RNA particles). The term “plurality of nucleic acid particles” or “plurality of nucleic acid-lipid particles” refers to a population of a certain number of particles. In certain embodiments, the term refers to a population of more than 10, 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, 10²¹, 10²², or 10²³ or more particles.

In certain embodiments, the composition comprises lipid nanoparticles (LNPs) (in particular, when the composition comprises a cationically ionizable lipid). In certain embodiments, the composition comprises lipoplexes (LPXs) (in particular, when the composition comprises a cationic lipid).

Nucleic acid particles (such as RNA particles, like LNPs comprising RNA (or “RNA LNPs”)) described herein have an average diameter that in some embodiments ranges from about 30 nm to about 1000 nm, from about 30 nm to about 800 nm, from about 30 nm to about 700 nm, from about 30 nm to about 600 nm, from about 30 nm to about 500 nm, from about 30 nm to about 450 nm, from about 30 nm to about 400 nm, from about 30 nm to about 350 nm, from about 30 nm to about 300 nm, from about 30 nm to about 250 nm, from about 30 nm to about 200 nm, from about 30 nm to about 190 nm, from about 30 nm to about 180 nm, from about 30 nm to about 170 nm, from about 30 nm to about 160 nm, from about 30 nm to about 150 nm, from about 50 nm to about 500 nm, from about 50 nm to about 450 nm, from about 50 nm to about 400 nm, from about 50 nm to about 350 nm, from about 50 nm to about 300 nm, from about 50 nm to about 250 nm, from about 50 nm to about 200 nm, from about 50 nm to about 190 nm, from about 50 nm to about 180 nm, from about 50 nm to about 170 nm, from about 50 nm to about 160 nm, or from about 50 nm to about 150 nm.

In certain embodiments, nucleic acid particles (such as RNA particles, like RNA LNPs) described herein have an average diameter that ranges from about 40 nm to about 800 nm, from about 50 nm to about 700 nm, from about 60 nm to about 600 nm, from about 70 nm to about 500 nm, from about 80 nm to about 400 nm, from about 150 nm to about 800 nm, from about 150 nm to about 700 nm, from about 150 nm to about 600 nm, from about 200 nm to about 600 nm, from about 200 nm to about 500 nm, or from about 200 nm to about 400 nm.

In certain embodiments, nucleic acid particles (such as RNA particles, like RNA LNPs) described herein may have an average diameter that in some embodiments ranges from about 30 nm to about 500 nm, such as from about 30 nm to about 200 nm, or from about 50 nm to about 150 nm.

In some embodiments, the nucleic acid (such as RNA) compositions described herein comprise particles with a size of at least 10 μm in an amount of less than 4000/ml, preferably at most 3500/ml, such as at most 3400/ml, at most 3300/ml, at most 3200/ml, at most 3100/ml, or at most 3000/ml.

It will be apparent to those of skill in the art that the plurality of particles can include any fraction of the foregoing ranges or any range therein.

Nucleic acid particles (such as RNA particles, like RNA LNPs) described herein, e.g. prepared by the methods described herein, preferably exhibit a polydispersity index less than about 0.5, less than about 0.4, less than about 0.3, less than about 0.2, less than about 0.1 or about 0.05 or less. By way of example, the nucleic acid particles (such as RNA LNPs) can exhibit a polydispersity index in a range of about 0.05 to about 0.2, such as about 0.05 to about 0.1.

Generally, the nucleic acid particles (such as RNA particles, like RNA LNPs) described herein are “nucleic acid-lipid particles” (such as “RNA-lipid particles”) that can be used to deliver the nucleic acid to a target site of interest (e.g., cell, tissue, organ, and the like). A nucleic acid-lipid particle is typically formed from a cationic or cationically ionizable lipid (such as the cationic or cationically ionizable lipids disclosed herein), one or more additional lipids (such as a phospholipid as disclosed herein (e.g., DSPC, DOPC, or DOPE, preferably DOPE), and a steroid (e.g., cholesterol or analogues thereof)), and optionally a polymer-conjugated lipid disclosed herein.

In some embodiments, the nucleic acid compositions described herein comprise (i) RNA LNPs (e.g., mRNA LNPs); (ii) a cationically ionizable lipid; and (iii) a mixture of a phospholipid (e.g., selected from the group consisting of DSPC, DOPC, and DOPE, preferably the phospholipid is DOPE) and a steroid (e.g., cholesterol); preferably each in the concentration given above. In some embodiments, the nucleic acid compositions do not comprise a PEG-lipid having at least 30 consecutive ethylene glycol repeating units. In some embodiments of these nucleic acid compositions, the buffer system comprised in the aqueous phase comprises histidine or HEPES and/or the aqueous phase further comprises a chelating agent.

Thus, in preferred embodiments, the nucleic acid compositions described herein comprise (i) RNA LNPs (e.g., mRNA LNPs); (ii) a cationically ionizable lipid; (iii) a steroid; (iv) a phospholipid; (v) optionally a polymer-conjugated lipid (preferably not a PEG-lipid having at least 30 consecutive ethylene glycol repeating units); and (vi) a chelating agent, such as EDTA, preferably each in the concentration given above. In some embodiments, the steroid is cholesterol; and the phospholipid lipid is selected from the group consisting of DSPC, DOPC, and DOPE, preferably the phospholipid is DOPE. In some embodiments of these nucleic acid compositions, the buffer system comprised in the aqueous phase comprises histidine or HEPES.

Thus, in some preferred embodiments, the nucleic acid compositions described herein comprise (i) RNA LNPs (e.g., mRNA LNPs); (ii) a cationically ionizable lipid; (iii) cholesterol; (iv) a phospholipid selected from the group consisting of DSPC, DOPC, and DOPE, preferably the phospholipid is DOPE; (v) optionally a polymer-conjugated lipid (preferably not a PEG-lipid having at least 30 consecutive ethylene glycol repeating units; optionally a polysarcosine- or pAEEA-conjugated lipid); and (vi) histidine as part of the buffer system, preferably each in the concentration given above. Optionally the nucleic acid compositions may further comprise a chelating agent, such as EDTA.

Thus, in some other preferred embodiments, the nucleic acid compositions described herein comprise (i) RNA LNPs (e.g., mRNA LNPs); (ii) a cationically ionizable lipid; (iii) cholesterol; (iv) a phospholipid selected from the group consisting of DSPC, DOPC, and DOPE, preferably the phospholipid is DOPE; (v) optionally a polymer-conjugated lipid (preferably not a PEG-lipid having at least 30 consecutive ethylene glycol repeating units; optionally a polysarcosine- or pAEEA-conjugated lipid); and (vi) HEPES as part of the buffer system, preferably each in the concentration given above.

In some particularly preferred embodiments, these nucleic acid compositions comprising HEPES may further comprise a chelating agent, such as EDTA.

In some embodiments, the nucleic acid compositions described herein comprise (i) RNA LPXs (e.g., mRNA LPXs); (ii) a cationic lipid; and (iii) a mixture of a neutral lipid (e.g., a phospholipid) and a steroid (e.g., cholesterol); preferably each in the concentration given above. In some embodiments, the neutral lipid is a phospholipid, preferably selected from the group consisting of DSPC, DOPC, and DOPE). In some embodiments, the LPXs do not comprise a PEG-lipid having at least 30 consecutive ethylene glycol repeating units. In some embodiments of these nucleic acid compositions, the buffer system comprised in the aqueous phase comprises histidine or HEPES and/or the aqueous phase further comprises a chelating agent.

Thus, in preferred embodiments, the nucleic acid compositions described herein comprise (i) RNA LPXs (e.g., mRNA LPXs); (ii) a cationic lipid; (iii) a steroid; (iv) a neutral lipid (e.g., a phospholipid); (v) optionally a polymer-conjugated lipid (preferably not a PEG-lipid having at least 30 consecutive ethylene glycol repeating units); and (vi) a chelating agent, such as EDTA, preferably each in the concentration given above. In some embodiments, the steroid is cholesterol; and the neutral lipid is a phospholipid, preferably selected from the group consisting of DSPC, DOPC, and DOPE. In some embodiments, the buffer system comprises histidine or HEPES.

Thus, in some preferred embodiments, the nucleic acid compositions described herein comprise (i) RNA LPXs (e.g., mRNA LPXs); (ii) a cationic lipid; (iii) cholesterol; (iv) a phospholipid (e.g., selected from the group consisting of DSPC, DOPC, and DOPE); (v) optionally a polymer-conjugated lipid (preferably not a PEG-lipid having at least 30 consecutive ethylene glycol repeating units; optionally a polysarcosine- or pAEEA-conjugated lipid); and (vi) histidine as part of the buffer system, preferably each in the concentration given above. Optionally the nucleic acid compositions may further comprise a chelating agent, such as EDTA.

Thus, in some other preferred embodiments, the nucleic acid compositions described herein comprise (i) RNA LPXs (e.g., mRNA LPXs); (ii) a cationic lipid; (iii) cholesterol; (iv) phospholipid (e.g., selected from the group consisting of DSPC, DOPC, and DOPE); (v) optionally a polymer-conjugated lipid (preferably not a PEG-lipid having at least 30 consecutive ethylene glycol repeating units; optionally a polysarcosine- or pAEEA-conjugated lipid); and (vi) HEPES as part of the buffer system, preferably each in the concentration given above. In some particularly preferred embodiments, these nucleic acid compositions comprising HEPES may further comprise a chelating agent, such as EDTA.

In each of the above preferred embodiments, the N/P ratio preferably is at least about 2. In some embodiments, the N/P value ranges from about 2 to about 20, such as from about 2 to about 12, from about 4 to about 12, from about 4 to about 10, from about 4 to about 8, or from about 5 to about 7. In some embodiments, the N/P ratio is about 6.

In each of the above preferred embodiments, the pH of the aqueous phase preferably is below 5.5 and/or above 4.0. In some embodiments, the pH of the aqueous phase is at least 4.0 and below 5.5, such as at least 4.1 and below 5.5, at least 4.2 and below 5.5, at least 4.3 and below 5.5, at least 4.4 and below 5.5, or at least 4.5 and below 5.5, such as at least 5.0 and below 5.5. In some embodiments, the pH of the aqueous phase is at least 4.0 and at most 5.4, such as at least 4.0 and at most 5.3, at least 4.0 and at most 5.2, at least 4.0 and at most 5.1, at least 4.0 and at most 5.0. In some embodiments, the pH of the aqueous phase is at least 4.1 and at most 5.4, at least 4.2 and at most 5.3, at least 4.3 and at most 5.2, at least 4.4 and at most 5.1, or at least 4.5 and at most 5.0. In some embodiments, the pH of the aqueous phase is at least 4.6 and at most 5.4, such as at least 4.7 and at most 5.3, at least 4.8 and at most 5.2, at least 4.9 and at most 5.1, e.g., about 5.0. The pH of the aqueous phase may be from about 4.0 to about 5.2, from about 4.5 to about 5.2, from about 4.8 to about 5.2, or from about 5.0 to about 5.2. In some embodiments, the pH of the aqueous phase is between about 4.0 and about 5.0, optionally between about 4.5 and 5.0. In some embodiments, the pH of the aqueous phase is at least 4.5 and below 5.5, optionally at least 4.5 and below 5.2. In some embodiments, the pH of the aqueous phase is at least 5.0 and below 5.5, e.g., at least 5.1 and below 5.5, at least 5.1 and at most 5.4, at least 5.2 and below 5.5, or at least 5.2 and at most 5.4.

In some embodiments, the pH of the aqueous phase is about 5.3.

In some embodiments, the nucleic acid (such as RNA) is present in the nucleic acid compositions (such as RNA compositions) described herein in a concentration from about 0.1 mg/l to about 500 mg/l, such as from about 0.5 mg/l to about 400 mg/l, from about 1 mg/l to about 300 mg/l, from about 2 mg/l to about 200 mg/l, from about 3 mg/l to about 150 mg/l, or from about 5 mg/l to about 100 mg/l.

Without intending to be bound by any theory, it is believed that the cationic or cationically ionizable lipid and the one or more additional lipids combine together with the nucleic acid (such as RNA) to form particles, which become particularly stable upon adjusting the pH (of the aqueous phase in which the particles are dispersed) to a range of about 4.0-5.5.

In some embodiments, nucleic acid-lipid particles (such as RNA-lipid particles) comprise more than one type of nucleic acid molecules (such as more than one type of RNA molecules), where the molecular parameters of the nucleic acid molecules may be similar or different from each other, like with respect to molar mass or fundamental structural elements such as molecular architecture, capping, coding regions or other features.

In some embodiments, the composition described herein is a liquid.

The present inventors have found that the nucleic acid in nucleic acid compositions which do not contain particles formed from the nucleic acid and lipids and whose pH is the range of about 4.0-5.5 are degraded, while, surprisingly, nucleic acid compositions which contain particles formed from the nucleic acid and lipids and whose pH is the range of about 4.0-5.5 are stable at a temperature range compliant to regular technologies in pharmaceutical practice, thereby providing a ready-to-use preparation.

Compositions described herein may also comprise a chelating agent. Chelating agents refer to chemical compounds that are capable of forming at least two coordinate covalent bonds with a metal ion, thereby generating a stable, water-soluble complex. Without wishing to be bound by theory, chelating agents reduce the concentration of free divalent ions, which may otherwise induce accelerated RNA degradation in the present disclosure. Examples of suitable chelating agents include, without limitation, ethylenediaminetetraacetic acid (EDTA), a salt of EDTA, desferrioxamine B, deferoxamine, dithiocarb sodium, penicillamine, pentetate calcium, a sodium salt of pentetic acid, succimer, trientine, nitrilotriacetic acid, trans-diaminocyclohexanetetraacetic acid (DCTA), diethylenetriaminepentaacetic acid (DTPA), and bis(aminoethyl)glycolether-N,N,N′,N′-tetraacetic acid. In certain embodiments, the chelating agent is EDTA or a salt of EDTA. In an exemplary embodiment, the chelating agent is EDTA disodium dihydrate. In some embodiments, the concentration of the chelating agent in the aqueous phase is between about 0.1 mM and about 20 mM, such as between about 0.2 mM and about 15 mM, between about 0.3 mM and about 12 mM, between about 0.4 mM and about 11 mM, or between about 0.5 mM and about 10 mM. In some embodiments, the concentration of the chelating agent in the aqueous phase is between about 0.5 mM and about 5 mM, such as between about 0.6 mM and about 4 mM, between about 0.7 mM and about 3.5 mM, between about 0.8 mM and about 3 mM, between about 0.9 mM and about 2.5 mM, or between about 1 mM and about 2 mM. In some embodiments, the concentration of the chelating agent in the aqueous phase is between about 0.5 mM and about 1.5 mM, such as about 1 mM.

In some embodiments, the aqueous phase of the nucleic acid (such as RNA) compositions (such as RNA LNP compositions) described herein do not comprise a chelating agent. For example, it is preferred that if the nucleic acid (such as RNA) compositions (such as RNA LNP compositions) described herein comprise a chelating agent, said chelating agent is only present in the particles, if present.

Compositions described herein may also comprise one or more tonicity agents. In some embodiments, the one or more tonicity agents are selected from the group consisting of salts and sugars. In some embodiments, the one or more tonicity agents are a salt, such as sodium chloride. In some embodiments, the one or more tonicity agents are a sugar, such as sucrose or glucose. In some embodiments, the concentration of the one or more tonicity agents in the aqueous phase is such that the composition is at most isotonic, in particular compared to human blood. For example, the compositions may comprise sodium chloride in a concentration of at least up to about 154 mM.

In some embodiments, the nucleic acid (such as RNA) compositions (such as RNA LNP compositions) described herein are substantially free of a cryoprotectant, for example they do not contain any cryoprotectant.

### Pharmaceutical Compositions

The nucleic acid (such as RNA) compositions described herein are useful as or for preparing pharmaceutical compositions or medicaments for therapeutic or prophylactic treatments.

The nucleic acid (such as RNA) compositions described herein may be administered in the form of any suitable pharmaceutical composition.

The term “pharmaceutical composition” relates to a composition comprising a therapeutically effective agent, preferably together with pharmaceutically acceptable carriers, diluents and/or excipients. Said pharmaceutical composition is useful for treating, preventing, or reducing the severity of a disease or disorder by administration of said pharmaceutical composition to a subject. In the context of the present disclosure, the pharmaceutical composition comprises RNA as described herein.

The pharmaceutical compositions of the present disclosure may comprise one or more adjuvants or may be administered with one or more adjuvants. The term “adjuvant” relates to a compound which prolongs, enhances or accelerates an immune response. Adjuvants comprise a heterogeneous group of compounds such as oil emulsions (e.g., Freund's adjuvants), mineral compounds (such as alum), bacterial products (such as Bordetella pertussis toxin), or immune-stimulating complexes. Examples of adjuvants include, without limitation, LPS, GP96, CpG oligodeoxynucleotides, growth factors, and cytokines, such as monokines, lymphokines, interleukins, chemokines. The chemokines may be IL-1, IL-2, IL-3, IL-4, IL-5, IL-6, IL-7, IL-8, IL-9, IL-10, IL-12, INFa, INF-γ, GM-CSF, LT-a. Further known adjuvants are aluminium hydroxide, Freund's adjuvant or oil such as Montanide® ISA51. Other suitable adjuvants for use in the present disclosure include lipopeptides, such as Pam3Cys, as well as lipophilic components, such as saponins, trehalose-6,6-dibehenate (TDB), monophosphoryl lipid-A (MPL), monomycoloyl glycerol (MMG), or glucopyranosyl lipid adjuvant (GLA).

The pharmaceutical compositions of the present disclosure may be in a “ready-to-use form” (i.e., in a form, in particular a liquid form, which can be immediately administered to a subject, e.g., without any processing such as thawing, reconstituting or diluting). The pharmaceutical compositions of the present disclosure are storable in a liquid form, which may also be a ready-to-use or administrable form. Ready to use injectables can be presented in containers such as vials, ampoules or syringes wherein the container may contain one or more doses.

In some embodiments, the pharmaceutical composition is in liquid form and can be stored at a temperature ranging from about 0° C. to about 20° C. For example, the liquid pharmaceutical compositions described herein can be stored at a temperature ranging from about 1° C. to about 15° C., such as from about 2° C. to about 10° C., or from about 2° C. to about 8° C., or at a temperature of about 5° C.

In some embodiments of the pharmaceutical compositions in liquid form, the pharmaceutical composition can be stored for at least 1 week, such as at least 2 weeks, at least 3 weeks, at least 4 weeks, at least 1 month, at least 2 months, at least 3 months, at least 6 months, at least 9 months, at least 12 months, or at least 24 months, preferably at least 4 weeks. For example, the liquid pharmaceutical composition can be stored for at least 4 weeks, preferably at least 1 month, more preferably at least 2 months, more preferably at least 3 months, more preferably at least 6 months at 5° C.

In some embodiments of the pharmaceutical compositions in liquid form, the nucleic acid (such as RNA) integrity after storing the liquid pharmaceutical composition is at least 90%, at least 95%, at least 97%, at least 98%, or substantially 100%, as compared to the nucleic acid (such as RNA) integrity as measured before storage (i.e., within 48 hours of initial preparation of the pharmaceutical composition) e.g., after storing the liquid composition for at least 1 week, such as at least 2 weeks, at least 3 weeks, at least 4 weeks, at least 1 month, at least 2 months, at least 3 months, at least 6 months, at least 9 months, at least 12 months, or at least 24 months, preferably at least 3 months) at about 5° C. The liquid pharmaceutical composition may have been stored for at least 4 weeks, preferably at least 1 month, more preferably at least 2 months, more preferably at least 3 months, more preferably at least 6 months at about 5° C. In some embodiments, the mRNA integrity after storing the liquid pharmaceutical composition at about 5° C. for at least 3 months, preferably at least 6 months, is at least 90%, preferably at least 95%, of the mRNA integrity as measured before storage (i.e., within 48 hours of initial preparation of the pharmaceutical composition). The % RNA integrity prior to storage may be at least about 50%, optionally at least about 60%, preferably at least about 70%, and the % RNA integrity after storage for the specified time period may then be at least 90%, preferably at least 95%, of said % RNA integrity value as measured before storage.

In some embodiments of the pharmaceutical composition in liquid form, the size (Z_{average}) (and/or size distribution and/or polydispersity index (PDI)) of the particles of the pharmaceutical composition, when stored, e.g., at 0° C. or higher for at least one week (preferably at least 1 month, more preferably at least 3 months), is such that the desired effect, e.g., to induce an immune response, can be achieved. For example, the size (Z_{average}) (and/or size distribution and/or polydispersity index (PDI)) of the particles of the pharmaceutical composition, when stored, e.g., at 0° C. or higher for at least one week (preferably at least 1 month, more preferably at least 3 months), is essentially equal to the size (Z_{average}) (and/or size distribution and/or PDI) of the particles of the initial pharmaceutical composition, i.e., before storage.

The pharmaceutical compositions according to the present disclosure are generally applied in a “pharmaceutically effective amount” and in “a pharmaceutically acceptable preparation”.

The term “pharmaceutically acceptable” refers to the non-toxicity of a material which does not interact with the action of the active component of the pharmaceutical composition.

The term “pharmaceutically effective amount” refers to the amount which achieves a desired reaction or a desired effect alone or together with further doses. In the case of the treatment of a particular disease, the desired reaction preferably relates to inhibition of the course of the disease. This comprises slowing down the progress of the disease and, in particular, interrupting or reversing the progress of the disease.

The desired reaction in a treatment of a disease may also be delay of the onset or a prevention of the onset of said disease or said condition. An effective amount of the particles or pharmaceutical compositions described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the particles or pharmaceutical compositions described herein may depend on various of such parameters. In the case that a reaction in a patient is insufficient with an initial dose, higher doses (or effectively higher doses achieved by a different, more localized route of administration) may be used.

In particular embodiments, a pharmaceutical composition of the present disclosure (e.g., an immunogenic composition, i.e., a pharmaceutical composition which can be used for inducing an immune response) is formulated as a single-dose in a container, e.g., a vial. In some embodiments, the immunogenic composition is formulated as a multi-dose formulation in a vial. In some embodiments, the multi-dose formulation includes at least 2 doses per vial. In some embodiments, the multi-dose formulation includes a total of 2-20 doses per vial, such as, for example, 2, 3, 4, 5, 6, 7, 8, 9, 10, 11, or 12 doses per vial. In some embodiments, each dose in the vial is equal in volume. In some embodiments, a first dose is a different volume than a subsequent dose.

A “stable” multi-dose formulation preferably exhibits no unacceptable levels of microbial growth, and substantially no or no breakdown or degradation of the active biological molecule component(s). As used herein, a “stable” immunogenic composition includes a formulation that remains capable of eliciting a desired immunologic response when administered to a subject.

The pharmaceutical compositions of the present disclosure may contain buffers (in particular, derived from the nucleic acid (such as RNA) compositions with which the pharmaceutical compositions have been prepared), preservatives, and optionally other therapeutic agents. In one embodiment, the pharmaceutical compositions of the present disclosure, in particular the ready-to-use pharmaceutical compositions, comprise one or more pharmaceutically acceptable carriers, diluents and/or excipients.

Suitable preservatives for use in the pharmaceutical compositions of the present disclosure include, without limitation, benzalkonium chloride, chlorobutanol, paraben and thimerosal.

The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants “Pharmaceutically acceptable salts” comprise, for example, acid addition salts which may, for example, be formed by using a pharmaceutically acceptable acid such as hydrochloric acid, acetic acid, lactic acid, 2-(N-morpholino)ethanesulfonic acid (MES), 3-(N-morpholino)propanesulfonic acid (MOPS), 2-[4-(2-hydroxyethyl)piperazin-1-yl]ethanesulfonic acid (HEPES) or benzoic acid. Furthermore, suitable pharmaceutically acceptable salts may include alkali metal salts (e.g., sodium or potassium salts); alkaline earth metal salts (e.g., calcium or magnesium salts); ammonium (NH₄); and salts formed with suitable organic ligands (e.g., quaternary ammonium and amine cations). Illustrative examples of pharmaceutically acceptable salts can be found in the prior art; see, for example, S. M. Berge et al., “Pharmaceutical Salts”, J. Pharm. Sci., 66, pp. 1-19 (1977)). Salts which are not pharmaceutically acceptable may be used for preparing pharmaceutically acceptable salts and are included in the present disclosure.

The term “diluent” relates a diluting and/or thinning agent. Moreover, the term “diluent” includes any one or more of fluid, liquid or solid suspension and/or mixing media. Examples of suitable diluents include ethanol and water.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carriers include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regard to the intended route of administration and standard pharmaceutical practice.

### Routes of Administration of Pharmaceutical Compositions

In one embodiment, the compositions described herein, such as the pharmaceutical compositions or ready-to-use pharmaceutical compositions described herein, may be administered intravenously, intraarterially, subcutaneously, intradermally, dermally, intranodally, intramuscularly or intratumorally.

In certain embodiments, the (pharmaceutical) composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In a preferred embodiment, the (pharmaceutical) compositions, in particular the ready-to-use pharmaceutical compositions, are formulated for systemic administration. In another preferred embodiment, the systemic administration is by intravenous administration. In another preferred embodiment, the (pharmaceutical) compositions, in particular the ready-to-use pharmaceutical compositions, are formulated for intramuscular administration.

### Use of Pharmaceutical Compositions

Nucleic acid (such as RNA) compositions described herein may be used in the therapeutic or prophylactic treatment of various diseases, in particular diseases in which provision of a peptide or protein to a subject results in a therapeutic or prophylactic effect. For example, provision of an antigen or epitope which is derived from a virus may be useful in the treatment or prevention of a viral disease caused by said virus. Provision of a tumor antigen or epitope may be useful in the treatment of a cancer disease wherein cancer cells express said tumor antigen. Provision of a functional protein or enzyme may be useful in the treatment of genetic disorder characterized by a dysfunctional protein, for example in lysosomal storage diseases (e.g. Mucopolysaccharidoses) or factor deficiencies. Provision of a cytokine or a cytokine-fusion may be useful to modulate tumor microenvironment. Nucleic acid (such as RNA) compositions described herein may be used in the therapeutic or prophylactic treatment of cancer.

The term “disease” (also referred to as “disorder” herein) refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality.

The term “infectious disease” refers to any disease which can be transmitted from individual to individual or from organism to organism, and is caused by a microbial agent. Infectious diseases are known in the art and include, for example, a viral disease, a bacterial disease, or a parasitic disease, which diseases are caused by a virus, a bacterium, and a parasite, respectively. In this regard, the infectious disease can be, for example, sexually transmitted diseases (e.g., chlamydia, gonorrhea, or syphilis), SARS, acquired immune deficiency syndrome (AIDS), measles, chicken pox, cytomegalovirus infections, genital herpes, hepatitis (such as hepatitis B or C), influenza (flu, such as human flu, swine flu, dog flu, horse flu, and avian flu), HPV infection, shingles, rabies, common cold, gastroenteritis, rubella, mumps, anthrax, cholera, diphtheria, foodborne illnesses, leprosy, meningitis, peptic ulcer disease, pneumonia, sepsis, septic shock, tetanus, tuberculosis, typhoid fever, urinary tract infection, Lyme disease, Rocky Mountain spotted fever, chlamydia, pertussis, tetanus, meningitis, scarlet fever, malaria, trypanosomiasis, Chagas disease, leishmaniasis, trichomoniasis, dientamoebiasis, giardiasis, amebic dysentery, coccidiosis, toxoplasmosis, sarcocystosis, rhinosporidiosis, and balantidiasis.

In some embodiments, nucleic acid (such as RNA) compositions described herein may be used in the therapeutic or prophylactic treatment of an infectious disease.

In the present context, the term “treatment”, “treating” or “therapeutic intervention” relates to the management and care of a subject for the purpose of combating a condition such as a disease or disorder.

The term is intended to include the full spectrum of treatments for a given condition from which the subject is suffering, such as administration of the therapeutically effective compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications.

The term “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably.

The terms “individual” and “subject” are used herein interchangeably. They refer to a human or another mammal (e.g., mouse, rat, rabbit, dog, cat, cattle, swine, sheep, horse or primate), or any other non-mammal-animal, including birds (chicken), fish or any other animal species that can be afflicted with or is susceptible to a disease or disorder (e.g., cancer, infectious diseases) but may or may not have the disease or disorder, or may have a need for prophylactic intervention such as vaccination, or may have a need for interventions such as by protein replacement. In many embodiments, the individual is a human being. Unless otherwise stated, the terms “individual” and “subject” do not denote a particular age, and thus encompass adults, elderlies, children, and newborns. In embodiments of the present disclosure, the “individual” or “subject” is a “patient”.

The term “patient” means an individual or subject for treatment, in particular a diseased individual or subject.

In some embodiments of the disclosure, the aim is to provide protection against an infectious disease by vaccination.

In some embodiments of the disclosure, the aim is to provide secreted therapeutic polypeptides, such as antibodies, bispecific antibodies, cytokines, cytokine fusion proteins, enzymes, to a subject, in particular a subject in need thereof.

In some embodiments of the disclosure, the aim is to provide a protein replacement therapy, such as production of erythropoietin, Factor VII, Von Willebrand factor, β-galactosidase, Alpha-N-acetylglucosaminidase, to a subject, in particular a subject in need thereof.

In some embodiments of the disclosure, the aim is to modulate/reprogram immune cells in the blood.

In some embodiments of the disclosure (in particular those relating to inhibitory RNA), the aim is to reduce or inhibit the expression of a peptide or polypeptide (such as the transcription and/or translation of a target mRNA). In some embodiments, the target mRNA comprises an ORF encoding a pharmaceutically active peptide or polypeptide, in particular a pharmaceutically active peptide or polypeptide whose expression (in particular increased expression, e.g., compared to the expression in a healthy subject) is associated with a disease. In some embodiments, the target mRNA comprises an ORF encoding a pharmaceutically active peptide or polypeptide whose expression (in particular increased expression, e.g., compared to the expression in a healthy subject) is associated with cancer.

In some embodiments, the nucleic acid (such as RNA) compositions described herein which contain nucleic acid (such as RNA) encoding a SARS-CoV-2 S protein, an immunogenic variant thereof, or an immunogenic fragment of the SARS-CoV-2 S protein or the immunogenic variant thereof (in the following simply “SARS-CoV-2 S nucleic acid compositions” which explicitly include SARS-CoV-2 S RNA compositions) following administration to a subject induce an antibody response, in particular a neutralizing antibody response, in the subject that targets a panel of different S protein variants such as SARS-CoV-2 S protein variants, in particular naturally occurring S protein variants. In some embodiments, the panel of different S protein variants comprises at least 5, at least 10, at least 15, or even more S protein variants. In some embodiments, such S protein variants comprise variants having amino acid modifications in the RBD domain and/or variants having amino acid modifications outside the RBD domain. In some embodiments, the SARS-CoV-2 S nucleic acid compositions described herein following administration to a subject induce an immune response (cellular and/or antibody response, in particular a neutralizing antibody response) in the subject that targets VOC-202012/01.

In some embodiments, the SARS-CoV-2 S nucleic acid compositions described herein following administration to a subject induce an immune response (cellular and/or antibody response, in particular a neutralizing antibody response) in the subject that targets 501.V2.

In some embodiments, the SARS-CoV-2 S nucleic acid compositions described herein following administration to a subject induce an immune response (cellular and/or antibody response, in particular a neutralizing antibody response) in the subject that targets “Cluster 5”.

In some embodiments, the SARS-CoV-2 S nucleic acid compositions described herein following administration to a subject induce an immune response (cellular and/or antibody response, in particular a neutralizing antibody response) in the subject that targets “B.1.1.28”.

In some embodiments, the SARS-CoV-2 S nucleic acid compositions described herein following administration to a subject induce an immune response (cellular and/or antibody response, in particular a neutralizing antibody response) in the subject that targets “B.1.1.248”.

In some embodiments, the SARS-CoV-2 S nucleic acid compositions described herein following administration to a subject induce an immune response (cellular and/or antibody response, in particular a neutralizing antibody response) in the subject that targets the Omicron (B.1.1.529) variant.

A person skilled in the art will know that one of the principles of immunotherapy and vaccination is based on the fact that an immunoprotective reaction to a disease is produced by immunizing a subject with an antigen or an epitope, which is immunologically relevant with respect to the disease to be treated.

Accordingly, pharmaceutical compositions described herein are applicable for inducing or enhancing an immune response. Pharmaceutical compositions described herein are thus useful in a prophylactic and/or therapeutic treatment of a disease involving an antigen or epitope.

The terms “immunization” or “vaccination” describe the process of administering an antigen to an individual with the purpose of inducing an immune response, for example, for therapeutic or prophylactic reasons.

Citation of documents and studies referenced herein is not intended as an admission that any of the foregoing is pertinent prior art. All statements as to the contents of these documents are based on the information available to the applicants and do not constitute any admission as to the correctness of the contents of these documents.

The description (including the following examples) is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### Itemized Embodiments

1. A composition comprising particles dispersed in an aqueous phase, wherein the aqueous phase comprises a buffer system and has a pH of about 4.0-5.5 and wherein the particles contain (i) nucleic acid; and (ii) a cationic or cationically ionizable lipid.
2. The composition of item 1, wherein the pH of the aqueous phase is below 5.5 and/or above 4.0, such as at least 4.1 and below 5.5, at least 4.2 and below 5.5, at least 4.3 and below 5.5, at least 4.4 and below 5.5, or at least 4.5 and below 5.5.
2a. The composition of item 1 or 2, wherein the pH of the aqueous phase is at least 5.0 and below 5.5, e.g., at least 5.1 and below 5.5, at least 5.1 and at most 5.4, at least 5.2 and below 5.5, or at least 5.2 and at most 5.4, or the pH of the aqueous phase is about 5.3.
3. The composition of item 1, 2, or 2a, wherein the concentration of the buffer system in the aqueous phase is between about 1 mM and about 50 mM, preferably between about 2 mM and about 40 mM, more preferably between about 3 mM and about 30 mM, more preferably between about 4 mM and about 25 mM, such as between about 5 mM and about 20 mM.
4. The composition of any one of items 1 to 3, wherein the buffer system comprises histidine.
5. The composition of any one of items 1 to 3, wherein the buffer system comprises HEPES.
6. The composition of any one of items 1 to 5, wherein the aqueous phase further comprises a chelating agent, such as EDTA.
6a. The composition of any one of items 1 to 3, wherein the buffer system comprises histidine and the aqueous phase further comprises a chelating agent, such as EDTA.
6b. The composition of any one of items 1 to 3, wherein the buffer system comprises HEPES and the aqueous phase further comprises a chelating agent, such as EDTA.
7. The composition of item 6, 6a, or 6b, wherein the concentration of the chelating agent in the aqueous phase is between about 0.1 mM and about 20 mM, such as between about 0.2 mM and about 15 mM, between about 0.3 mM and about 12 mM, between about 0.4 mM and about 11 mM, or between about 0.5 mM and about 10 mM.
7a. The composition of any one of items 1 to 3, wherein the buffer system comprises histidine and the aqueous phase is substantially free of EDTA, preferably substantially free of any chelating agent.
8. The composition of any one of items 1 to 7a, wherein the aqueous phase further comprises one or more tonicity agents, e.g., a salt, such as sodium chloride, and/or a sugar, such as sucrose or glucose.
9. The composition of item 8, wherein the concentration of the one or more tonicity agents in the aqueous phase is such that the composition is at most isotonic, in particular compared to human blood.
10. The composition of any one of items 1 to 9, wherein the particles have a size of from about 30 nm to about 500 n.
11. The composition of any one of items 1 to 10, wherein the particles are selected from lipid nanoparticles (LNPs), liposomes, lipoplexes (LPXs), and combinations of two or more thereof.
12. The composition of any one of items 1 to 11, wherein water is the main component in the composition and/or the total amount of solvent(s) other than water contained in the composition is less than about 0.5% (v/v).
13. The composition of any one of items 1 to 12, wherein the concentration of the nucleic acid in the composition is about 0.1 mg/l to about 500 mg/l, such as about 0.5 mg/l to about 400 mg/l, about 1 mg/l to about 300 mg/l, about 2 mg/l to about 200 mg/l, about 3 mg/l to about 150 mg/l, or about 5 mg/l to about 100 mg/l.
14. The composition of any one of items 1 to 13, wherein the N/P ratio is at least about 2, such as between about 2 and about 12, between about 4 and about 10, between about 4 and about 8, or between about 5 and about 7, e.g., about 6.
15. The composition of any one of items 1 to 14, wherein the cationically ionizable lipid comprises a head group which includes at least one nitrogen atom which is capable of being protonated under physiological conditions.
16. The composition of any one of items 1 to 15, wherein the cationically ionizable lipid has the structure of Formula (X)

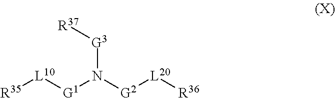

or a pharmaceutically acceptable salt, tautomer, or stereoisomer thereof, wherein:
   one of L¹⁰ and L²⁰ is —O(C═O)—, —(C═O)O—, —C(═O)—, —O—, —S(O)ₓ—, —S—S—, —C(═O)S—, SC(═O)—,
   —NRaC(═O)—, —C(═O)NR^{a}—, NRaC(═O)NR^{a}—, —OC(═O)NR^{a}— or —NRaC(═O)O—, and the other of L¹⁰ and
   L²⁰ is —O(C═O)—, —(C═O)O—, —C(═O)—, —O—, —S(O)ₓ—, —S—S—, —C(═O)S—, SC(═O)—, —NRaC(═O)—, —C(═O)NR^{a}—, NR^{a}C(═O)NR^{a}—, —OC(═O)NR^{a}— or —NRaC(═O)O— or a direct bond;
   G¹ and G² are each independently unsubstituted C₁-C₁₂ alkylene or C₂₋₁₂ alkenylene;
   G³ is C₁₋₂₄ alkylene, C₂₋₂₄ alkenylene, C₃₋₈ cycloalkylene, or C₃-s cycloalkenylene;
   R^{a} is H or C₁₋₁₂ alkyl;
   R³⁵ and R³⁶ are each independently C₆₋₂₄ alkyl or C₆₋₂₄ alkenyl;
   R³⁷ is H, OR⁵⁰, CN, —C(═O)OR⁴⁰, —OC(═O)R⁴⁰ or NR⁵⁰C(═O)R⁴⁰.
   R⁴⁰ is C₁₋₁₂ alkyl;
   R⁵⁰ is H or C₁₋₆ alkyl; and
   x is 0, 1 or 2.
17. The composition of any one of items 1 to 15, wherein the cationically ionizable lipid has the structure of Formula (XI):

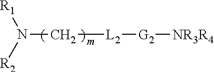

wherein
   each of R₁ and R₂ is independently R₅ or —G₁—L₁—R₆, wherein at least one of R₁ and R₂ is —G₁—L₁—R₆;
   each of R₃ and R₄ is independently selected from the group consisting of C₁₋₆ alkyl, C₂₋₆ alkenyl, aryl, and C₃₋₁₀ cycloalkyl;
   each of R₅ and R₆ is independently a non-cyclic hydrocarbyl group having at least 10 carbon atoms; each of G₁ and G₂ is independently unsubstituted C₁₋₁₂ alkylene or C₂₋₁₂ alkenylene;
   each of L₁ and L₂ is independently selected from the group consisting of —O(C═O)—, —(C═O)O—, —C(═O)—, —O—, —S(O)ₓ—, —S—S—, —C(═O)S—, —SC(═O)—, —NRaC(═O)—, —C(═O)NR^{a}—, —NRaC(═O)NR^{a}—, —OC(═O)NR^{a}— and —NRaC(═O)O—;
   Ra is H or C₁₋₁₂ alkyl;
   m is 0, 1, 2, 3, or 4; and
   x is 0, 1 or 2.
17a. The composition of any one of items 1 to 15, wherein the cationically ionizable lipid has the structure of Formula (TL-J):

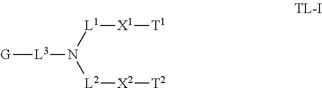

or a pharmaceutically acceptable salt thereof, wherein:
   each of L¹ and L² is independently an optionally substituted C₁-C₃₀ aliphatic group (e.g., an optionally substituted C₁-C₃₀ alkylene, C₂-C₃₀ alkenylene, C₂-C₃₀ alkynylene, or C₃-C₃₀ cycloalkylene group); L³ is a bond, optionally substituted C₁-C₁₀ aliphatic group (e.g., an optionally substituted C₁-C₁₀ alkylene, C₂-C₁₀ alkenylene, C₂-C₁₀ alkynylene, or C₃-C₁₀ cycloalkylene group), or optionally substituted 2- to 10-membered heteroaliphatic group (e.g., an optionally substituted 2- to 10-membered alkylene, alkenylene, alkynylene, or heterocycloalkylene group, wherein in each of the optionally substituted 2- to 10-membered alkylene, alkenylene, and alkynylene groups one or more (e.g., 1, 2, 3, or 4) carbon atoms are replaced by heteroatoms of O, N, Si, Se, P, or S, preferably O, S, or N); each of X¹ and X² is independently selected from a bond, —OC(O)—, —C(O)O—, —S(O)₂N(R¹)—, —N(R¹)S(O)₂—, —S(O)—, —S(O)₂—, —S(O)₂C(R¹)₂—, —OC(S)C(R¹)₂—, —C(R¹)₂C(S)O—, and —S—, wherein one or both of X¹ or X² is selected from —S(O)₂N(R¹)—, —N(R¹)S(O)₂, —S(O)—, —S(O)₂—, —S(O)₂C(R¹)₂—, —OC(S)C(R¹)₂—, —C(R¹)₂C(S)O—, and —S—;
   each R¹ is independently optionally substituted C₁-C₂₀ aliphatic group (e.g., an optionally substituted C₁-C₂₀ alkyl, C₂-C₂₀ alkenyl, C₂-C₂₀ alkynyl, or C₃-C₂₀ cycloalkyl group) or H;
   each of T¹ and T² is independently an optionally substituted C₃-C₃₀ aliphatic group (e.g., an optionally substituted C₃-C₃₀ alkyl, C₃-C₃₀ alkenyl, C₃-C₃₀ alkynyl, or C₃-C₃₀ cycloalkyl group); G is —N(R²)C(S)N(R²)₂, —N⁺(R³)₃, —OH, —N(R²)₂, —N(R⁵)C(O)R³, —N(R⁵)S(O)₂R₃, —N(R⁵)C(O)N(R³)₂, —CH(N—R²), or —R⁴;
   each R² is independently selected from the group consisting of H, optionally substituted C₁-C₆ aliphatic group (e.g., an optionally substituted C₁-C₆ alkyl, C₂-C₆ alkenyl, C₂-C₆ alkynyl, or C₃-C₆ cycloalkyl group) or OR³; or two instances of R² come together with the atoms to which they are attached to form an optionally substituted 4- to 12-membered heterocycle ring or an optionally substituted 4- to 12-membered heteroaryl ring;
   each R³ is independently selected from the group consisting of H and optionally substituted C₁-C₁₀ aliphatic group (e.g., an optionally substituted C₁-C₁₀ alkyl, C₂-C₁₀ alkenyl, C₂-C₁₀ alkynyl, or C₃-C₁₀ cycloalkyl group);
   R⁴ is optionally substituted 4- to 12-membered heterocycle, optionally substituted 4- to 12 membered heteroaryl, C₆-C₁₂ aryl substituted with one or more of —(CH₂)₀₋₆—OH or —(CH₂)₀₋₆—N(R⁵)₂, or C₃-C₁2 cycloaliphatic (e.g., C₃-C₁₂ alkyl, C₃-C₁₂ alkenyl, C₃-C₁₂ alkynyl, or C₃-C₁₂ cycloalkyl) substituted with one or more of oxo, —(CH₂)₀₋₆—OH, or —(CH₂)₀₋₆—N(R⁵)₂; and
   each R⁵ is independently selected from H and optionally substituted C₁-C₆ aliphatic group (e.g., an optionally substituted C₁-C₆ alkyl, C₂-C₆ alkenyl, C₂-C₆ alkynyl, or C₃-C₆ cycloalkyl group).
17b. The composition of any one of items 1 to 15, wherein the cationically ionizable lipid has the structure of Formula (AXL-I):

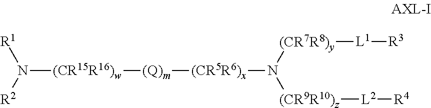

or a pharmaceutically acceptable salt thereof, wherein:
   each R¹ and R² is independently selected from the group consisting of H, an optionally substituted C₁₋₁₂alkyl, optionally substituted C₂₋₁₂ alkenyl, optionally substituted C₂₋₁₂ alkynyl, optionally substituted C₃₋₆ cycloalkyl, optionally substituted C₄₋₆ heterocycloalkyl, optionally substituted C₄₋₆ alkylcycloalkyl, optionally substituted C₄₋₆ aryl, optionally substituted C₃0.6 heteroaryl, optionally substituted C₄₋ₛ aryloxy, optionally substituted C₇₋₁₀ arylalkyl; optionally substituted C₅₋₁₀ heteroarylalkyl group, optionally substituted amine; or R¹ and R² can together form an optionally substituted cycloalkyl or heterocycloalkyl ring;
   each R³ and R⁴ is independently selected from the group consisting of optionally substituted C₁₋₂₂ alkyl, optionally substituted C₂₋₂₂ alkenyl, and optionally substituted C₂₋₂₂ alkynyl;
   each R⁵, R⁶, R⁷, R⁸, R⁹, R¹⁰, R¹¹, and R¹⁶ is independently selected from the group consisting of H, OH, halogen, phenyl, benzyl, optionally substituted C₁₋₂₂ alkyl, optionally substituted C₂₋₂₂ alkenyl, optionally substituted C₂₋₂₂ alkynyl;
   each of w, y, and z is independently an integer from 0-10;
   each Q is independently an atom selected from 0, NH, NR¹, NR¹NR¹, S, or a disulfide bond;
   m is 0 or 1; and
   each of L¹ and L² is independently selected from the group consisting of —C(═O)—; —OC(═O)—; —OC(═O)O—; —C(═O)O—; —C(═O)O(CR⁶R⁷)ₘ—; —NH—C(═O)—; —C(═O)NH—; —SO—; —SO₂—; —SO₃—; —NSO₂—; —SO₂N—; —NH(C₁₋₈ alkyl)-; —N(C₁₋₈ alkyl)₂—; —NH(C₆ aryl)-; —N(C₆ aryl)₂—; —NHC(═O)NH—; —NHC(═O)O—; —OC(═O)NH—; —NHC(═O)NR¹—; —NHC(═O)O—; —OC(═O)NR¹—; —C(═O)R¹—; —CO(C₁₋₈ alkyl)-; —CO(C₆ aryl)-; —CO₂(C₁₋₈alkyl)-; —CO₂(C₆ aryl)-; —SO₂(C₁₋₈ alkyl)-; and —SO₂(C₆ aryl)-.
18. The composition of any one of items 1 to 15, wherein the cationic or cationically ionizable lipid comprises:
   [(4-hydroxybutyl)azanediyl]di(hexane-6,1-diyl)bis(2-hexyldecanoate) (ALC-315); 1,2-dioleoyloxy-3-dimethylaminopropane (DODMA); 2,2-dilinoleyl-4-dimethylaminoethyl-[1,3]-dioxolane (DLin-KC2-DMA);
   heptatriaconta-6,9,28,31-tetraen-19-yl-4-(dimethylamino)butanoate (D-Lin-MC3-DMA); 1,2-dilinoleyloxy-N,N-dimethylaminopropane (DLin-DMA);
   di((Z)-non-2-en-1-yl)-9-((4-(dimethylaminobutanoyl)oxy)heptadecanedioate (L319); bis-(2-butyloctyl) 10-(N-(3-(dimethylamino)propyl)nonanamido)-nonadecanedioate (A9); (heptadecan-9-yl 8-{(2-hydroxyethyl)[6-oxo-6-(undecyloxy)octyl]amino}-octanoate) (L5); heptadecan-9-yl 8-{(2-hydroxyethyl)[6-oxo-6-(undecyloxy)hexyl]amino}-octanoate) (SM-102); O-[N-{(9Z,12Z)-octadeca-9,12-dien-1-yl)}-N-{7-pentadecylcarbonyloxyoctyl}-amino]4-(dimethylamino)butanoate (HY501);
   2-(di-((9Z,12Z)-octadeca-9,12-dien-1-yl)amino)ethyl 4-(dimethylamino)butanoate (EA-2); 4-((di-((9Z,12Z)-octadeca-9,12-dien-1-yl)amino)oxy)-N,N-dimethyl-4-oxobutan-4-amine (HYAM-2); ((2-(4-(dimethylamino)butanoyl)oxy)ethyl)azanediylbis(octane 8,1-diyl)bis(2-hexyldecanoate) (EA-405);
   (2-(4-(dimethylamino)butanoyl)oxy)azanediylbis(octane 8,1-diyl)bis(2-hexyldecanoate) (HY-405); palmitoyl-oleoyl-nor-arginine (PONA);
   guanidino-di[(heptadecyl)methyl]carboxylic acid (GUADACA);
   4-methylpyridinium-di(heptadecyl)methylcarboxylic acid (MPDACA);
   1,2-dioleoyl-3 trimethylammonium propane (DOTAP);
   1,2-dioleoyl-3-dimethylammomium propane (DODAP); or
   1,2-di-O-octadecenyl-3-trimethylammonium propane (DOTMA);
   or a mixture of any thereof.
18a. The composition of any one of items 1 to 15, wherein the cationic or cationically ionizable lipid comprises: 7,7′-((4-hydroxybutyl)azanediyl)bis(N-hexyl-N-octylheptane-1-sulfonamide) (BNT-51); 7,7′-((4-(3,3-dimethylthioureido)butyl)azanediyl)bis(N-hexyl-N-octylheptane-1-sulfonamide) (BNT-52); BNT-76; di(heptadecan-9-yl) 3,3′-((2-(4-methylpiperazin-1-yl)ethyl)azanediyl)dipropionate (BHD-C2C2-PipZ); bis(2-octyldodecyl) 3,3′-((2-(1-methylpyrrolidin-2-yl)ethyl)azanediyl)dipropionate (BODD-C2C2-1Me-Pyr); bis(2-octyldodecyl) 3,3′-((2-(pyrrolidin-1-yl)ethyl)azanediyl)dipropionate (BODD-C2C2-Pyr); bis(2-octyldodecyl) 3,3′-(((1-methylpiperidin-3-yl)methyl)azanediyl)dipropionate (BODD-C2C1-1Me-3PipD); bis(2-octyldodecyl) 3,3′-(((1-methylpiperidin-4-yl)methyl)azanediyl)dipropionate (BODD-C2C1-1Me-PipD); bis(2-octyldodecyl) 3,3′-((2-(dimethylamino)ethyl)azanediyl)dipropionate (BODD-C2C2-DMA); bis(2-octyldodecyl) 3,3′-((4-(4-methylpiperazin-1-yl)butyl)azanediyl)dipropionate (BODD-C2C4-PipZ); bis(2-octyldodecyl) 3,3′-((4-(pyrrolidin-1-yl)butyl)azanediyl)dipropionate (BODD-C2C4-Pyr); bis(2-hexyldecyl) 3,3′-((4-(4-methylpiperazin-1-yl)butyl)azanediyl)dipropionate (BHD-C2C4-PipZ); or a mixture of any thereof.
18b. The composition of any one of items 1 to 15, wherein the cationic or cationically ionizable lipid comprises: [(4-hydroxybutyl)azanediyl]di(hexane-6,1-diyl)bis(2-hexyldecanoate) (ALC-315); 1,2-dioleoyloxy-3-dimethylaminopropane (DODMA); 2,2-dilinoleyl-4-dimethylaminoethyl-[1,3]-dioxolane (DLin-KC2-DMA); heptatriaconta-6,9,28,31-tetraen-19-yl-4-(dimethylamino)butanoate (D-Lin-MC3-DMA); 1,2-dilinoleyloxy-N,N-dimethylaminopropane (DLin-DMA); di((Z)-non-2-en-1-yl)-9-((4-(dimethylaminobutanoyl)oxy)heptadecanedioate (L319); bis-(2-butyloctyl) 10-(N-(3-(dimethylamino)propyl)nonanamido)-nonadecanedioate (A9); (heptadecan-9-yl 8-{(2-hydroxyethyl)[6-oxo-6-(undecyloxy)octyl]amino}-octanoate) (L5); heptadecan-9-yl 8-{(2-hydroxyethyl)[6-oxo-6-(undecyloxy)hexyl]amino}-octanoate) (SM-102); O-[N-{(9Z,12Z)-octadeca-9,12-dien-1-yl)}-N-{7-pentadecylcarbonyloxyoctyl}-amino]4-(dimethylamino)butanoate (HY501); 2-(di-((9Z,12Z)-octadeca-9,12-dien-1-yl)amino)ethyl 4-(dimethylamino)butanoate (EA-2); 4-((di-((9Z,12Z)-octadeca-9,12-dien-1-yl)amino)oxy)-N,N-dimethyl-4-oxobutan-4-amine (HYAM-2); ((2-(4-(dimethylamino)butanoyl)oxy)ethyl)azanediylbis(octane 8,1-diyl)bis(2-hexyldecanoate) (EA-405); (2-(4-(dimethylamino)butanoyl)oxy)azanediylbis(octane 8,1-diyl)bis(2-hexyldecanoate) (HY-405); palmitoyl-oleoyl-nor-arginine (PONA); guanidino-di[(heptadecyl)methyl]carboxylic acid (GUADACA); 4-methylpyridinium-di(heptadecyl)methylcarboxylic acid (MPDACA); 1,2-dioleoyl-3 trimethylammonium propane (DOTAP); 1,2-dioleoyl-3-dimethylammomium propane (DODAP); 1,2-di-O-octadecenyl-3-trimethylammonium propane (DOTMA); 7,7′-((4-hydroxybutyl)azanediyl)bis(N-hexyl-N-octylheptane-1-sulfonamide) (BNT-51); 7,7′-((4-(3,3-dimethylthioureido)butyl)azanediyl)-bis(N-hexyl-N-octylheptane-1-sulfonamide) (BNT-52); BNT-76; di(heptadecan-9-yl) 3,3′-((2-(4-methylpiperazin-1-yl)ethyl)azanediyl)dipropionate (BHD-C2C2-PipZ); bis(2-octyldodecyl) 3,3′-((2-(1-methylpyrrolidin-2-yl)ethyl)azanediyl)dipropionate (BODD-C2C2-1Me-Pyr); bis(2-octyldodecyl) 3,3′-((2-(pyrrolidin-1-yl)ethyl)azanediyl)dipropionate (BODD-C2C2-Pyr); bis(2-octyldodecyl) 3,3′-(((1-methylpiperidin-3-yl)methyl)azanediyl)dipropionate (BODD-C2C1-1Me-3PipD); bis(2-octyldodecyl) 3,3′-(((1-methylpiperidin-4-yl)methyl)azanediyl)dipropionate (BODD-C2C1-1Me-PipD); bis(2-octyldodecyl) 3,3′-((2-(dimethylamino)ethyl)azanediyl)dipropionate (BODD-C2C2-DMA); bis(2-octyldodecyl) 3,3′-((4-(4-methylpiperazin-1-yl)butyl)azanediyl)dipropionate (BODD-C2C4-PipZ); bis(2-octyldodecyl) 3,3′-((4-(pyrrolidin-1-yl)butyl)azanediyl)dipropionate (BODD-C2C4-Pyr); bis(2-hexyldecyl) 3,3′-((4-(4-methylpiperazin-1-yl)butyl)azanediyl)dipropionate (BHD-C2C4-PipZ); or a mixture of any thereof.
19. The composition of any one of items 1 to 18, 18a, and 18b, wherein the cationic or cationically ionizable lipid comprises from about 20 mol % to about 80 mol %, preferably from about 25 mol % to about 65 mol %, more preferably from about 30 mol % to about 50 mol %, such as from about 40 mol % to about 50 mol %, of the total lipid present in the composition.
19a. The composition of any one of items 1 to 18, 18a, and 18b, wherein the particles contain a cationically ionizable lipid.
20. The composition of any one of items 1 to 19 and 19a, wherein the particles further comprise one or more additional lipids, preferably selected from the group consisting of polymer-conjugated lipids, neutral lipids, steroids, and combinations thereof.
21. The composition of item 20, wherein the polymer-conjugated lipid comprises a pegylated lipid, wherein the pegylated lipid preferably (i) is selected from the group consisting of DSPE-PEG, DOPE-PEG, DPPE-PEG, and DMPE-PEG; or (ii) has the following structure:

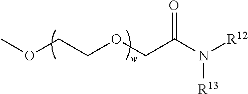

or a pharmaceutically acceptable salt, tautomer or stereoisomer thereof, wherein:
   R¹² and R¹³ are each independently a straight or branched, saturated or unsaturated alkyl chain containing from 10 to 30 carbon atoms, wherein the alkyl chain is optionally interrupted by one or more ester bonds; and w has a mean value ranging from 30 to 60.
22. The composition of item 20, wherein the polymer-conjugated lipid comprises a polysarcosine-lipid conjugate or a conjugate of polysarcosine and a lipid-like material, wherein the polysarcosine-lipid conjugate or conjugate of polysarcosine and a lipid-like material preferably is a member selected from the group consisting of a polysarcosine-diacylglycerol conjugate, a polysarcosine-dialkyloxypropyl conjugate, a polysarcosine-phospholipid conjugate, a polysarcosine-ceramide conjugate, and a mixture thereof.
22a. The composition of item 20, wherein the polymer-conjugated lipid comprises a poly(oxazoline) (POX)-conjugated lipid; a poly(oxazine) (POZ)-conjugated lipid; a POX/POZ-conjugated lipid; a poly(vinyl pyrrolidone) (PVP)-conjugated lipid; a poly(N-(2-hydroxypropyl)-methacrylamide) (pHPMA)-conjugated lipid; a poly(dehydroalanine) (pDha)-conjugated lipid; or an oligo ethylene glycol (OEG)-conjugated lipid (such as a poly(aminoethoxy ethoxy acetic acid) (pAEEA)-conjugated lipid or a poly(2-methylaminoethoxy ethoxy acetic acid) (pmAEEA)-conjugated lipid).
23. The composition of any one of items 20 to 22a, wherein the polymer-conjugated lipid comprises from about 0.5 mol % to about 5 mol %, preferably from about 1 mol % to about 5 mol %, more preferably from about 1 mol % to about 4.5 mol % of the total lipid present in the composition.
24. The composition of any one of items 20 to 23, wherein the neutral lipid is a phospholipid, preferably selected from the group consisting of phosphatidylcholines, phosphatidylethanolamines, phosphatidylglycerols, phosphatidic acids, phosphatidylserines and sphingomyelins, more preferably selected from the group consisting of distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), and diphytanoyl-phosphatidylethanolamine (DPyPE).
24a. The composition of any one of items 20 to 24, wherein the neutral lipid is DSPC or DOPC.
24b. The composition of any one of items 20 to 24, wherein the neutral lipid is DOPE.
25. The composition of any one of items 20 to 24a and 24b, wherein the neutral lipid comprises from about 5 mol % to about 40 mol %, preferably from about 5 mol % to about 20 mol %, more preferably from about 5 mol % to about 15 mol % of the total lipid present in the composition.
26. The composition of any one of items 20 to 25, wherein the steroid comprises a sterol such as cholesterol.
27. The composition of any one of items 20 to 26, wherein the steroid comprises from about 10 mol % to about 65 mol %, preferably from about 20 mol % to about 60 mol %, more preferably from about 30 mol % to about 50 mol % of the total lipid present in the composition.
28. The composition of any one of items 20 to 27, wherein the particles comprise the cationic or cationically ionizable lipid, a neutral lipid (e.g., a phospholipid), and a steroid, and optionally a polymer-conjugated lipid.
28a. The composition of any one of items 20 to 27, wherein the particles comprise the cationically ionizable lipid, a neutral lipid (e.g., a phospholipid), and a steroid, and optionally a polymer-conjugated lipid.
28b. The composition of any one of items 20 to 27, wherein the particles comprise the cationically ionizable lipid, a neutral lipid (e.g., DOPE), and a steroid (e.g., cholesterol).
28c. The composition of item 28b, wherein the particles comprise LNPs.
28d. The composition of any one of items 20 to 27, wherein the particles comprise the cationic lipid, a neutral lipid (e.g., a phospholipid), and a steroid, and optionally a polymer-conjugated lipid.
28e. The composition of item 28d, wherein the particles comprise LPXs.
29. The composition of item 28, 28a, 28b, 28c, 28d, or 28e, wherein the cationic or cationically ionizable lipid comprises from about 30 mol % to about 50 mol %, such as from about 40 mol % to about 50 mol %, of the total lipid present in the composition; the neutral lipid (e.g., phospholipid) comprises from about 5 mol % to about 15 mol % of the total lipid present in the composition; and the steroid comprises from about 30 mol % to about 50 mol % of the total lipid present in the composition; and, if present, the polymer-conjugated lipid comprises from about 1 mol % to about 4.5 mol % of the total lipid present in the composition.
30. The composition of any one of items 1 to 29, wherein the nucleic acid is RNA, such as mRNA or self-replicating RNA.
31. The composition of item 30, wherein the RNA (i) comprises a modified nucleoside in place of uridine, wherein the modified nucleoside is preferably selected from pseudouridine (ψ), N1-methyl-pseudouridine (mlψ), and 5-methyl-uridine (m5U); (ii) has a coding sequence which is codon-optimized; and/or (iii) has a coding sequence whose G/C content is increased compared to the wild-type coding sequence.
32. The composition of item 30 or 31, wherein the RNA comprises at least one of the following, preferably all of the following: a 5′ cap; a 5′ UTR; a 3′ UTR; and a poly-A sequence.
33. The composition of item 32, wherein the poly-A sequence comprises at least 100 A nucleotides, wherein the poly-A sequence preferably is an interrupted sequence of A nucleotides.
34. The composition of item 32 or 33, wherein the 5′ cap is a cap1 or cap2 structure.
35. The composition of any one of items 30 to 34, wherein the RNA encodes one or more polypeptides, wherein preferably the one or more polypeptides are pharmaceutically active polypeptides and/or comprise an epitope for inducing an immune response against an antigen in a subject.
36. The composition of item 35, wherein the pharmaceutically active polypeptide and/or the antigen or epitope is derived from or is a protein of a pathogen, an immunogenic variant of the protein, or an immunogenic fragment of the protein or the immunogenic variant thereof.
37. The composition of any one of items 1 to 36, wherein the composition is in liquid form, preferably at a temperature of about 0° C. to about 10° C., such as about 2° C. to about 8° C.
38. A method for delivering nucleic acid to cells of a subject, the method comprising administering to a subject a composition of any one of items 1 to 37.
39. A method for delivering a therapeutic peptide or polypeptide to a subject, the method comprising administering to a subject a composition of any one of items 1 to 37, wherein the nucleic acid encodes the therapeutic peptide or polypeptide.
40. A method for treating or preventing a disease or disorder in a subject, the method comprising administering to a subject a composition of any one of items 1 to 37, wherein delivering the nucleic acid to cells of the subject is beneficial in treating or preventing the disease or disorder.
41. A method for treating or preventing a disease or disorder in a subject, the method comprising administering to a subject a composition of any one of items 1 to 37, wherein the nucleic acid encodes a therapeutic peptide or polypeptide and wherein delivering the therapeutic peptide or polypeptide to the subject is beneficial in treating or preventing the disease or disorder.
42. The method of any one of items 38 to 41, wherein the subject is a mammal.
43. The method of item 42, wherein the mammal is a human.
44. The composition of any one of items 1 to 37 for use in therapy.
45. A method of preparing the composition of any one of items 1-37, the method comprising:
   (a) providing the nucleic acid in a solution comprising water and a first buffer system;
   (b) providing an organic solution comprising the cationic or cationically ionizable lipid and, if present, one or more additional lipids;
   (c) mixing the nucleic acid solution provided under (a) with the organic solution provided under (b), thereby preparing an intermediate formulation comprising the particles dispersed in an aqueous phase comprising the first buffer system; and
   (d) filtrating and/or diluting the intermediate formulation prepared under (c) using a final aqueous buffer solution comprising the final buffer system, thereby preparing the composition.

### EXAMPLES

The following Examples are provided to illustrate the invention. It has been challenging to obtain RNA lipid nanoparticle or RNA lipoplex formulations with longer sufficient shelf life at refrigerated temperatures (5±3° C.), to allow ideal storage and transport conditions. Instead, for stabilization, such formulations typically needed to be frozen at much lower temperatures, such as −80° C., which poses substantial challenges on the cold chain. Protocols for stabilizing lipid nanoparticles and lipoplexes are here described, which are compliant to regular technologies in pharmaceutical practice, for storage at refrigerated conditions (5±3° C.). This has been achieved by identification of certain advantageous processing and buffer conditions. In particular, liquid LNP and LPX formulations have been found to exhibit lower rates of RNA degradation when stored in low pH buffer conditions, such as pH 5.0-5.5.

### Methods

### Manufacturing of the RNA Lipid Particles by ‘One-Step’ Process

In some cases, RNA lipid particle formulations were prepared by a so called ‘one-step’ aqueous-ethanol mixing protocol. Briefly, RNA in aqueous buffer conditions of HEPES 10 mM, EDTA 0.1 mM, pH 7.0 is mixed with ethanolic lipid mix comprising of DODMA, DOPE (or another helper lipid, such as DOPC or DSPC, as indicated), cholesterol and C₁₆—PEG₂₀₀₀-ceramide in molar ratio of 40:10:48:2, respectively in the volume ratio of 3 parts of RNA and 1 part of lipid mix. The mixing is achieved using standard syringe pump based set-up with a total flow of 250 ml/ml (187.5 ml/min for aqueous component and 62.5 ml/min for ethanolic component) using Y mixing element. The lipid particle raw colloid is further diluted in 1:1 volume ratio with water and further subjected to tangential flow filtration against water for removal of ethanol, upstream buffers and any process-related impurities. After completion of the diafiltration, the formulation is up-concentrated by factor of 2. Subsequently, the lipid particle composition is diluted with buffer of choice, optionally with 40% w/v sugars (glucose, galactose, sucrose, maltose, trehalose and mannose) or 500 mM sodium chloride, so that the final concentration of RNA in the lipid particle formulations is 0.2 mg/ml. Unless otherwise specified, the RNA-lipid particles are prepared at N/P ratio of 4:1.

### Manufacturing of the RNA Lipid Particles by ‘Two-Step’ Process

In other cases, RNA lipid particle formulations were prepared by a so-called ‘two-step’ aqueous-aqueous protocol. Briefly, RNA in aqueous buffer conditions of HEPES 10 mM, EDTA 0.1 mM, pH 7.0 is mixed with pre-formed lipid particles composed of DODMA, DOPE, cholesterol and C16-PEG2000-ceramide in molar ratio of 40:10:48:2, respectively in aqueous solution of 5 mM acetic acid in the volume ratio of 1:1. The mixing is achieved using standard syringe pump based set-up with a total flow of 250 ml/ml (125 ml/min for each phase) using Y mixing element. The RNA lipid particle stock is then diluted with respective buffer of choice supplemented with 50% w/v sucrose so that the final concentration of RNA in the lipid particle formulations is 0.1 mg/ml. Unless otherwise specified, the RNA lipid particles are prepared at N/P ratio of 4:1.

### Characterization of the RNA Formulations

RNA integrity was determined using capillary gel electrophoresis using an Agilent 5400 Fragment Analyzer System (Agilent, Germany). RNA integrity analysis was done by smear analysis using a Prosize 3.0 software (Agilent, Germany).

Long Term Stability Studies Long term stability studies were conducted by storing the formulations at 5° C.±3° C. for the indicated time period (typically 1 month, 3 months, 6 months, 12 months, or more, as indicated) with periodic assessment of the RNA integrity. At each time point as indicated, the integrity of the RNA within the formulation is assessed using the methods as described above.

### Example 1

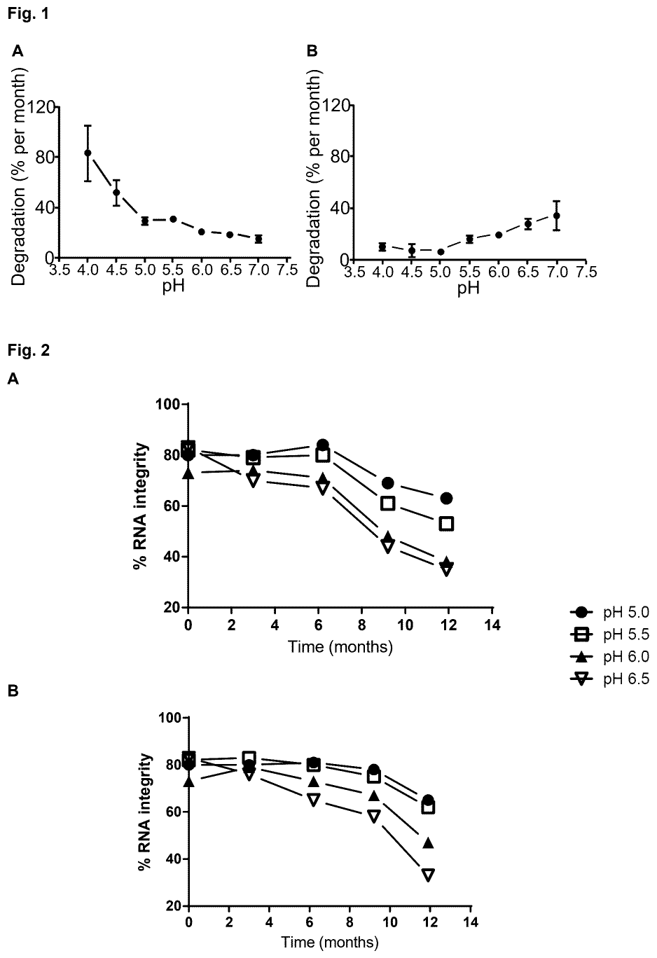

The stability of non-encapsulated saRNA across a range of pH was compared with the stability of saRNA encapsulated in lipid particles across the same pH range to identify the optimum pH for liquid storage stability (FIG. 1). RNA lipid particle formulations were prepared as described above by the ‘two-step’ aqueous-aqueous protocol. Specifically for Example 1, saRNA in aqueous conditions (purified water) was mixed with pre-formed lipid particles composed of DODMA, DOPE, cholesterol and C16-PEG2000-ceramide in molar ratio of 40:10:48:2, respectively in aqueous solution of 5 mM acetic acid in the volume ratio of 1:1. The mixing was performed using standard syringe pump based set-up with a total flow rate of 250 mL/min (125 mL/min for each phase) using Y mixing element. The RNA lipid particle stock is then dialyzed against water for injection and then diluted with the indicated buffer of choice: 20 mM histidine, 1 mM EDTA, at indicated pH. The final concentration of RNA in the lipid particle formulations was approx. 0.1 mg/ml and N/P ratio of 4:1. The non-encapsulated (i.e., naked or free) saRNA formulation was prepared by diluting saRNA in purified water against the buffer of choice, to a final buffer condition of 20 mM histidine, 1 mM EDTA, at indicated pH.

The RNA integrity was measured over time for the non-encapsulated saRNA at a range of different pH values and for the saRNA encapsulated in lipid particles across the same pH range. FIG. 1A shows the degradation of the non-encapsulated saRNA per month over time at the different pH conditions, while FIG. 1B shows the degradation of the saRNA encapsulated in lipid particles. As evident, the stability maxima of the non-encapsulated RNA is close to physiological pH conditions (e.g., approx. pH 7.5). However, in contrast and unexpectedly, the stability maxima of the RNA encapsulated in lipid particles is below pH 5.5. This demonstrates that a pH range of 4.0 to 5.5 is beneficial in terms of RNA stability for RNA lipid particle formulations.

### Example 2

The effect of pH was investigated on the storage stability of liquid formulations of (i) RNA lipid particles prepared by the ‘one-step’ aqueous-ethanol mixing protocol (LP-1, FIG. 2A) and (ii) RNA lipid particles prepared by the ‘two-step’ aqueous-aqueous protocol (LP-2, FIG. 2B). RNA LP-1 or RNA LP-2 were prepared, as described above, in histidine buffer at pH ranging from 5.0 to 6.5. The LP-1 were manufactured with the same process as a single batch. The batch was subjected to tangential flow filtration against water to remove all molecularly dissolved moieties from the bulk phase, such as ions and ethanol. Subsequently fractions of the batch were diluted with histidine buffers at different pH ranging from 5.0 to 6.5, resulting in a buffer concentration of 20 mM histidine and an RNA concentration of 0.2 mg/mL. The LP-2 were manufactured using aqueous-aqueous protocol: aqueous solution of RNA was mixed with aqueous dispersion of pre-formed lipid particles in 5 mM acetic acid at a N/P ratio of 4:1. Subsequently, the bulk RNA lipid particle formulations were further diluted with histidine buffers at different pH ranging from 5.0 to 6.5, resulting in a buffer concentration of 20 mM histidine and a final RNA concentration of 0.1 mg/mL.

The LP-1 or LP-2 formulations were stored for 12 months at 5° C.±3° C., and the RNA integrity was assessed at time points of 3, 6, 9 and 12 months. As shown in FIG. 2A, the RNA degradation within LP-1 is significantly slower when stored in histidine buffer, pH 5.0 for up to 12 months, and the RNA degradation rate increases with increase in pH of the histidine buffer. Similarly, as shown in FIG. 2B, the RNA degradation within LP-2 is also significantly slower when stored in histidine buffer, pH 5.0 for up to 12 months, and the RNA degradation rate increases with increase in pH of the histidine buffer. Thus, for both LP-1 and LP-2, enhanced RNA stability was observed in liquid formulations stored at pH 5.0-5.5, with stability maxima being observed at pH 5.0.

### Example 3

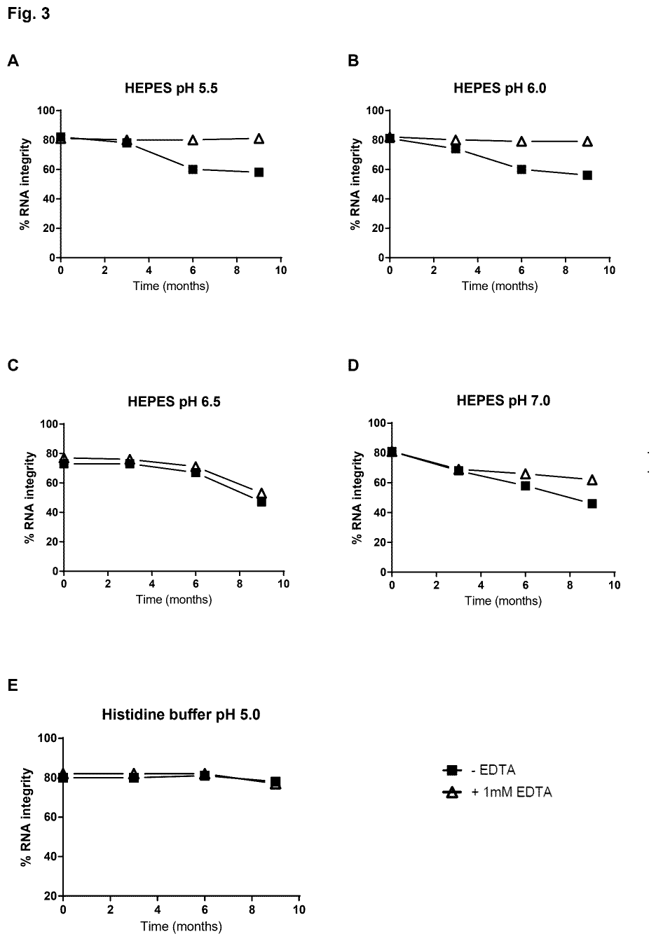

The storage stability of liquid formulations of RNA lipid particles prepared by the ‘two-step’ aqueous-aqueous protocol (LP-2) was also investigated using different buffer systems across a pH range of 5.0 to 7.0. FIG. 3 shows the RNA stability of LP-2 formulations stored at 5° C.±3° C. in 20 mM HEPES buffer (FIG. 3A-D) or 20 mM histidine buffer (FIG. 3E) at different pH values, either in the absence or presence of EDTA at a concentration of 1 mM. The LP-2 formulations were manufactured as described above using aqueous-aqueous protocol: aqueous solution of RNA was mixed with aqueous dispersion of pre-formed lipid particles in 5 mM acetic acid at a N/P ratio of 4:1. Subsequently, the bulk RNA lipid particle formulations were further diluted with different buffers, resulting in a buffer concentration of 20 mM HEPES/histidine (as indicated) and a final RNA concentration of 0.1 mg/mL. The RNA degradation within LP-2 is significantly slower when stored in HEPES buffer supplemented with 1 mM EDTA at pH range from 5.5 to 7.0, in comparison to the HEPES buffer without EDTA at the same pH range (FIG. 3A-D). RNA degradation rates were slowest for LP-2 formulations stored in HEPES buffer at pH 5.5 in the presence of 1 mM EDTA (FIG. 3A). No additional stabilization effect was observed for LP-2 stored in histidine buffer at pH 5.0, upon supplementation with 1 mM EDTA (FIG. 3E). Thus, enhanced RNA stability was observed in liquid LP-2 formulations stored in histidine or HEPES buffer at pH 5.5 or below. Particularly in HEPES buffer, RNA stability was further significantly enhanced by addition of 1 mM EDTA.

### Example 4

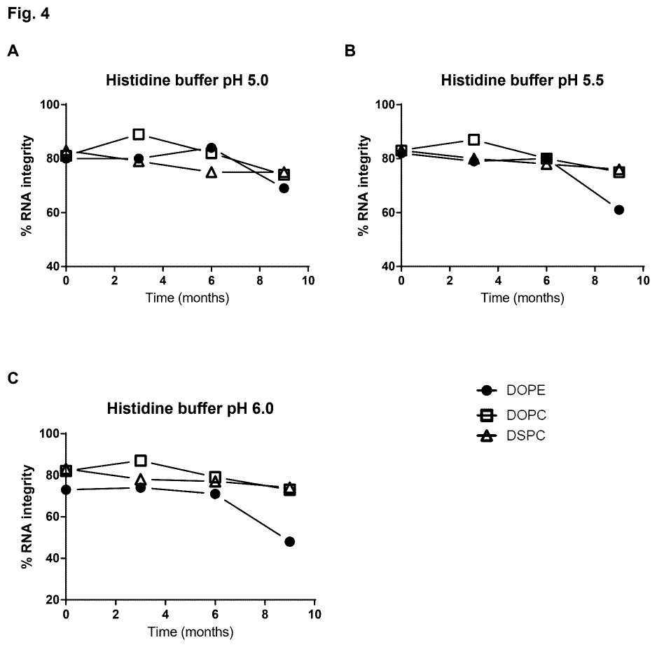

As described above, liquid formulations of RNA lipid particles prepared by the ‘one-step’ aqueous-ethanol mixing protocol (LP-1) were found to be particularly storage stable, showing reduced rates of RNA degradation, when stored at pH 5.5 or below, and especially at pH 5.0, for example in histidine buffer. Additional experiments were performed to confirm whether such low pH conditions were stabilizing, independent of the lipid composition of the LP-1. FIG. 4 shows the results from stability assessments of LP-1 with different lipid compositions stored at 5° C.±3° C. in histidine buffer at pH 5.0 (FIG. 4A), pH 5.5 (FIG. 4B) or pH 6.0 (FIG. 4C). All LP-1 formulations were manufactured with the same process, as described above. Where indicated, the helper lipid DOPE in the lipid composition was replaced with DOPC or DSPC at an identical molar ratio, in order to investigate the effect of helper lipid substitution on the stability of the LP-1. The batches were subjected to tangential flow filtration against water to remove all molecularly dissolved moieties from the bulk phase, such as ions and ethanol. Subsequently fractions of the batch were diluted with different buffers, resulting in a buffer concentration of 20 mM histidine and an RNA concentration of 0.2 mg/mL.

After 9 months of storage, the RNA degradation within LP-1 containing DOPC or DSPC as helper lipid was found to be significantly slower in comparison to those LP-1 containing DOPE as helper lipid. This indicates that LP-1 formulations containing DOPE as helper lipid may be less storage stable in liquid form. The RNA degradation rates within LP-1 containing DOPC or DSPC as helper lipid was comparable across the 12 months of storage, indicating that storage at pH 5.0-5.5, particularly with histidine buffer, is suitable for RNA lipid particles with different lipid compositions.

1. A composition comprising particles dispersed in an aqueous phase, wherein the aqueous phase comprises a buffer system and has a pH of about 4.0-5.5 and wherein the particles contain (i) nucleic acid; and (ii) a cationic or cationically ionizable lipid.

2. The composition of claim 1, wherein the pH of the aqueous phase is below 5.5 and/or above 4.0, such as at least 4.1 and below 5.5, at least 4.2 and below 5.5, at least 4.3 and below 5.5, at least 4.4 and below 5.5, or at least 4.5 and below 5.5.

3. The composition of claim 1 or 2, wherein the concentration of the buffer system in the aqueous phase is between about 1 mM and about 50 mM, preferably between about 2 mM and about 40 mM, more preferably between about 3 mM and about 30 mM, more preferably between about 4 mM and about 25 mM, such as between about 5 mM and about 20 mM.

4. The composition of any one of claims 1 to 3, wherein the buffer system comprises histidine.

5. The composition of any one of claims 1 to 3, wherein the buffer system comprises HEPES.

6. The composition of any one of claims 1 to 5, wherein the aqueous phase further comprises a chelating agent, such as EDTA.

7. The composition of claim 6, wherein the concentration of the chelating agent in the aqueous phase is between about 0.1 mM and about 20 mM, such as between about 0.2 mM and about 15 mM, between about 0.3 mM and about 12 mM, between about 0.4 mM and about 11 mM, or between about 0.5 mM and about 10 mM.

8. The composition of any one of claims 1 to 7, wherein the aqueous phase further comprises one or more tonicity agents, e.g., a salt, such as sodium chloride, and/or a sugar, such as sucrose or glucose.

9. The composition of claim 8, wherein the concentration of the one or more tonicity agents in the aqueous phase is such that the composition is at most isotonic, in particular compared to human blood.

10. The composition of any one of claims 1 to 9, wherein the particles have a size of from about 30 nm to about 500 nm.

11. The composition of any one of claims 1 to 10, wherein the particles are selected from lipid nanoparticles (LNPs), liposomes, lipoplexes (LPXs), and combinations of two or more thereof.

12. The composition of any one of claims 1 to 11, wherein water is the main component in the composition and/or the total amount of solvent(s) other than water contained in the composition is less than about 0.5% (v/v).

13. The composition of any one of claims 1 to 12, wherein the concentration of the nucleic acid in the composition is about 0.1 mg/l to about 500 mg/l, such as about 0.5 mg/l to about 400 mg/l, about 1 mg/l to about 300 mg/l, about 2 mg/l to about 200 mg/l, about 3 mg/l to about 150 mg/l, or about 5 mg/l to about 100 mg/l.

14. The composition of any one of claims 1 to 13, wherein the N/P ratio is at least about 2, such as between about 2 and about 12, between about 4 and about 10, between about 4 and about 8, or between about 5 and about 7, e.g., about 6.

15. The composition of any one of claims 1 to 14, wherein the cationically ionizable lipid comprises a head group which includes at least one nitrogen atom which is capable of being protonated under physiological conditions.

16. The composition of any one of claims 1 to 15, wherein the cationically ionizable lipid has the structure of Formula (X) 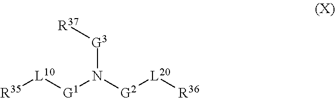
or a pharmaceutically acceptable salt, tautomer, or stereoisomer thereof, wherein:
one of L¹⁰ and L²⁰ is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹⁰ and L²⁰ is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₂₋₁₂ alkenylene;
G³ is C₁₋₂₄ alkylene, C₂₋₂₄ alkenylene, C₃₋₈ cycloalkylene, or C₃₋₈ cycloalkenylene;
R^{a} is H or C₁₋₁₂ alkyl;
R³⁵ and R³⁶ are each independently C₆₋₂₄ alkyl or C₆₋₂₄ alkenyl;
R³⁷ is H, OR⁵⁰, CN, —C(═O)OR⁴⁰, —OC(═O)R⁴⁰ or —NR⁵⁰C(═O)R⁴⁰;
R⁴⁰ is C₁₋₁₂ alkyl;
R⁵⁰ is H or C₁₋₆ alkyl; and
x is 0, 1 or 2.

17. The composition of any one of claims 1 to 15, wherein the cationically ionizable lipid has the structure of Formula (XI): 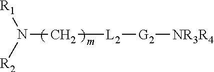
wherein
each of R₁ and R₂ is independently R₅ or —G₁—L₁—R₆, wherein at least one of R₁ and R₂ is —G₁—L₁—R₆;
each of R₃ and R₄ is independently selected from the group consisting of C₁₋₆ alkyl, C₂₋₆ alkenyl, aryl, and C₃₋₁₀ cycloalkyl;
each of R₅ and R₆ is independently a non-cyclic hydrocarbyl group having at least 10 carbon atoms;
each of G₁ and G₂ is independently unsubstituted C₁₋₁₂ alkylene or C₂₋₁₂ alkenylene;
each of L₁ and L₂ is independently selected from the group consisting of —O(C═O)—, —(C═O)O—, —C(═O)—, —O—, —S(O)ₓ—, —S—S—, —C(═O)S—, —SC(═O)—, —NRaC(═O)—, —C(═O)NR^{a}— —NRaC(═O)NR^{a}—, —OC(═O)NR^{a}— and —NRaC(═O)O—;
R^{a} is H or C₁₋₁₂ alkyl;
m is 0, 1, 2, 3, or 4; and
x is 0, 1 or 2.

18. The composition of any one of claims 1 to 15, wherein the cationic or cationically ionizable lipid comprises [(4-hydroxybutyl)azanediyl]di(hexane-6,1-diyl)bis(2-hexyldecanoate) (ALC-315);
1,2-dioleoyloxy-3-dimethylaminopropane (DODMA);
2,2-dilinoleyl-4-dimethylaminoethyl-[1,3]-dioxolane (DLin-KC2-DMA);
heptatriaconta-6,9,28,31-tetraen-19-yl-4-(dimethylamino)butanoate (D-Lin-MC3-DMA);
1,2-dilinoleyloxy-N,N-dimethylaminopropane (DLin-DMA);
di((Z)-non-2-en-1-yl)-9-((4-(dimethylaminobutanoyl)oxy)heptadecanedioate (L319);
bis-(2-butyloctyl) 10-(N-(3-(dimethylamino)propyl)nonanamido)-nonadecanedioate (A9);
(heptadecan-9-yl 8-{(2-hydroxyethyl)[6-oxo-6-(undecyloxy)octyl]amino}-octanoate) (L5);
heptadecan-9-yl 8-{(2-hydroxyethyl)[6-oxo-6-(undecyloxy)hexyl]amino}-octanoate) (SM-102);
O-[N-{(9Z,12Z)-octadeca-9,12-dien-1-yl)}-N-{7-pentadecylcarbonyloxyoctyl}-amino]4-(dimethylamino)butanoate (HY501);
2-(di-((9Z,12Z)-octadeca-9,12-dien-1-yl)amino)ethyl 4-(dimethylamino)butanoate (EA-2);
4-((di-((9Z,12Z)-octadeca-9,12-dien-1-yl)amino)oxy)-N,N-dimethyl-4-oxobutan-4-amine (HYAM-2);
((2-(4-(dimethylamino)butanoyl)oxy)ethyl)azanediylbis(octane 8,1-diyl)bis(2-hexyldecanoate) (EA-405);
(2-(4-(dimethylamino)butanoyl)oxy)azanediylbis(octane 8,1-diyl)bis(2-hexyldecanoate) (HY-405);
palmitoyl-oleoyl-nor-arginine (PONA);
guanidino-di[(heptadecyl)methyl]carboxylic acid (GUADACA);
4-methylpyridinium-di(heptadecyl)methylcarboxylic acid (MPDACA);
1,2-dioleoyl-3 trimethylammonium propane (DOTAP);
1,2-dioleoyl-3-dimethylammomium propane (DODAP); or
1,2-di-O-octadecenyl-3-trimethylammonium propane (DOTMA);
7,7′-((4-hydroxybutyl)azanediyl)bis(N-hexyl-N-octylheptane-1-sulfonamide) (BNT-51);
7,7′-((4-(3,3-dimethylthioureido)butyl)azanediyl)bis(N-hexyl-N-octylheptane-1-sulfonamide) (BNT-52);
di(heptadecan-9-yl) 3,3′-((2-(4-methylpiperazin-1-yl)ethyl)azanediyl)dipropionate (BHD-C2C2-PipZ);
bis(2-octyldodecyl) 3,3′-((2-(1-methylpyrrolidin-2-yl)ethyl)azanediyl)dipropionate (BODD-C2C2-1Me-Pyr)
bis(2-hexyldecyl) 3,3′-((4-(4-methylpiperazin-1-yl)butyl)azanediyl)dipropionate (BHD-C2C4-PipZ) or a mixture of any thereof.

19. The composition of any one of claims 1 to 18, wherein the cationic or cationically ionizable lipid comprises from about 20 mol % to about 80 mol %, preferably from about 25 mol % to about 65 mol %, more preferably from about 30 mol % to about 50 mol %, such as from about 40 mol % to about 50 mol %, of the total lipid present in the composition.

20. The composition of any one of claims 1 to 19, wherein the particles further comprise one or more additional lipids, preferably selected from the group consisting of polymer-conjugated lipids, neutral lipids, steroids, and combinations thereof.

21. The composition of claim 20, wherein the polymer-conjugated lipid comprises a pegylated lipid, wherein the pegylated lipid preferably (i) is selected from the group consisting of DSPE-PEG, DOPE-PEG, DPPE-PEG, and DMPE-PEG; or (ii) has the following structure: 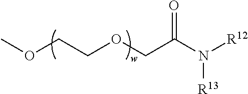
or a pharmaceutically acceptable salt, tautomer or stereoisomer thereof, wherein: R¹² and R¹³ are each independently a straight or branched, saturated or unsaturated alkyl chain containing from 10 to 30 carbon atoms, wherein the alkyl chain is optionally interrupted by one or more ester bonds; and w has a mean value ranging from 30 to 60.

22. The composition of claim 20, wherein the polymer-conjugated lipid comprises a polysarcosine-lipid conjugate or a conjugate of polysarcosine and a lipid-like material, wherein the polysarcosine-lipid conjugate or conjugate of polysarcosine and a lipid-like material preferably is a member selected from the group consisting of a polysarcosine-diacylglycerol conjugate, a polysarcosine-dialkyloxypropyl conjugate, a polysarcosine-phospholipid conjugate, a polysarcosine-ceramide conjugate, and a mixture thereof.

23. The composition of any one of claims 20 to 22, wherein the polymer-conjugated lipid comprises from about 0.5 mol % to about 5 mol %, preferably from about 1 mol % to about 5 mol %, more preferably from about 1 mol % to about 4.5 mol % of the total lipid present in the composition.

24. The composition of any one of claims 20 to 23, wherein the neutral lipid is a phospholipid, preferably selected from the group consisting of phosphatidylcholines, phosphatidylethanolamines, phosphatidylglycerols, phosphatidic acids, phosphatidylserines and sphingomyelins, more preferably selected from the group consisting of distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), and diphytanoyl-phosphatidylethanolamine (DPyPE).

25. The composition of any one of claims 20 to 24, wherein the neutral lipid comprises from about 5 mol % to about 40 mol %, preferably from about 5 mol % to about 20 mol %, more preferably from about 5 mol % to about 15 mol % of the total lipid present in the composition.

26. The composition of any one of claims 20 to 25, wherein the steroid comprises a sterol such as cholesterol.

27. The composition of any one of claims 20 to 26, wherein the steroid comprises from about 10 mol % to about 65 mol %, preferably from about 20 mol % to about 60 mol %, more preferably from about 30 mol % to about 50 mol % of the total lipid present in the composition.

28. The composition of any one of claims 20 to 27, wherein the particles comprise the cationic or cationically ionizable lipid, a neutral lipid (e.g., a phospholipid), and a steroid, and optionally a polymer-conjugated lipid.

29. The composition of claim 28 or 28, wherein the cationic or cationically ionizable lipid comprises from about 30 mol % to about 50 mol %, such as from about 40 mol % to about 50 mol %, of the total lipid present in the composition; the neutral lipid (e.g., phospholipid) comprises from about 5 mol % to about 15 mol % of the total lipid present in the composition; and the steroid comprises from about 30 mol % to about 50 mol % of the total lipid present in the composition; and, if present, the polymer-conjugated lipid comprises from about 1 mol % to about 4.5 mol % of the total lipid present in the composition.

30. The composition of any one of claims 1 to 29, wherein the nucleic acid is RNA, such as mRNA or self-replicating RNA.

31. The composition of claim 30, wherein the RNA (i) comprises a modified nucleoside in place of uridine, wherein the modified nucleoside is preferably selected from pseudouridine (ψ), N1-methyl-pseudouridine (m1ψ), and 5-methyl-uridine (m5U); (ii) has a coding sequence which is codon-optimized; and/or (iii) has a coding sequence whose G/C content is increased compared to the wild-type coding sequence.

32. The composition of claim 30 or 31, wherein the RNA comprises at least one of the following, preferably all of the following: a 5′ cap; a 5′ UTR; a 3′ UTR; and a poly-A sequence.

33. The composition of claim 32, wherein the poly-A sequence comprises at least 100 A nucleotides, wherein the poly-A sequence preferably is an interrupted sequence of A nucleotides.

34. The composition of claim 32 or 33, wherein the 5′ cap is a cap1 or cap2 structure.

35. The composition of any one of claims 30 to 34, wherein the RNA encodes one or more polypeptides, wherein preferably the one or more polypeptides are pharmaceutically active polypeptides and/or comprise an epitope for inducing an immune response against an antigen in a subject.

36. The composition of claim 35, wherein the pharmaceutically active polypeptide and/or the antigen or epitope is derived from or is a protein of a pathogen, an immunogenic variant of the protein, or an immunogenic fragment of the protein or the immunogenic variant thereof.

37. The composition of any one of claims 1 to 36, wherein the composition is in liquid form, preferably at a temperature of about 0° C. to about 10° C., such as about 2° C. to about 8° C.

38. A method for delivering nucleic acid to cells of a subject, the method comprising administering to a subject a composition of any one of claims 1 to 37.

39. A method for delivering a therapeutic peptide or polypeptide to a subject, the method comprising administering to a subject a composition of any one of claims 1 to 37, wherein the nucleic acid encodes the therapeutic peptide or polypeptide.

40. A method for treating or preventing a disease or disorder in a subject, the method comprising administering to a subject a composition of any one of claims 1 to 37, wherein delivering the nucleic acid to cells of the subject is beneficial in treating or preventing the disease or disorder.

41. A method for treating or preventing a disease or disorder in a subject, the method comprising administering to a subject a composition of any one of claims 1 to 37, wherein the nucleic acid encodes a therapeutic peptide or polypeptide and wherein delivering the therapeutic peptide or polypeptide to the subject is beneficial in treating or preventing the disease or disorder.

42. The method of any one of claims 38 to 41, wherein the subject is a mammal.

43. The method of claim 42, wherein the mammal is a human.

44. The composition of any one of claims 1 to 37 for use in therapy.

45. A method of preparing the composition of any one of claims 1-37, the method comprising:
(a) providing the nucleic acid in a solution comprising water and a first buffer system;
(b) providing an organic solution comprising the cationic or cationically ionizable lipid and, if present, one or more additional lipids;
(c) mixing the nucleic acid solution provided under (a) with the organic solution provided under (b), thereby preparing an intermediate formulation comprising the particles dispersed in an aqueous phase comprising the first buffer system; and
(d) filtrating and/or diluting the intermediate formulation prepared under (c) using a final aqueous buffer solution comprising the final buffer system,
thereby preparing the composition.
